(19) United States
(12) Patent Application Publication
(10) Pub. No.: US 2026/0097139 A1
(43) Pub. Date: Apr. 9, 2026

(54) GONADOTROPIN-RELEASING HORMONE RECEPTOR (GnRHR) TARGETED THERAPEUTICS AND USES THEREOF

(71) Applicant: Radionetics Oncology, Inc., San Diego, CA (US)

(72) Inventors: Jian ZHAO, San Diego, CA (US); Mi CHEN, San Diego, CA (US); Yunfei ZHU, San Diego, CA (US)

(21) Appl. No.: 19/136,120

(22) PCT Filed: Dec. 11, 2023
(86) PCT No.: PCT/US2023/083397

§ 371 Date: Jun. 5, 2025

Related U.S. Application Data
(60) Provisional application No. 63/387,203, filed on Dec. 13, 2022.

(51) Int. Cl. A61K 51/04

(52) U.S. Cl. CPC A61K 51/0497; A61K 51/0459; A61K 2121/00; A61K 2123/00

(57) ABSTRACT

Described herein are radiotherapeutics that target tumor cells expressing the gonadotropin-releasing hormone receptor (GnRHR) and their use in the treatment and/or diagnosis of cancer.

### CROSS-REFERENCE TO RELATED APPLICATIONS

This application claims the benefit of U.S. Provisional Patent Application No. 63/387,203, filed Dec. 13, 2022, which is incorporated herein by reference in its entirety.

### FIELD OF THE INVENTION

Described herein are radiotherapeutics that target tumor cells expressing the gonadotropin-releasing hormone receptor (GnRHR) and methods of using such radiotherapeutics as cancer therapeutics, diagnostics, or both.

### BACKGROUND OF THE INVENTION

Neoplasms are abnormal growth of cells and cause enormous medical burdens, including morbidity and mortality, in humans. Neoplasms include benign or noncancerous neoplasms which do not display malignant features and are generally unlikely to become dangerous (e.g., adenomas). Malignant neoplasms display features such as genetic mutations, loss of normal function, rapid division, and ability metastasize (invade) to other tissues, and neoplasms of uncertain or unknown behavior. Malignant neoplasms (i.e., cancerous solid tumors) are the leading cause of death in industrialized countries. Noncancerous neoplasms including benign adenomas can also cause significant morbidity and mortality. Although standard treatments can achieve significant effects in tumor growth inhibition and even tumor elimination, the applied drugs exhibit only minor selectivity for the malignant tissue over healthy tissue and their severe side effects limit their efficacy and use. Specific targeting of neoplastic cells without affecting healthy tissue is a major desire for effective solid tumor therapy.

As one of 3 main classes of cell surface receptors, G protein-coupled receptors (GPCRs) are frequently overexpressed in tumor cells and are considered promising targets for selective tumor therapy. Specifically, GnRHR is overexpressed multiple cancer types, including, but not limited to, ovarian cancers, breast carcinomas, endometrial cancers, and prostate cancers. Targeted delivery of radionuclides to tumors with small molecule GnRHR-targeting ligands offers a novel approach to treat and diagnose various cancers, including, but not limited to ovarian cancer, breast carcinomas, endometrial cancer and prostate cancer.

### SUMMARY OF THE INVENTION

Described herein are radiopharmaceuticals for use in the diagnosis and/or treatment of tumors. The present disclosure provides an alternative and improved method for the treatment of tumors by targeting tumors that overexpress the gonadotropin-releasing hormone receptor (GnRHR). In some embodiments, the radiopharmaceuticals disclosed herein are useful in the treatment of tumors that overexpress GnRHR. In some other embodiments, the radiopharmaceuticals disclosed herein are useful in the identification of tissues or organs in a subject comprising tumors overexpressing GnRHR. The radiopharmaceuticals disclosed herein are also useful in vivo imaging of a subject for the presence of and distribution of tumors that overexpress GnRHR in the subject.

In one aspect, described herein is a compound of Formula (I), or a pharmaceutically acceptable salt thereof,

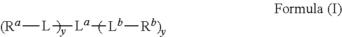

wherein:
L^{a} is absent or a branched linker;
each L is independently selected from absent or a linker;
each R^{a} is independently selected from a chelating moiety or a radionuclide complex thereof;
each R^{b} is independently a small molecule modulator of the gonadotropin-releasing hormone receptor (GnRHR); and
each y is independently 1, 2 or 3.

In some embodiments, each y is 1.

In some embodiments, R^{b} is a small molecule antagonist of GnRHR. In some embodiments, R^{b} comprises a furan pyrimidine, a pyridine-chloro-benzamide, a pyridine-chloro-benzenesulfamide, a uracil, a thieno[2,3-d]pyrimidine-2,4(1H,3H)-dione, or a thieno[3,4-d]pyrimidine-2,4(1H,3H)-dione. In some embodiments, R^{b} comprises a furan pyrimidine. In some embodiments, R^{b} comprises a pyridine-chloro-benzamide or a pyridine-chloro-benzenesulfamide.

In another aspect, described herein is a compound of Formula (II), or a pharmaceutically acceptable salt thereof:

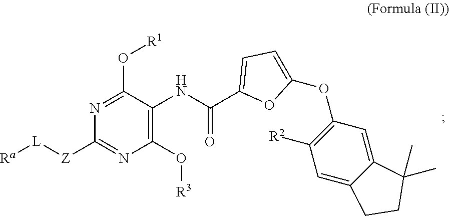

wherein:
R¹ is H, substituted or unsubstituted —C₁-C₄ alkyl, or substituted or unsubstituted —C₁-C₄ haloalkyl;
R² is H, halo, substituted or unsubstituted —C₁-C₆ alkyl, or substituted or unsubstituted —C₁-C₄ haloalkyl;
R³ is H, substituted or unsubstituted —C₁-C₄ alkyl, or substituted or unsubstituted —C₁-C₄ haloalkyl;
Z is absent, —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—;
R⁴ is H or unsubstituted C-₁-C₄ alkyl;
L is a linker; and
R^{a} is a chelating moiety or a radionuclide complex thereof.

In another aspect, described herein is a compound of Formula (IIIa) or (IIIb), or a pharmaceutically acceptable salt thereof:

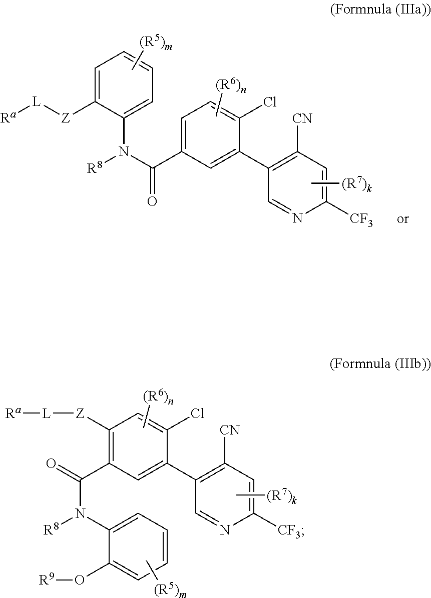

wherein:
each R⁵ is independently selected from F, Cl, Br, I, —CN, substituted or unsubstituted —C₁-C₆ alkyl, or substituted or unsubstituted —C₁-C₆ alkoxy;
each R⁶ is independently selected from F, Cl, Br, I, —CN, substituted or unsubstituted —C₁-C₆ alkyl, or substituted or unsubstituted —C₁-C₆ alkoxy;
each R⁷ is independently selected from F, Cl, Br, I, —CN, substituted or unsubstituted —C₁-C₆ alkyl, or substituted or unsubstituted —C₁-C₆ alkoxy;
R⁸ is H or substituted or unsubstituted —C₁-C₄ alkyl;
R⁹ is H or substituted or unsubstituted —C₁-C₄ alkyl;
Z is absent, —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—;
R⁴ is H or unsubstituted —C₁-C₄ alkyl;
L is a linker; and
R^{a} is a chelating moiety or a radionuclide complex thereof;
m is 0, 1, 2, 3, or 4;
n is 0, 1, 2, or 3; and
k is 0, 1, or 2.

In another aspect, described herein is a compound of Formula (IIIc), or a pharmaceutically acceptable salt thereof:

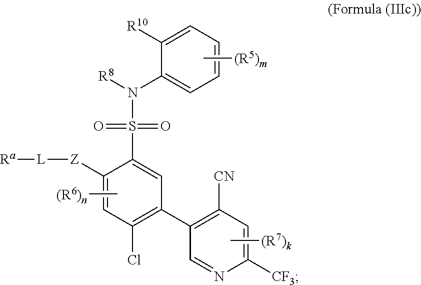

wherein:
each R⁵ is independently selected from F, Cl, Br, I, —CN, substituted or unsubstituted —C₁-C₆ alkyl, or substituted or unsubstituted —C₁-C₆ alkoxy;
each R⁶ is independently selected from F, Cl, Br, I, —CN, substituted or unsubstituted —C₁-C₆ alkyl, or substituted or unsubstituted —C₁-C₆ alkoxy;
each R⁷ is independently selected from F, Cl, Br, I, —CN, substituted or unsubstituted —C₁-C₆ alkyl, or substituted or unsubstituted —C₁-C₆ alkoxy;
R⁸ is H or —C₁-C₄ alkyl;
R¹⁰ is H, —C₁-C₄ alkyl, or —O—C₁-C₄ alkyl; or
R⁸ and R¹⁰ are taken together with the intervening atoms connecting R⁸ to R¹⁰, to form a substituted or unsubstituted 4- to 7-membered heterocyclic ring;
Z is absent, —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—;
R⁴ is H or unsubstituted —C₁-C₄ alkyl
L is a linker;
R^{a} is a chelating moiety or a radionuclide complex thereof;
m is 0, 1, 2, 3, or 4;
n is 0, 1, 2, or 3; and
k is 0, 1, or 2.

In some embodiments, the compound has the structure of Formula (IIId), or a pharmaceutically acceptable salt thereof:

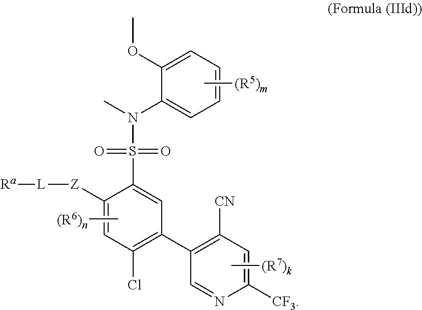

In some embodiments, the compound has the structure of Formula (IIIe), or a pharmaceutically acceptable salt thereof:

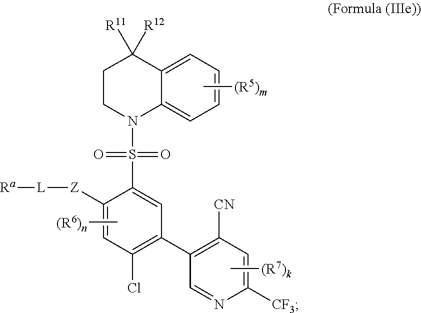

wherein R¹¹ and R¹² are each independently selected from H, F, or —C₁-C₄ alkyl; or
R¹¹ and R¹² can come together to form a substituted or unsubstituted —C₃-C₆ cycloalkyl ring.

In some embodiments, the chelating moiety of R^{a} is independently selected from the group consisting of: 1,4,7,10-tetraazacyclododecane-1,4,7,10-tetraacetic acid (DOTA); 2,2′,2″-(10-(2-amino-2-oxoethyl)-1,4,7,10-tetraazacyclododecane-1,4,7-triyl)triacetic acid (PSC); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

In some embodiments, the chelating moiety of R^{a} is independently selected from the group consisting of:

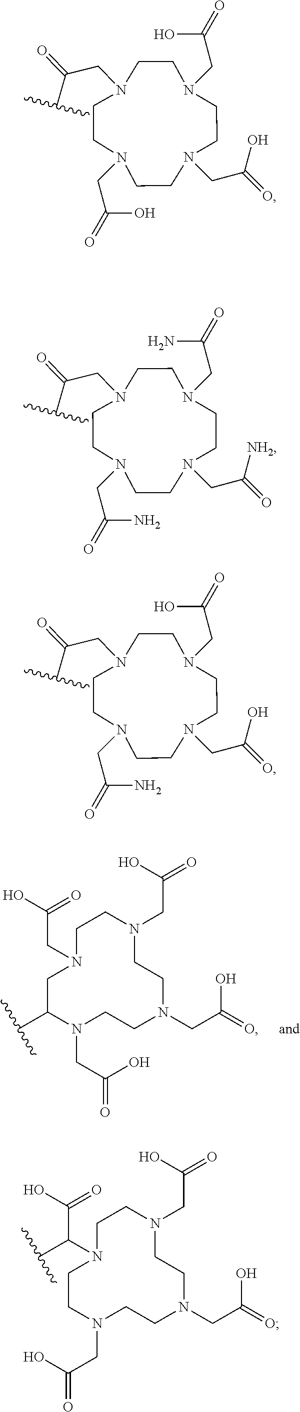 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, each L is independently selected from: -L²-, -L³-, -L⁴-, -L⁵-, -L⁶-, -L⁷-, -L²-L³-, -L²-L³-R¹⁹, -L²-L⁴-, -L²-L⁷-, -L⁴-L⁶-, -L⁴-L⁷-, -L⁶-L⁷-, -L²-L⁴-L⁷-, -L²-L⁵-L⁷-, -L²-L⁶-L⁷-, -L³-L⁴-L⁷-, -L⁴-L⁵-L⁷-, or -L²-L³-L⁴-L⁵-L⁶-L⁷-; L² is absent, substituted or unsubstituted —C₁-C₂₀ alkylene, substituted or unsubstituted —C₁-C₂₀ alkylene-NR¹³—, substituted or unsubstituted —C₁-C₂₀ alkylene-C(═O)—, substituted or unsubstituted —C₁-C₂₀ alkylene-C(═O)NR¹³—, substituted or unsubstituted —C₁-C₂₀ alkylene-NR¹³C(═O)—, substituted or unsubstituted —C₁-C₂₀ alkylene-C(═O)NR¹³CH₂NR¹³—, substituted or unsubstituted —C₁-C₂₀ alkylene-NR¹³C(═O)CH₂NR¹³—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NR¹³—(CH₂CH₂O)_{w}—, —(CH₂CH₂O)_{w}—CH₂CH₂NR¹³—, —CH₂CH₂NHC(═O)—(CH₂CH₂O)_{w}, —(CH₂CH₂O)_{w}—CH₂CH₂NR¹³C(═O)—, —CH₂CH₂C(═O)NR¹³—(CH₂CH₂O)_{w}—, —CH₂CH₂NR¹³C(═O)CH₂—(OCH₂CH₂)_{w} or —(CH₂CH₂O)_{w}—CH₂CH₂C(═O)NR¹³—; R¹³ is H or unsubstituted —C₁-C₄ alkyl; w is 1, 2, 3, 4, 5, 6, 7 or 8; z is 1, 2, 3, 4, 5, 6, 7, 8, 9, or 10; L³ is absent, a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl; L⁴ is absent, substituted or unsubstituted 2 to 10 membered heteroalkylene, —CH₂—(OCH₂CH₂)ᵥ—, —(CH₂CH₂O)ᵥ—CH₂CH₂—, —(CH₂CH₂O)ᵥCH₂CH₂NR¹²C(═O)(CH₂CH₂O)ᵥCH₂CH₂—, —(CH₂CH₂O)ᵥCH₂CH₂C(═O)NR¹⁴(CH₂CH₂O)ᵥCH₂CH₂—, —C(═O)CH₂CH₂, —CH₂CH₂C(═O)—, —CH₂CH₂NR¹⁴CH₂CH₂, —CH₂CH₂NHC(═O)—CH—CH₂CH₂C(═O)NHR¹⁴, or —C₁-C₆ alkylene that is optionally substituted with 1 or 2 groups independently selected from —OR¹⁵, —NR¹⁵₂, —CO₂R^{5′}, —O(CH₂CH₂O)ₛ—CH₃, —NR¹⁵(CH₂CH₂O)ₛ—CH₃, —NR¹⁵C(═O)(CH₂CH₂O)ₛ—CH₃, or —CH₂OCH₂CH₂CO₂R¹⁵; R¹⁴ is H, —C₁-C₆ alkyl, or a sugar alcohol or derivative thereof, each R¹⁵ is independently selected from H or unsubstituted —C₁-C₄ alkyl; v is 1, 2, 3, 4, 5, 6, 7 or 8; s is 1, 2, 3, 4, 5, or 6; L⁵ is absent, —O—, —S—, —S(═O)—, —S(═O)₂, —NR¹⁶—, —CH(═NH)—, —CH(═N—NH)—, —CCH₃(═NH)—, —CCH₃(═N—NH)—, —C(═O)NR¹⁶—, —NR¹⁶C(═O), —NR¹⁶C(═O)O—, —NR¹⁶C(═O)NR¹⁶—, —OC(═O)NR¹⁶—, or —NHC(═O)—C₁-C₆ alkylene that is optionally substituted with 1 or 2 groups independently selected from —OR¹⁵, —NR¹⁵₂ or —CO₂R¹⁵; each R¹⁶ is independently selected from H or unsubstituted —C₁-C₄ alkyl; L⁶ is absent or -L⁸-L⁹-L¹⁰-; L⁸ is absent, —(CH₂)ᵣ—, —NR¹⁷—, —NR¹⁷—(CH₂)ᵣ—, —(CH₂)ᵣ—C(═O)—, —C(═O)—(CH₂)ᵣ—, —(CH₂)ᵣ—NR¹⁷—, —(CH₂)ᵣ—NR¹⁷C(═O)—, —(CH₂)ᵣ—C(═O)NR¹⁷—, —CH(NHR¹⁷)—(CH₂)ᵣ—C(═O)—, —NR¹⁷C(═O)—(CH₂)ᵣ—, and —C(═O)NR¹⁷—(CH₂)ᵣ—; r is 0, 1, 2, or 3; L⁹ is substituted or unsubstituted cycloalkylene, substituted or unsubstituted heterocycloalkylene, substituted or unsubstituted arylene, or substituted or unsubstituted heteroarylene; L¹⁰ is absent, —(CH₂)_{q}—, —NR¹⁸—, —NR¹⁸—(CH₂)_{q}—, —(CH₂)_{q}—C(═O)—, —C(═O)—(CH₂)_{q}—, —(CH₂)_{q}—NR¹⁸—, —(CH₂)_{q}—NR¹⁸C(═O)—, —(CH₂)_{q}—C(═O)NR¹⁸—, —CH(NHR¹⁸)—(CH₂)_{q}—C(═O)—, —NR¹⁸C(═O)—(CH₂)_{q}—, —C(═O)NR¹⁸—(CH₂)_{q}—, and —NR¹⁸—(CH₂)_{q}—NR¹⁸; q is 0, 1, 2, or 3; R¹⁷ and R¹⁸ are each independently selected from H, —C₁-C₆ alkyl, —C₁-C₆ alkyl-CO₂H, —(CH₂CH₂O)ₚ—CH₃, —C(═O)—(CH₂CH₂O)ₚ—CH₃, or —(CH₂CH₂O)ₚ—CH₂CH₂CO₂H; p is 1, 2, 3, 4, 5, or 6; L⁷ is absent, —NH—, —N(CH₃)—, —O—NH—, or substituted or unsubstituted N-heterocycloalkylene, or —O—NH=(substituted or unsubstituted N-heterocycloalkylene); R¹⁹ is —C(═O)(CH₂)ᵢ-substituted or unsubstituted phenyl; and i is 1, 2, 3, 4, 5, or 6.

In some embodiments, each L is independently selected from: -L²-, -L³-, -L⁴-, -L⁵-, -L⁶-, -L⁷-, -L²-L³-, -L²-L³-R¹⁹, -L²-L⁴-, -L²-L⁷-, -L⁴-L⁶-, -L⁴-L⁷-, -L⁶-L⁷-, -L²-L⁴-L⁷-, -L²-L⁵-L⁷-, -L²-L⁶-L⁷-, -L³-L⁴-L⁷-, -L⁴-L⁵-L⁷-, or -L²-L³-L⁴-L⁵-L⁶-L⁷-; L² is substituted or unsubstituted —C₁-C₂₀ alkylene-C(═O)NR¹³—, substituted or unsubstituted —C₁-C₂₀ alkylene-NR¹³C(═O)—, or —(CH₂CH₂O)_{w}—CH₂CH₂—; R¹³ is H or unsubstituted —C₁-C₄ alkyl; w is 1, 2, 3, 4, 5, 6, 7 or 8; L³ is absent or a natural or unnatural amino acid or peptide that is formed from two or more independently selected natural and unnatural amino acids; L⁴ is absent, —(CH₂CH₂O)ᵥ—CH₂CH₂—, CH₂CH₂NR¹⁴CH₂CH₂, or —C₁-C₆ alkylene that is optionally substituted with 1 or 2 groups independently selected from —OR¹⁵, —NR¹⁵₂ or —CO₂R¹⁵; R¹⁴ is H or —C₁-C₆ alkyl; each R¹⁵ is independently selected from H or unsubstituted —C₁-C₄ alkyl; v is 1, 2, 3, 4, 5, 6, 7 or 8; L⁵ is absent or —NHC(═O)—C₁-C₆ alkylene that is optionally substituted with 1 or 2 groups independently selected from —OR¹⁵, —NR¹⁵₂ or —CO₂R¹⁵; L⁶ is absent; L⁷ is —NH—; R¹⁹ is —C(═O)(CH₂)ᵢ-substituted or unsubstituted phenyl; and i is 1, 2, 3, 4, 5, or 6.

In some embodiments, the radionuclide of the radionuclide complex is a lanthanide or an actinide. In some embodiments, the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium. In some embodiments, the radionuclide of the radionuclide complex is a diagnostic or therapeutic radionuclide. In some embodiments, the radionuclide of the radionuclide complex is an Auger electron-emitting radionuclide, α-emitting radionuclide, 3-emitting radionuclide, or γ-emitting radionuclide. In some embodiments, the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 70-gallium (⁷⁰Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

Also described herein is a pharmaceutical composition comprising a compound described herein (e.g., a compound of Formula (I), (II), (IIIa), (IIIb), (IIIc), (IIId), or (IIIe)), or a pharmaceutically acceptable salt thereof, and at least one pharmaceutically acceptable excipient. In some embodiments, the pharmaceutical composition is formulated for administration to a mammal by intravenous administration or subcutaneous administration. In some embodiments, the pharmaceutical composition is formulated for administration to a mammal by intravenous administration.

In another aspect, described herein is a method for the treatment of cancer comprising administering to a mammal with cancer an effective amount of a compound described herein (e.g., a compound of Formula (I), (II), (IIIa), (IIIb), (IIIc), (IIId), or (IIIe)), or a pharmaceutically acceptable salt thereof, or an effective amount of pharmaceutical composition comprising a compound described herein (e.g., a compound of Formula (I), (II), (IIIa), (IIIb), (IIIc), (IIId), or (IIIe)), or a pharmaceutically acceptable salt thereof. In some embodiments, the cancer comprises tumors and the tumor overexpress the gonadotropin-releasing hormone receptor (GnRHR). In some embodiments, the cancer is ovarian cancer, breast cancer, endometrial cancer, or prostate cancer. In some embodiments, the cancer is ovarian cancer. In some embodiments, the cancer is breast cancer. In some embodiments, the cancer is endometrial cancer. In some embodiments, the cancer is prostate cancer.

In another aspect, described herein is a method for treating tumors in a mammal with a radionuclide comprising administering to the mammal a compound described herein (e.g., a compound of Formula (I), (II), (IIIa), (IIIb), (IIIc), (IIId), or (IIIe)), or a pharmaceutically acceptable salt thereof, or a pharmaceutical composition comprising a compound described herein (e.g., a compound of Formula (I), (II), (IIIa), (IIIb), (IIIc), (IIId), or (IIIe)), or a pharmaceutically acceptable salt thereof. In some embodiments, the mammal has been diagnosed with ovarian cancer. In some embodiments, the mammal has been diagnosed with breast cancer. In some embodiments, the mammal has been diagnosed with endometrial cancer. In some embodiments, the mammal has been diagnosed with prostate cancer.

In another aspect, described herein is a method of targeting delivery of a radionuclide to tumors in a mammal comprising administering to a mammal with tumors a compound described herein (e.g., a compound of Formula (I), (II), (IIIa), (IIIb), (IIIc), (IIId), or (IIIe)), or a pharmaceutically acceptable salt thereof, or a pharmaceutical composition comprising a compound described herein (e.g., a compound of Formula (I), (II), (IIIa), (IIIb), (IIIc), (IIId), or (IIIe)), or a pharmaceutically acceptable salt thereof; wherein the tumors overexpress the gonadotropin-releasing hormone receptor (GnRHR).

In another aspect, described herein is a method for identifying tissues or organs in a mammal with tumors expressing the gonadotropin-releasing hormone receptor (GnRHR) comprising administering to the mammal a compound described herein (e.g., a compound of Formula (I), (II), (IIIa), (IIIb), (IIIc), (IIId), or (IIIe)), or a pharmaceutically acceptable salt thereof, or a pharmaceutical composition comprising a compound described herein (e.g., a compound of Formula (I), (II), (IIIa), (IIIb), (IIIc), (IIId), or (IIIe)), or a pharmaceutically acceptable salt thereof, and performing positron emission tomography (PET) analysis, single-photon emission computerized tomography (SPECT), or magnetic resonance imaging (MRI); wherein R^{a} is a chelating moiety-diagnostic radionuclide complex.

In yet another aspect, described herein is a method for the in vivo imaging of tissues or organs in a mammal with tumors expressing the gonadotropin-releasing hormone receptor (GnRHR) comprising administering to the mammal a compound described herein (e.g., a compound of Formula (I), (II), (IIIa), (IIIb), (IIIc), (IIId), or (IIIe)), or a pharmaceutically acceptable salt thereof, or a pharmaceutical composition comprising a compound described herein (e.g., a compound of Formula (I), (II), (IIIa), (IIIb), (IIIc), (IIId), or (IIIe)), or a pharmaceutically acceptable salt thereof; and performing positron emission tomography (PET) analysis, single-photon emission computerized tomography (SPECT), or magnetic resonance imaging (MRI); wherein R^{a} is a chelating moiety-diagnostic radionuclide complex.

In any of the embodiments disclosed herein, the mammal is a human.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Cancer, a disease in which some cells undergo a genetic change in the control of their growth and replication that results in uncontrolled growth and spreading, is one of the leading causes of death worldwide. General types of cancers include solid tumors (cancers that typically originate in organs), carcinomas (cancers that originate in skin or tissues that line organs), sarcomas (cancers of connective tissues such as bones), leukemias (cancers of bone marrow), and lymphomas and myelomas (cancers of the immune system). Neoplasms are abnormal growth of cells that result in solid tumors which may be benign (i.e. do not display malignant features and are generally unlikely to become dangerous such as adenomas), malignant (i.e. display features such as genetic mutations, loss of normal function, rapid division, and ability metastasize (invade) to other tissues), and of uncertain or unknown behavior. State-of-the-art treatment of neoplasms is accomplished by a combination of surgical procedures, chemotherapy, and radiation therapy. Surgical procedures can be curative under some conditions, but often requires multiple interventions as well as combination with radiation and chemotherapy. Chemotherapy proves to be a potent weapon in the fight against cancer in many cases. Chemotherapy is typically performed by systemic administration of potent cytotoxic drugs, but these compounds often lack tumor selectivity and therefore also kill healthy cells in the body. The resulting non-specific toxicity is the cause of severe side effects of chemotherapy which does not target the cancerous cells specifically over other cells. Radiotherapy is the use of high-energy radiation to kill cells. The source of radiation may be external-beam radiation (applied using an external source), internal radiation (placement of a radioactive material near the target cells), or radiotherapy from the systemic administration of a radioactive material. Like chemotherapy, many radiation therapy options also lack tumor cell identification properties needed to achieve the ultimate goal of targeted tumor therapy with drug molecules or radionuclides.

Described herein are radiopharmaceuticals that selectively deliver radionuclides to malignant cells that overexpress GnRHR for use in cancer detection, image guided cancer surgery, and selective tumor killing.

GPCRs are generally poorly antigenic making them difficult targets for antibody-based strategies. The large size of antibodies can impact homogenous uptake and they may be unable to penetrate deep in solid tumors. Additionally, antibodies may present difficulties during production, including inter-batch variability.

Peptides are intrinsically sensitive to proteolytic enzymes and peptidases present in most tissues may rapidly degrade the peptides into multiple fragments which no longer have significant affinity to the intended receptors. In addition, peptides may cause unwanted immunogenic responses complicating later stages of development by masking the therapeutic effect and impacting the safety assessment.

When peptide ligands are linked to radionuclide payloads, the resulting conjugates often degrade apart rapidly in blood plasma and produce cytotoxic or radioactive peptide fragments which may nonspecifically bind to both tumor and normal tissue. Such premature breakdown of peptide radionuclide conjugates reduce the amount of radionuclide payloads distributed to targeted tumors, lowering treatment efficacy, and possibly increasing toxicity. In addition, peptides are most likely exclusively excreted via kidney, which may limit their applications. Marked kidney uptake of some peptide-based therapeutics has limited their routine use.

High affinity, small molecule ligands that bind GPCRs have been described and are cell permeable and can access populations of receptors in endoplasmic reticulum and endosomes. Owing to the low molecular weight of small molecules, vascular permeability and tumor penetration should be improved compared to high molecular weight conjugates based on peptides and antibodies. The affinity of small molecule ligands in many cases surpasses that of FDA approved antibodies by orders of magnitude.

### The Gonadotropin-Releasing Hormone Receptor (GnRHR)

Gonadotropin-releasing hormone (GnRH) or luteinizing hormone releasing hormone (LHRH) is a central regulator of the reproductive system in humans. It was first isolated from mammalian hypothalamic tissue as a linear decapeptide (pGlu-His-Trp-Ser-Tyr-Gly-Leu-Arg-Pro-Gly-NH2). The GnRH peptide is produced in hypothalamic neurons and released in a pulsed fashion into the portal blood-stream supplying the pituitary gland to stimulate the synthesis and secretion of gonadotropic hormones, luteinizing hormone (LH) and follicle-stimulating hormone (FSH).

Gonadotropin-releasing hormone (GnRH) receptor belong to the class A of G-protein coupled receptors (GPCRs). These receptors are involved in the control of a diverse set of behavioral processes including appetite, circadian rhythm, and anxiety. Among the GPCR family members, the gonadotropin-releasing hormone (GnRH) receptor is a well-established target in the clinical practice of cancer treatment. GnRH receptors are expressed not only in the pituitary and in normal peripheral tissues, but also in various tumor cells like melanoma, prostate and endometrial carcinomas, leiomyomas, leiomyosarcomas, breast cancer, choriocarcinoma, epithelial and stromal tumors of the ovary. Several human tumor types, including ovarian, prostate, breast, endometrial, and lung cancer, overexpress or even uniquely express this receptor with respect to the surrounding nonmalignant cells. Therefore, GnRH ligands carrying radionuclide cargoes offer a new modality in the imaging and treatment of cancers.

### Breast Cancer

Breast cancer is a type of cancer that starts in the breast. It can start in one or both breasts, in various parts of the breast. There are many types of breast cancer, and a breast cancer's type is determined by the specific cells in the breast that become cancer.

### Breast Cancer Types

Most breast cancers are carcinomas, which are tumors that start in the epithelial cells that line organs and tissues throughout the body. When carcinomas form in the breast, they are usually a more specific type called adenocarcinoma, which starts in cells in the ducts (the milk ducts) or the lobules (glands in the breast that make milk).

The type of breast cancer can also refer to whether the cancer has spread or not. In situ breast cancer (ductal carcinoma in situ or DCIS) is a pre-cancer that starts in a milk duct and has not grown into the rest of the breast tissue. The term invasive (or infiltrating) breast cancer is used to describe any type of breast cancer that has spread (invaded) into the surrounding breast tissue.

### Breast Cancer Staging

The staging system most often used for breast cancer is the American Joint Committee on Cancer (AJCC) TNM system. The most recent AJCC system, effective January 2018, has both clinical and pathologic staging systems for breast cancer:

The pathologic stage (also called the surgical stage) is determined by examining tissue removed during an operation.

Sometimes, if surgery is not possible right away or at all, the cancer will be given a clinical stage instead. This is based on the results of a physical exam, biopsy, and imaging tests. The clinical stage is used to help plan treatment. Sometimes, though, the cancer has spread further than the clinical stage estimates, and may not predict the patient's outlook as accurately as a pathologic stage.

In both staging systems, 7 key pieces of information are used:
i. The extent (size) of the tumor (T);
ii. The spread to nearby lymph nodes (N);
iii. The spread (metastasis) to distant sites (M);
iv. Estrogen Receptor (ER) status;
v. Progesterone Receptor (PR) status;
vi. HER2 status; and
vii. Grade of the cancer (G).

In addition, Oncotype Dx® Recurrence Score results may also be considered in the stage in certain situations. Once all of these factors have been determined, this information is combined in a process called stage grouping to assign an overall stage.

### Breast Cancer Treatment

Tumors can form in the breasts. The types of treatment used to treat breast tumors include: surgery, radiation therapy, chemotherapy, hormone therapy, targeted drug therapy and immunotherapy.

There are two main types of surgery to remove breast cancer: breast-conserving surgery and mastectomy. Breast-conserving surgery is surgery to remove the cancer as well as some surrounding normal tissue. Only the part of the breast containing the cancer is removed. How much breast is removed depends on where and how big the tumor is, as well as other factors. This surgery is also called a lumpectomy, quadrantectomy, partial mastectomy, or segmental mastectomy. Mastectomy is a surgery in which the entire breast is removed, including all of the breast tissue and sometimes other nearby tissues. There are several different types of mastectomies. Some women may also have both breasts removed in a double mastectomy. Sometimes surgery is done to remove the nearby lymph nodes and other tissue where the cancer has spread.

Radiation therapy uses high-energy x-rays or other types of radiation to kill cancer cells or keep them from growing. There are two types of radiation therapy: external radiation therapy uses a machine outside the body to send radiation toward the area of the body with cancer; internal radiation therapy uses a radioactive substance sealed in needles, seeds, wires, or catheters that are placed directly into or near the cancer. Additionally, targeted radiopharmaceuticals can provide targeted radiation to the site of the tumor. Chemotherapy is a cancer treatment that uses drugs to stop the growth of cancer cells, either by killing the cells or by stopping them from dividing.

Thus, a need exists for treatment options for breast tumors. Described herein are radiopharmaceuticals that target delivery of radionuclides to breast tumors, which overexpress the GnRHR. Targeted therapies usually cause less harm to normal cells than chemotherapy or radiation therapy do.

Solid Tumors: Benign and/or Malignant Neoplasms (Cancer)

In one aspect, the GnRHR radiopharmaceuticals described herein are used to treat benign and/or malignant neoplasms (solid tumors), wherein the neoplasm comprises cells that overexpress GnRHR on the cell surface.

The term “neoplasm” as used herein, refers to an abnormal growth of cells that may proliferate in an uncontrolled way and may have the ability to metastasize (spread).

Neoplasms include solid tumors, adenomas, carcinomas, sarcomas, leukemias and lymphomas, at any stage of the disease with or without metastases.

A solid tumor is an abnormal mass of tissue that usually does not contain cysts or liquid areas. Solid tumors may be benign (not cancer), or malignant (cancer). Different types of solid tumors are named for the type of cells that form them. Examples of solid tumors are sarcomas, carcinomas, and lymphomas. Leukemias (cancers of the blood) generally do not form solid tumors.

Solid tumors are cancers that typically originate in organs, such as the bladder, bowel, brain, breast, endometrium, heart, kidney, lung, liver, uterus, ovaries, pancreas or other endocrine organs (thyroid), and prostate.

In some embodiments, the GnRHR radiopharmaceuticals described herein are used to treat an adenoma. An adenoma is a tumor that is not cancer. It starts in gland-like cells of the epithelial tissue (thin layer of tissue that covers organs, glands, and other structures within the body). An adenoma can grow from many glandular organs, including the adrenal glands, pituitary gland, thyroid, prostate, and others Even though benign, they have the potential to cause serious health complications by compressing other structures (mass effect) and by producing large amounts of hormones in an unregulated, non-feedback-dependent manner (causing paraneoplastic syndromes). Over time adenomas may transform to become malignant, at which point they are called adenocarcinomas.

Adenomas may be found in the colon (e.g. adenomatous polyps, which have a tendency to become malignant and to lead to colon cancer), kidneys (e.g. renal adenomas may be precursor lesions to renal carcinomas), adrenal glands (e.g. adrenal adenomas; some secrete hormones such as cortisol, causing Cushing's syndrome, aldosterone causing Conn's syndrome, or androgens causing hyperandrogenism), thyroid (e.g. thyroid adenoma), pituitary (e.g. pituitary adenomas, such as prolactinoma, Cushing's disease and acromegaly), parathyroid (e.g. an adenoma of a parathyroid gland may secrete inappropriately high amounts of parathyroid hormone and thereby cause primary hyperparathyroidism), liver (e.g. hepatocellular adenoma), breast (e.g. fibroadenomas), appendix (e.g. cystadenoma), bronchial (e.g. bronchial adenomas may cause carcinoid syndrome, a type of paraneoplastic syndrome), prostate (e.g. prostate adenoma), sebaceous gland (e.g. sebaceous adenoma), and salivary glands.

Metastasis is the spread of malignant cells to new areas of the body, often by way of the lymph system or bloodstream. A metastatic tumor is one that has spread from the primary site of origin, or where it started, into different areas of the body. Metastatic tumors comprise malignant cells that may express cell surface GnRHR.

Tumors formed from cells that have spread are called secondary tumors. Tumors may have spread to areas near the primary site, called regional metastasis, or to parts of the body that are farther away, called distant metastasis.

In some embodiments, the tumor to be treated comprises tumor cells expressing GnRHR, wherein the tumor is a primary or metastatic tumor. In some embodiments, the tumor to be treated comprises tumor cells expressing GnRHR, wherein the tumor is a primary or metastatic tumor of breast origin. In some embodiments, the tumor to be treated comprises tumor cells expressing GnRHR, wherein the tumor is a primary or metastatic tumor of endometrial origin. In some embodiments, the tumor to be treated comprises tumor cells expressing GnRHR, wherein the tumor is a primary or metastatic tumor of ovarian origin. In some embodiments, the tumor to be treated comprises tumor cells expressing GnRHR, wherein the tumor is a primary or metastatic tumor of prostate origin.

In some embodiments, the GnRHR radiopharmaceuticals described herein are used to treat a carcinoma. Carcinomas include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelial carcinoma, and nasopharyngeal carcinoma, etc.

In some embodiments, the GnRHR radiopharmaceuticals described herein are used to treat a sarcoma. Sarcomas include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Solid tumors include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma. Benign solid tumors include adenomas.

Primary and metastatic tumors include, e.g., lung cancer (including, but not limited to, lung adenocarcinoma, squamous cell carcinoma, large cell carcinoma, bronchioloalveolar carcinoma, non-small-cell carcinoma, small cell carcinoma, mesothelioma); breast cancer (including, but not limited to, ductal carcinoma, lobular carcinoma, inflammatory breast cancer, clear cell carcinoma, mucinous carcinoma); colorectal cancer (including, but not limited to, colon cancer, rectal cancer); anal cancer; pancreatic cancer (including, but not limited to, pancreatic adenocarcinoma, islet cell carcinoma, neuroendocrine tumors); prostate cancer; ovarian carcinoma (including, but not limited to, ovarian epithelial carcinoma or surface epithelial-stromal tumor including serous tumor, endometrioid tumor and mucinous cystadenocarcinoma, sex-cord-stromal tumor); liver and bile duct carcinoma (including, but not limited to, hepatocellular carcinoma, cholangiocarcinoma, hemangioma); esophageal carcinoma (including, but not limited to, esophageal adenocarcinoma and squamous cell carcinoma); non-Hodgkin's lymphoma; bladder carcinoma; carcinoma of the uterus (including, but not limited to, endometrial adenocarcinoma, uterine papillary serous carcinoma, uterine clear-cell carcinoma, uterine sarcomas and leiomyosarcomas, mixed mullerian tumors); glioma, glioblastoma, medulloblastoma, and other tumors of the brain; kidney cancers (including, but not limited to, renal cell carcinoma, clear cell carcinoma, Wilm's tumor); cancer of the head and neck (including, but not limited to, squamous cell carcinomas); cancer of the stomach (including, but not limited to, stomach adenocarcinoma, gastrointestinal stromal tumor); multiple myeloma; testicular cancer; germ cell tumor; neuroendocrine tumor; cervical cancer; carcinoids of the gastrointestinal tract, breast, and other organs; and signet ring cell carcinoma.

### Representative Gonadotropin-Releasing Hormone Receptor (GnRHR) Targeting Ligands

In some embodiments, the GnRHR radiopharmaceuticals described herein have an affinity to GnRHR that is at least 10-fold, at least 50-fold, at least 100-fold, at least 200-fold, at least 500-fold, or at least 1000-fold greater than the affinity for other non-target receptors.

In some embodiments, the GnRHR radiopharmaceuticals described herein preferentially accumulates in tumor tissues that express the targeted GnRHR. In some embodiments, the GnRHR radiopharmaceuticals described herein preferentially accumulate in tissues or organs comprising tumor cells that express GnRHR as compared to tissues or organ(s) lacking tumor cells that express GnRHR. In some embodiments, the GnRHR radiopharmaceuticals described herein preferentially accumulate at least 1-fold, at least 2-fold, 3-fold, at least 4-fold, at least 5-fold, or greater than 5-fold more in tissues or organ(s) comprising tumor cells that express GnRHR as compared to tissues or organs lacking tumor cells that express GnRHR. It is understood that the compound may accumulate in certain tissues and organs involved in the metabolism and/or excretion of therapeutics, including but not limited to the kidneys and liver.

In one aspect, described herein is a compound of Formula (I), or a pharmaceutically acceptable salt thereof,

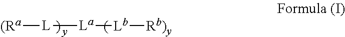

wherein:
L^{a} is absent or a branched linker;
each L is independently selected from absent or a linker;
each L^{b} is independently selected from absent or a linker;
each R^{a} is independently selected from a chelating moiety or a radionuclide complex thereof;
each R^{b} is independently a small molecule modulator of the gonadotropin-releasing hormone receptor (GnRHR); and
each y is independently 1, 2 or 3.

In some embodiments, each y is 1.

In some embodiments, R^{b} is a small molecule antagonist of GnRHR.

In some embodiments, R^{b} comprises a furan pyrimidine, a pyridine-chloro-benzamide, a pyridine-chloro-benzenesulfamide, a uracil, a thieno[2,3-d]pyrimidine-2,4(1H,3H)-dione, or a thieno[3,4-d]pyrimidine-2,4(1H,3H)-dione. In some embodiments, R^{b} comprises a furan pyrimidine. In some embodiments, R^{b} comprises a pyridine-chloro-benzamide or a pyridine-chloro-benzenesulfamide. In some embodiments, R^{b} is a small molecule agonist of GnRHR.

In some embodiments, L^{a} is a linker. In some embodiments, L^{a} is absent.

In some embodiments, L^{b} is a linker. In some embodiments, L^{b} is absent.

In some embodiments, L is a linker.

In some embodiments, the compound has the structure of Formula (II), or a pharmaceutically acceptable salt thereof. In some embodiments, described herein is a compound of Formula (II), or a pharmaceutically acceptable salt thereof:

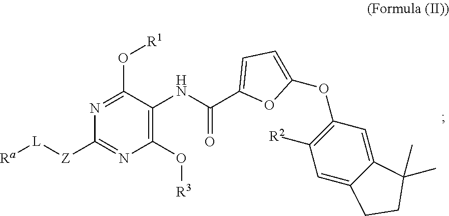

wherein:
R¹ is H, substituted or unsubstituted —C₁-C₄ alkyl or —C₁-C₄ haloalkyl;
R² is H, halo, substituted or unsubstituted —C₁-C₆ alkyl, or —C₁-C₄ haloalkyl;
R³ is H, substituted or unsubstituted —C₁-C₄ alkyl or —C₁-C₄ haloalkyl;
Z is —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—;
R⁴ is H or unsubstituted —C₁-C₄ alkyl;
L is a linker; and
R^{a} is a chelating moiety or a radionuclide complex thereof.

In some embodiments, R¹ is H. In some embodiments, R¹ is substituted or unsubstituted —C₁-C₄ alkyl. In some embodiments, R¹ is —C₁-C₄ haloalkyl. In some embodiments, R¹ is —CH₃. In some embodiments, R¹ is —CH₂CH₃. In some embodiments, R¹ is —CF₃.

In some embodiments, R¹ is H; Z is absent, —O—, —S—, or —N(R⁴)—; and R⁴ is H or —C₁-C₄ alkyl. In some embodiments, R¹ is —CH₃; Z is absent, —O—, —S—, or —N(R⁴)—; and R⁴ is H or —C₁-C₄ alkyl.

In some embodiments, R² is H. In some embodiments, R² is F. In some embodiments, R² is Cl. In some embodiments, R² is Br. In some embodiments, R² is I. In some embodiments, R² is substituted or unsubstituted —C₁-C₄ alkyl. In some embodiments, R² is —C₁-C₄ haloalkyl. In some embodiments, R² is —CH₃. In some embodiments, R² is —CH₂CH₃. In some embodiments, R² is —CF₃.

In some embodiments, R³ is H. In some embodiments, R³ is substituted or unsubstituted —C₁-C₄ alkyl. In some embodiments, R³ is —C₁-C₄ haloalkyl. In some embodiments, R³ is —CH₃. In some embodiments, R³ is —CH₂CH₃. In some embodiments, R³ is —CF₃.

In some embodiments, R³ is H. In some embodiments, R³ is substituted or unsubstituted —C₁-C₄ alkyl. In some embodiments, R³ is —CH₃. In some embodiments, R³ is —CH₂CH₃.

In some embodiments, Z is absent, —O—, —S—, or —N(R⁴)—. In some embodiments, Z is —C₁-C₆ alkylene. In some embodiments, Z is —C₁-C₆ alkylene-O—. In some embodiments, Z is —O—C₁-C₆ alkylene-. In some embodiments, Z is —C(═O)NR⁴—. In some embodiments, Z is —NR⁴C(═O)—. In some embodiments, Z is —O—. In some embodiments, Z is —NR⁴—. In some embodiments, Z is —NH—. In some embodiments, Z is —S—. In some embodiments, Z is —S(═O)—. In some embodiments, Z is —SO₂—. In some embodiments, Z is —NHC(═O)NH—.

In some embodiments, R⁴ is H. In some embodiments, R⁴ is unsubstituted —C₁-C₄ alkyl. In some embodiments, R⁴ is —CH₃.

In some embodiments, the compound has the structure of Formula (IIIa) or (IIIb), or a pharmaceutically acceptable salt thereof:

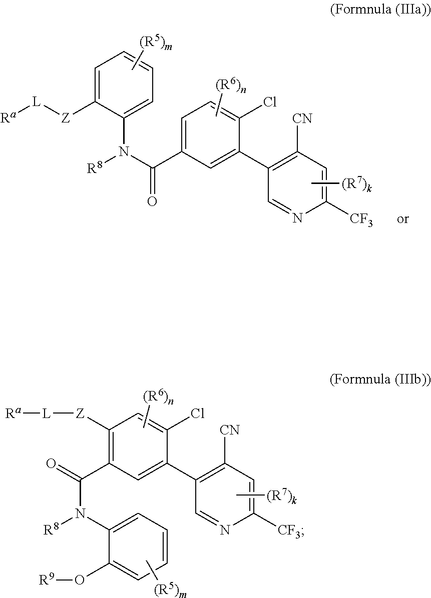

wherein:
each R⁵ is independently selected from F, Cl, Br, I, —CN, substituted or unsubstituted —C₁-C₆ alkyl, or substituted or unsubstituted —C₁-C₆ alkoxy;
each R⁶ is independently selected from F, Cl, Br, I, —CN, substituted or unsubstituted —C₁-C₆ alkyl, or substituted or unsubstituted —C₁-C₆ alkoxy;
each R⁷ is independently selected from F, Cl, Br, I, —CN, substituted or unsubstituted —C₁-C₆ alkyl, or substituted or unsubstituted —C₁-C₆ alkoxy;
R⁸ is H or substituted or unsubstituted —C₁-C₄ alkyl;
R⁹ is H or substituted or unsubstituted —C₁-C₄ alkyl;
Z is —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—R⁴ is H or unsubstituted —C₁-C₄ alkyl;
L is a linker; and
R^{a} is a chelating moiety or a radionuclide complex thereof;
m is 0, 1, 2, 3, or 4;
n is 0, 1, 2, or 3; and
k is 0, 1, or 2.

In some embodiments, the compound has the structure of Formula (IIIa), or a pharmaceutically acceptable salt thereof:

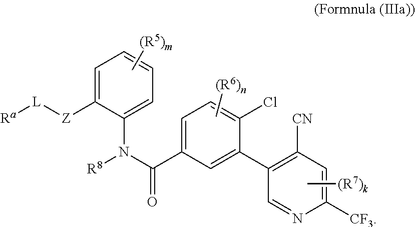

In some embodiments, the compound has the structure of Formula (IIIb), or a pharmaceutically acceptable salt thereof:

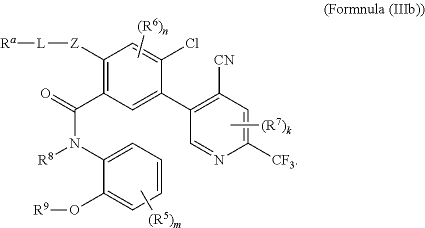

In some embodiments, each R⁵ is independently selected from F, Cl, Br, I, —CH₃, or —OCH₃. In some embodiments, each R⁵ is F. In some embodiments, each R⁵ is Cl. In some embodiments, each R⁵ is Br. In some embodiments, each R⁵ is I. In some embodiments, each R⁵ is —CN. In some embodiments, each R⁵ is independently selected from substituted or unsubstituted —C₁-C₆ alkyl. In some embodiments, each R⁵ is independently selected from substituted or unsubstituted —C₁-C₆ alkoxy.

In some embodiments, each R⁶ is independently selected from F, Cl, Br, I, —CH₃, or —OCH₃. In some embodiments, each R⁶ is F. In some embodiments, each R⁶ is Cl. In some embodiments, each R⁶ is Br. In some embodiments, each R⁶ is I. In some embodiments, each R⁶ is —CN. In some embodiments, each R⁶ is independently selected from substituted or unsubstituted —C₁-C₆ alkyl. In some embodiments, each R⁶ is independently selected from substituted or unsubstituted —C₁-C₆ alkoxy.

In some embodiments, each R⁷ is independently selected from F, Cl, Br, I, —CH₃, or —OCH₃. In some embodiments, each R⁷ is F. In some embodiments, each R⁷ is Cl. In some embodiments, each R⁷ is Br. In some embodiments, each R⁷ is I. In some embodiments, each R⁷ is —CN. In some embodiments, each R⁷ is independently selected from substituted or unsubstituted —C₁-C₆ alkyl. In some embodiments, each R⁷ is independently selected from substituted or unsubstituted —C₁-C₆ alkoxy.

In some embodiments, R⁸ is H. In some embodiments, R⁸ is substituted or unsubstituted —C₁-C₄ alkyl. In some embodiments, R⁸ is —CH₃.

In some embodiments, R⁹ is H. In some embodiments, R⁹ is substituted or unsubstituted —C₁-C₄ alkyl. In some embodiments, R⁹ is substituted —C₁-C₄ alkyl, substituted with 1, 2, or 3 —OH, —NH₂ or —CO₂H. In some embodiments, R⁹ is —C₁-C₄ alkyl, substituted with 1 —OH, —NH₂ or —CO₂H. In some embodiments, R⁹ is unsubstituted —C₁-C₄ alkyl. In some embodiments, R⁹ is —CH₂CH₂CO₂H. In some embodiments, R⁹ is —CH₃.

In another aspect, described herein is a compound of Formula (IIIc), or a pharmaceutically acceptable salt thereof:

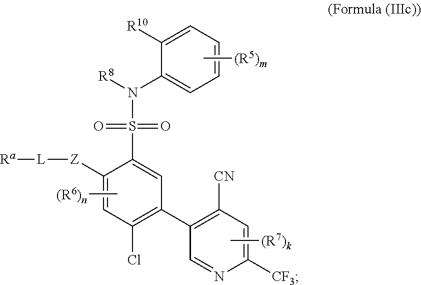

wherein:
each R⁵ is independently selected from F, Cl, Br, I, —CN, substituted or unsubstituted —C₁-C₆ alkyl, or substituted or unsubstituted —C₁-C₆ alkoxy;
each R⁶ is independently selected from F, Cl, Br, I, —CN, substituted or unsubstituted —C₁-C₆ alkyl, or substituted or unsubstituted —C₁-C₆ alkoxy;
each R⁷ is independently selected from F, Cl, Br, I, —CN, substituted or unsubstituted —C₁-C₆ alkyl, or substituted or unsubstituted —C₁-C₆ alkoxy;
R⁸ is —C₁-C₄ alkyl;
R¹⁰ is H, —OH, —C₁-C₄ alkyl, or —O—C₁-C₄ alkyl; or
R⁸ and R¹⁰ are taken together with the intervening atoms connecting R⁸ to R¹⁰, to form a substituted or unsubstituted 4- to 7-membered heterocyclic ring;
Z is —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—;
R⁴ is H or unsubstituted —C₁-C₄ alkyl;
L is a linker;
R^{a} is a chelating moiety or a radionuclide complex thereof;
m is 0, 1, 2, 3, or 4;
n is 0, 1, 2, or 3; and
k is 0, 1, or 2.

In some embodiments, R⁸ is —CH₃.

In some embodiments, R¹⁰ is H. In some embodiments, R¹⁰ is —OH. In some embodiments, R¹⁰ is —C₁-C₄ alkyl. In some embodiments, R¹⁰ is —CH₃. In some embodiments, R¹⁰ is —CH₂CH₃. In some embodiments, R¹⁰ is —O—C₁-C₄ alkyl. In some embodiments, R¹⁰ is —OCH₃.

In some embodiments, R⁸ and R¹⁰ are taken together with the intervening atoms connecting R⁸ to R¹⁰ to form a substituted or unsubstituted 5-membered heterocyclic ring, substituted or unsubstituted 6-membered heterocyclic ring, or a substituted or unsubstituted 7-membered heterocyclic ring, wherein the heterocyclic ring is optionally substituted with 1 to 3 substituents independently selected from F, Cl, —C₁-C₄ alkyl, or —O—C₁-C₄ alkyl. In some embodiments, R⁸ and R¹⁰ are taken together with the intervening atoms connecting R⁸ to R¹⁰ to form a 5-membered heterocyclic ring optionally substituted with 1 to 3 F, Cl, —CH₃, or —OCH₃. In some embodiments, R⁸ and R¹⁰ are taken together with the intervening atoms connecting R⁸ to R¹⁰ to form a 6-membered heterocyclic ring, optionally substituted with 1 to 3 F, Cl, —CH₃, or —OCH₃. In some embodiments, R⁸ and R¹⁰ are taken together with the intervening atoms connecting R⁸ to R¹⁰ to form a 6-membered heterocyclic ring, optionally substituted with 1 to 3 F, Cl, —CH₃, or —OCH₃.

In some embodiments, R⁸ and R¹⁰ are taken together with the intervening atoms connecting R⁸ to R¹⁰ to form

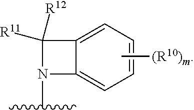

In some embodiments, R⁸ and R¹⁰ are taken together with the intervening atoms connecting R⁸ to R¹⁰ to form

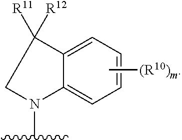

In some embodiments, R⁸ and R¹⁰ are taken together with the intervening atoms connecting R⁸ to R¹⁰ to form

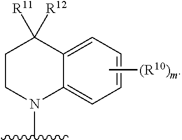

In some embodiments, R⁸ and R¹⁰ are taken together with the intervening atoms connecting R⁸ to R¹⁰ to form

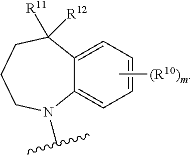

In some embodiments, R¹¹ and R¹² are each independently selected from H, F, or —C₁-C₄ alkyl In some embodiments, R¹¹ and R¹² are H. In some embodiments, R¹¹ and R¹² are F. In some embodiments, R¹¹ and R¹² are —CH₃. In some embodiments, R¹¹ and R¹² come together to form a substituted or unsubstituted —C₃-C₆ cycloalkyl ring. In some embodiments, R¹¹ and R¹² come together to form an unsubstituted cyclopropyl ring.

In some embodiments, R⁸ and R¹⁰ come together to form

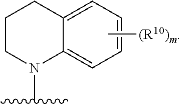

In some embodiments, R⁸ and R¹⁰ come together to form

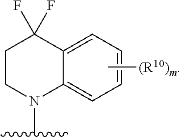

In some embodiments, R⁸ and R¹⁰ come together to form

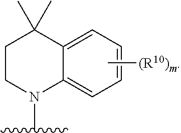

In some embodiments, the compound has the structure of Formula (IIId), or a pharmaceutically acceptable salt thereof:

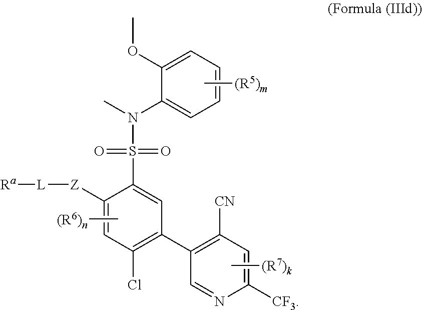

In some embodiments, the compound has the structure of Formula (IIIe), or a pharmaceutically acceptable salt thereof:

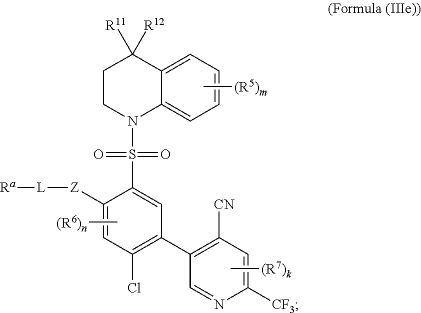

wherein R¹¹ and R¹² are each independently selected from H, F, or —C₁-C₄ alkyl; or
R¹¹ and R¹² can come together to form a substituted or unsubstituted —C₃-C₆ cycloalkyl ring.

In some embodiments, m is 0. In some embodiments, m is 1. In some embodiments, m is 2. In some embodiments, m is 3. In some embodiments, m is 4.

In some embodiments, n is 0. In some embodiments, n is 1. In some embodiments, n is 2. In some embodiments, n is 3.

In some embodiments, k is 0. In some embodiments, k is 1. In some embodiments, k is 2.

In some embodiments, Z is absent, —O—, —S—, or —N(R⁴)—. In some embodiments, Z is —C₁-C₆ alkylene. In some embodiments, Z is —C₁-C₆ alkylene-O—. In some embodiments, Z is —O—C₁-C₆ alkylene-. In some embodiments, Z is —C(═O)NR⁴—. In some embodiments, Z is —NR⁴C(═O)—. In some embodiments, Z is —O—. In some embodiments, Z is —NR⁴—. In some embodiments, Z is —NH—. In some embodiments, Z is —S—. In some embodiments, Z is —S(═O)—. In some embodiments, Z is —SO₂—. In some embodiments, Z is —NHC(═O)NH—.

In some embodiments, Z is absent. In some embodiments, Z is —O—, —S—, —NH—, or —N(—CH₃)—. In some embodiments, Z is —O—. In some embodiments, Z is —S—. In some embodiments, Z is —NH—. In some embodiments, Z is —N(—CH₃)—.

In some embodiments, the chelating moiety of R^{a} is independently selected from the group consisting of: cyclen, DO2A, DO3A, HP-DO3A, DO3A-Nprop, DO3AP, DO3AP^{PrA}DO3AP^{ABn}, DO3AM^{nBu}, BT-DO3A, DOTA, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NODA-MPAA, TACN, TACN-TM, NOTP, Sarcophagine (Sar), DiAmSar, SarAr, AmBaSar, cis-DO2A2P, trans-DO2A2P, DOTEP, p-NO₂-Bn-DOTA, BAT, DO3TMP-Monoamide, CHX-A″-DTPA, c-DEPA, PCTA, p-NO₂-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, H₆phospha, p-NH₂-Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), or SBTG2DAP.

In some embodiments, the chelating moiety of R^{a} is independently selected from the group consisting of:
1,4,7,10-tetraazacyclododecane-1,4,7,10-tetraacetic acid (DOTA);
1,4,7,10-tetraazacyclododecane-1,4,7-triacetic acid (DO3A);
1,4,7,10-tetraazacyclododecane-1,7-diacetic acid (DO2A);
α,α′,α″,α′″-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7,10-tetraacetic acid (p-OH-Bn-DOTA);
6,6′-(((pyridine-2,6-diylbis(methylene))bis((carboxymethyl)azanediyl))bis(methylene))dipicolinic acid (H₄pypa);
H₄pypa-benzyl;
6,6′,6″,6′″-(((pyridine-2,6-diylbis(methylene))bis(azanetriyl))tetrakis(methylene))-tetrapicolinic acid (H₄py4pa);
H₄py4pa-benzyl;
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2′″-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl;
3,6,9,12-tetrakis(carboxymethyl)-3,6,9,12-tetraazatetradecanedioic acid (TTHA);
or a radionuclide complex thereof.

In some embodiments, the chelating moiety of R^{a} is independently selected from the group consisting of: 1,4,7,10-tetraazacyclododecane-1,4,7,10-tetraacetic acid (DOTA); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; 6,6′-(((pyridine-2,6-diylbis(methylene))bis((carboxymethyl)azanediyl))bis(methylene))dipicolinic acid (H₄pypa); 6,6′,6″,6′″-(((pyridine-2,6-diylbis(methylene))bis(azanetriyl))tetrakis(methylene))-tetrapicolinic acid (H₄py4pa); 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); 3,6,9,12-tetrakis(carboxymethyl)-3,6,9,12-tetraazatetradecanedioic acid (TTHA); or a radionuclide complex thereof.

In some embodiments, R^{a} is 1,4,7,10-tetraazacyclododecane-1,4,7,10-tetraacetic acid (DOTA) or a radionuclide complex thereof. In some embodiments, R^{a} is 1,4,7,10-tetraazacyclododecane-1,4,7-triacetic acid (DO3A) or a radionuclide complex thereof. In some embodiments, R^{a} is 1,4,7,10-tetraazacyclododecane-1,7-diacetic acid (DO2A) or a radionuclide complex thereof. In some embodiments, R^{a} is α,α′,α″,α′″-tetramethyl-1,4,7,10-tetraazacyclododecane-1,4,7,10-tetraacetic acid (DOTMA) or a radionuclide complex thereof. In some embodiments, R^{a} is 1,4,7,10-tetrakis(carbamoylmethyl)-1,4,7,10-tetraazacyclododecane (DOTAM) or a radionuclide complex thereof. In some embodiments, R^{a} is 1,4,7,10-tetraazacyclododecane-1,4,7,10-tetrapropionic acid (DOTPA) or a radionuclide complex thereof. In some embodiments, R^{a} is 2,2′,2″-(10-(2-amino-2-oxoethyl)-1,4,7,10-tetraazacyclododecane-1,4,7-triyl)triacetic acid or a radionuclide complex thereof. In some embodiments, R^{a} is 6,6′-(((pyridine-2,6-diylbis(methylene))bis((carboxymethyl)azanediyl))bis(methylene))dipicolinic acid (H₄pypa) or a radionuclide complex thereof. In some embodiments, R^{a} is 6,6′,6″,6′″-(((pyridine-2,6-diylbis(methylene))bis(azanetriyl))tetrakis(methylene))-tetrapicolinic acid (H₄py4pa) or a radionuclide complex thereof. In some embodiments, R^{a} is 2,2′,2″-(1,4,7-triazacyclononane-1,4,7-triyl)triacetic acid (NOTA). In some embodiments, R^{a} is 6,6′-((1,4,10,13-tetraoxa-7,16-diazacyclooctadecane-7,16-diyl)bis(methylene))dipicolinic acid (macropa). In some embodiments, R^{a} is 2,2′,2″,2′″-(1,10-dioxa-4,7,13,16-tetraazacyclooctadecane-4,7,13,16-tetrayl)tetraacetic acid (crown). In some embodiments, R^{a} is 6,6′-((ethane-1,2-diylbis((carboxymethyl)azanediyl))bis(methylene))dipicolinic acid (H₄octapa) or a radionuclide complex thereof. In some embodiments, R^{a} is 3,6,9,12-tetrakis(carboxymethyl)-3,6,9,12-tetraazatetradecanedioic acid (TTHA) or a radionuclide complex thereof.

In some embodiments, the chelating moiety of R^{a} is independently selected from the group consisting of: 1,4,7,10-tetraazacyclododecane-1,4,7,10-tetraacetic acid (DOTA) or 1,4,7,10-tetraazacyclododecane-1,4,7-triacetic acid (DO3A); or a radionuclide complex thereof.

In some embodiments, the chelating moiety of R^{a} is independently selected from the group consisting of:

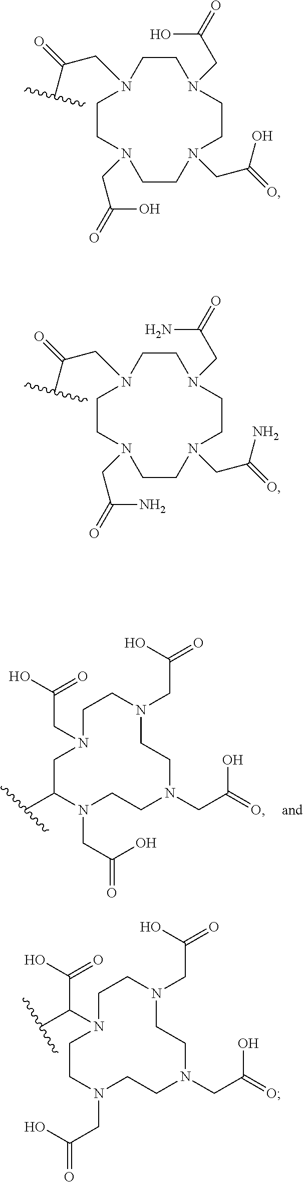 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof. In some embodiments, R^{a} is

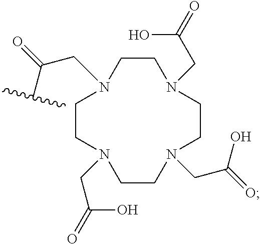 [SMILES: *C(=O)CN1CCN(CC(=O)O)CCN(CC(=O)O)CCN(CC(=O)O)CC1]

or a radionuclide complex thereof. In some embodiments, R^{a} is

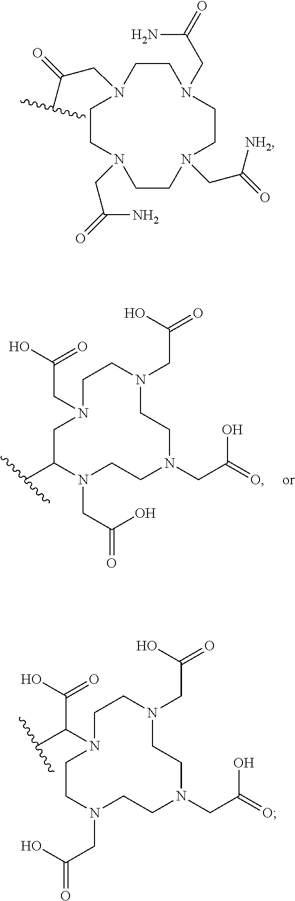 [SMILES: *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof. In some embodiments, R^{a} is

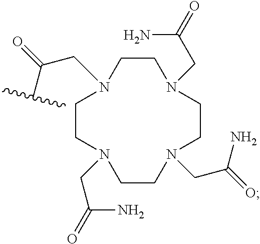 [SMILES: *C(=O)CN1CCN(CC(N)=O)CCN(CC(N)=O)CCN(CC(N)=O)CC1]

or a radionuclide complex thereof. In some embodiments, R^{a} is

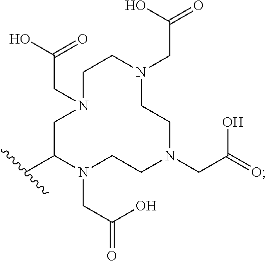 [SMILES: *C1CN(CC(=O)O)CCN(CC(=O)O)CCN(CC(=O)O)CCN1CC(=O)O]

or a radionuclide complex thereof. In some embodiments, R^{a} is

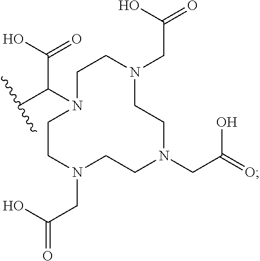 [SMILES: *C(C(=O)O)N1CCN(CC(=O)O)CCN(CC(=O)O)CCN(CC(=O)O)CC1]

or a radionuclide complex thereof.

### Radionuclide Complexes

Radiopharmaceuticals have increasingly become very useful tools for physicians to diagnose, stage, treat, and monitor the progression of several diseases, especially cancer. The primary difference between radiopharmaceuticals and other pharmaceutical drugs is that radiopharmaceuticals contain a radionuclide. The nuclear decay properties of the radionuclide determine whether a radiopharmaceutical will be used clinically as a diagnostic agent or as a therapeutic agent. Diagnostic radiopharmaceuticals require radionuclides that emit either gamma (γ) rays or positrons (β+), which subsequently annihilate with nearby electrons to produce two 511 keV annihilation photons emitted approximately 180° away from each other. Gamma ray-emitting radionuclides (e. g. ^{99m}Tc, ¹¹¹In, ²⁰¹Tl, etc.) are useful for single photon emission computed tomography (SPECT), while positron-emitting radionuclides (e. g. ¹⁸F, ⁸⁹Zr, ⁶⁸Ga, etc.) are useful for positron emission tomography (PET).

In contrast, therapeutic radiopharmaceuticals require radionuclides that emit particulate radiation, such as alpha (α) particles, beta (β−) particles, or Auger electrons. These particles, which strongly interact with target tissues (e. g. cancerous tumor) and lead to extensive localized ionization, can damage chemical bonds in DNA molecules and potentially induce cytotoxicity.

For most nuclear medicine applications, it is desired that a diagnostic radiopharmaceutical is paired with a therapeutic radiopharmaceutical. This concept is commonly known as “theranostics”. As a first step in the theranostic concept, a target molecule labeled with a diagnostic radionuclide is used for quantitative imaging of a tumor imaging biomarker, either by positron emission tomography (PET) or single photon emission computed tomography (SPECT). When it is demonstrated that, with this targeted molecule, a tumoricidal radiation absorbed dose can be delivered to tumor and metastases, as a second step, via administration of the same or a similar target molecule labeled with a therapeutic radionuclide.

In some embodiments, the chemical and pharmacokinetic behaviors of both the diagnostic and therapeutic radiopharmaceuticals match. In some embodiments, the diagnostic and therapeutic radionuclides are a chemically identical radioisotope pair (also known as a “matched pair”). One example of a matched pair for theranostic radiopharmaceutical applications is the ¹²³I/¹³¹I pair, where ¹²³I-labeled compounds are used for diagnosis, while ¹³¹I-labeled compounds are used for therapy. Other theranostic matched pairs include ⁴⁴Sc/⁴⁷Sc, ⁶⁴Cu/⁶⁷Cu, ⁷²As/⁷⁷As, ⁸⁶Y/⁹⁰Y, and ²⁰³Pb/²¹²Pb, among others. Alternatively, radionuclide pairs from different elements can be utilized for theranostic radiopharmaceutical development when their chemistry is very similar (e. g. ^{99m}Tc/^{186/188}Re) and there is no significant difference in the pharmacokinetic behavior between the diagnostic and therapeutic analogues. Another example is the ⁶⁸Ga/¹⁷⁷Lu pair, where ⁶⁸Ga is used for diagnosis and ¹⁷⁷Lu is used for therapy. For example, gastroenteropancreatic endocrine tumors express high amounts of sst2 receptor that can be targeted with somatostatin receptor scintigraphy for diagnostic purposes with a ⁶⁸Ga sst2 ligand conjugate ([⁶⁸Ga]Ga-DOTA-TATE (NETSPOT™) or [⁶⁸Ga]Ga-DOTA-TOC (DOTA-(D-Phe1,Tyr3)-octreotide, SomaKit TOC®)), followed by treatment with a ¹⁷⁷Lu sst2 ligand conjugate ([¹⁷⁷Lu]Lu-DOTA-TATE) for endoradiotherapy.

Chelating Moieties used to Generate Metal (Radionuclide) Complexes

The compounds described herein comprise at least one R^{a} group, wherein R^{a} is a chelating moiety capable of chelating a radionuclide (Z′), or radionuclide complex thereof. In some embodiments, any suitable group or atom(s) of the chelator are used to connect, via an optional linker, to the GnRHR targeting ligand.

In some embodiments, the chelator is capable of binding a radioactive atom. In some embodiments, the binding is direct, e.g., the chelator makes hydrogen bonds or electrostatic interactions with a radioactive atom. In some embodiments, the binding is indirect, e.g., the chelator binds to a molecule that comprises a radioactive atom. In some embodiments, the chelator is or comprises a macrocycle.

In some embodiments, the chelator comprises one or more amine groups. In some embodiments, the metal chelator comprises two or more amine groups. In some embodiments, the chelator comprises three or more amine groups. In some embodiments, the chelator comprises four or more amine groups. In some embodiments, the chelator includes 4 or more N atoms, 4 or more carboxylic acid groups, or a combination thereof. In some embodiments, the chelator does not comprise S. In some embodiments, the chelator comprises a ring. In some embodiments, the ring comprises an O and/or a N atom. In some embodiments, the chelator is a ring that includes 3 or more N atoms, 3 or more carboxylic acid groups, or a combination thereof. In some embodiments, the chelator is polydentate ligand, bidentate ligand, or monodentate ligand. Polydentate ligands range in the number of atoms used to bond to a metal atom or ion. EDTA, a hexadentate ligand, is an example of a polydentate ligand that has six donor atoms with electron pairs that can be used to bond to a central metal atom or ion. Bidentate ligands have two donor atoms which allow them to bind to a central metal atom or ion at two points. Ethylenediamine (en) and the oxalate ion (ox) are examples of bidentate ligands.

In some embodiments, a chelator described herein comprises a cyclic chelating agent or an acyclic chelating agent. In some embodiments, a chelator described herein comprises a cyclic chelating agent. In some embodiments, a chelator described herein comprises an acyclic chelating agent.

In some embodiments, a chelator described herein comprises cyclen, DO2A, DO3A, HP-DO3A, DO3A-Nprop, DO3AP, DO3AP^{PrA}, DO3AP^{ABn}, DO3AM^{nBu}, BT-DO3A, DOTA, PSC, DOTAGA, DOTA(GA)₂, DOTAM, DOTA-4AMP, DOTMA, DOTP, CB-DO2A, DOTPA, DOTMP, DOTAMAP, TRITA, L^{py}, cyclam, TETA, CB-Cyclam, CB-TE2A, TE2A, NOTA, NODAGA, NODA-MPAA, TACN, TACN-TM, NOTP, Sarcophagine (Sar), DiAmSar, SarAr, AmBaSar, cis-DO2A2P, trans-DO2A2P, DOTEP, p-NO₂-Bn-DOTA, BAT, DO3TMP-Monoamide, CHX-A″-DTPA, c-DEPA, PCTA, p-NO₂-Bn-PCTA, TRAP, TRAPH, TRAP-OH, TRAP-Ph, NOPO, AAZTA, DATAM, HEHA, PEPA, DTA, EDTMP, DTPMP, NTA, EDTA, DTPA, CyDTPA, DFO, DFO*, deferiprone, TTHA, HBED, HBED-CC, HBED-CC TFP, H₄pypa, H₄py4pa, CP256, THP, YM103, t-Bu-calix[4]arene-tetracarboxylic acid, CHX-A″-DTPA, H₆phospha, p-NH₂-Bn-CHXA″-DTPA, DEDPA, H₄octox, H₄octapa, H₄CHXoctapa, HYNIC, macropa, crown, macropid, HOPO, Bis(2-mercaptoacetamide), Bis(aminothiolate), or SBTG2DAP.

In some embodiments, a chelator described herein comprises DOTA, DOTAGA, DOTA(GA)₂, NOTA, NODAGA, TRITA, TETA, DOTA-MA, HP-DO3A, DOTMA, DOTA-pNB, DOTP, DOTMP, DOTEP, DOTMPE, F-DOTPME, DOTPP, DOTBzP, DOTA-monoamide, BAT, DO3TMP-Monoamide, and CHX-A″-DTPA.

In some embodiments, a chelator described herein comprises DTA, CyEDTA, EDTMP, DTPMP, DTPA, CyDTPA, Cy2DTPA, DTPA-MA, DTPA-BA, and BOPA.

In some embodiments, a chelator described herein comprises DOTA, PSC, DOTAGA, DOTA(GA)₂, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP.

In some embodiments, a chelator described herein comprises DOTA, DOTAGA, DOTA(GA)₂, DOTP, DOTMA, DOTAM, DTPA, NTA, EDTA, DO3A, DO2A, NOC, NOTA, TETA, TACN, DiAmSar, CB-Cyclam, CB-TE2A, DOTA-4AMP, or NOTP.

In some embodiments, a chelator described herein comprises HP-DO3A, BT-DO3A, DO3A-Nprop, DO3AP, DO2A2P, DOA3P, DOTP, DOTPMB, DOTAMAE, DOTAMAP, DO3AM^{Bu}, DOTMA, TCE-DOTA, DEPA, PCTA, p-NO₂-Bn-PCTA, p-NO₂-Bn-DOTA, symPC2APA, symPCA2PA, asymPC2APA, asymPCA2PA, TRAP, AAZTA, DATA^{m}, THP, HEHA, HBED, or HBED-CC TFP.

In some embodiments, a chelator described herein comprises DOTA, NOTA, NODAGA, DOTAGA, HBED, HBED-CC TFP, H2DEPDPA, DFO-B, Deferiprone, CP256, YM103, TETA, CB-TE2A, TE2A, Sar, DiAmSar, TRAPH, TRAP-Pr, TRAP-OH, TRAP-Ph, NOPO, DEADPA, PCTA, EDTA, PEPA, HEHA, DTPA, EDTMP, AAZTA, DO3AP, DO3AP^{PrA}, DO3AP^{ABn}, or DOTAM.

In some embodiments, the chelator is or comprises DOTA, HBED-CC, DOTAGA, DOTA(GA)₂, NOTA, and DOTAM. In some embodiments, the chelator is or comprises NODAGA, NOTA, DOTAGA, DOTA(GA)₂, TRAP, NOPO, NCTA, DFO, DTPA, and HYNIC.

In some embodiments, the chelator comprises a macrocycle, e.g., a macrocycle comprising an O and/or a N atom, DOTA, HBED-CC, DOTAGA, DOTA(GA)₂, NOTA, DOTAM, one or more amines, one or more ethers, one or more carboxylic acids, EDTA, DTPA, TETA, DO3A, PCTA, or desferrioxamine.

In some embodiments, a metal chelator described herein comprises one of the following structures:

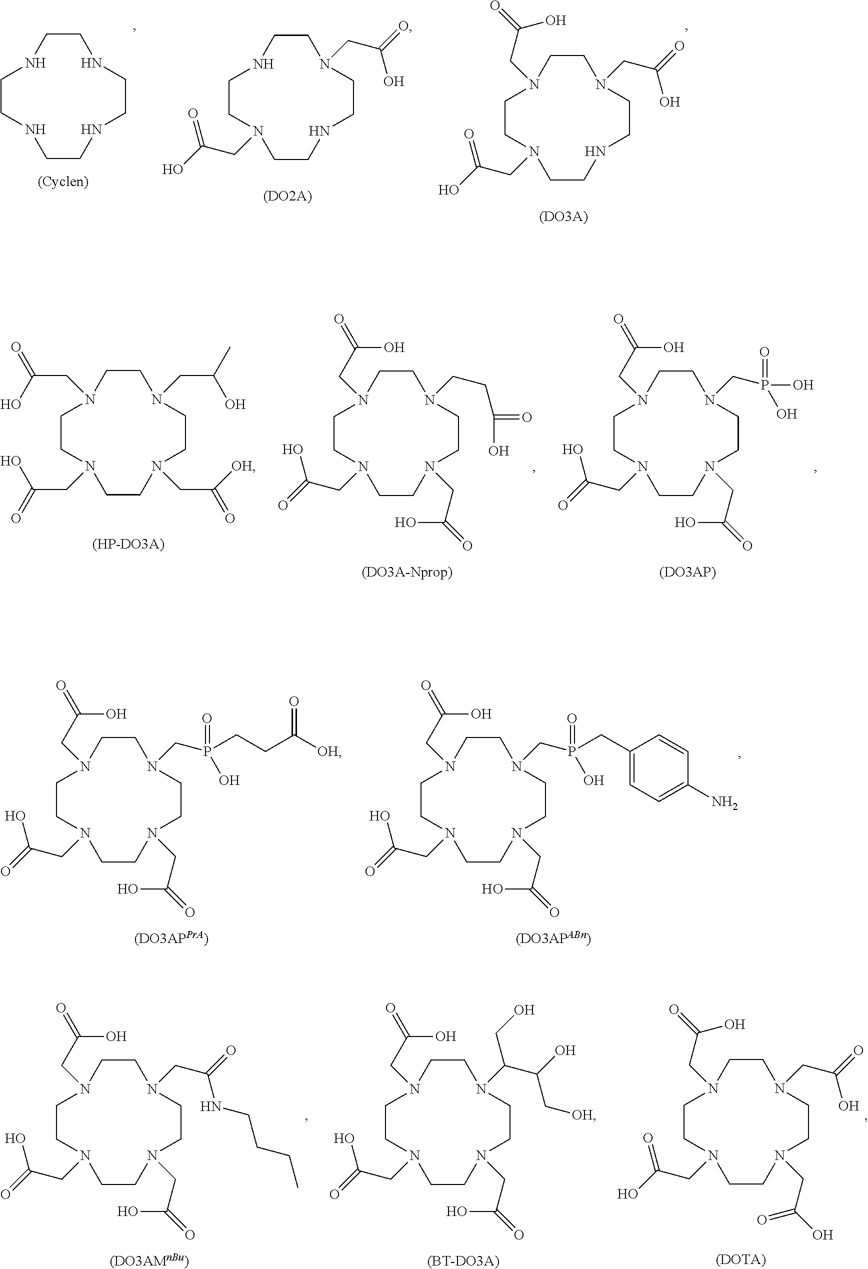

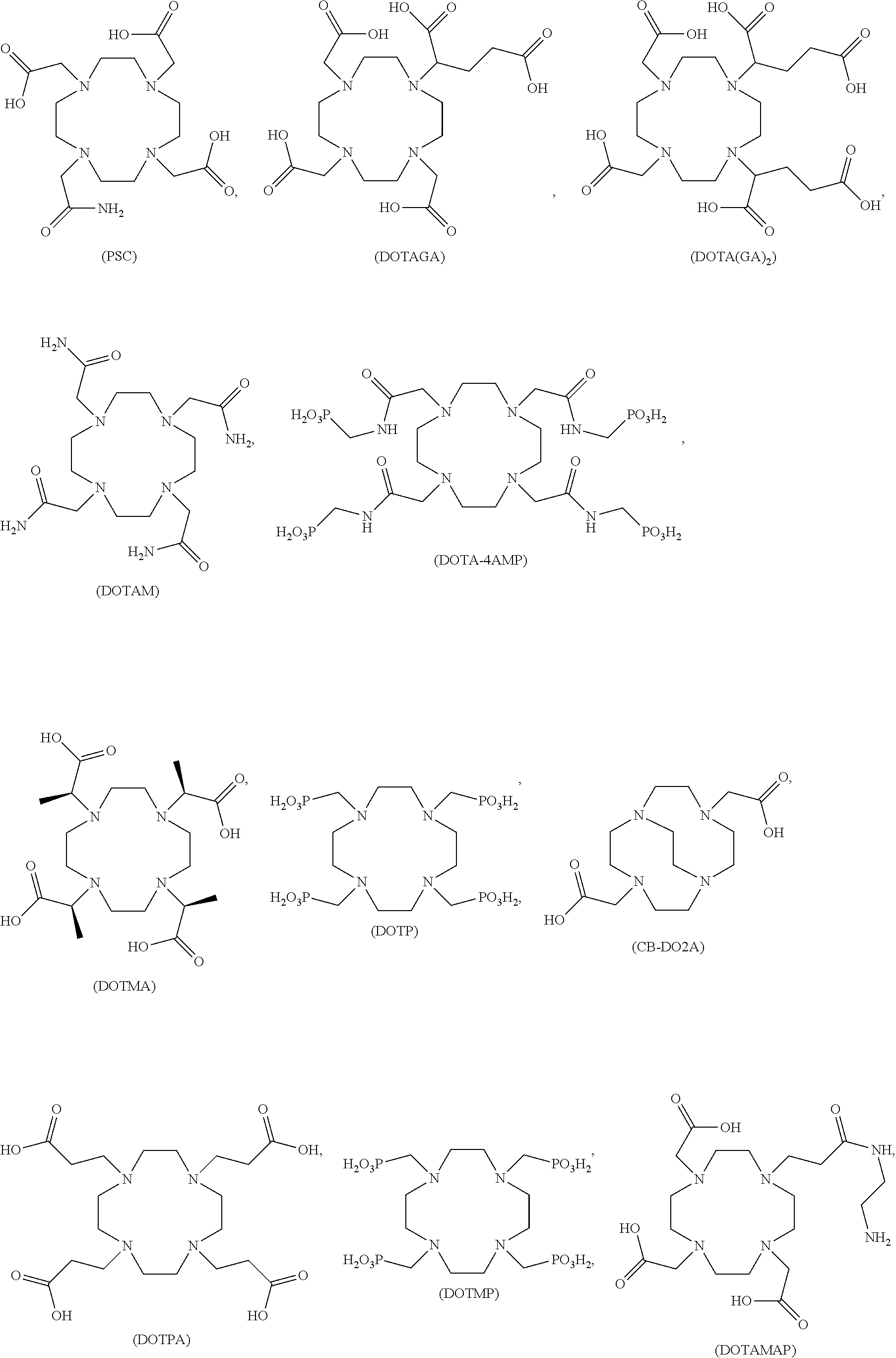

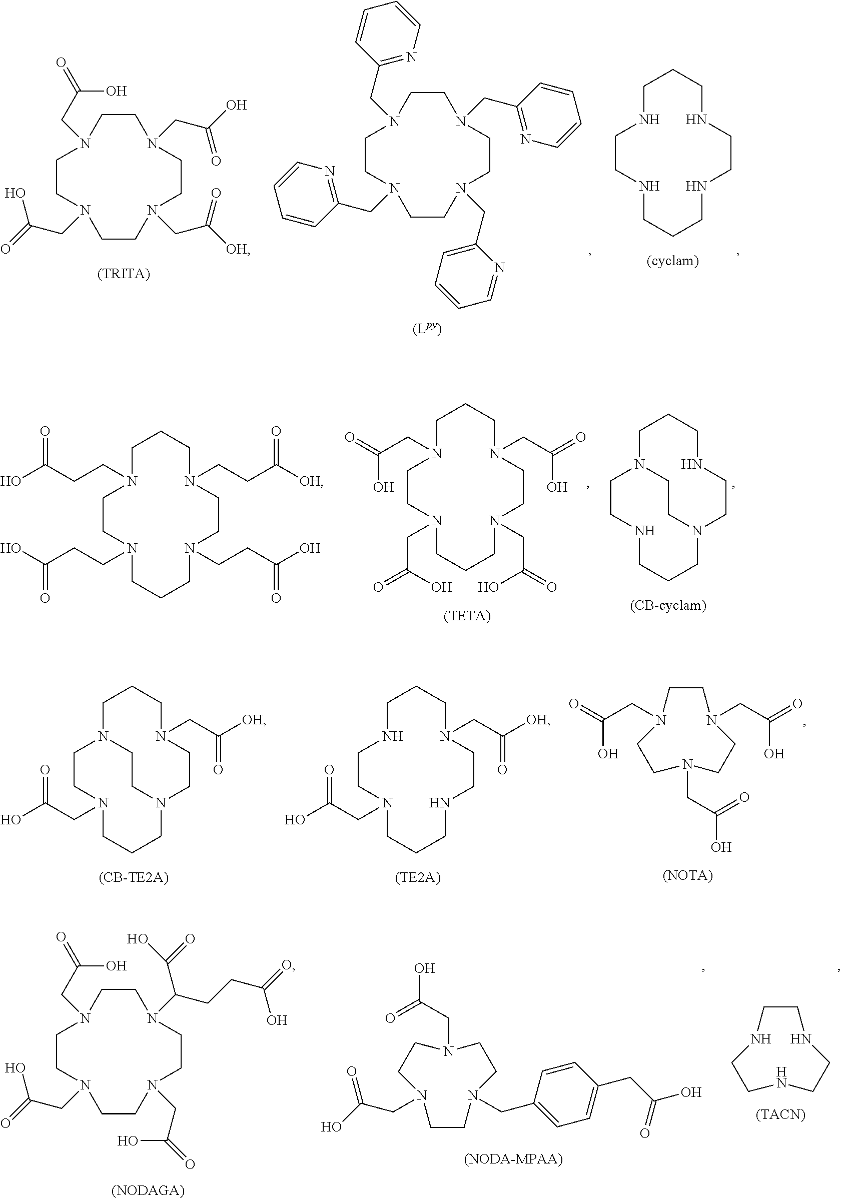

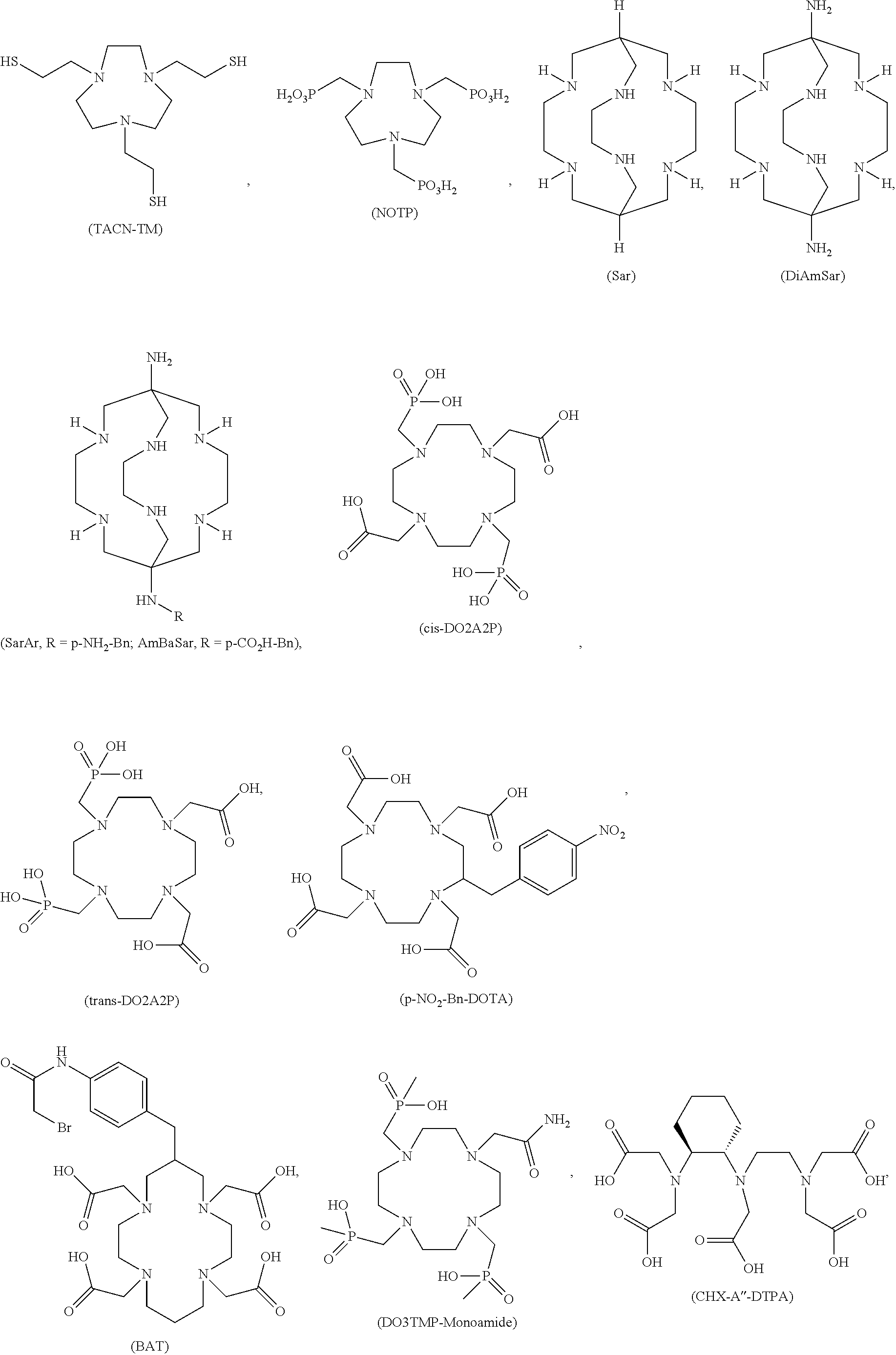

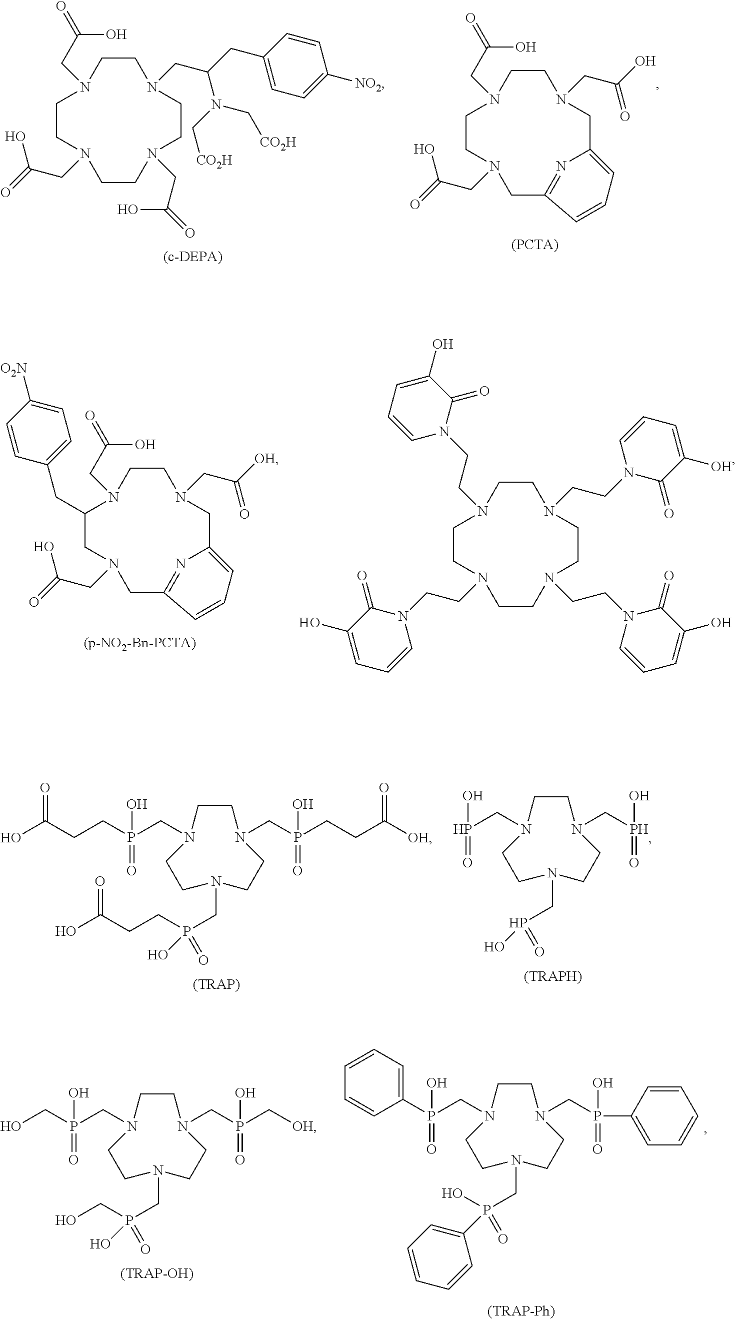

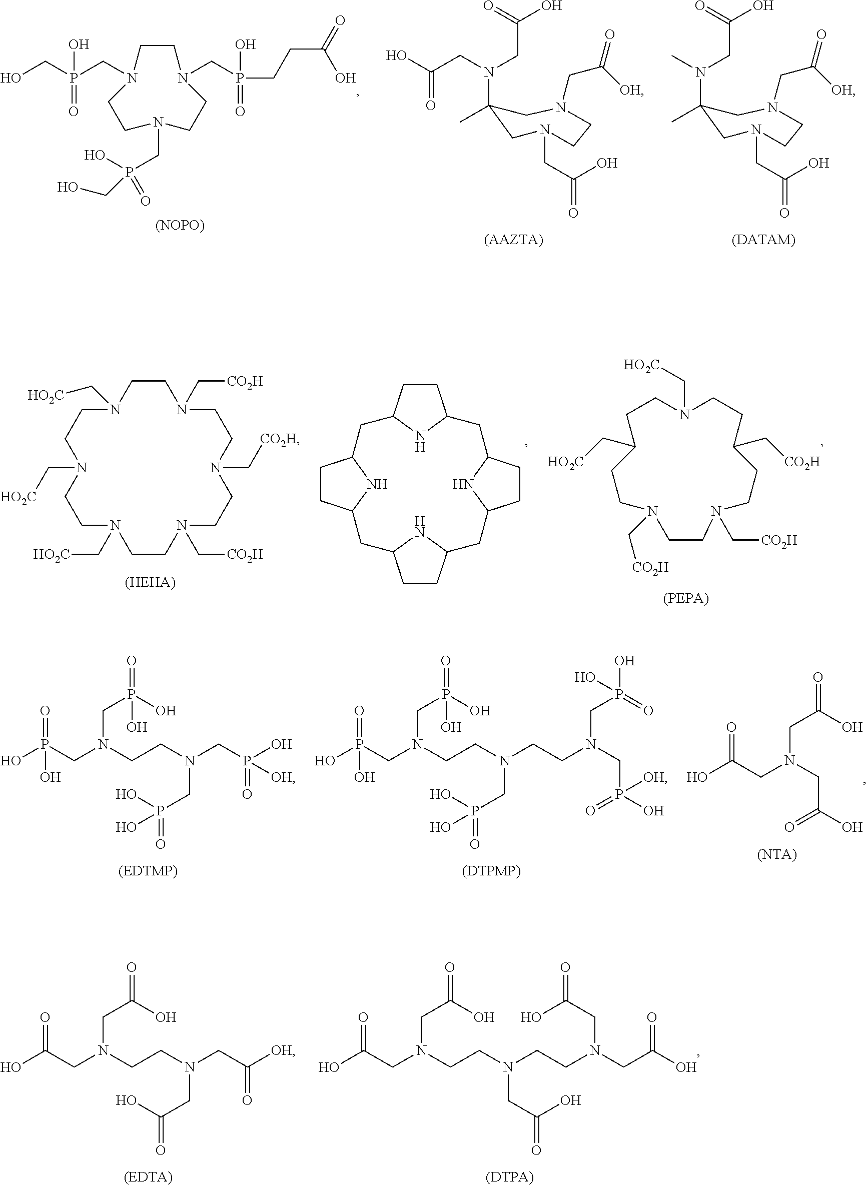

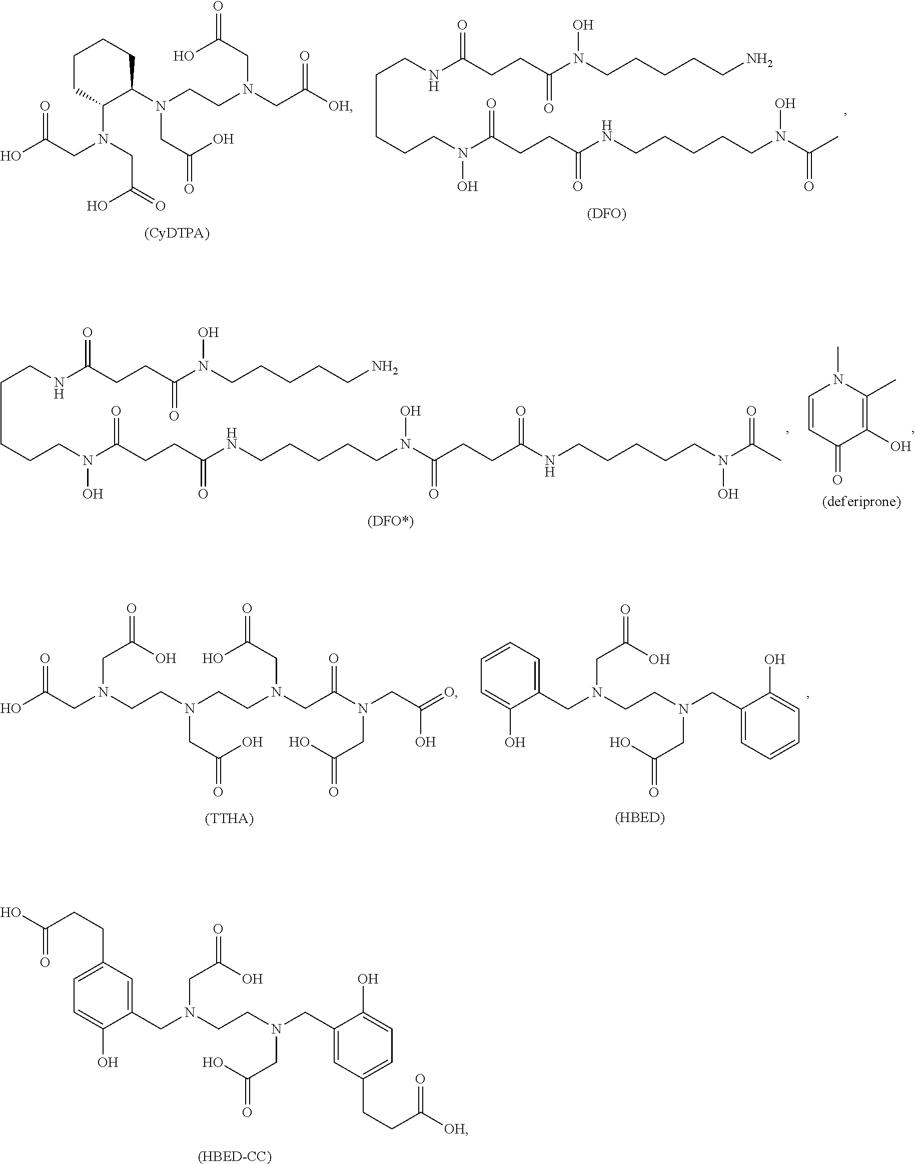

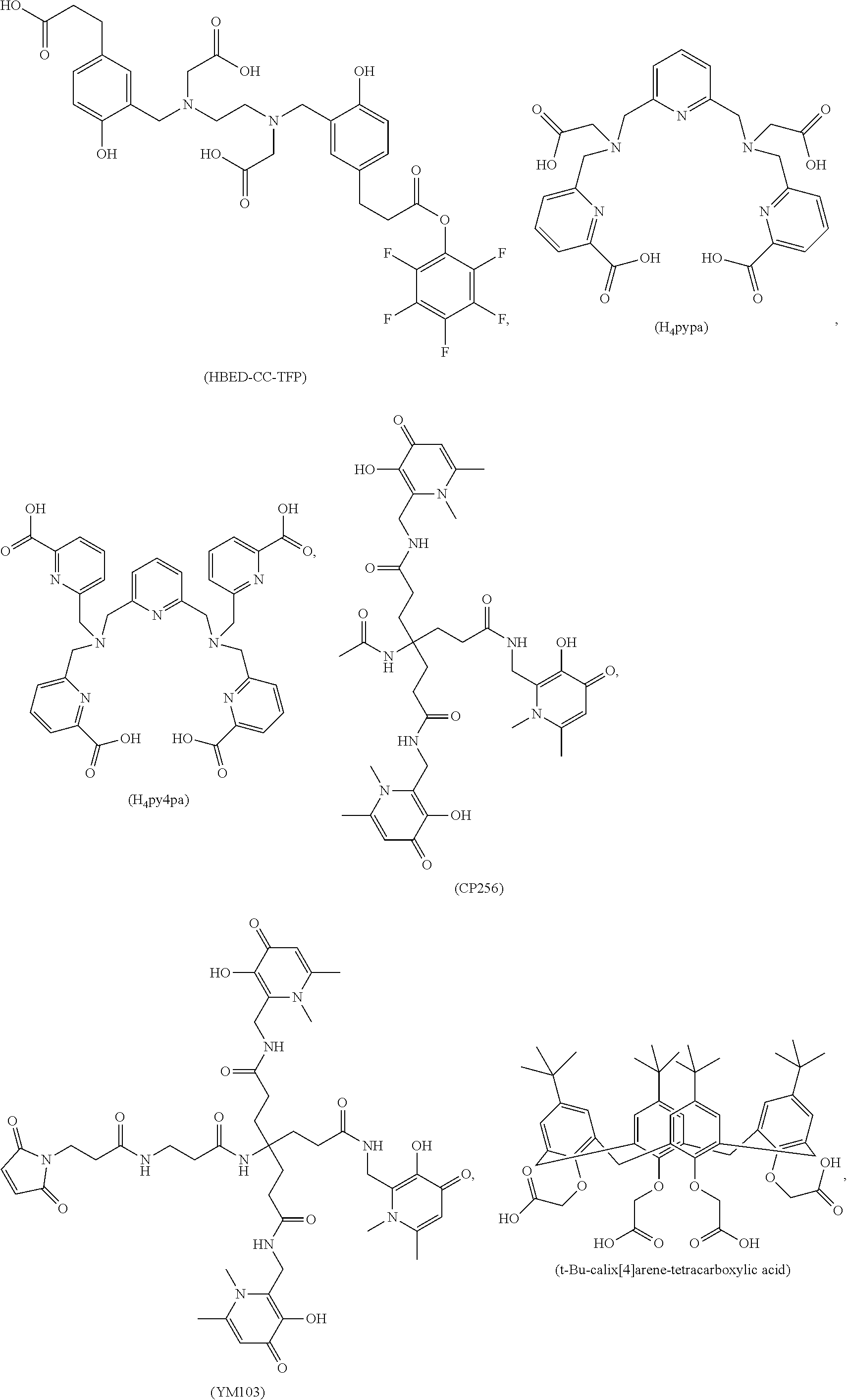

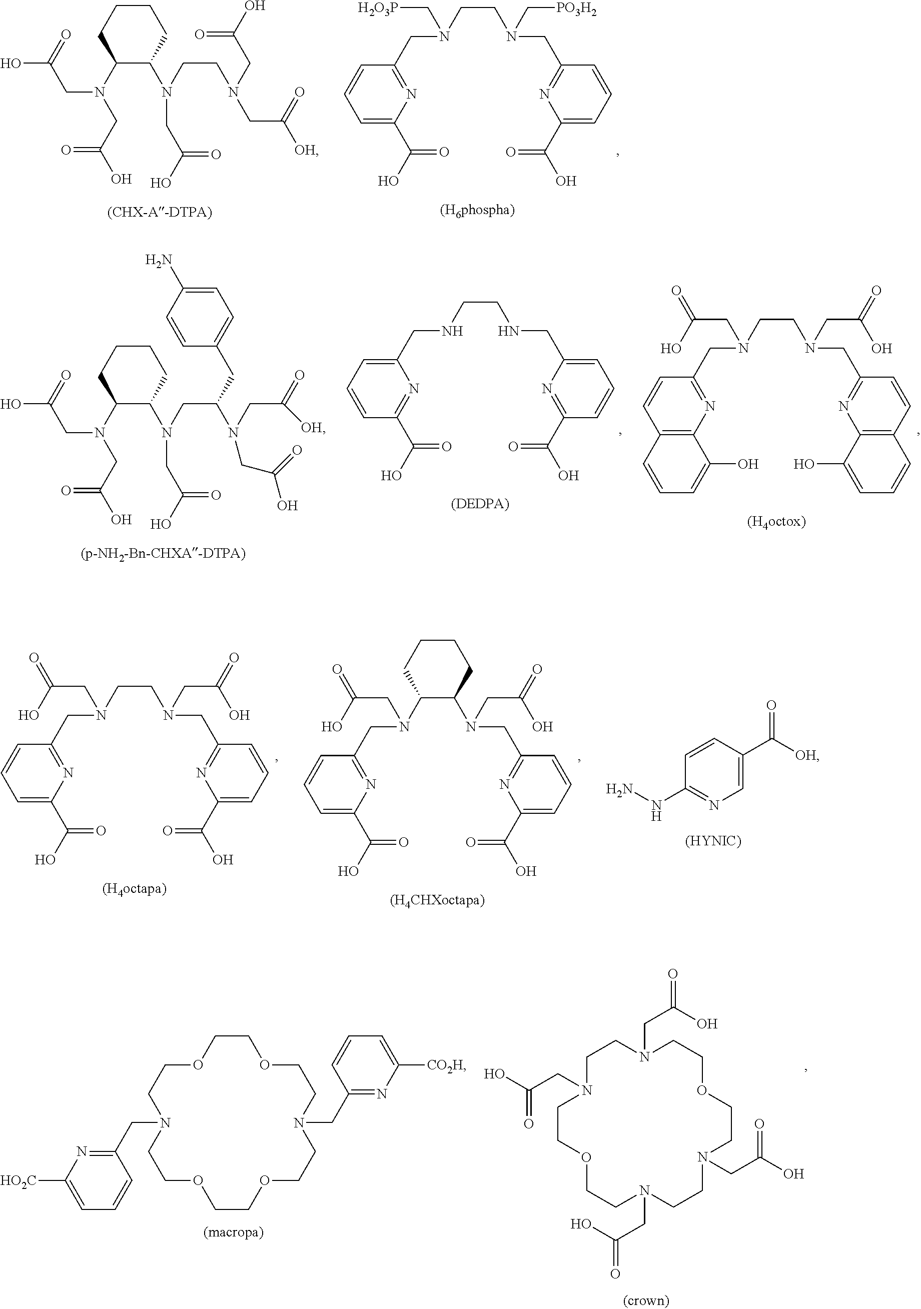

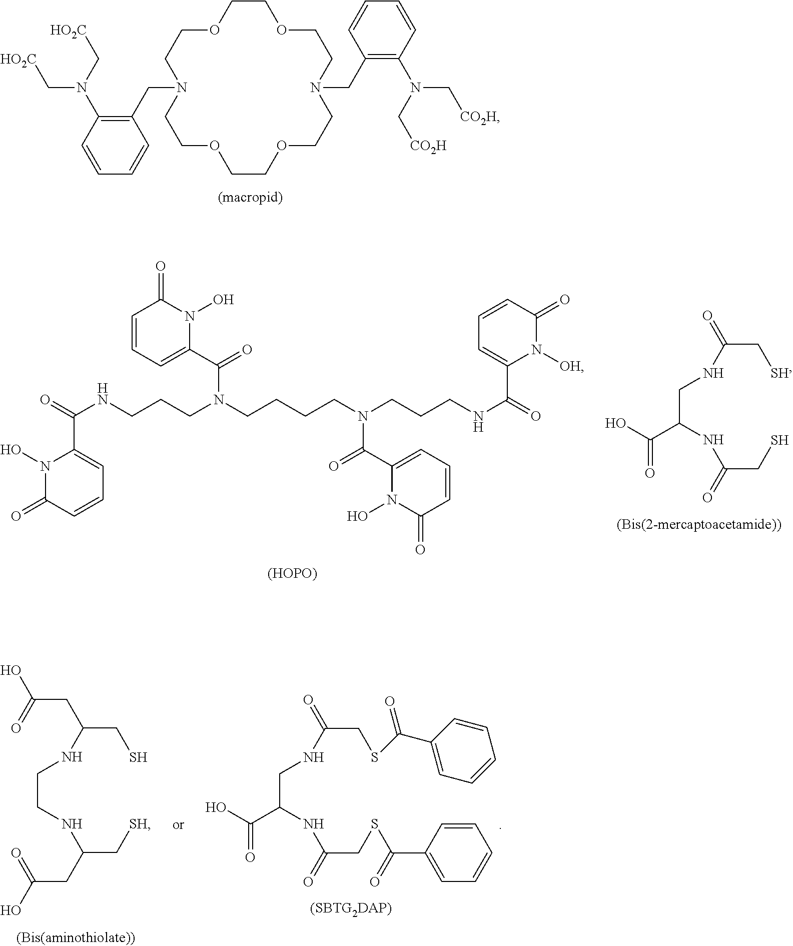 [SMILES: O=C(O)CN(CC(=O)O)c1ccccc1CN1CCOCCOCCN(Cc2ccccc2N(CC(=O)O)CC(=O)O)CCOCCOCC1 ; O=C(NCCCN(CCCCN(CCCNC(=O)c1cccc(=O)n1O)C(=O)c1cccc(=O)n1O)C(=O)c1cccc(=O)n1O)c1cccc(=O)n1O ; O=C(CS)NCC(NC(=O)CS)C(=O)O ; "(HOPO)" ; O=C(O)CC(CS)NCCNC(CS)CC(=O)O ; O=C(CSC(=O)c1ccccc1)NCC(NC(=O)CSC(=O)c1ccccc1)C(=O)O]

In some embodiments, the chelating moiety R^{a} comprises a radionuclide and DOTA. In some embodiments, the chelating moiety R^{a} comprises a radionuclide and a DOTA derivative. In some embodiments, the chelating moiety comprises two independent chelators, and at least one or both are DOTA.

In some embodiments, the chelating moiety comprises a radionuclide and a chelator configured to bind the radionuclide (Z′), wherein the chelator comprises DOTA, DOTP, DOTMA, DOTAM, DTPA, NOTA, NTA, NODAGA, EDTA, DO3A, DO2A, NOC, TETA, CB-TE2A, DiAmSar, CB-Cyclam, DOTA-4AMP, H₄pypa, H₄octox, H₄octapa, p-NO₂-Bn-neunpa, or NOTP.

In some embodiments, the metal chelator described herein comprises macropa or crown. In some embodiments, the metal chelator described herein comprises macropa. In some embodiments, the metal chelator described herein comprises crown. In some embodiments, the metal chelator described herein comprises

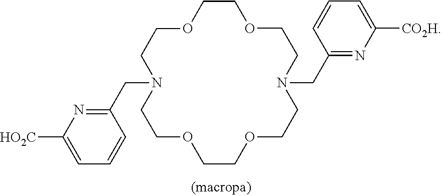 [SMILES: O=C(O)c1cccc(CN2CCOCCOCCN(Cc3cccc(C(=O)O)n3)CCOCCOCC2)n1]

In some embodiments, the metal chelator described herein comprises

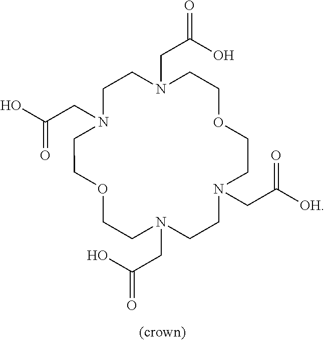 [SMILES: O=C(O)CN1CCOCCN(CC(=O)O)CCN(CC(=O)O)CCOCCN(CC(=O)O)CC1]

In some embodiments, R^{a} is a chelating moiety selected from the group consisting of: 1,4,7,10-tetraazacyclododecane-1,4,7,10-tetraacetic acid (DOTA); 2,2′,2″-(10-(2-amino-2-oxoethyl)-1,4,7,10-tetraazacyclododecane-1,4,7-triyl)triacetic acid (PSC); 1,4,7,10-tetraazacyclododecane-1,4,7-triacetic acid (DO3A); 1,4,7,10-tetraazacyclododecane-1,7-diacetic acid (DO2A); α,α′,α″,α′″-tetramethyl-1,4,7,10-tetraazacyclododecane-1,4,7,10-tetraacetic acid (DOTMA); 1,4,7,10-tetrakis(carbamoylmethyl)-1,4,7,10-tetraazacyclododecane (DOTAM); 1,4,7,10-tetraazacyclododecane-1,4,7,10-tetrapropionic acid (DOTPA); 2,2′,2″-(10-(2-amino-2-oxoethyl)-1,4,7,10-tetraazacyclododecane-1,4,7-triyl)triacetic acid; benzyl-1,4,7,10-tetraazacyclododecane-1,4,7,10-tetraacetic acid (Bn-DOTA); p-hydroxy-benzyl-1,4,7,10-tetraazacyclododecane-1,4,7,10-tetraacetic acid (p-OH-Bn-DOTA); 6,6′-(((pyridine-2,6-diylbis(methylene))bis((carboxymethyl)azanediyl))bis(methylene))dipicolinic acid (H₄pypa); H₄pypa-benzyl; 6,6′,6″,6′″-(((pyridine-2,6-diylbis(methylene))bis(azanetriyl))tetrakis(methylene))-tetrapicolinic acid (H₄py4pa); H₄py4pa-benzyl; 2,2′,2″-(1,4,7-triazacyclononane-1,4,7-triyl)triacetic acid (NOTA); 6,6′-((1,4,10,13-tetraoxa-7,16-diazacyclooctadecane-7,16-diyl)bis(methylene))dipicolinic acid (macropa); 2,2′,2″,2′″-(1,10-dioxa-4,7,13,16-tetraazacyclooctadecane-4,7,13,16-tetrayl)tetraacetic acid (crown); 6,6′-((ethane-1,2-diylbis((carboxymethyl)azanediyl))bis(methylene))dipicolinic acid (H₄octapa); H₄octapa-benzyl; and 3,6,9,12-tetrakis(carboxymethyl)-3,6,9,12-tetraazatetradecanedioic acid (TTHA); or a radionuclide complex thereof.

In some embodiments, R^{a} is a chelating moiety selected from the group consisting of: 1,4,7,10-tetraazacyclododecane-1,4,7,10-tetraacetic acid (DOTA) and 1,4,7,10-tetraazacyclododecane-1,4,7-triacetic acid (DO3A); or a radionuclide complex thereof.

In some embodiments, R^{a} is a chelating moiety selected from the group consisting of:

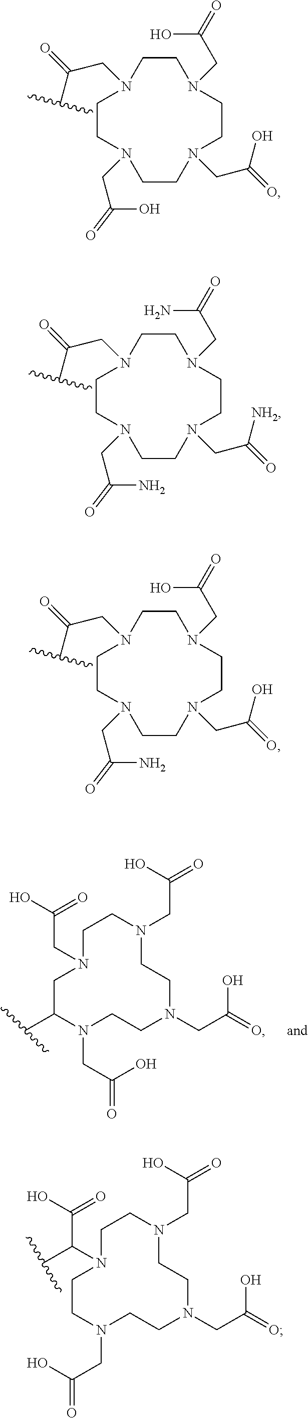 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, R^{a} is a chelating moiety selected from the group consisting of:

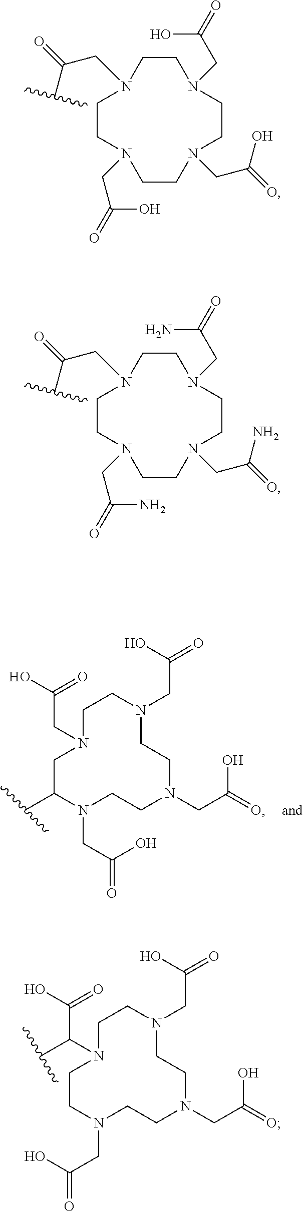 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C1CN(CC(=O)O)CCN(CC(=O)O)CCN(CC(=O)O)CCN1CC(=O)O ; *C(C(=O)O)N1CCN(CC(=O)O)CCN(CC(=O)O)CCN(CC(=O)O)CC1]

or a radionuclide complex thereof.

In some embodiments, R^{a} is:

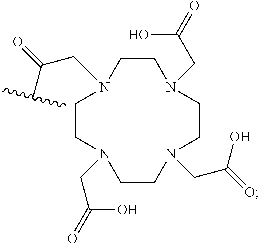 [SMILES: *C(=O)CN1CCN(CC(=O)O)CCN(CC(=O)O)CCN(CC(=O)O)CC1]

or a radionuclide complex thereof.

In some embodiments, R^{a} is:

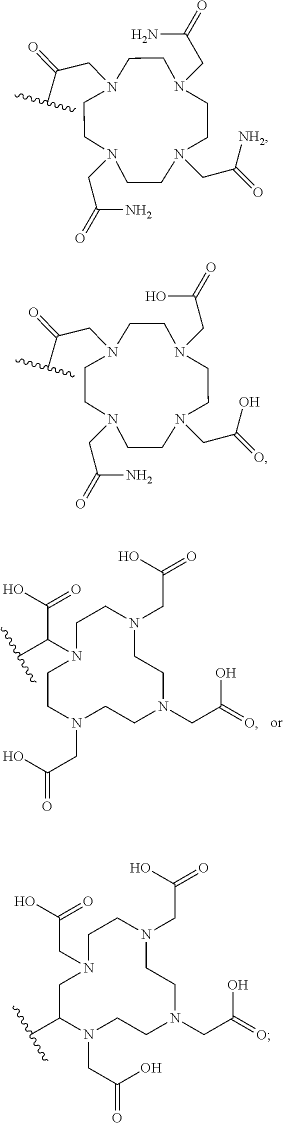 [SMILES: *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O]

or a radionuclide complex thereof.

In some embodiments, R^{a} is:

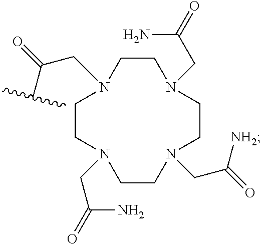 [SMILES: *C(=O)CN1CCN(CC(N)=O)CCN(CC(N)=O)CCN(CC(N)=O)CC1]

or a radionuclide complex thereof.
In some embodiments, R^{a} is:

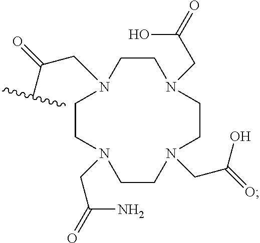 [SMILES: *C(=O)CN1CCN(CC(N)=O)CCN(CC(=O)O)CCN(CC(=O)O)CC1]

or a radionuclide complex thereof. In some embodiments, R^{a} is:

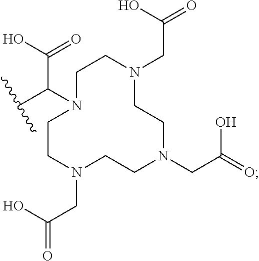 [SMILES: *C(C(=O)O)N1CCN(CC(=O)O)CCN(CC(=O)O)CCN(CC(=O)O)CC1]

or a radionuclide complex thereof. In some embodiments, R^{a} is:

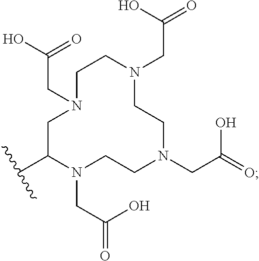 [SMILES: *C1CN(CC(=O)O)CCN(CC(=O)O)CCN(CC(=O)O)CCN1CC(=O)O]

or a radionuclide complex thereof.

In some embodiments, R^{a} is:

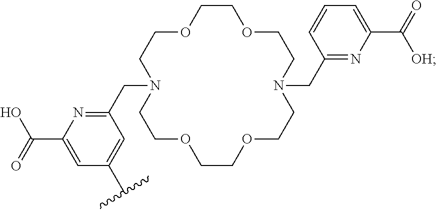 [SMILES: *c1cc(CN2CCOCCOCCN(Cc3cccc(C(=O)O)n3)CCOCCOCC2)nc(C(=O)O)c1]

or a radionuclide complex thereof. In some embodiments, R^{a} is:

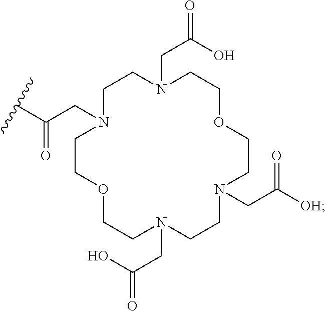 [SMILES: *C(=O)CN1CCOCCN(CC(=O)O)CCN(CC(=O)O)CCOCCN(CC(=O)O)CC1]

or a radionuclide complex thereof.

In some embodiments, R^{a} is:

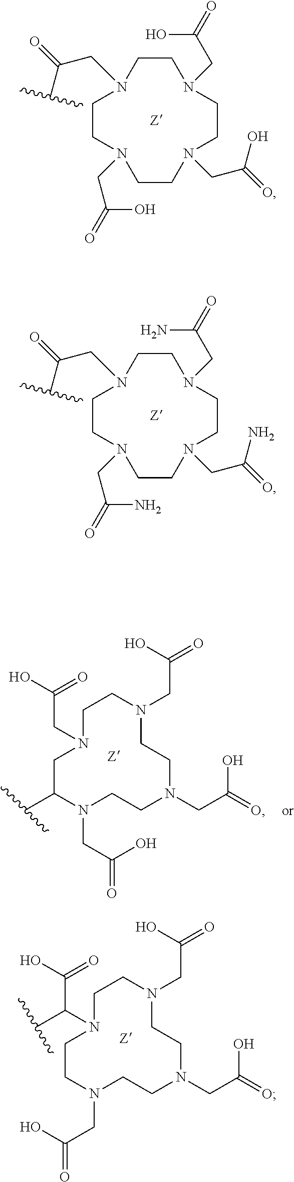

wherein Z′ is a diagnostic or therapeutic radionuclide.

In some embodiments, R^{a} is:

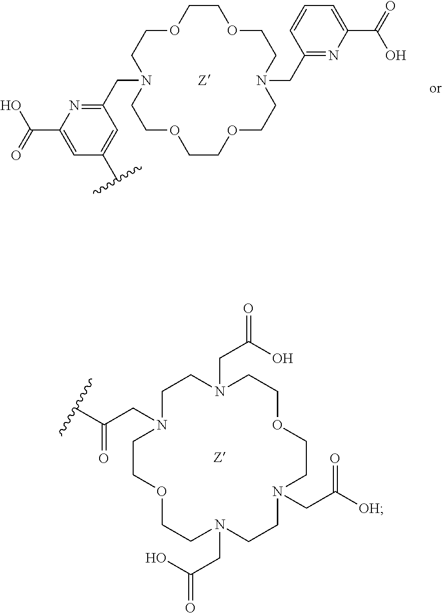

wherein Z′ is a diagnostic or therapeutic radionuclide.

In some embodiments, Z′ is an Auger electron-emitting radionuclide, α-emitting radionuclide, β-emitting radionuclide, or γ-emitting radionuclide. In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt). In some embodiments, Z′ is an α-emitting radionuclide that is 225-actinium (²²⁵Ac), 213-bismuth (²¹³Bi), 223-Radium (²²³Ra), or 212-lead (²¹²Pb). In some embodiments, Z′ is a β-emitting radionuclide that is 90-yttrium (⁹⁰Y) 177-lutetium (¹⁷⁷Lu), iodine-131 (¹³¹I), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn). In some embodiments, Z′ is a γ-emitting radionuclide that is 60-cobalt (⁶⁰Co), 103-palladium (¹⁰³Pd), 137-cesium (¹³⁷Cs), 169-ytterbium (¹⁶⁹Yb), 192-iridium (¹⁹²Ir), or 226-radium (²²⁶Ra).

In some embodiments, R^{a} comprises a radionuclide (Z′) and a chelator configured to bind the radionuclide (Z′), wherein the radionuclide is suitable for positron emission tomography (PET) analysis, single-photon emission computerized tomography (SPECT), or magnetic resonance imaging (MRI). In some embodiments, the radionuclide is copper-64 (⁶⁴Cu), gallium-68 (⁶⁸Ga), 111-indium (¹¹¹In), or technetium-99m (^{99m}Tc).

### Metals (Radionuclides)

In some embodiments, Z′ is an Auger electron-emitting radionuclide. In some embodiments, Z′ is an α-emitting radionuclide. In some embodiments, Z′ is a β-emitting radionuclide. In some embodiments, Z′ is a γ-emitting radionuclide. In some embodiments, the type of radionuclide used in a non-peptide targeted therapeutic compound can be tailored to the specific type of cancer, the type of targeting moiety (e.g., non-peptide ligand), etc. Radionuclides that undergo α-decay emit α-particles (helium ions with a +2 charge) from their nuclei. As a result of α-decay the daughter nuclide has 2 protons less and 2 neutrons less than the parent nuclide. This means that in α-decay, the proton number is reduced by 2 while the nucleon number is reduced by 4. Radionuclides that undergo β-decay emit β-particles (electrons) from their nuclei. During β-decay, one of the neutrons changes into a proton and an electron. The proton remains in the nucleus while the electron is emitted as a β-particle. This means that in β-decay, the nucleus loses a neutron but gains a proton. In γ-decay, a nucleus in an excited state (higher energy state) emits a γ-ray photon to change to a lower energy state. There is no change in the proton number and nucleon number during the γ-decay. The emission of γ-rays often accompanies the emission of α-particles and β-particles.

Auger electrons (AEs) are very low energy electrons that are emitted by radionuclides that decay by electron capture (EC) (e.g. ¹¹¹In, ⁶⁷Ga, ^{99m}Tc, ^{195m}Pt, ¹²⁵I and ¹²³I). This energy is deposited over nanometer-micrometer distances, resulting in high linear energy transfer that is potent for causing lethal damage in cancer cells. Thus, AE-emitting radiotherapeutic agents have great potential for treatment of cancer.

β-Particles are electrons emitted from the nucleus. They typically have a longer range in tissue (of the order of 1-5 mm) and are the most frequently used.

α-Particles are helium nuclei (two protons and two neutrons) that are emitted from the nucleus of a radioactive atom. Depending on their emission energy, they can travel 50-100 μm in tissue. They are positively charged and are orders of magnitude larger than electrons. The amount of energy deposited per path length travelled (designated ‘linear energy transfer’) of α-particles is approximately 400 times greater than that of electrons. This leads to substantially more damage along their path than that caused by electrons. An α-particle track leads to a preponderance of complex and largely irreparable DNA double-strand breaks. The absorbed dose required to achieve cytotoxicity relates to the number of α-particles traversing the cell nucleus. With use of this as a measure, cytotoxicity may be achieved with a range of 1 to 20 α-particle traversals of the cell nucleus. The resulting high potency, combined with the short range of α-particles (which reduces normal organ toxicity), has led to substantial interest in developing α-particle-emitting agents. The α-particle emitters typically used include bismuth-212, lead-212, bismuth-213, actinium-225, radium-223 and thorium-227.

In some embodiments, Z′ is a diagnostic or therapeutic radionuclide.

### Representative Radionuclides

| Isotope | Radionuclide t_{1/2} (h) | Decay mode |
|---|---|---|
| ⁶⁰Cu | 0.4 | β+ (93%), EC (7%) |
| ⁶¹Cu | 3.3 | β+ (62%), EC (38%) |
| ⁶²Cu | 0.16 | β+ (98%), EC (2%) |
| ⁶⁴Cu | 12.7 | β+ (19%), EC (41%), |
| ⁶⁷Cu | 61.9 | β− (40%) |
| ⁶⁶Ga | 9.5 | β+ (56%), EC (44%) |
| ⁶⁷Ga | 78.2 | EC (100%) |
| ⁶⁸Ga | 1.1 | β+ (90%), EC (10%) |
| ⁴⁴Sc | 3.9 | β+ (94%), EC (6%) |
| ⁴⁷Sc | 80.2 | β− (100%) |
| ¹¹¹In | 67.2 | EC (100%) |
| ^{114m}In | 49.5 | EC (100%) |
| | d | |
| ¹¹⁴In (daughter) | 73 | β− (100%) |
| | s | |
| ¹⁷⁷Lu | 159.4 | β− (100%) |
| ⁸⁶Y | 14.7 | β+ (33%), EC (66%) |
| ⁹⁰Y | 64.1 | β− (100%) |
| ⁸⁹Zr | 78.5 | β+ (23%), EC (77%) |
| ²¹²Bi | 1.1 | α (36%), β− (64%) |
| ²¹³Bi | 0.76 | α (2.2%), β− (97.8%) |
| ²¹²Pb (daughter is ²¹²Bi) | 10.6 | β− (100%) |
| ²²⁵Ac | 240 | α (100%) |
| ²²⁷Th | 448.8 | α |
| ²¹¹At | 7.2 | α |

In some embodiments, Z′ is an Auger electron-emitting radionuclide. In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt).

In some embodiments, Z′ is an α-emitting radionuclide. In some embodiments, Z′ is an C-emitting radionuclide that is 225-actinium (²²⁵Ac), 213-bismuth (²¹³Bi), 223-Radium (²²³Ra), or 212-lead (²¹²Pb).

In some embodiments, Z′ is an β-emitting radionuclide. In some embodiments, Z′ is a β-emitting radionuclide that is 90-yttrium (⁹⁰Y), 177-lutetium (¹⁷⁷Lu), 186-rhenium (¹⁸⁶Re), 188-rhenium (¹¹⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn).

In some embodiments, Z′ is a γ-emitting radionuclide. In some embodiments, Z′ is a 7-emitting radionuclide that is 60-cobalt (⁶⁰Co), 103-palladium (¹⁰³Pd), 137-cesium (¹³⁷Cs), 169-ytterbium (¹⁶⁹Yb) 192-iridium (¹⁹²Ir), or 226-radium (²²⁶Ra).

In some embodiments, Z′ is an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt); or Z′ is an α-emitting radionuclide that is 225-actinium (²²⁵Ac), 213-bismuth (²¹³Bi), 223-Radium (²²³Ra), or 212-lead (²¹²Pb); or Z′ is a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); Z′ is a γ-emitting radionuclide that is 60-cobalt (⁶⁰Co), 103-palladium (¹⁰³Pd), 137-cesium (¹³⁷Cs), 169-ytterbium (¹⁶⁹Yb) 192-iridium (¹⁹²Ir), or 226-radium (²²⁶Ra).

In some embodiments, Z′ is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

In some embodiments, Z′ is ⁹⁴Tc, ⁹⁰In, ¹¹In, ⁶⁷Ga, ⁶⁸Ga, ⁸⁶Y, ⁹⁰Y, ¹⁷⁷Lu, ¹⁶¹Tb, ¹⁸⁶Re, ¹⁸⁸Re, ⁶⁴Cu, ⁶⁷Cu ⁵⁵Co, ⁵⁷Co, ⁴³Sc, ⁴⁴Sc, ⁴⁷Sc, ²²⁵Ac, ²¹³Bi, ²¹²Bi, ²¹²Pb, ²²⁷Th, ¹⁵³Sm, ¹⁶⁶Ho, ¹⁵²Gd, ¹⁵³Gd, ¹⁵⁷Gd, and ¹⁶⁶Dy.

In some embodiments, Z′ is ⁶⁷Cu, ⁶⁴Cu, ⁹⁰Y, ¹⁰⁹Pd, ¹¹¹Ag, ¹⁴⁹Pm, ¹⁵³Sm, ¹⁶⁶Ho, ^{99m}Tc, ⁶⁷Ga, ⁶⁸Ga, ¹¹¹In, ⁹⁰Y, ¹⁷⁷Lu, ¹⁸⁶Re, ¹⁸⁸Re, ¹⁹⁷Au, ¹⁹⁸Au, ¹⁹⁹Au, ¹⁰⁵Rh, ¹⁶⁵Ho, ¹⁶¹Tb, ¹⁴⁹Pm, ⁴⁴Sc, ⁴⁷Sc, ⁷⁰As, ⁷¹As, ⁷²As, ⁷³As, ⁷⁴As, ⁷⁶As, ⁷⁷As, ²¹²Pb, ²¹²Bi, ²¹³Bi, ²²⁵Ac, ^{117m}Sn, ⁶⁷Ga, ²⁰¹Tl, ¹⁶⁰Gd, ¹⁴⁸Nd, and ⁸⁹Sr.

In some embodiments, Z′ is ⁶⁸Ga, ⁴³Sc, ⁴⁴Sc, ⁴⁷Sc, ¹⁷⁷Lu, ¹⁶¹Tb, ²²⁵Ac, ²¹³Bi, ²¹²Bi, or ²¹²Pb. In some embodiments, Z′ is ⁶⁷Ga, ^{99m}Tc, ¹¹¹In, or ²⁰¹Tl.

In some embodiments, the radionuclide (Z′) is ⁴⁴Sc, ⁶⁴Cu, ⁶⁷Ga, ⁶⁸Ga, ⁸⁶Y, ⁸⁹Zr, ^{99m}Tc, ¹¹¹In, or ¹⁷⁷Lu.

In some embodiments, Z′ is ⁴⁴Sc, ⁶⁴Cu, ⁶⁸Ga, ⁸⁶Y, or ⁸⁹Zr. In some embodiments, Z′ is ⁶⁷Ga, ^{99m}Tc, ¹¹¹In, ¹⁷⁷Lu.

In some embodiments, Z′ is ⁶⁷Cu, ⁹⁰Y, ¹¹¹In, ¹⁷⁷Lu, ²²⁵Ac, ²¹²Pb, or ²¹³Bi.

In some embodiments, Z′ is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

In some embodiments, Z′ is 111-indium (¹¹¹In). In some embodiments, Z′ is 115-indium (¹¹⁵In). In some embodiments, Z′ is 67-gallium (⁶⁷Ga). In some embodiments, Z′ is 68-gallium (⁶⁸Ga). In some embodiments, Z′ is 69-gallium (⁶⁹Ga), 71-gallium (⁷¹Ga), or a mixture thereof. In some embodiments, Z′ is 225-actinium (²²⁵Ac). In some embodiments, Z′ is 175-lutetium (¹⁷⁵Lu). In some embodiments, Z′ is 177-lutetium (¹⁷⁷Lu). In some embodiments, Z′ is 204-lead (²⁰⁴Pb), 206-lead (²⁰⁶Pb), 207-lead (²⁰⁷Pb), 208-lead (²⁰⁸Pb), or a mixture thereof. In some embodiments, Z′ is 212-lead (²¹²Pb). In some embodiments, Z′ is 64-copper (⁶⁴Cu). In some embodiments, Z′ is 63-copper (⁶³Cu), 65-copper (⁶⁵Cu), or a mixture thereof. In some embodiments, Z′ is 67-copper (⁶⁷Cu).

In some embodiments, Z′ is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

### Exemplary Chelator and Radionuclide Complexes

Radionuclides have useful emission properties that can be used for diagnostic imaging techniques, such as single photon emission computed tomography (SPECT, e.g. ⁶⁷Ga, ^{99m}Tc, ¹¹¹In, ¹⁷⁷Lu) and positron emission tomography (PET, e.g. ⁶⁸Ga, ⁶⁴Cu, ⁴⁴Sc, ⁸⁶Y, ⁸⁹Zr), as well as therapeutic applications (e.g. ⁴⁷Sc, ¹¹⁴mIn, ¹⁷⁷Lu, ⁹⁰Y, ^{212/213}Bi, ²¹²Pb, ²²⁵Ac, ^{186/188}Re). A fundamental component of a radiometal-based radiopharmaceutical is the chelator, the ligand system that binds the radiometal ion in a tight stable coordination complex so that it can be properly directed to a desirable molecular target in vivo. Guidance for selecting the optimal match between chelator and radiometal for a particular use is provided in the art (e.g., see Price et al., “Matching chelators to radiometals for radiopharmaceuticals”, Chem. Soc. Rev., 2014, 43, 260-290).

In some embodiments, R^{a} is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py4pa; H₄py4pa-benzyl; H₄octapa; H₄octapa-benzyl; and TTHA; or a radionuclide complex thereof.

In some embodiments, R^{a} is:

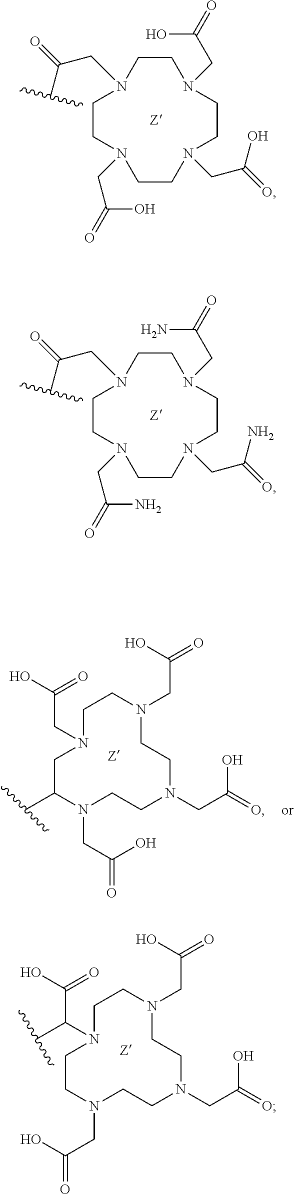

wherein Z′ is a diagnostic or therapeutic radionuclide.

In some embodiments, the radionuclide (Z′) is ⁴⁴Sc, ⁶⁴Cu, ⁶⁷Ga, ⁶⁸Ga, ⁸⁶Y, ⁸⁹Zr, ^{99m}Tc, ¹¹¹In, or ¹⁷⁷Lu. In some embodiments, the radionuclide (Z′) is ⁴⁴Sc, ⁶⁴Cu, ⁶⁸Ga, ⁸⁶Y, or ⁸⁹Zr. In some embodiments, the radionuclide (Z′) is ⁶⁷Ga, ^{99m}Tc, ¹¹¹In, ¹⁷⁷Lu.

In some embodiments, the radionuclide (Z′) is ⁶⁷Cu, ⁹⁰Y, ¹¹¹In, ¹⁷⁷Lu, ²²⁵Ac, ²¹²Pb, or 213Bi.

In some embodiments, the radionuclide (Z′) is 111-indium (¹¹¹In), 115-indium (¹¹⁵1n), 67-gallium (⁶⁷Ga), 68-gallium (⁶⁸Ga), 69-gallium (⁶⁹Ga), 70-gallium (⁷⁰Ga), 71-gallium (⁷¹Ga), 225-actinium (²²⁵Ac), 175-lutetium (¹⁷⁵Lu), 177-lutetium (¹⁷⁷Lu), 206-lead (²⁰⁶Pb), 207-lead (²⁰⁷Pb), 208-lead (²⁰⁸Pb), 212-lead (²¹²Pb), 60-copper (⁶⁰Cu), 61-copper (⁶¹Cu), 62-copper (⁶²Cu), 63-copper (⁶³Cu), 64-copper (⁶⁴Cu), 65-copper (⁶⁵Cu), or 67-copper (⁶⁷Cu).

In some embodiments, the radionuclide (Z′) is 111-indium (¹¹¹In) or 115-indium (¹¹⁵In), or a mixture thereof. In some embodiments, the radionuclide (Z′) is 67-gallium (⁶⁷Ga), 68-gallium (⁶⁸Ga), 69-gallium (⁶⁹Ga), 70-gallium (⁷⁰Ga), or 71-gallium (⁷¹Ga), or a mixture thereof. In some embodiments, the radionuclide (Z′) is 225-actinium (²²⁵Ac). In some embodiments, the radionuclide (Z′) is 175-lutetium (¹⁷⁵Lu) or 177-lutetium (¹⁷⁷Lu), or a mixture thereof. In some embodiments, the radionuclide (Z′) is 206-lead (²⁰⁶Pb), 207-lead (²⁰⁷Pb), 208-lead (²⁰⁸Pb), or 212-lead (²¹²Pb), or a mixture thereof. In some embodiments, the radionuclide (Z′) is 60-copper (⁶⁰Cu), 61-copper (⁶¹Cu), 62-copper (⁶²Cu), 63-copper (⁶³Cu), 64-copper (⁶⁴Cu), 65-copper (⁶⁵Cu), or 67-copper (⁶⁷Cu), or a mixture thereof.

In some embodiments, the radionuclide (Z′) is 111-indium (¹¹¹In), 115-indium (¹¹⁵In), 67-gallium (⁶⁷Ga), 68-gallium (⁶⁸Ga), 225-actinium (²²⁵Ac), 175-lutetium (¹⁷⁵Lu) or 177-lutetium (¹⁷⁷Lu).

In some embodiments, the radionuclide (Z′) is 90-yttrium (⁹⁰Y), 177-lutetium (¹⁷⁷Lu), 186-rhenium (¹⁸⁶Re), 188-rhenium (¹⁸⁸Re), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), or technetium-99m (^{99m}Tc).

### Emission Tomography

In some embodiments, R^{a} comprises a chelated radionuclide that is suitable for positron emission tomography (PET) analysis or single-photon emission computerized tomography (SPECT). In some embodiments, R^{a} comprises a chelated radionuclide that is suitable for single-photon emission computerized tomography (SPECT). In some embodiments, R^{a} comprises a chelated radionuclide that is suitable for positron emission tomography (PET) analysis. In some embodiments, R^{a} comprises a chelated radionuclide that is suitable for positron emission tomography imaging, positron emission tomography with computed tomography imaging, or positron emission tomography with magnetic resonance imaging (MRI).

In some embodiments, R^{a} is a chelating moiety selected from the group consisting of: DOTA; DO3A; DO2A; DOTMA; DOTAM; DOTPA; Bn-DOTA; p-OH-Bn-DOTA; H₄pypa; H₄pypa-benzyl; H₄py4pa; H₄py4pa-benzyl; H₄octapa; H₄octapa-benzyl; and TTHA; or a radionuclide complex thereof. In some embodiments, the radionuclide is copper-64 (⁶⁴Cu), gallium-68 (⁶⁸Ga), or technetium-99m (^{99m}Tc).

In some embodiments, a conjugate described herein is designed to have a prescribed elimination profile. The elimination profile can be designed by adjusting the sequence and length of the non-peptide ligand, the property of the linker, the type of radionuclide, etc. In some embodiments, the conjugate has an elimination half-life of about 5 minutes to about 12 hours. In some embodiments, the conjugate has an elimination half-life of about 10 minutes to about 8 hours. In some embodiments, the conjugate has an elimination half-life of at least about 15 minutes, at least about 30 minutes, at least about 1 hour, at least about 2 hours, at least about 3 hours, at least about 4 hours, at least about 5 hours, at least about 6 hours, at least about 8 hours. In some embodiments, the conjugate has an elimination half-life of at most about 15 minutes, at most about 30 minutes, at most about 1 hour, at most about 2 hours, at most about 3 hours, at most about 4 hours, at most about 5 hours, at most about 6 hours, or at most about 8 hours. In some embodiments, the elimination half-life is determined in rats. In some embodiments, the elimination half-life is determined in humans.

A herein described conjugate can have an elimination half-life in a tumor and non-tumor tissue of the subject. The elimination half-life in a tumor can be the same as or different from (either longer or shorter than) the elimination half-life in a non-tumor issue. In some embodiments, the elimination half-life of the conjugate in a tumor is about 15 minutes to about 1 day. In some embodiments, the elimination half-life of the conjugate in a tumor is at least 1.1, at least 1.2, at least 1.3, at least 1.4, at least 1.5, at least 2.0, at least 2.5, at least 3.0, at least 4.0, or at least 5.0-fold of the elimination half-life of the conjugate in a non-tumor tissue of the subject.

As used herein, the “elimination half-life” can refer to the time it takes from the maximum concentration after administration to half maximum concentration. In some embodiments, the elimination half-life is determined after intravenous administration. In some embodiments, the elimination half-life is measured as biological half-life, which is the half-life of the pharmaceutical in the living system. In some embodiments, the elimination half-life is measured as effective half-life, which is the half-life of a radiopharmaceutical in a living system taking into account the half-life of the radionuclide.

Response and toxicity prediction is essential for the rational implementation of cancer therapy. The biological effects of radionuclide therapy are mediated by a well-defined physical quantity, the absorbed dose (D), which is defined as the energy absorbed per unit mass of tissue.

Radiation dosimetry is the measurement, calculation and assessment of the ionizing radiation dose absorbed by an object, usually the human body, and may be thought of as the ability to perform the equivalent of a pharmacodynamic study in treated patients in real time. This applies both internally, due to ingested or inhaled radioactive substances, or externally due to irradiation by sources of radiation. Dosimetry analysis may be performed as part of patient treatment to calculate tumor versus normal organ absorbed dose and therefore the likelihood of treatment success.

A conjugate described herein can have a prescribed time-integrated activity coefficient (i.e., ã) in a tumor or non-tumor tissues of a subject. As used herein, a represents the cumulative number of nuclear transformations occurring in a source tissue over a dose-integration period per unit administered activity. The ã value of a conjugate can be tuned by modifications of the NPDC. The ã value can be determined using a method known in the art. In some embodiments, the ã value of the conjugate in a tumor is from about 10 minutes to about 1 day. The ã value of the conjugate in a tumor can be the same as the ã value of the conjugate in a non-tumor tissue of the subject. The ã value of the conjugate in a tumor can be longer or shorter than the ã value of the conjugate in a non-tumor tissue of the subject. In some embodiments, the ã value of the conjugate in a tumor is at least 1.1, at least 1.2, at least 1.3, at least 1.4, at least 1.5, at least 2.0, at least 2.5, at least 3.0, at least 4.0, or at least 5.0-fold of the ã value of the conjugate in a non-tumor tissue of the subject.

A conjugate described herein can have and value in an organ of a subject. In some embodiments, the conjugate has an ã value in a kidney of the subject of at most 24 hours. In some embodiments, the ã value of the conjugate in a kidney of the subject is at most 18 hours, 15 hours, 12 hours, 10 hours, 8 hours, 6 hours, or 5 hours. In some embodiments, the ã value of the conjugate in a kidney of the subject is about 30 minutes to about 24 hours. In some embodiments, the ã value of the conjugate in a kidney of the subject is about 2 to 24 hours. In some embodiments, the ã value of the conjugate in a kidney of the subject is more than 24 hours. In some embodiments, the ã value of the conjugate in a liver of the subject is at most 24 hours. In some embodiments, the ã value of the conjugate in a liver of the subject is at most 18 hours, 15 hours, 12 hours, 10 hours, 8 hours, 6 hours, or 5 hours. In some embodiments, the ã value of the conjugate in a liver of the subject is about 30 minutes to about 24 hours. In some embodiments, the ã value of the conjugate in a liver of the subject is about 2 to 24 hours. In some embodiments, the ã value of the conjugate in a liver of the subject is more than 24 hours.

### Linkers

In some embodiments, the linker has a prescribed length thereby linking the gonadotropin-releasing hormone receptor (GnRHR) targeting ligand and the chelating moiety or a radionuclide complex thereof (R^{a}) while allowing an appropriate distance therebetween.

In some embodiments, the linker is flexible. In some embodiments, the linker is rigid.

In some embodiments, the linker comprises a linear structure. In some embodiments, the linker comprises a non-linear structure. In some embodiments, the linker comprises a branched structure. In some embodiments, the linker comprises a cyclic structure.

In some embodiments, the linker comprises one or more linear structures, one or more non-linear structures, one or more branched structures, one or more cyclic structures, one or more flexible moieties, one or more rigid moieties, or combinations thereof.

In some embodiments, a linker comprises one or more amino acid residues. In some embodiments, the linker comprises 1 to 3, 1 to 5, 1 to 10, 5 to 10, or 5 to 20 amino acid residues. In some embodiments, one or more amino acids of the linker are unnatural amino acids.

In some embodiments, the linker comprises a peptide linkage. The peptide linkage comprises L-amino acids and/or D-amino acids. In some embodiments, D-amino acids are preferred in order to minimize immunogenicity and nonspecific cleavage by background peptidases or proteases. Cellular uptake of oligo-D-arginine sequences is known to be as good as or better than that of oligo-L-arginines.

In some embodiments, a linker has 1 to 100 atoms, 1 to 50 atoms, 1 to 30 atoms, 1 to 20 atoms, 1 to 15 atoms, 1 to 10 atoms, or 1 to 5 atoms in length. In some embodiments, the linker has 1 to 10 atoms in length. In some embodiments, the linker has 1 to 20 atoms in length.

In some embodiments, a linker can comprise flexible and/or rigid regions. Exemplary flexible linker regions include those comprising Gly and Ser residues (“GS” linker), glycine residues, alkylene chain, PEG chain, etc. Exemplary rigid linker regions include those comprising alpha helix-forming sequences, proline-rich sequences, and regions rich in double and/or triple bonds.

In some embodiments, the cleavable linker comprises one or more of substituted or unsubstituted alkylene, substituted or unsubstituted cycloalkylene, substituted or unsubstituted heterocycloalkylene, substituted or unsubstituted arylene, and substituted or unsubstituted heteroarylene.

In some embodiments, the linker comprises a click chemistry residue. In some embodiments, the linker is attached to a non-peptide ligand, to a metal chelator or both via click chemistry. For example, in some embodiments, a non-peptide ligand comprises an azide group that reacts with an alkyne moiety of the linker. For another example, in some embodiments, a non-peptide ligand comprises an alkyne group that reacts with an azide of the linker. The metal chelator and the linker can be attached similarly. In some embodiments, the linker comprises an azide moiety, an alkyne moiety, or both. In some embodiments, the linker comprises a triazole moiety.

In some embodiments, L is independently selected from: -L²-, -L³-, -L⁴-, -L⁵-, -L⁶-, -L⁷-, -L²-L³-, -L²-L³-R¹⁹, -L²-L⁴-, -L²-L⁷-, -L⁴-L⁶-, -L⁴-L⁷-, -L⁶-L⁷-, -L²-L⁴-L⁷-, -L²-L⁵- L⁷-, -L²-L⁶-L⁷-, -L³-L⁴-L⁷-, -L⁴-L⁵-L⁷-, or -L²-L³-L⁴-L⁵-L⁶-L⁷-; L² is absent, substituted or unsubstituted —C₁-C₂₀ alkylene, substituted or unsubstituted —C₁-C₂₀ alkylene-NR¹³—, substituted or unsubstituted —C₁-C₂₀ alkylene-C(═O)—, substituted or unsubstituted —C₁-C₂₀ alkylene-C(═O)NR¹³—, substituted or unsubstituted —C₁-C₂₀ alkylene-NR¹³C(═O)—, substituted or unsubstituted —C₁-C₂₀ alkylene-C(═O)NR¹³CH₂NR¹³—, substituted or unsubstituted —C₁-C₂₀ alkylene-NR¹³C(═O)CH₂NR¹³—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NR¹³—(CH₂CH₂O)_{w}—, —(CH₂CH₂O)_{w}—CH₂CH₂NR¹³—, —CH₂CH₂NHC(═O)—(CH₂CH₂O)_{w}, —(CH₂CH₂O)_{w}—CH₂CH₂NR¹³C(═O)—, —CH₂CH₂C(═O)NR¹³—(CH₂CH₂O)_{w}—, —CH₂CH₂NR¹³C(═O)CH₂—(OCH₂CH₂)_{w} or —(CH₂CH₂O)_{w}—CH₂CH₂C(═O)NR¹³—; R¹³ is H or unsubstituted —C₁-C₄ alkyl; w is 1, 2, 3, 4, 5, 6, 7 or 8; z is 1, 2, 3, 4, 5, 6, 7, 8, 9, or 10; L³ is absent, a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl; L⁴ is absent, substituted or unsubstituted 2 to 10 membered heteroalkylene, —CH₂—(OCH₂CH₂)ᵥ—, —(CH₂CH₂O)ᵥ—CH₂CH₂—, —(CH₂CH₂O)ᵥCH₂CH₂NR¹⁴C(═O)(CH₂CH₂O)ᵥCH₂CH₂—, —(CH₂CH₂O)ᵥCH₂CH₂C(═O)NR¹⁴(CH₂CH₂O)ᵥCH₂CH₂—, —C(═O)CH₂CH₂, —CH₂CH₂C(═O)—, —CH₂CH₂NR¹⁴CH₂CH₂, —CH₂CH₂NHC(═O)—CH—CH₂CH₂C(═O)NHR¹⁴, or —C₁-C₆ alkylene that is optionally substituted with 1 or 2 groups independently selected from —OR¹⁵, —NR¹⁵₂, —CO₂R¹⁵, —O(CH₂CH₂O)ₛ—CH₃, —NR¹⁵(CH₂CH₂O)ₛ—CH₃, —NR¹⁵C(═O)(CH₂CH₂O)ₛ—CH₃, or —CH₂OCH₂CH₂CO₂R¹⁵; R¹⁴ is H, —C₁-C₆ alkyl, or a sugar alcohol or derivative thereof, each R¹⁵ is independently selected from H or unsubstituted —C₁-C₄ alkyl; v is 1, 2, 3, 4, 5, 6, 7 or 8; s is 1, 2, 3, 4, 5, or 6; L⁵ is absent, —O—, —S—, —S(═O)—, —S(═O)₂, —NR¹⁶—, —CH(═NH)—, —CH(═N—NH)—, —CCH₃(═NH)—, —CCH₃(═N—NH)—, —C(═O)NR¹⁶—, —NR¹⁶C(═O), —NR¹⁶C(═O)O—, —NR¹⁶C(═O)NR¹⁶—, —OC(═O)NR¹⁶—, or —NHC(═O)—C₁-C₆ alkylene that is optionally substituted with 1 or 2 groups independently selected from —OR¹⁵, —NR¹⁵₂ or —CO₂R¹⁵; each R¹⁶ is independently selected from H or unsubstituted —C₁-C₄ alkyl; L⁶ is absent or -L⁸-L⁹-L¹⁰-; L⁸ is absent, —(CH₂)ᵣ—, —NR¹⁷—, —NR¹⁷—(CH₂)ᵣ—, —(CH₂)ᵣ—C(═O)—, —C(═O)—(CH₂)ᵣ—, —(CH₂)ᵣ—NR¹⁷—, —(CH₂)ᵣ—NR¹⁷C(═O)—, —(CH₂)ᵣ—C(═O)NR¹⁷—, —CH(NHR¹⁷)—(CH₂)ᵣ—C(═O)—, —NR¹⁷C(═O)—(CH₂)ᵣ—, and —C(═O)NR¹⁷—(CH₂)ᵣ—; r is 0, 1, 2, or 3; L⁹ is substituted or unsubstituted cycloalkylene, substituted or unsubstituted heterocycloalkylene, substituted or unsubstituted arylene, or substituted or unsubstituted heteroarylene; L¹⁰ is absent, —(CH₂)_{q}—, —NR¹⁸—, —NR¹⁸—(CH₂)_{q}—, —(CH₂)_{q}—C(═O)—, —C(═O)—(CH₂)_{q}—, —(CH₂)_{q}—NR¹⁸—, —(CH₂)_{q}—NR¹⁸C(═O)—, —(CH₂)_{q}—C(═O)NR¹⁸—, —CH(NHR¹⁸)—(CH₂)_{q}—C(═O)—, —NR¹⁸C(═O)—(CH₂)_{q}—, —C(═O)NR¹⁸—(CH₂)_{q}—, and —NR¹⁸—(CH₂)_{q}—NR¹⁸; q is 0, 1, 2, or 3; R¹⁷ and R¹⁸ are each independently selected from H, —C₁-C₆ alkyl, —C₁-C₆ alkyl-CO₂H, —(CH₂CH₂O)ₚ—CH₃, —C(═O)—(CH₂CH₂O)ₚ—CH₃, or —(CH₂CH₂O)ₚ—CH₂CH₂CO₂H; p is 1, 2, 3, 4, 5, or 6; L⁷ is absent, —NH—, —N(CH₃)—, —O—NH—, or substituted or unsubstituted N-heterocycloalkylene, or —O—NH=(substituted or unsubstituted N-heterocycloalkylene); R¹⁹ is —C(═O)(CH₂)ᵢ-substituted or unsubstituted phenyl; and i is 1, 2, 3, 4, 5, or 6.

In some embodiments, each L is independently selected from: -L²-, -L³-, -L⁴-, -L⁵-, -L⁶-, -L⁷-, -L²-L³-, -L²-L³-R¹⁹, -L²-L⁴-, -L²-L⁷-, -L⁴-L⁶-, -L⁴-L⁷-, -L⁶-L⁷-, -L²-L⁴-L⁷-, -L²-L⁵-L⁷-, -L²-L⁶-L⁷-, -L³-L⁴-L⁷-, -L⁴-L⁵-L⁷-, or -L²-L³-L⁴-L⁵-L⁶-L⁷-L² is substituted or unsubstituted —C₁-C₂₀ alkylene-C(═O)NR¹³—, substituted or unsubstituted —C₁-C₂M alkylene-NR¹³C(═O)—, or —(CH₂CH₂O)_{w}—CH₂CH₂—; R¹³ is H or unsubstituted —C₁-C₄ alkyl; w is 1, 2, 3, 4, 5, 6, 7 or 8; L³ is absent or a natural or unnatural amino acid or peptide that is formed from two or more independently selected natural and unnatural amino acids; L⁴ is absent, —(CH₂CH₂O)ᵥ—CH₂CH₂—, —CH₂CH₂NR¹⁴CH₂CH₂, or —C₁-C₆ alkylene that is optionally substituted with 1 or 2 groups independently selected from —OR¹⁵, —NR¹⁵₂ or —CO₂R¹⁵; R¹⁴ is H or —C₁-C₆ alkyl; each R^{15′} is independently selected from H or unsubstituted —C₁-C₄ alkyl; v is 1, 2, 3, 4, 5, 6, 7 or 8; L⁵ is absent or —NHC(═O)—C₁-C₆ alkylene that is optionally substituted with 1 or 2 groups independently selected from —OR¹⁵, —NR¹⁵₂ or —CO₂R¹⁵; L⁶ is absent; L⁷ is —NH—; R¹⁹ is —C(═O)(CH₂)ᵢ-substituted or unsubstituted phenyl; and i is 1, 2, 3, 4, 5, or 6.

In some embodiments, L is -L²-L³-, -L²-L³-R¹⁹, -L²-L⁷-, -L⁴-L⁶-, -L⁴-L⁷-, -L⁶-L⁷-, or -L²-L⁴-L⁷-. In some embodiments, L is -L²-. In some embodiments, L is -L³-. In some embodiments, L is -L⁴-. In some embodiments, L is -L⁵-. In some embodiments, L is -L⁶-. In some embodiments, L is -L⁷-. In some embodiments, L is -L²-L³-. In some embodiments, L is -L²-L³-R¹⁹. In some embodiments, L is -L²-L⁴-. In some embodiments, L is -L²-L⁷-. In some embodiments, L is -L⁴-L⁶-. In some embodiments, L is -L⁴-L⁷-. In some embodiments, L is -L⁶-L⁷-. In some embodiments, L is -L²-L⁴-L⁷-. In some embodiments, L is -L²-L⁵-L⁷-. In some embodiments, L is -L²-L⁶-L⁷-. In some embodiments, L is -L³-L⁴-L⁷-. In some embodiments, L is -L⁴-L⁵-L⁷-. In some embodiments, L is -L²-L³-L⁴-L⁵-L⁶-L⁷-.

In some embodiments, L² is substituted or unsubstituted —C₁-C₂₀ alkylene-C(═O)NR¹³—, substituted or unsubstituted —C₁-C₂₀ alkylene-NR¹³C(═O)—, or —(CH₂CH₂O)_{w}—CH₂CH₂—. In some embodiments, L² is unsubstituted —C₁-C₆alkylene-C(═O)NH—, unsubstituted —C₁-C₆ alkylene-NHC(═O)—, or —(CH₂CH₂O)_{w}—CH₂CH₂—. In some embodiments, L² is absent. In some embodiments, L² is substituted or unsubstituted —C₁-C₂₀ alkylene. In some embodiments, L² is substituted or unsubstituted —C₁-C₂₀ alkylene-NR³—. In some embodiments, L² is substituted or unsubstituted —C₁-C₂₀ alkylene-C(═O)—. In some embodiments, L² is substituted or unsubstituted —C₁-C₂₀ alkylene-C(═O)NR¹³—. In some embodiments, L² is unsubstituted —C₁-C₆ alkylene-C(═O)NH—. In some embodiments, L² is substituted or unsubstituted —C₁-C₂₀ alkylene-NR¹³C(═O)—. In some embodiments, L² is unsubstituted —C₁-C₆ alkylene-NHC(═O)—. In some embodiments, L² is substituted or unsubstituted —C₁-C₂₀ alkylene-C(═O)NR¹³CH₂NR¹³—. In some embodiments, L² is substituted or unsubstituted —C₁-C₂₀ alkylene-NR¹³C(═O)CH₂NR¹³—. In some embodiments, L² is substituted or unsubstituted 2 to 20 membered heteroalkylene. In some embodiments, L² is —(CH₂CH₂O)_{z}—. In some embodiments, L² is —(OCH₂CH₂)_{z}—. In some embodiments, L² is —(CH₂CH₂O)_{w}—CH₂CH₂—. In some embodiments, L² is —CH₂CH₂NR¹³—(CH₂CH₂O)_{w}—. In some embodiments, L² is —(CH₂CH₂O)_{w}—CH₂CH₂NR¹³—. In some embodiments, L² is —(CH₂CH₂O)_{w}—CH₂CH₂NH—. In some embodiments, L² is —CH₂CH₂NHC(═O)—(CH₂CH₂O)_{w}. In some embodiments, L² is —(CH₂CH₂O)_{w}—CH₂CH₂NR¹³C(═O)—. In some embodiments, L² is —CH₂CH₂C(═O)NR³—(CH₂CH₂O)_{w}—. In some embodiments, L² is —CH₂CH₂NR¹³C(═O)CH₂—(OCH₂CH₂)_{w}. In some embodiments, L² is —(CH₂CH₂O)_{w}—CH₂CH₂C(═O)NR¹³.

In some embodiments, w is 1. In some embodiments, w is 2. In some embodiments, w is 3. In some embodiments, w is 4. In some embodiments, w is 5. In some embodiments, w is 6. In some embodiments, w is 7. In some embodiments, w is 8.

In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8.

In some embodiments, L³ is absent. In some embodiments, L³ is a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl. In some embodiments, L³ is a natural amino acid, an unnatural amino acid, or peptide that is formed from two or more independently selected amino acids selected from the group consisting of alanine (Ala), arginine (Arg), asparagine (Asn), aspartate (Asp), cysteine (Cys), cysteic acid, glutamine (Gln), glutamate (Glu), glycine (Gly), leucine (Leu), lysine (Lys), methionine (Met), phenylalanine (Phe), proline (Pro), serine (Ser), sarcosine, tyrosine (Tyr), and valine (Val), wherein when two or more amino acids are present then the N atom of the amide linking the amino acids is optionally substituted with —CH₃. In some embodiments, the peptide is formed from one or more independently selected L-amino acids. In some embodiments, the peptide is formed from one or more independently selected D-amino acids. In some embodiments, the peptide is formed from one or more independently selected L-amino acids and one or more independently selected D-amino acids. In some embodiments, L³ is a tripeptide. In some embodiments, L³ is a dipeptide. In some embodiments, L³ is Lys-Glu. In some embodiments, L³ is Lys-Glu, wherein R^{a} is attached to the linker via the nitrogen atom of the Lysine side chain.

In some embodiments, L⁴ is —(CH₂CH₂O)ᵥ—CH₂CH₂—, —CH₂CH₂NR¹⁴CH₂CH₂, or —C₁-C₆ alkylene that is optionally substituted with 1 or 2 groups independently selected from —OR¹⁵ or —NR¹⁵₂.

In some embodiments, L⁴ is absent. In some embodiments, L⁴ is substituted or unsubstituted 2 to 10 membered heteroalkylene. In some embodiments, L⁴ is —CH₂—(OCH₂CH₂)ᵥ—. In some embodiments, L⁴ is —(CH₂CH₂O)ᵥ—CH₂CH₂—. In some embodiments, L⁴ is —(CH₂CH₂O)ᵥCH₂CH₂NR¹⁴C(═O)(CH₂CH₂O)ᵥCH₂CH₂—. In some embodiments, L⁴ is —(CH₂CH₂O)ᵥCH₂CH₂C(═O)NR¹⁴(CH₂CH₂O)ᵥCH₂CH₂—. In some embodiments, L⁴ is —C(═O)CH₂CH₂, —CH₂CH₂C(═O)—. In some embodiments, L⁴ is —CH₂CH₂NR¹⁴CH₂CH₂. In some embodiments, L⁴ is —CH₂CH₂NHC(═O)—CH—CH₂CH₂C(═O)NHR¹⁴. In some embodiments, L⁴ is —C₁-C₆ alkylene that is optionally substituted with 1 or 2 groups independently selected from —OR¹⁵, —NR¹⁵₂, —CO₂R¹⁵, —O(CH₂CH₂O)ₛ—CH₃, —NR¹⁵(CH₂CH₂O)ₛ—CH₃, —NR¹⁵C(═O)(CH₂CH₂O)ₛ—CH₃, or —CH₂OCH₂CH₂CO₂R¹⁵. In some embodiments, L⁴ is —C₁-C₆ alkylene that is substituted with 1 or 2 groups independently selected from —OR¹⁵ or —NR⁵2. In some embodiments, v is 1 or 2.

In some embodiments, R¹⁴ is H. In some embodiments, R¹⁴ is —CH₃.

In some embodiments, R¹⁵ is H. In some embodiments, R¹⁵ is —CH₃.

In some embodiments, L⁵ is absent. In some embodiments, L⁵ is —O—. In some embodiments, L⁵ is —S—. In some embodiments, L⁵ is —S(═O)—. In some embodiments, L⁵ is —S(═O)₂. In some embodiments, L⁵ is —NR¹⁶—. In some embodiments, L⁵ is —CH(═NH)—. In some embodiments, L⁵ is —CH(═N—NH)—. In some embodiments, L⁵ is —CCH₃(═NH)—. In some embodiments, L⁵ is —CCH₃(═N—NH)—. In some embodiments, L⁵ is —C(═O)NR¹⁶—. In some embodiments, L⁵ is —NR¹⁶C(═O). In some embodiments, L⁵ is —NR¹⁶C(═O)O—. In some embodiments, L⁵ is —NR¹⁶C(═O)NR¹⁶—. In some embodiments, L⁵ is —OC(═O)NR¹⁶—. In some embodiments, L⁵ is —NHC(═O)—C₁-C₆ alkylene that is optionally substituted with 1 or 2 groups independently selected from —OR¹⁵, —NR¹⁵₂ or —CO₂R¹⁵. In some embodiments, L⁵ is —NHC(═O)—C₁-C₆ alkylene that is optionally substituted with 1 or 2 —OH or —NH₂.

In some embodiments, R¹⁶ is H. In some embodiments, R¹⁶ is —CH₃.

In some embodiments, L⁶ is absent. In some embodiments, wherein L⁶ is -L⁸-L⁹-L¹⁰-.

In some embodiments, L⁸ is absent. In some embodiments, L⁸ is —(CH₂)ᵣ. In some embodiments, L⁸ is —(CH₂)ᵣ—. In some embodiments, L⁸ is —NR¹⁷—. In some embodiments, L⁸ is —NR¹⁷—(CH₂)ᵣ—. In some embodiments, L⁸ is —(CH₂)ᵣ—C(═O)—. In some embodiments, L⁸ is —C(═O)—(CH₂)ᵣ—. In some embodiments, L⁸ is —(CH₂)ᵣ—NR¹⁷—. In some embodiments, L⁸ is —(CH₂)ᵣ—NR¹⁷C(═O)—. In some embodiments, L⁸ is —(CH₂)ᵣ—C(═O)NR¹⁷—. In some embodiments, L⁸ is —CH(NHR¹⁷)—(CH₂)ᵣ—C(═O)—. In some embodiments, L⁸ is —NR¹⁷C(═O)—(CH₂)ᵣ—. In some embodiments, L⁸ is —C(═O)NR¹⁷—(CH₂)ᵣ—.

In some embodiments, r is 1 or 2.

In some embodiments, L⁹ is a substituted or unsubstituted heterocycloalkylene. In some embodiments, L⁹ is a substituted or unsubstituted 3 to 8 membered heterocycloalkylene. In some embodiments, L⁹ is a substituted or unsubstituted 4 to 6 membered heterocycloalkylene. In some embodiments, L⁹ is azetidinylene, pyrrolidinylene, piperidinylene or piperazinylene. In some embodiments, L⁹ is a monosaccharide. In some embodiments, L⁹ is

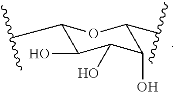 [SMILES: *C1OC(*)C(O)C(O)C1O]

In some embodiments, L⁹ is a substituted or unsubstituted cycloalkylene. In some embodiments, L⁹ is a substituted or unsubstituted C₄-C₈ cycloalkylene. In some embodiments, L⁹ is

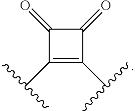 [SMILES: *c1c(*)c(=O)c1=O]

In some embodiments, L⁹ is a substituted or unsubstituted arylene. In some embodiments, L⁹ is phenylene. In some embodiments, L⁹ is a substituted or unsubstituted heteroarylene.

In some embodiments, L¹⁰ is absent. In some embodiments, L¹⁰ is —(CH₂)_{q}—. In some embodiments, L¹⁰ is —NR¹⁸—. In some embodiments, L¹⁰ is —NR¹⁸—(CH₂)_{q}—. In some embodiments, L¹⁰ is —(CH₂)_{q}—C(═O)—. In some embodiments, L¹⁰ is —C(═O)—(CH₂)_{q}—. In some embodiments, L¹⁰ is —(CH₂)_{q}—NR¹⁸—. In some embodiments, L¹⁰ is —(CH₂)_{q}—NR¹⁸C(═O)—. In some embodiments, L¹⁰ is —(CH₂)_{q}—C(═O)NR¹⁸—. In some embodiments, L¹⁰ is —CH(NHR¹⁸)—(CH₂)_{q}—C(═O)—. In some embodiments, L¹⁰ is —NR¹⁸C(═O)—(CH₂)_{q}—. In some embodiments, L¹⁰ is —C(═O)NR¹⁸—(CH₂)_{q}—. In some embodiments, L¹⁰ is —NR¹⁸—(CH₂)_{q}—NR¹⁸—. In some embodiments, R¹⁸ is H. In some embodiments, q is 1 or 2. In some embodiments, L¹⁰ is —CH₂—.

In some embodiments, L⁷ is absent. In some embodiments, L⁷ is —NH—.

In some embodiments, R¹⁹ is —C(═O)(CH₂)ᵢ-substituted or unsubstituted phenyl. In some embodiments, R¹⁹ is —C(═O)(CH₂)ᵢ-4-halo-phenyl. In some embodiments, R¹⁹ is

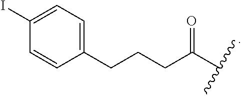 [SMILES: *C(=O)CCCc1ccc(I)cc1]

In some embodiments, i is 1. In some embodiments, i is 2. In some embodiments, i is 3. In some embodiments, i is 4. In some embodiments, i is 5. In some embodiments, i is 6.

In some embodiments, v is 1, 2, 3, 4, 5, 6, 7 or 8. In some embodiments, v is 1. In some embodiments, v is 2. In some embodiments, v is 3. In some embodiments, v is 4. In some embodiments, v is 5. In some embodiments, v is 6. In some embodiments, v is 7. In some embodiments, v is 8.

In some embodiments, s is 1. In some embodiments, s is 2. In some embodiments, s is 3. In some embodiments, s is 4. In some embodiments, s is 5. In some embodiments, s is 6.

In some embodiments, r is 0. In some embodiments, r is 1. In some embodiments, r is 2. In some embodiments, r is 3.

In some embodiments, q is 0. In some embodiments, q is 1. In some embodiments, q is 2. In some embodiments, q is 3.

In some embodiments, L is -L²-L⁷-; L² is —(CH₂CH₂O)_{w}—CH₂CH₂—; and L⁷-NH—.

In some embodiments, L is -L²-L⁴-L⁷-; L² is substituted or unsubstituted —C₁-C₂₀ alkylene-C(═O)NR¹³— or substituted or unsubstituted —C₁-C₂₀ alkylene-NR¹³C(═O)—; L⁴ is —(CH₂CH₂O)ᵥ—CH₂CH₂—, —CH₂CH₂NR¹⁴CH₂CH₂—, or optionally substituted —C₁-C₆ alkylene; and L⁷ is —NH—.

In some embodiments, L-R^{a} is -L²-L⁷-R^{a}; L² is —(CH₂CH₂O)_{w}—CH₂CH₂—; and L⁷-NH—.

In some embodiments, L-R^{a} is -L²-L⁴-L⁷-R^{a}; L² is substituted or unsubstituted —C₁-C₂₀ alkylene-C(═O)NR¹³— or substituted or unsubstituted —C₁-C₂₀ alkylene-NR¹³C(═O)—; L⁴ is —(CH₂CH₂O)ᵥ—CH₂CH₂—, —CH₂CH₂NR¹⁴CH₂CH₂—, or optionally substituted —C₁-C₆ alkylene; and L⁷ is —NH—.

In some embodiments, -L- is:

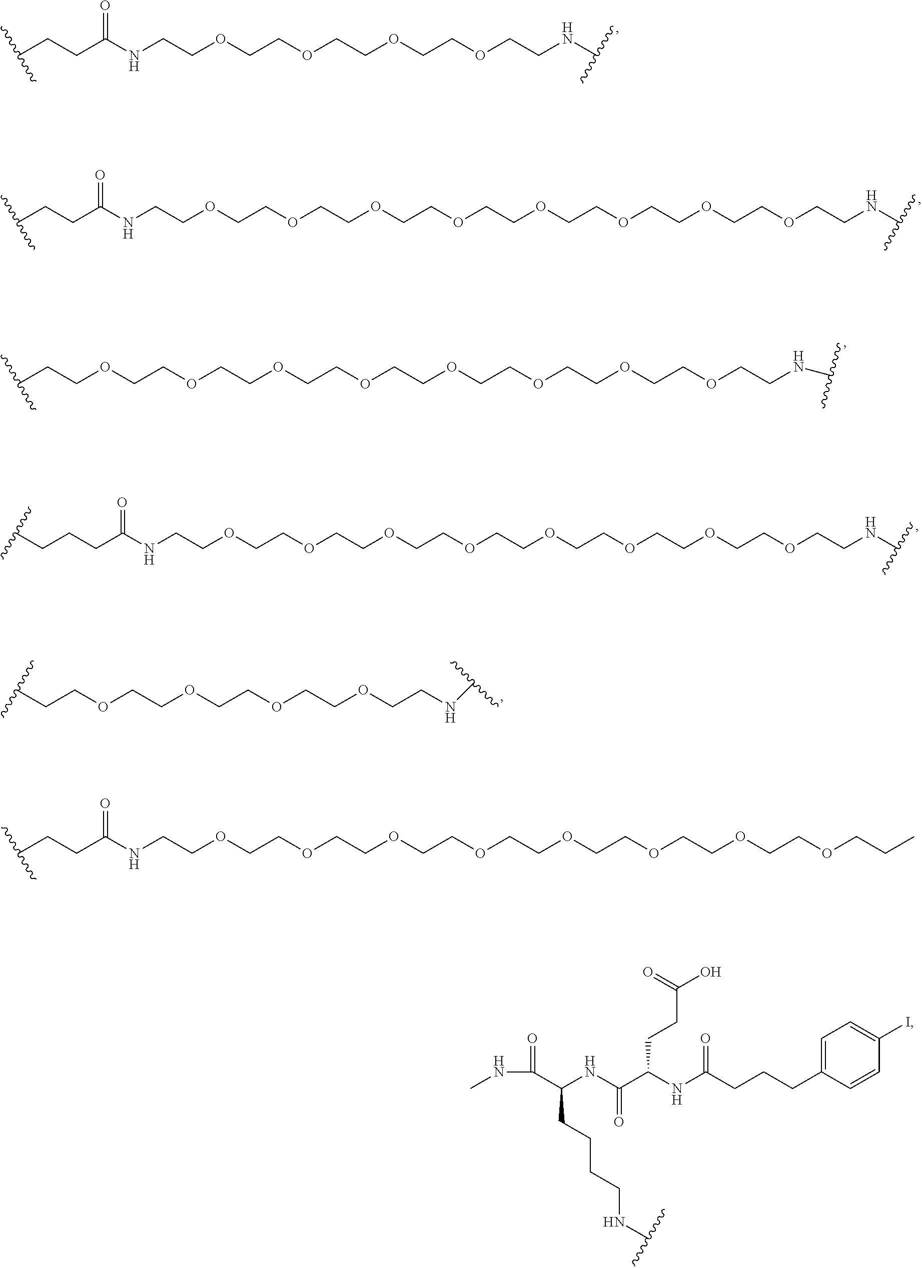

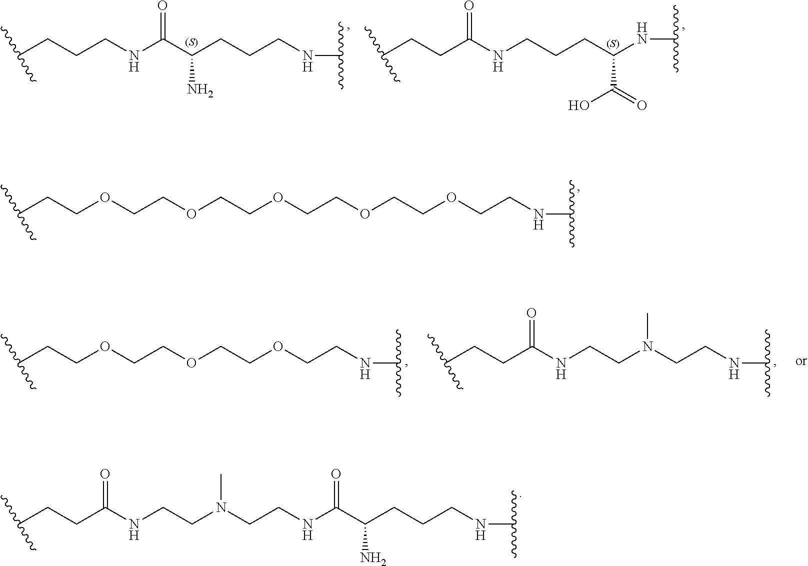

In some embodiments, -L- is:

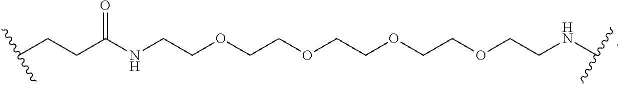 [SMILES: *CCC(=O)NCCOCCOCCOCCOCCN*]

In some embodiments, -L- is:

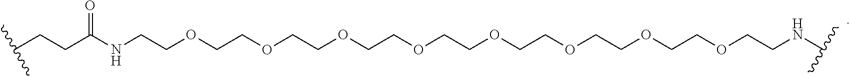 [SMILES: *CCC(=O)NCCOCCOCCOCCOCCOCCOCCOCCOCCN*]

In some embodiments, -L- is:

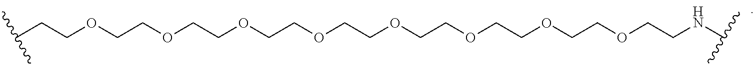 [SMILES: *CCOCCOCCOCCOCCOCCOCCOCCOCCN*]

In some embodiments, -L- is:

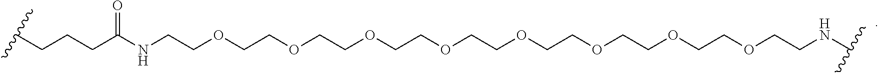 [SMILES: *CCCC(=O)NCCOCCOCCOCCOCCOCCOCCOCCOCCN*]

In some embodiments, -L- is:

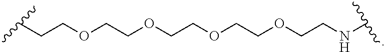 [SMILES: *CCOCCOCCOCCOCCN*]

In some embodiments, -L- is:

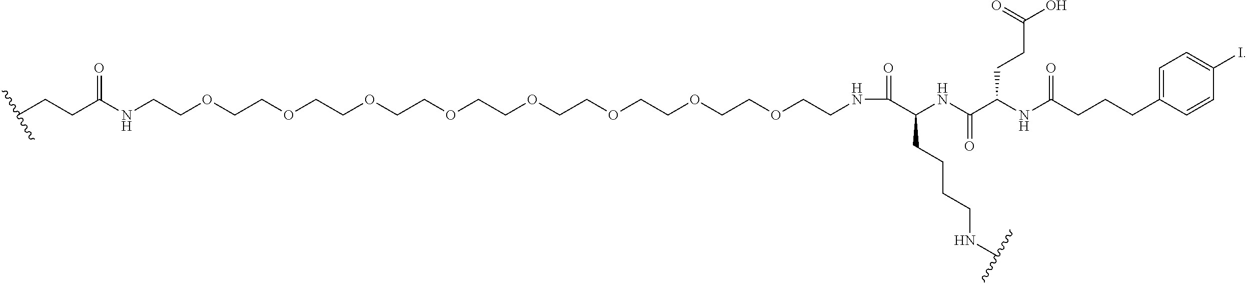 [SMILES: *CCC(=O)NCCOCCOCCOCCOCCOCCOCCOCCOCCNC(=O)[C@H](CCCCN*)NC(=O)[C@H](CCC(=O)O)NC(=O)CCCc1ccc(I)cc1]

In some embodiments, -L- is:

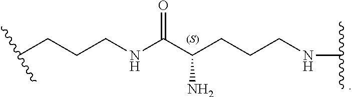

In some embodiments, -L- is:

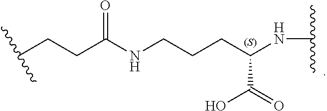

In some embodiments, -L- is:

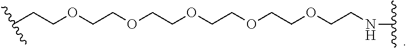 [SMILES: *CCOCCOCCOCCOCCOCCN*]

In some embodiments, -L- is:

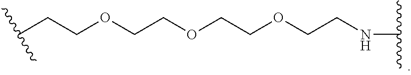 [SMILES: *CCOCCOCCOCCN*]

In some embodiments, -L- is:

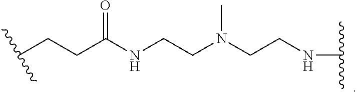 [SMILES: *CCC(=O)NCCN(C)CCN*]

In some embodiments, -L- is:

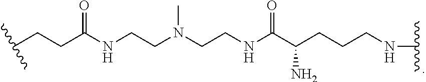 [SMILES: *CCC(=O)NCCN(C)CCNC(=O)[C@@H](N)CCCN*]

### Representative Linker and Chelating Moieties

In some embodiments, -L-R^{a} is:

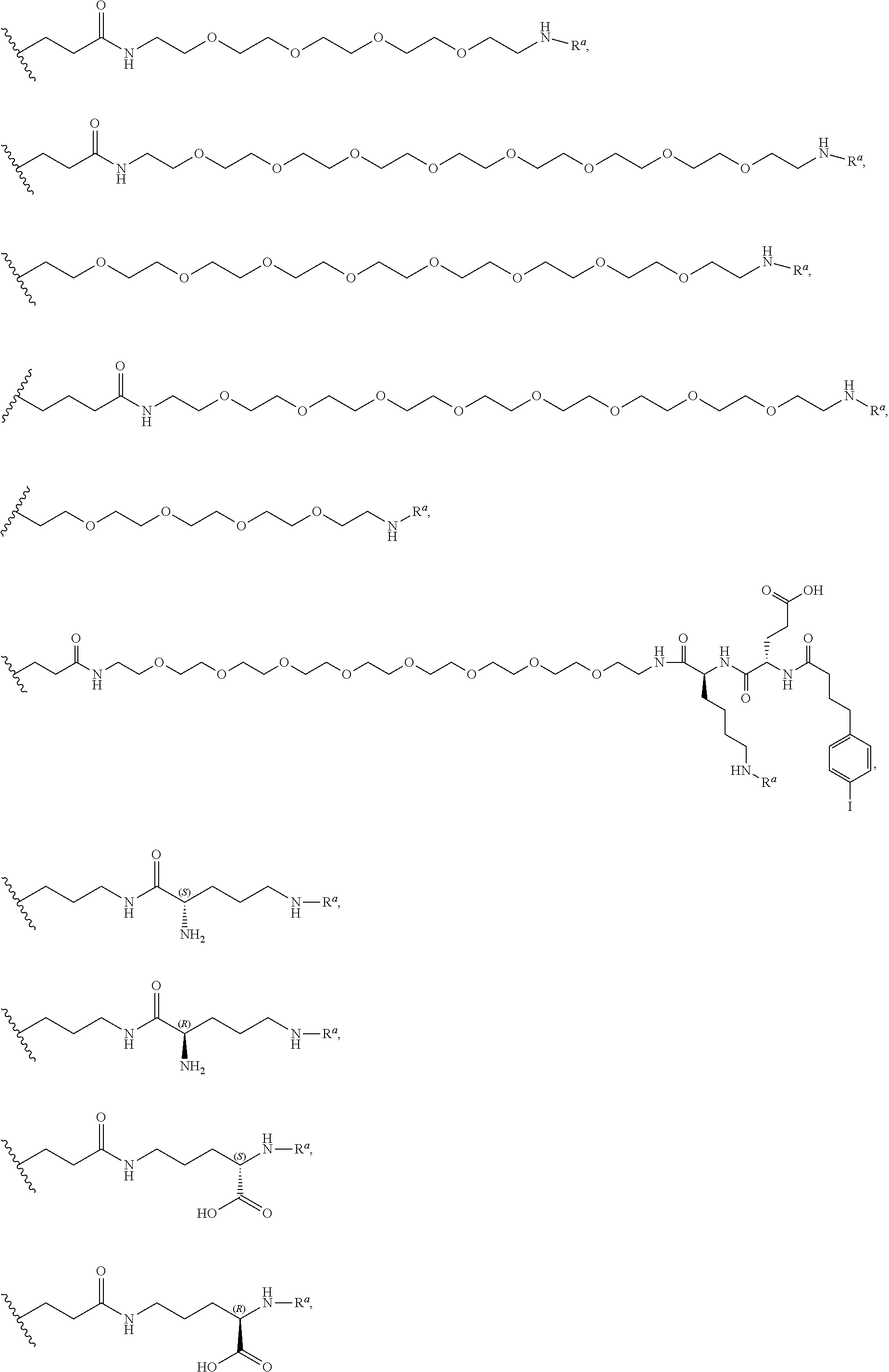

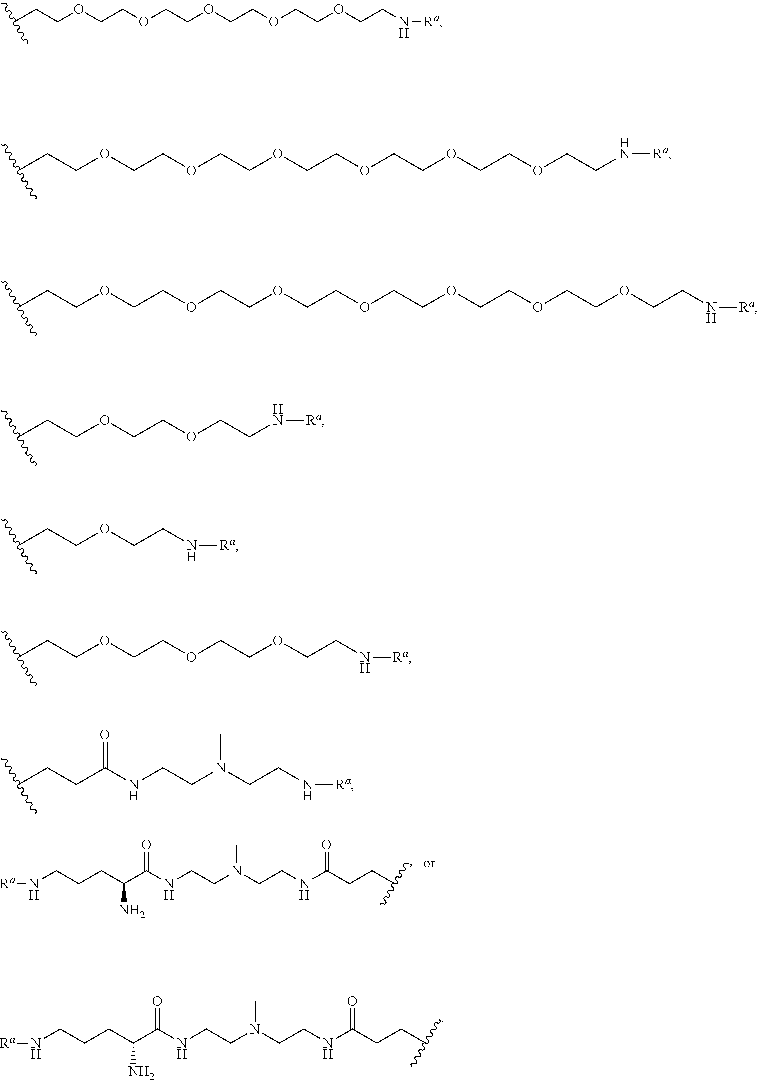

In some embodiments, -L-R^{a} is:

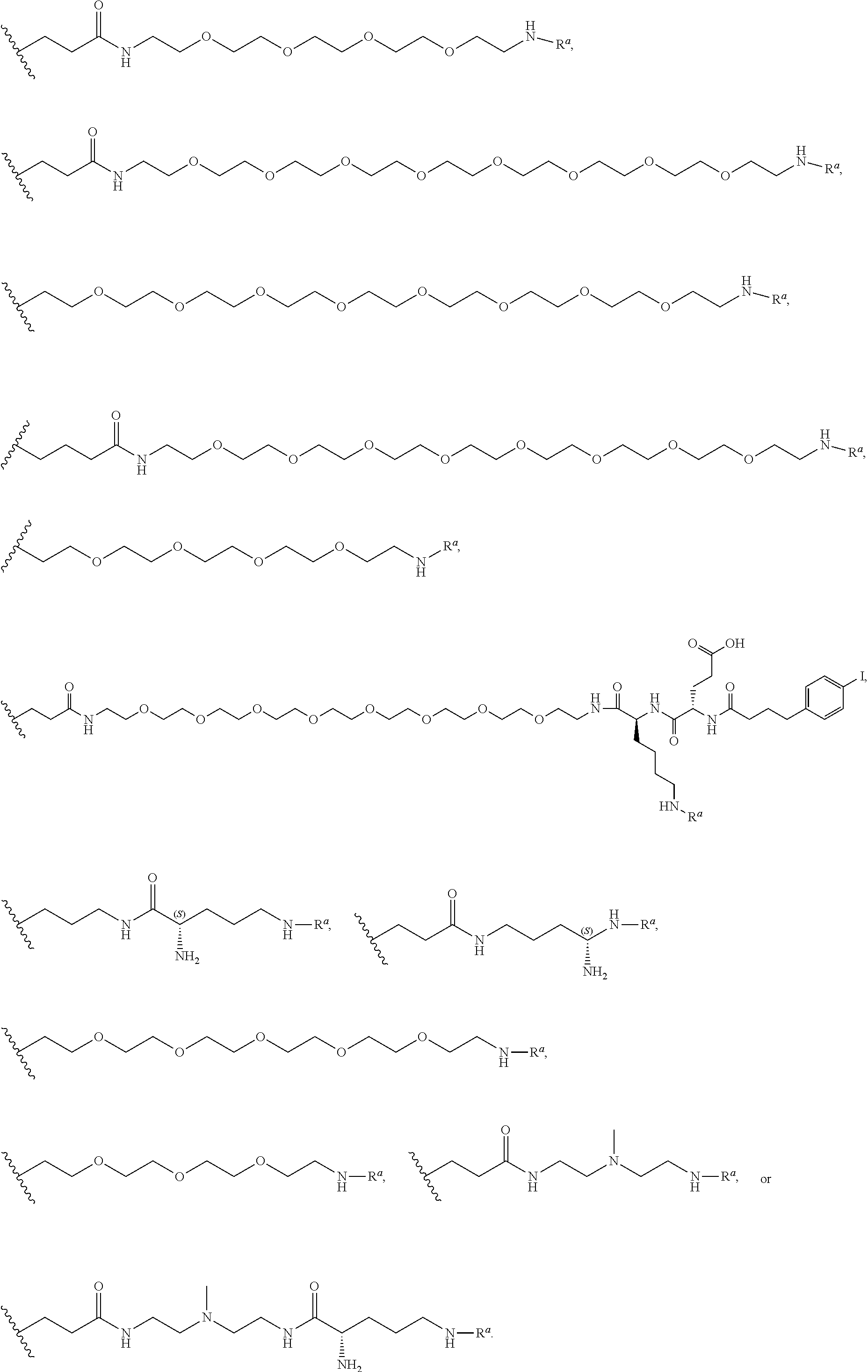

In some embodiments, -L-R^{a} is

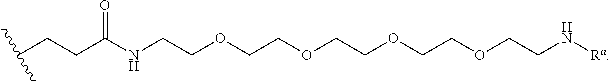

In some embodiments, -L-R^{a} is

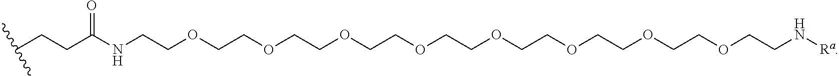

In some embodiments, -L-R^{a} is

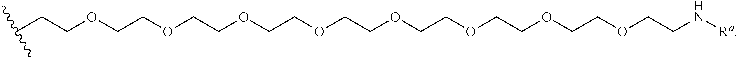

In some embodiments, -L-R^{a} is

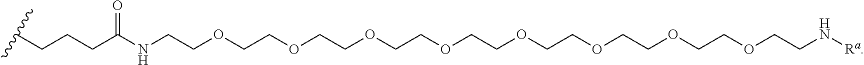

In some embodiments, -L-R^{a} is

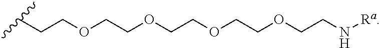

In some embodiments, -L-R^{a} is

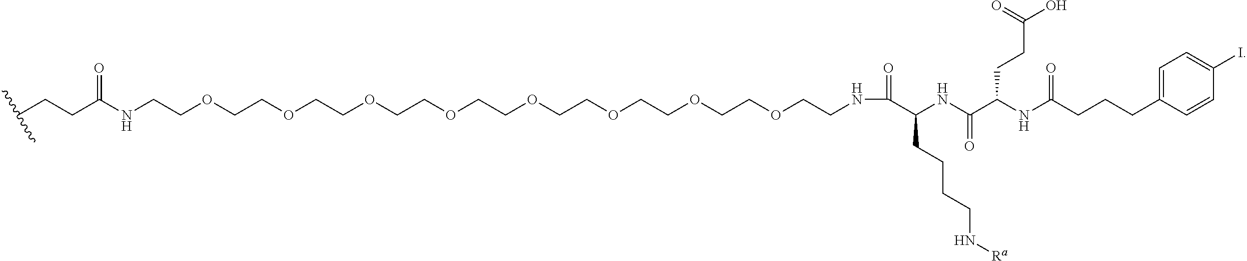

In some embodiments, -L-R^{a} is

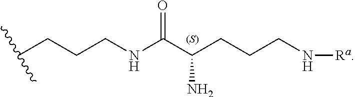

In some embodiments, -L-R^{a} is

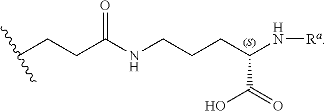

In some embodiments, -L-R^{a} is

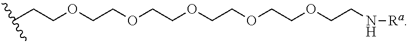

In some embodiments, -L-R^{a} is

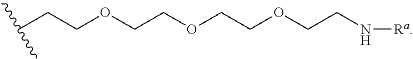

In some embodiments, -L-R^{a} is

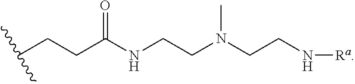

In some embodiments, -L-R^{a} is

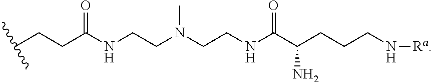

In some embodiments, -L-W is:

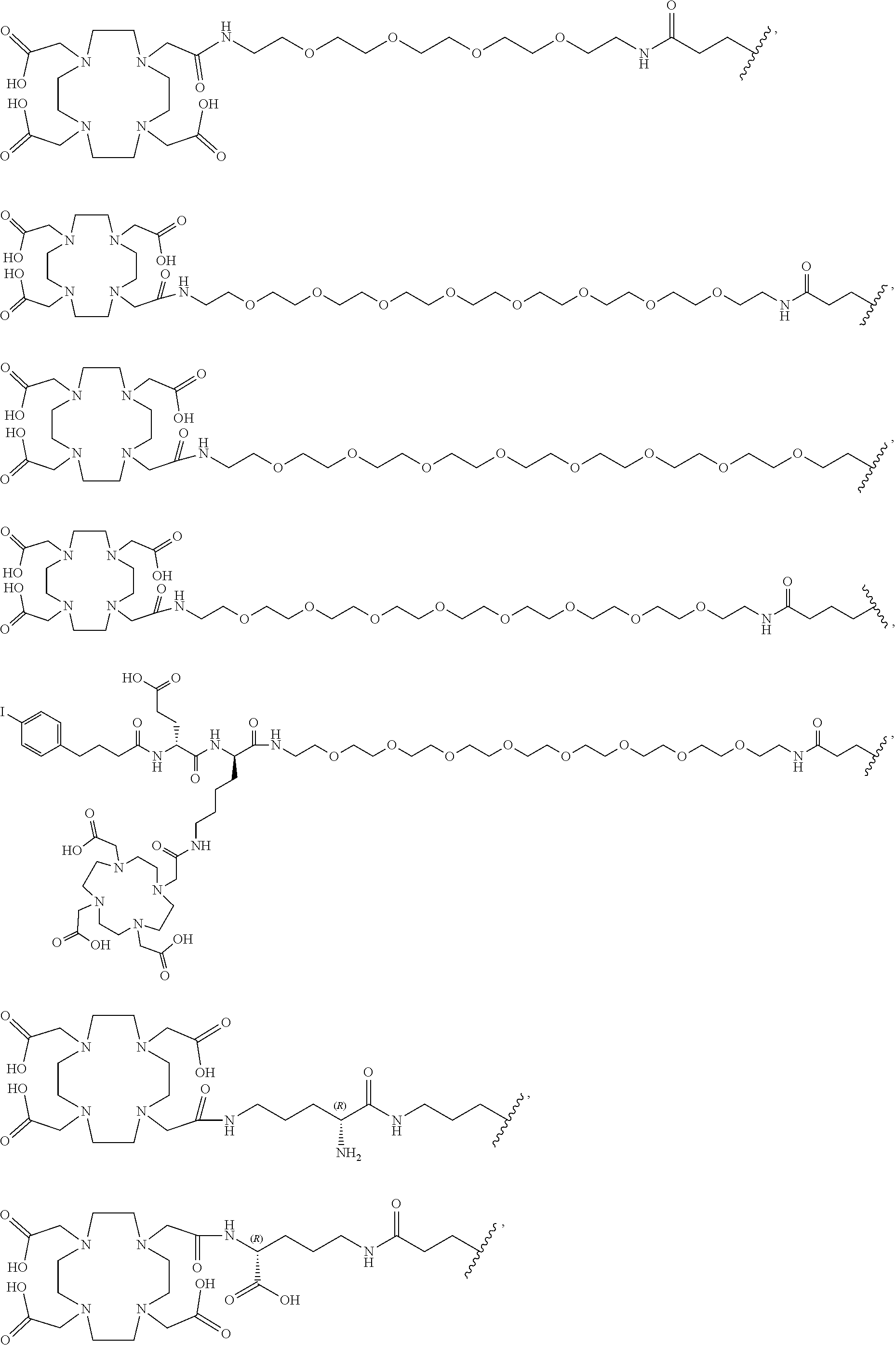

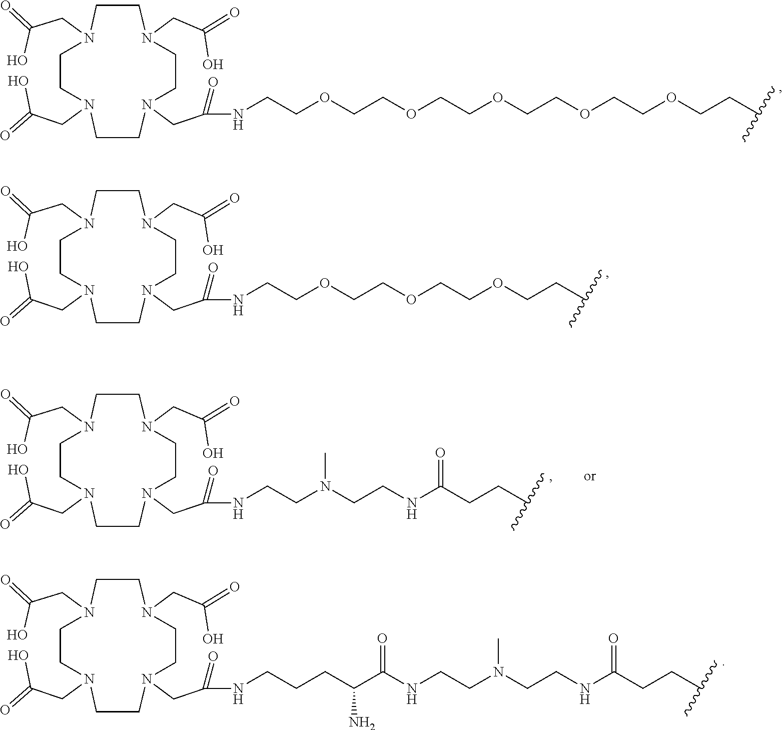 [SMILES: *CCOCCOCCOCCOCCOCCNC(=O)CN1CCN(CC(=O)O)CCN(CC(=O)O)CCN(CC(=O)O)CC1 ; *CCOCCOCCOCCNC(=O)CN1CCN(CC(=O)O)CCN(CC(=O)O)CCN(CC(=O)O)CC1 ; *CCC(=O)NCCN(C)CCNC(=O)CN1CCN(CC(=O)O)CCN(CC(=O)O)CCN(CC(=O)O)CC1 ; *CCC(=O)NCCN(C)CCNC(=O)[C@H](N)CCCNC(=O)CN1CCN(CC(=O)O)CCN(CC(=O)O)CCN(CC(=O)O)CC1]

In some embodiments, -L-R^{a} is

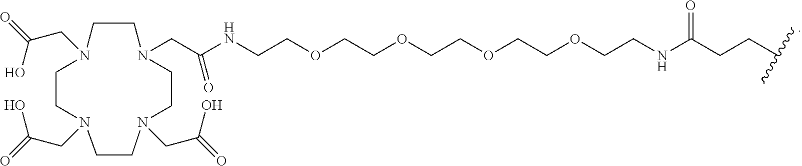 [SMILES: *CCC(=O)NCCOCCOCCOCCOCCNC(=O)CN1CCN(CC(=O)O)CCN(CC(=O)O)CCN(CC(=O)O)CC1]

In some embodiments, -L-R^{a} is

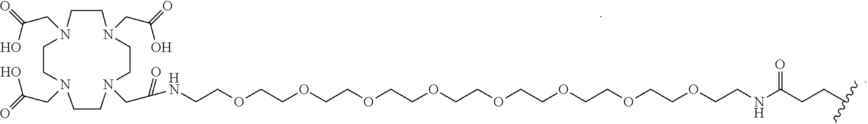 [SMILES: *CCC(=O)NCCOCCOCCOCCOCCOCCOCCOCCOCCNC(=O)CN1CCN(CC(=O)O)CCN(CC(=O)O)CCN(CC(=O)O)CC1]

In some embodiments, -L-R^{a} is

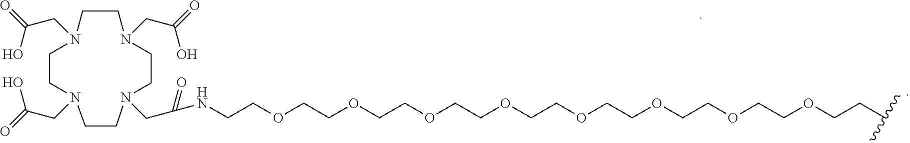 [SMILES: *CCOCCOCCOCCOCCOCCOCCOCCOCCNC(=O)CN1CCN(CC(=O)O)CCN(CC(=O)O)CCN(CC(=O)O)CC1]

In some embodiments, -L-R^{a} is

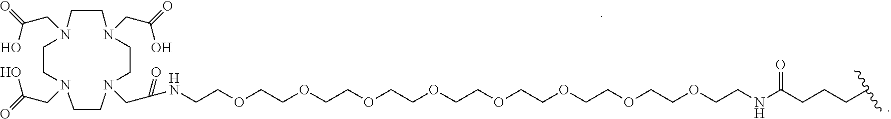 [SMILES: *CCCC(=O)NCCOCCOCCOCCOCCOCCOCCOCCOCCNC(=O)CN1CCN(CC(=O)O)CCN(CC(=O)O)CCN(CC(=O)O)CC1]

In some embodiments, -L-R^{a} is

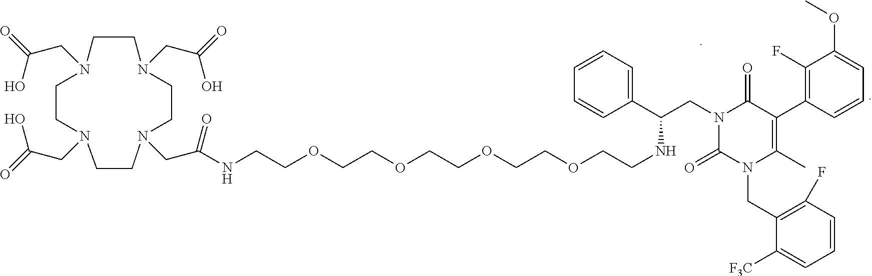 [SMILES: COc1cccc(-c2c(C)n(Cc3c(F)cccc3C(F)(F)F)c(=O)n(C[C@H](NCCOCCOCCOCCOCCNC(=O)CN3CCN(CC(=O)O)CCN(CC(=O)O)CCN(CC(=O)O)CC3)c3ccccc3)c2=O)c1F]

In some embodiments, -L-R^{a} is

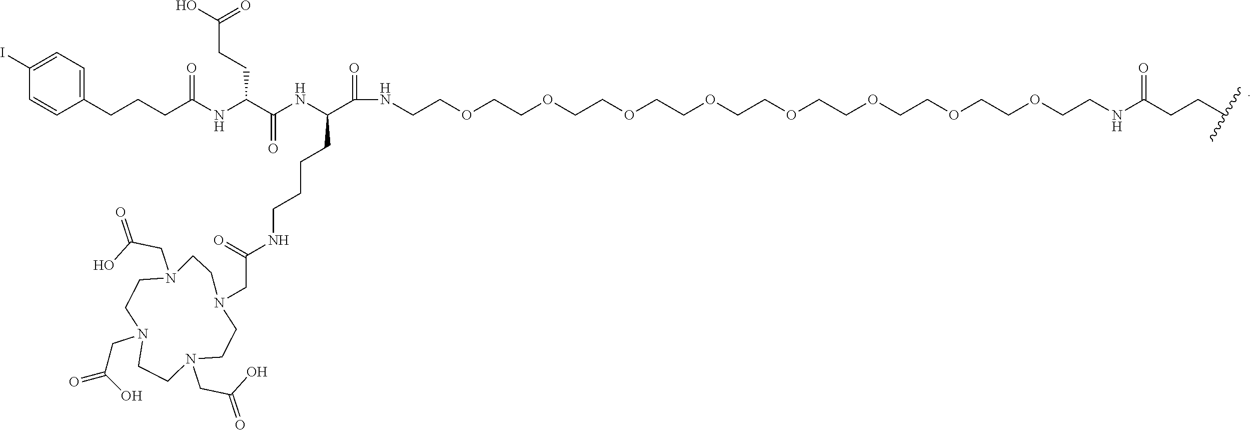 [SMILES: *CCC(=O)NCCOCCOCCOCCOCCOCCOCCOCCOCCNC(=O)[C@@H](CCCCNC(=O)CN1CCN(CC(=O)O)CCN(CC(=O)O)CCN(CC(=O)O)CC1)NC(=O)[C@@H](CCC(=O)O)NC(=O)CCCc1ccc(I)cc1]

In some embodiments, -L-R^{a} is

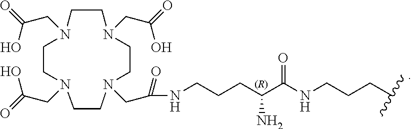

In some embodiments, -L-R^{a} is

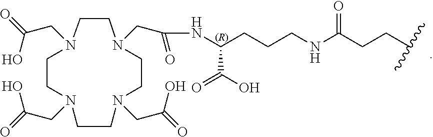

In some embodiments, -L-R^{a} is

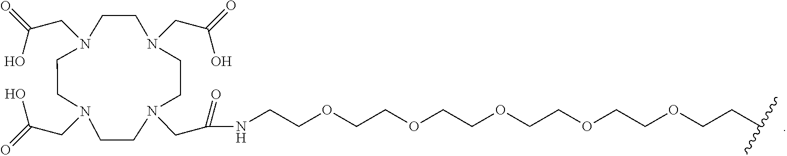 [SMILES: *CCOCCOCCOCCOCCOCCNC(=O)CN1CCN(CC(=O)O)CCN(CC(=O)O)CCN(CC(=O)O)CC1]

In some embodiments, -L-R^{a} is

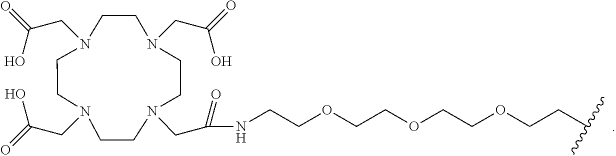 [SMILES: *CCOCCOCCOCCNC(=O)CN1CCN(CC(=O)O)CCN(CC(=O)O)CCN(CC(=O)O)CC1]

In some embodiments, -L-R is

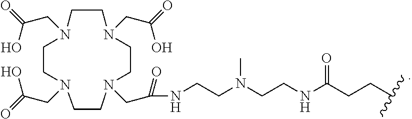 [SMILES: *CCC(=O)NCCN(C)CCNC(=O)CN1CCN(CC(=O)O)CCN(CC(=O)O)CCN(CC(=O)O)CC1]

In some embodiments, -L-R^{a} is

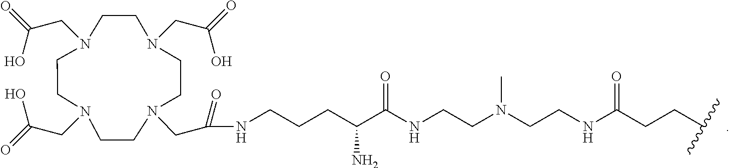 [SMILES: *CCC(=O)NCCN(C)CCNC(=O)[C@H](N)CCCNC(=O)CN1CCN(CC(=O)O)CCN(CC(=O)O)CCN(CC(=O)O)CC1]

### Representative Compounds

In some embodiments, the compound of Formula (II) has one of the following structures, or a pharmaceutically acceptable salt thereof:

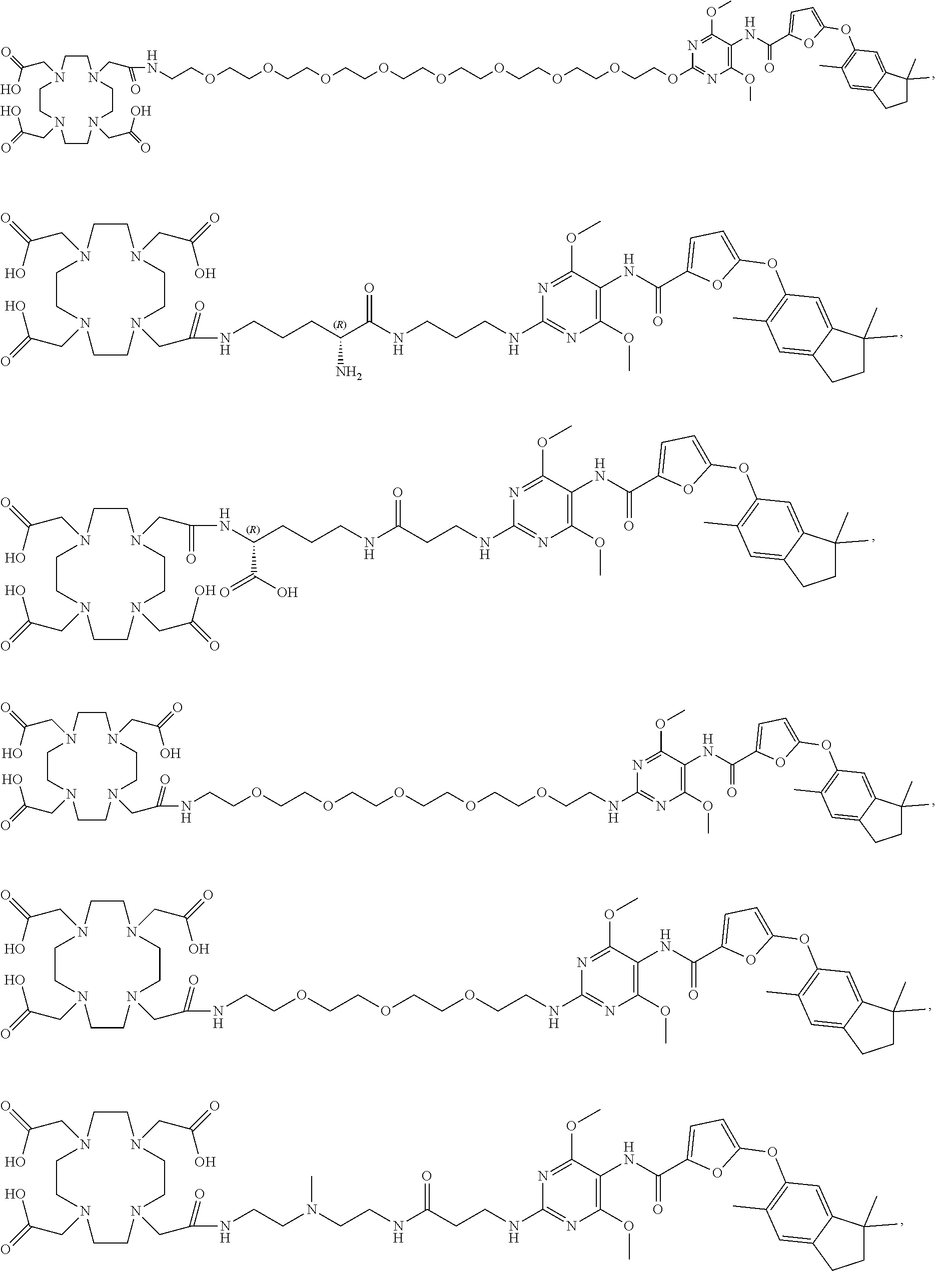

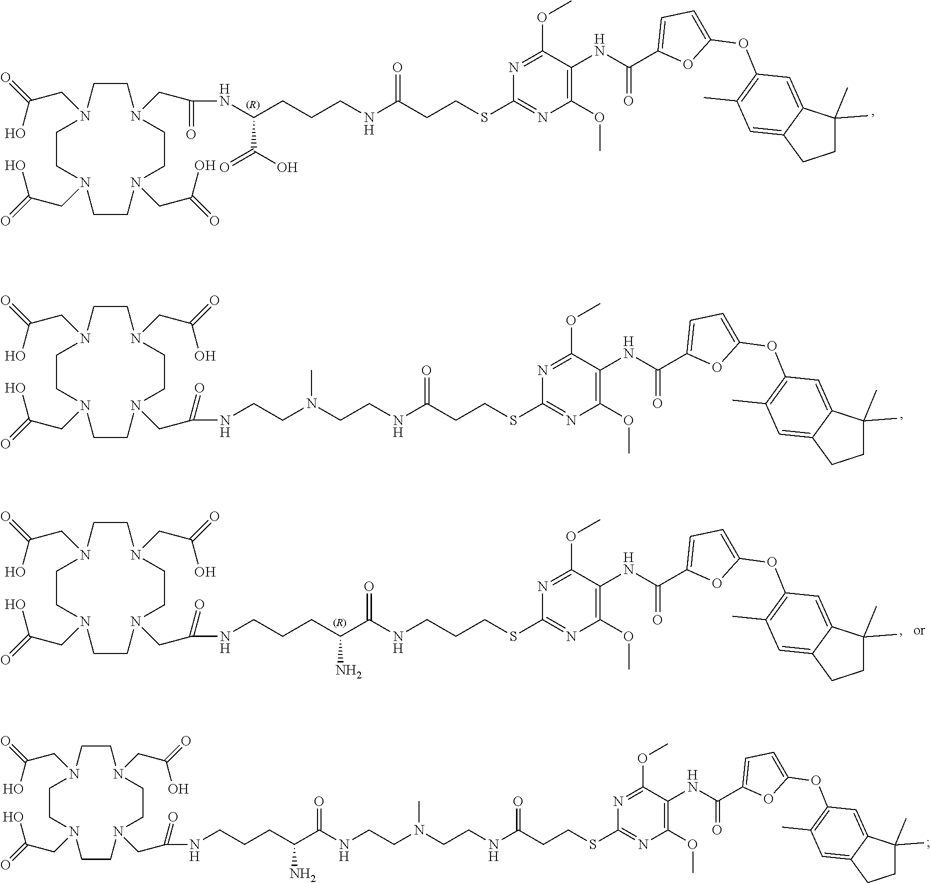

or a radionuclide complex thereof.

In some embodiments, the compound of Formula (IIIa) or (IIIb) has one of the following structures, or a pharmaceutically acceptable salt thereof:

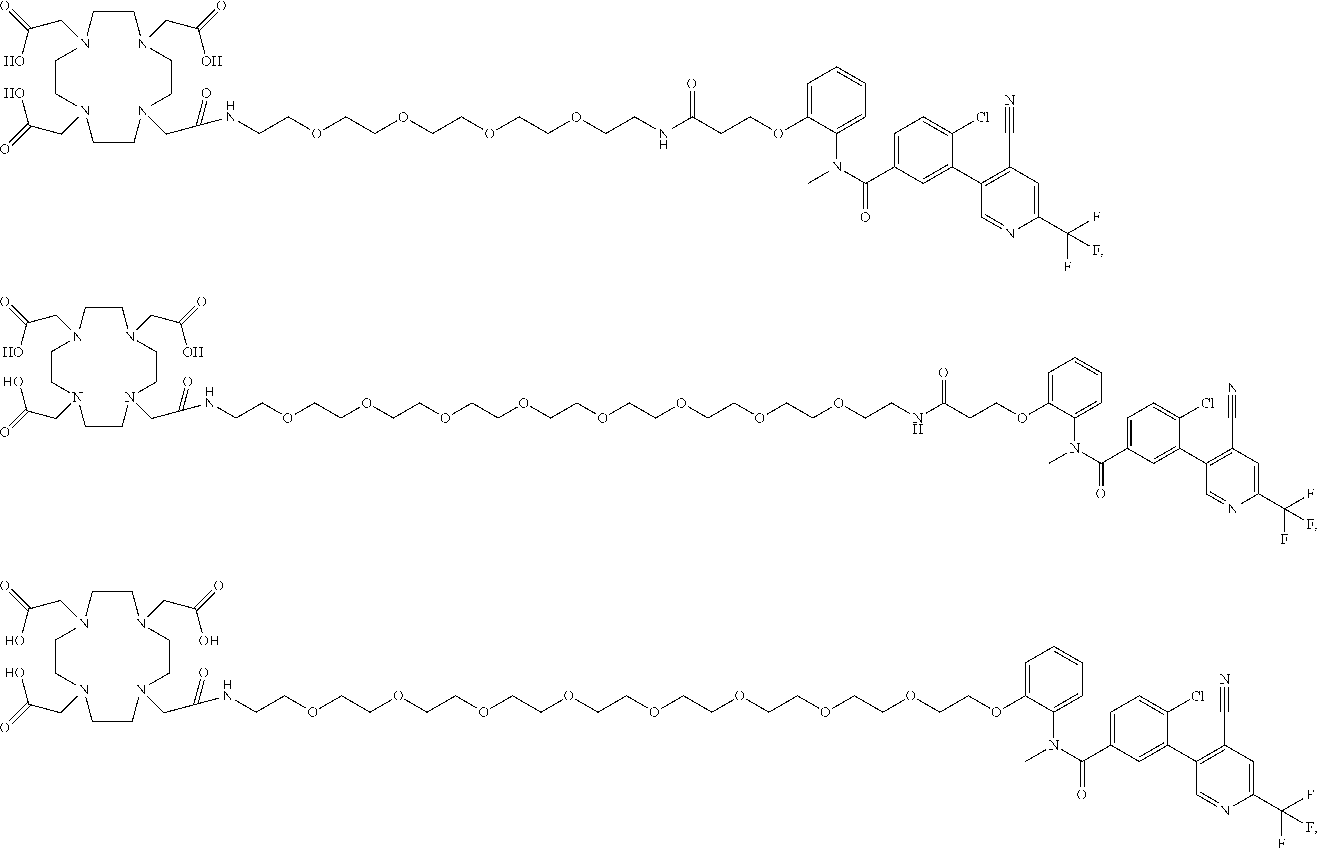 [SMILES: CN(C(=O)c1ccc(Cl)c(-c2cnc(C(F)(F)F)cc2C#N)c1)c1ccccc1OCCC(=O)NCCOCCOCCOCCOCCNC(=O)CN1CCN(CC(=O)O)CCN(CC(=O)O)CCN(CC(=O)O)CC1 ; CN(C(=O)c1ccc(Cl)c(-c2cnc(C(F)(F)F)cc2C#N)c1)c1ccccc1OCCC(=O)NCCOCCOCCOCCOCCOCCOCCOCCOCCNC(=O)CN1CCN(CC(=O)O)CCN(CC(=O)O)CCN(CC(=O)O)CC1 ; CN(C(=O)c1ccc(Cl)c(-c2cnc(C(F)(F)F)cc2C#N)c1)c1ccccc1OCCOCCOCCOCCOCCOCCOCCOCCOCCNC(=O)CN1CCN(CC(=O)O)CCN(CC(=O)O)CCN(CC(=O)O)CC1]

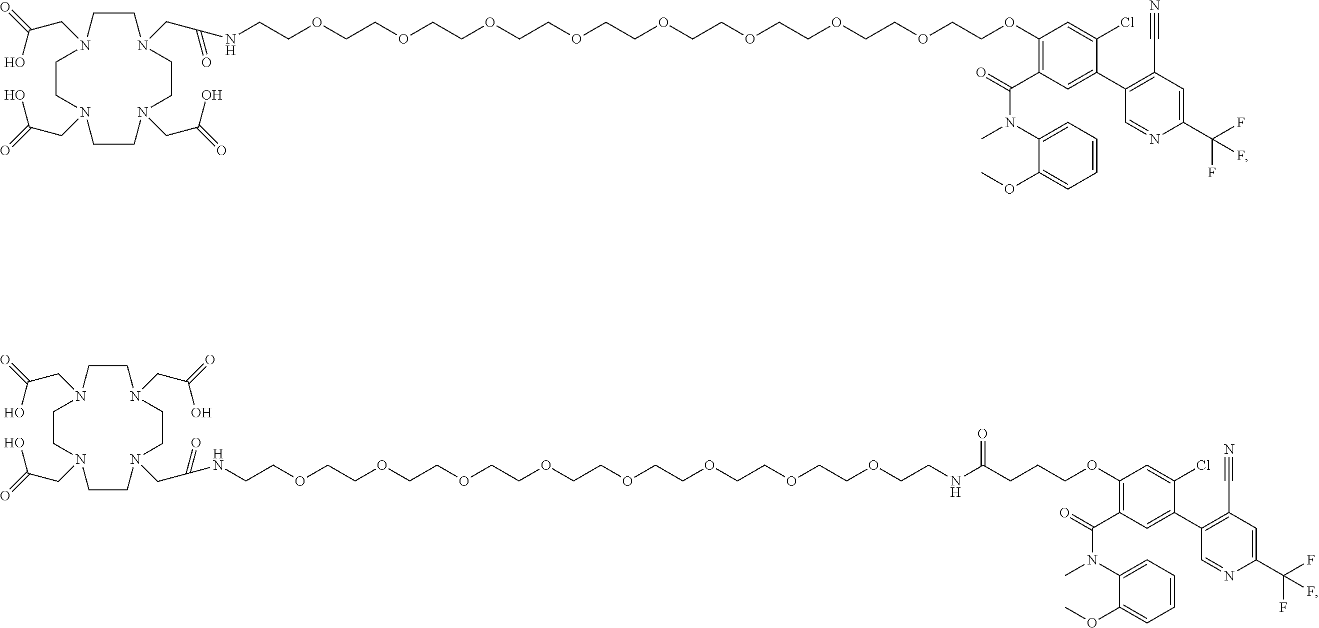 [SMILES: COc1ccccc1N(C)C(=O)c1cc(-c2cnc(C(F)(F)F)cc2C#N)c(Cl)cc1OCCOCCOCCOCCOCCOCCOCCOCCOCCNC(=O)CN1CCN(CC(=O)O)CCN(CC(=O)O)CCN(CC(=O)O)CC1 ; COc1ccccc1N(C)C(=O)c1cc(-c2cnc(C(F)(F)F)cc2C#N)c(Cl)cc1OCCCC(=O)NCCOCCOCCOCCOCCOCCOCCOCCOCCNC(=O)CN1CCN(CC(=O)O)CCN(CC(=O)O)CCN(CC(=O)O)CC1]

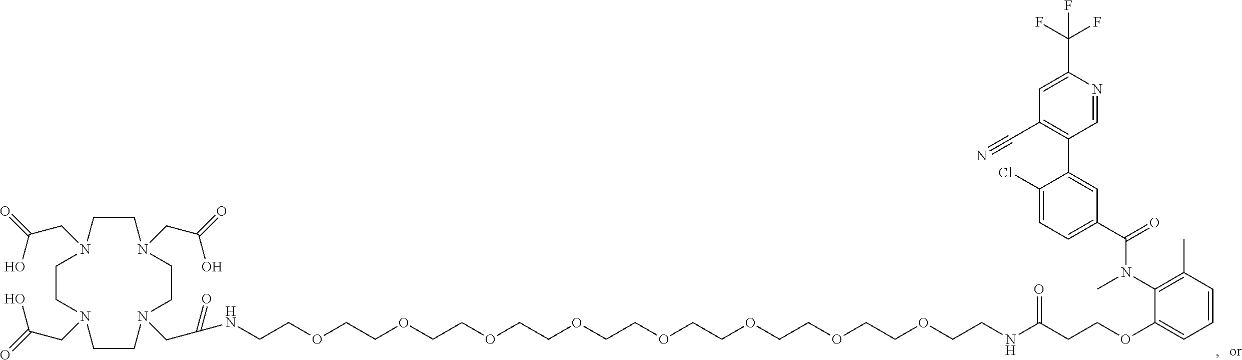 [SMILES: Cc1cccc(OCCC(=O)NCCOCCOCCOCCOCCOCCOCCOCCOCCNC(=O)CN2CCN(CC(=O)O)CCN(CC(=O)O)CCN(CC(=O)O)CC2)c1N(C)C(=O)c1ccc(Cl)c(-c2cnc(C(F)(F)F)cc2C#N)c1]

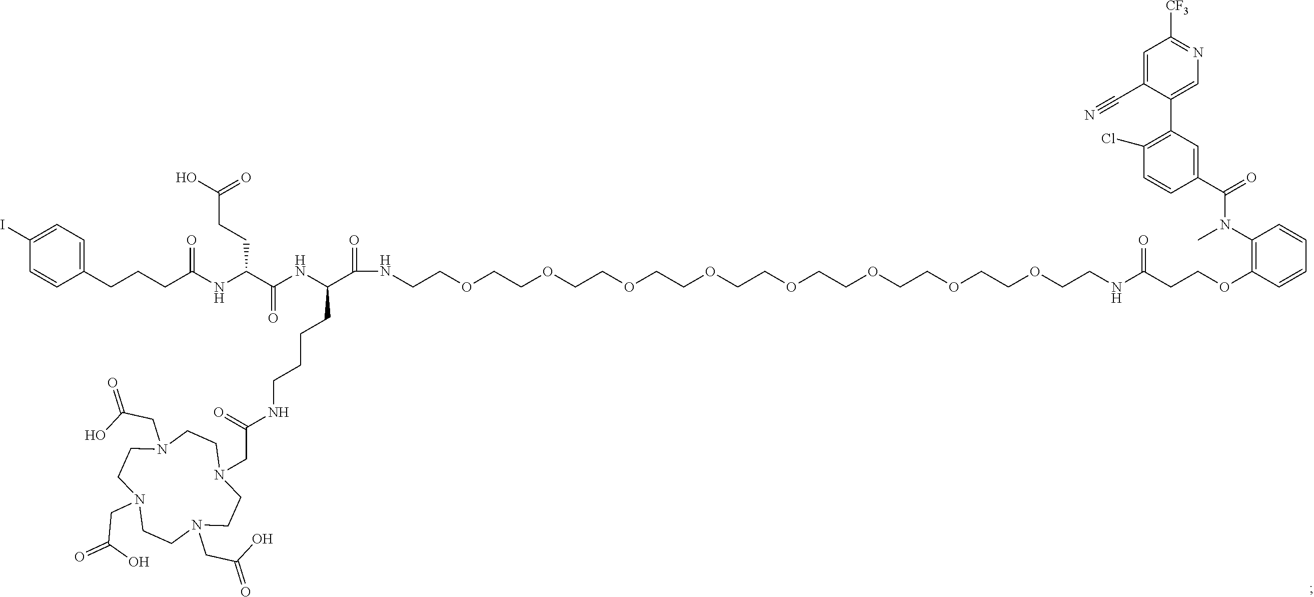 [SMILES: CN(C(=O)c1ccc(Cl)c(-c2cnc(C(F)(F)F)cc2C#N)c1)c1ccccc1OCCC(=O)NCCOCCOCCOCCOCCOCCOCCOCCOCCNC(=O)[C@@H](CCCCNC(=O)CN1CCN(CC(=O)O)CCN(CC(=O)O)CCN(CC(=O)O)CC1)NC(=O)[C@@H](CCC(=O)O)NC(=O)CCCc1ccc(I)cc1]

or a radionuclide complex thereof.

In some embodiments, the compound of Formula (IIIc) has one of the following structures, or a pharmaceutically acceptable salt thereof:

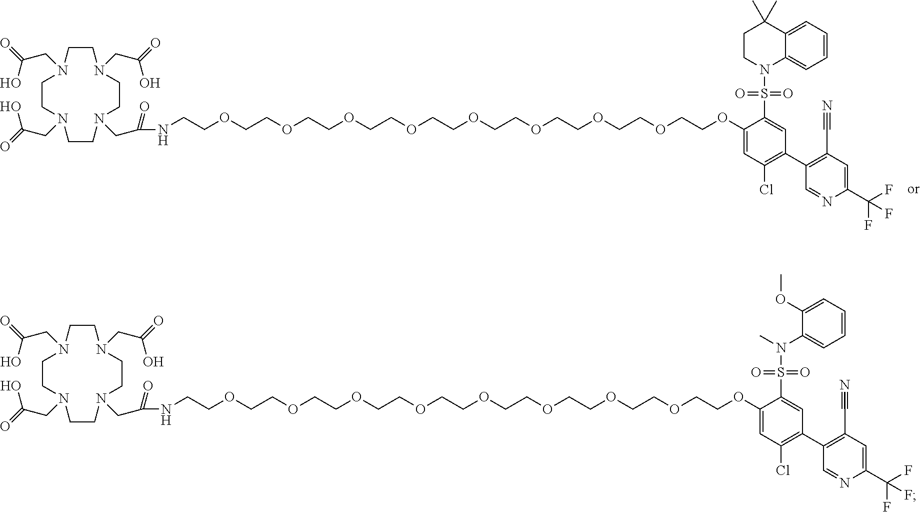 [SMILES: CC1(C)CCN(S(=O)(=O)c2cc(-c3cnc(C(F)(F)F)cc3C#N)c(Cl)cc2OCCOCCOCCOCCOCCOCCOCCOCCOCCNC(=O)CN2CCN(CC(=O)O)CCN(CC(=O)O)CCN(CC(=O)O)CC2)c2ccccc21 ; COc1ccccc1N(C)S(=O)(=O)c1cc(-c2cnc(C(F)(F)F)cc2C#N)c(Cl)cc1OCCOCCOCCOCCOCCOCCOCCOCCOCCNC(=O)CN1CCN(CC(=O)O)CCN(CC(=O)O)CCN(CC(=O)O)CC1]

or a radionuclide complex thereof.

In some embodiments, the compound of Formula (II) is compound 4, a pharmaceutically acceptable salt thereof, or radionuclide complex thereof. In some embodiments, the compound of Formula (II) is compound 11, a pharmaceutically acceptable salt thereof, or a radionuclide complex thereof. In some embodiments, the compound of Formula (II) is compound 12, a pharmaceutically acceptable salt thereof, or a radionuclide complex thereof. In some embodiments, the compound of Formula (II) is compound 13, a pharmaceutically acceptable salt thereof, or a radionuclide complex thereof. In some embodiments, the compound of Formula (II) is compound 14, a pharmaceutically acceptable salt thereof, or a radionuclide complex thereof. In some embodiments, the compound of Formula (II) is compound 15, a pharmaceutically acceptable salt thereof, or a radionuclide complex thereof.

In some embodiments, the compound of Formula (IIIa) is compound 1, a pharmaceutically acceptable salt thereof, or a radionuclide complex thereof. In some embodiments, the compound of Formula (IIIa) is compound 2, a pharmaceutically acceptable salt thereof, or a radionuclide complex thereof. In some embodiments, the compound of Formula (IIIa) is compound 3, a pharmaceutically acceptable salt thereof, or a radionuclide complex thereof. In some embodiments, the compound of Formula (IIIa) is compound 8, a pharmaceutically acceptable salt thereof, or a radionuclide complex thereof. In some embodiments, the compound of Formula (IIIa) is compound 10, a pharmaceutically acceptable salt thereof, or a radionuclide complex thereof.

In some embodiments, the compound of Formula (IIIb) is compound 5, a pharmaceutically acceptable salt thereof, or a radionuclide complex thereof. In some embodiments, the compound of Formula (IIIb) is compound 6, a pharmaceutically acceptable salt thereof, or a radionuclide complex thereof.

In some embodiments, the compound of Formula (IIIc) is compound 7, a pharmaceutically acceptable salt thereof, or a radionuclide complex thereof. In some embodiments, the compound of Formula (IIIc) is compound 9, a pharmaceutically acceptable salt thereof, or a radionuclide complex thereof.

Any combination of the groups described above for the various variables is contemplated herein. Throughout the specification, groups and substituents thereof are chosen by one skilled in the field to provide stable moieties and compounds.

### Synthesis of Compounds

Compounds described herein are synthesized using standard synthetic techniques or using methods known in the art in combination with methods described herein.

Unless otherwise indicated, conventional methods of mass spectroscopy, NMR, HPLC are employed.

Compounds are prepared using standard organic chemistry techniques such as those described in, for example, March's Advanced Organic Chemistry, 6^{th} Edition, John Wiley and Sons, Inc. Alternative reaction conditions for the synthetic transformations described herein may be employed such as variation of solvent, reaction temperature, reaction time, as well as different chemical reagents and other reaction conditions.

In one aspect, compounds described herein are in the form of pharmaceutically acceptable salts. In addition, the compounds described herein can exist in unsolvated as well as solvated forms with pharmaceutically acceptable solvents such as water, ethanol, and the like. The solvated forms of the compounds presented herein are also considered to be disclosed herein.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible, and this capability can be manipulated as one aspect of delayed and sustained release behaviors. Also, because the salt-forming molecule can be in equilibrium with a neutral form, passage through biological membranes can be adjusted.

In some embodiments, pharmaceutically acceptable salts are obtained by reacting a compound of Formula (I), (II), (IIIa), (IIIb), (IIIc), (IIId), or (IIIe) with an acid. In some embodiments, the acid is an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; propionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, a compound of Formula (I), (II), (IIIa), (IIIb), (IIIc), (IIId), or (IIIe), is prepared as a chloride salt, sulfate salt, bromide salt, mesylate salt, maleate salt, citrate salt or phosphate salt.

In some embodiments, pharmaceutically acceptable salts are obtained by reacting a compound of Formula (I), (II), (IIIa), (IIIb), (IIIc), (IIId), or (IIIe), with a base. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

In some embodiments, any one of the hydrogen atoms on the organic radicals (e.g., alkyl groups, aromatic rings) of compounds described herein are replaced with deuterium.

In some embodiments, the compounds of Formula (I), (II), (IIIa), (IIIb), (IIIc), (IIId), or (IIIe), possess one or more stereocenters and each stereocenter exists independently in either the R or S configuration. The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds described herein, are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers is carried out using covalent diastereomeric derivatives of the compounds described herein. In another embodiment, diastereomers are separated by separation/resolution techniques based upon differences in solubility. In other embodiments, separation of stereoisomers is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis.

In some embodiments, compounds described herein are prepared as prodrugs. A “prodrug” refers to an agent that is converted into the parent drug in vivo. Prodrugs are often useful because, in some situations, they are easier to administer than the parent drug. They are, for instance, bioavailable by oral administration whereas the parent is not. Further or alternatively, the prodrug also has improved solubility in pharmaceutical compositions over the parent drug. In some embodiments, the design of a prodrug increases the effective water solubility. See for example Design of Prodrugs, Bundgaard, A. Ed., Elsevier, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference.

A “metabolite” of a compound disclosed herein is a derivative of that compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulfhydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein can be affected by any method that enables delivery of the compounds to the site of action. These methods include, though are not limited to, delivery via parenteral routes (including injection or infusion, and subcutaneous).

In some embodiments, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and contain optional agents as excipients such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use.

### Methods of Treatment

In some embodiments, the methods comprise administering to a subject a therapeutically effective amount of a compound of Formula (I), (II), (IIIa), (IIIb), (IIIc), (IIId), or (IIIe), or a pharmaceutically acceptable salt or solvate thereof. In some embodiments, the compound of Formula (I), (II), (IIIa), (IIIb), (IIIc), (IIId), or (IIIe), or pharmaceutically acceptable salt or solvate thereof is administered in a pharmaceutical composition. In some embodiments, the subject has cancer. In some embodiments, the cancer is a solid tumor. In some embodiments, the subject has a noncancerous tumor. In some embodiments, the subject has an adenoma.

In some embodiments, the treatment is sufficient to reduce or inhibit the growth of the subject's tumor, reduce the number or size of metastatic lesions, reduce tumor load, reduce primary tumor load, reduce invasiveness, prolong survival time, or maintain or improve the quality of life, or combinations thereof.

In some embodiments, provided herein are methods for killing a tumor cell comprising contacting the tumor cell with a compound of Formula (I), (II), (IIIa), (IIIb), (IIIc), (IIId), or (IIIe), or a pharmaceutically acceptable salt or solvate thereof. In some embodiments, the compound of Formula (I), (II), (IIIa), (IIIb), (IIIc), (IIId), or (IIIe), or pharmaceutically acceptable salt or solvate thereof releases a number of alpha particles by natural radioactive decay. In some embodiments, the released alpha particles are sufficient to kill the tumor cell. In some embodiments, the released alpha particles are sufficient to stop cell growth. In some embodiments, the tumor cell is a malignant tumor cell. In some embodiments, the tumor cell is a benign tumor cell. In some embodiments, the method comprises killing a tumor cell with a beta-particle emitting radionuclide. In some embodiments, the method comprises killing a tumor cell with an alpha-particle emitting radionuclide. In some embodiments, the method comprises killing a tumor cell with a gamma-particle emitting radionuclide.

In one aspect, provided herein are methods and compositions for treating cancers. In some embodiments, the cancer is ovarian cancer. In some embodiments, the cancer is breast cancer. In some embodiments, the cancer is endometrial cancer. In some embodiments, the cancer is prostate cancer.

In one aspect, provided herein are methods and compositions for treating an adenoma.

In one aspect, provided herein are methods and compositions for treating a carcinoma.

In one aspect, provided herein is a method for identifying tissues or organs in a mammal that overexpress GnRHR comprising: (i) administering to the mammal a GnRHR radiopharmaceutical described herein, or a pharmaceutically acceptable salt thereof; and (ii) performing single-photon emission computerized tomography (SPECT) or positron emission tomography (PET) analysis on the mammal. In some embodiments, the method comprises: (i) administering to the mammal a GnRHR radiopharmaceutical described herein, or a pharmaceutically acceptable salt thereof; and (ii) performing positron emission tomography (PET) analysis on the mammal.

In some embodiments, the mammal was diagnosed with cancer. In some embodiments, the mammal was diagnosed with ovarian cancer. In some embodiments, the mammal was diagnosed with breast cancer. In some embodiments, the mammal was diagnosed with endometrial cancer. In some embodiments, the mammal was diagnosed with prostate cancer. In some embodiments, the tissues in the mammal that overexpress GnRHR are tumors.

In some embodiments, a GnRHR radiopharmaceutical described herein, or a pharmaceutically acceptable salt thereof are used in a method for in vivo imaging of a subject. In some embodiments, the method includes the steps of:
(i) administering to the mammal GnRHR radiopharmaceutical described herein, or a pharmaceutically acceptable salt thereof;
(ii) waiting a sufficient amount of time to allow the GnRHR radiopharmaceutical, to accumulate at a tissue or cell site to be imaged; and
(iii) imaging the cells or tissues with a non-invasive imaging technique.

In some embodiments, the non-invasive imaging technique is single-photon emission computerized tomography (SPECT) or positron emission tomography (PET) analysis. In some embodiments, the non-invasive imaging technique is single-photon emission computerized tomography (SPECT). In some embodiments, the non-invasive imaging technique is selected from positron emission tomography imaging, or positron emission tomography with computed tomography imaging, and positron emission tomography with magnetic resonance imaging.

### Methods of Dosing and Treatment Regimens

In one embodiment, the GnRHR radiopharmaceutical described herein, or a pharmaceutically acceptable salt thereof, are used in the preparation of medicaments for the treatment of tumors in a mammal. Methods for treating any of the diseases or conditions described herein in a mammal in need of such treatment, involves administration of pharmaceutical compositions that include at least one compound of Formula (I), (II), (IIIa), (IIIb), (IIIc), (IIId), or (IIIe), or a pharmaceutically acceptable salt thereof, in therapeutically effective amounts to said mammal.

In certain embodiments, the compositions containing the compound(s) described herein are administered for diagnostic and/or therapeutic treatments.

The amount of a given agent that corresponds to such an amount varies depending upon factors such as the particular conjugate, specific cancer or tumor to be treated (and its severity), the identity (e.g., weight, sex) of the subject or host in need of treatment, but nevertheless is determined according to the particular circumstances surrounding the case, including, e.g., the specific conjugate being administered, the route of administration, the condition being treated, and the subject or host being treated. Optimal doses are generally determined using experimental models and/or clinical trials. The optimal dose depends upon the body mass, weight, or blood volume of the subject.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans.

The amount of a compound of Formula (I), (II), (IIIa), (IIIb), (IIIc), (IIId), or (IIIe), or pharmaceutically acceptable salts thereof, that are administered are sufficient to deliver a therapeutically effective dose to the particular subject. In some embodiments, dosages of a compound of Formula (I), (II), (IIIa), (IIIb), (IIIc), (IIId), or (IIIe), are between about 0.1 pg and about 50 mg per kilogram of body weight, 1 μg and about 50 mg per kilogram of body weight, or between about 0.1 and about 10 mg/kg of body weight. Therapeutically effective dosages can also be determined at the discretion of a physician. By way of example only, the dose of a compound of Formula (I), (II), (IIIa), (IIIb), (IIIc), (IIId), or (IIIe), or a pharmaceutically acceptable salt thereof described herein for methods of treating a disease as described herein is about 0.001 mg/kg to about 1 mg/kg body weight of the subject per dose. In some embodiments, the dose is about 0.001 mg to about 1000 mg per dose for the subject being treated. In some embodiments, a compound of Formula (I), (II), (IIIa), (IIIb), (IIIc), (IIId), or (IIIe), or a pharmaceutically acceptable salt thereof described herein is administered to a subject at a dosage of from about 0.01 mg to about 500 mg, from about 0.01 mg to about 100 mg, or from about 0.01 mg to about 50 mg.

In some embodiments, a compound of Formula (I), (II), (IIIa), (IIIb), (IIIc), (IIId), or (IIIe), or a pharmaceutically acceptable salt thereof described herein is administered to a subject at a dosage of about 0.01 picomole to about 1 mole, about 0.1 picomole to about 0.1 mole, about 1 nanomole to about 0.1 mole, or about 0.01 micromole to about 0.1 millimole.

In some embodiments, a compound of Formula (I), (II), (IIIa), (IIIb), (IIIc), (IIId), or (IIIe), or a pharmaceutically acceptable salt thereof described herein is administered to a subject at a dosage of about 0.01 Gbq to about 1000 Gbq, about 0.5 Gbq to about 100 Gbq, or about 1 Gbq to about 50 Gbq.

In some embodiments, the dose is administered once a day, 1 to 3 times a week, 1 to 4 times a month, or 1 to 12 times a year.

In any of the aforementioned aspects are further embodiments in which the effective amount of the GnRHR radiopharmaceutical described herein, or a pharmaceutically acceptable salt thereof, is: (a) systemically administered to the mammal; and/or (b) intravenously administered to the mammal; and/or (c) administered by injection to the mammal.

In certain instances, it is appropriate to administer at least one GnRHR radiopharmaceutical described herein, or a pharmaceutically acceptable salt thereof, in combination with one or more other therapeutic agents.

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

As used herein and in the appended claims, singular articles such as “a” and “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein, “about” will be understood by persons of ordinary skill in the art and will vary to some extent depending upon the context in which it is used. If there are uses of the term which are not clear to persons of ordinary skill in the art, given the context in which it is used, “about” will mean up to plus or minus 10% of the particular term.

As used herein, C₁-Cₓ includes C₁-C₂, C₁-C₃ . . . C₁-Cₓ. By way of example only, a group designated as “C₁-C₆” indicates that there are one to six carbon atoms in the moiety, i.e., groups containing 1 carbon atom, 2 carbon atoms, 3 carbon atoms or 4 carbon atoms. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl.

An “alkyl” group refers to an aliphatic hydrocarbon group. The alkyl group is branched or straight chain. In some embodiments, the “alkyl” group has 1 to 10 carbon atoms, i.e., a —C₁-C₁₀ alkyl. Whenever it appears herein, a numerical range such as “1 to 10” refers to each integer in the given range; e.g., “1 to 10 carbon atoms” means that the alkyl group consists of 1 carbon atom, 2 carbon atoms, 3 carbon atoms, etc., up to and including 10 carbon atoms, although the present definition also covers the occurrence of the term “alkyl” where no numerical range is designated. In some embodiments, an alkyl is a —C₁-C₆ alkyl. In one aspect the alkyl is methyl, ethyl, propyl, iso-propyl, n-butyl, iso-butyl, sec-butyl, or t-butyl. Typical alkyl groups include, but are in no way limited to, methyl, ethyl, propyl, isopropyl, butyl, isobutyl, sec-butyl, tertiary butyl, pentyl, neopentyl, or hexyl. In some embodiments, the alkyl group is an “alkenyl” or “alkynyl” group.

An “alkylene” group refers to a divalent alkyl radical. Any of the above-mentioned monovalent alkyl groups may be an alkylene by abstraction of a second hydrogen atom from the alkyl. In some embodiments, an alkylene is a —C₁-C₆ alkylene. In other embodiments, an alkylene is a —C₁-C₄ alkylene. Typical alkylene groups include, but are not limited to, —CH₂—, —CH₂CH₂—, —CH₂CH₂CH₂—, —CH₂CH₂CH₂CH₂—, and the like. In some embodiments, an alkylene is —CH₂—. In some embodiments, an alkylene is —CH₂CH₂—.

An “alkoxy” group refers to an (alkyl)O— group, where alkyl is as defined herein.

The term “alkenyl” refers to a type of alkyl group in which at least one carbon-carbon double bond is present. In one embodiment, an alkenyl group has the formula: —C(R)═CR₂, wherein R refers to the remaining portions of the alkenyl group, which may be the same or different. In some embodiments, each R is independently H or an alkyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)=CH₂, —CH═CHCH₃, —C(CH₃)=CHCH₃, and —CH₂CH═CH₂.

The term “alkynyl” refers to a type of alkyl group in which at least one carbon-carbon triple bond is present. In one embodiment, an alkenyl group has the formula —C≡C—R, wherein R refers to the remaining portion of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH.

The term “heteroalkyl” refers to an alkyl group in which one or more skeletal atoms of the alkyl are selected from an atom other than carbon, e.g., oxygen, nitrogen (e.g., —NH—, —N(alkyl)-), sulfur, or combinations thereof. In some embodiments, the “heteroalkyl” group has 2 to 10 atoms in the backbone, which include a combination of carbon atoms and heteroatoms (e.g. N, O, S), i.e., a 2 to 10-membered heteroalkyl. In some embodiments, the heteroalkyl is attached to the rest of the molecule at a carbon atom of the heteroalkyl. In one embodiment, a heteroalkyl is a 2 to 8 membered heteroalkyl.

A “heteroalkylene” group refers to a divalent alkyl radical derived from heteroalkyl, as exemplified, but not limited by, —CH₂—CH₂—O—CH₂—CH₂— and —CH₂—O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(═O)O— represents both —C(═O)O— and —OC(═O)—. Additionally, the formula —C(═O)NH-represents both —C(═O)NH— and —NHC(═O)—.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

As used herein, the term “aryl” refers to an aromatic ring wherein each of the atoms forming the ring is a carbon atom. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀ aryl. Depending on the structure, an aryl group is a monoradical or a diradical (i.e., an arylene group).

The term “cycloalkyl” refers to a monocyclic or polycyclic aliphatic, non-aromatic radical, wherein each of the atoms forming the ring (i.e., skeletal atoms) is a carbon atom. In some embodiments, cycloalkyls are spirocyclic or bridged cycloalkyls. In some embodiments, cycloalkyls are optionally fused with an aromatic ring, and the point of attachment is at a carbon that is not an aromatic ring carbon atom. Cycloalkyl groups include groups having from 3 to 12 ring atoms. In some embodiments, cycloalkyl groups are selected from cyclopropyl, cyclobutyl, cyclopentyl, cyclopentenyl, cyclohexyl, cyclohexenyl, cycloheptyl, cyclooctyl, spiro[2.2]pentyl, norbornyl and bicycle[1.1.1]pentyl. In some embodiments, a cycloalkyl is a C₃-C₆ cycloalkyl. In some embodiments, a cycloalkyl is a C₃-C₄ cycloalkyl. In some embodiments, a cycloalkyl is a C₅-C₆ cycloalkyl.

The term “halo” or, alternatively, “halogen” or “halide” means fluoro, chloro, bromo or iodo. In some embodiments, halo is fluoro, chloro, or bromo.

The term “fluoroalkyl” refers to an alkyl in which one or more hydrogen atoms are replaced by a fluorine atom. In one aspect, a fluoroalkyl is a —C₁-C₆ fluoroalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 12 atoms in its ring system, and with the proviso that any ring does not contain two adjacent O or S atoms. Non-aromatic heterocyclic groups (also known as heterocycloalkyls) include rings having 3 to 12 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. Illustrative examples of heteroaryl groups include monocyclic heteroaryls and bicyclic heteroaryls. Monocyclic heteroaryls include pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, pyridazinyl, triazinyl, oxadiazolyl, thiadiazolyl, and furazanyl. Bicyclic heteroaryls include indolizine, indole, benzofuran, benzothiophene, indazole, benzimidazole, purine, quinolizine, quinoline, isoquinoline, cinnoline, phthalazine, quinazoline, quinoxaline, 1,8-naphthyridine, and pteridine. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, a heteroaryl contains 1 O atom. In some embodiments, a heteroaryl contains 1 S atom in the ring. In some embodiments, heteroaryl is a 5 to 10-membered heteroaryl. In some embodiments, a monocyclic heteroaryl is a 5 to 6 membered heteroaryl. In some embodiments, a monocyclic heteroaryl is a 5-membered heteroaryl. In some embodiments, a monocyclic heteroaryl is a 6-membered heteroaryl. In some embodiments, bicyclic heteroaryl is a 10-membered heteroaryl.

A “heterocycloalkyl” group refers to a cycloalkyl group that includes at least one heteroatom selected from nitrogen, oxygen and sulfur. In some embodiments, a heterocycloalkyl is fused with an aryl or heteroaryl. In some embodiments, the heterocycloalkyl is oxazolidinonyl, pyrrolidinyl, tetrahydrofuranyl, tetrahydrothienyl, tetrahydropyranyl, tetrahydrothiopyranyl, piperidinyl, morpholinyl, thiomorpholinyl, piperazinyl, piperidin-2-onyl, pyrrolidine-2,5-dithionyl, pyrrolidine-2,5-dionyl, pyrrolidinonyl, imidazolidinyl, imidazolidin-2-onyl, or thiazolidin-2-onyl. In one aspect, a heterocycloalkyl is a 3 to 12 membered heterocycloalkyl. In another aspect, a heterocycloalkyl is a 5 to 10-membered heterocycloalkyl. In some embodiments, a heterocycloalkyl is a 5-membered heterocycloalkyl. In some embodiments, a heterocycloalkyl is a 6-membered heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 1-4 nitrogen (N) atoms in the ring. In some embodiments, a heterocycloalkyl contains 0-2 N atoms, 0-2 oxygen (O) atoms and 0-1 sulfur (S) atoms in the ring.

The term “bond” or “single bond” refers to a chemical bond between two atoms, or two moieties when the atoms joined by the bond are considered to be part of a larger substructure. In one aspect, when a group described herein is a bond, the referenced group is absent thereby allowing a bond to be formed between the remaining identified groups.

The term “moiety” refers to a specific segment or functional group of a molecule. Chemical moieties are often recognized chemical entities embedded in or appended to a molecule.

The term “optionally substituted” or “substituted” means that the referenced group is optionally substituted with one or more additional group(s) individually and independently selected from halogen, —CN, —NH₂, —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄ alkyl), —C(═O)NH₂, —C(═O)NH(C₁-C₄ alkyl), —C(═O)N(C₁-C₄ alkyl)₂, —S(═O)₂NH₂, —S(═O)₂NH(C₁-C₄alkyl), —S(═O)₂N(C₁-C₄ alkyl)₂, —C₁-C₄ alkyl, C₃-C₆ cycloalkyl, —C₁-C₄ fluoroalkyl, —C₁-C₄ heteroalkyl, —C₁-C₄ alkoxy, —C₁-C₄ fluoroalkoxy, —SC₁-C₄ alkyl, —S(═O)C₁-C₄ alkyl, and —S(═O)₂C₁-C₄ alkyl. In some embodiments, optional substituents are independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, substituted groups are substituted with one or two of the preceding groups. In some embodiments, an optional substituent on an aliphatic carbon atom (acyclic or cyclic) includes oxo (═O).

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an agonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion). Those of skill in the art are familiar with administration techniques that can be employed with the compounds and methods described herein.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

### EXAMPLES

The following examples are provided for illustrative purposes only and not to limit the scope of the claims provided herein.

### Abbreviations

ACN or MeCN or CH₃CN: acetonitrile;
BBr₃: boron tribromide;
brine: saturated NaCl solution;
Cs₂CO₃: cesium carbonate;
DBAD: Di-tert-butyl azodicarboxylate
DCM: dichloromethane;
DIEA or DIPEA: N,N-diisopropylethylamine;
DMF: dimethylformamide;
DMSO: dimethyl sulfoxide;
DOTA: 2,2′,2″,2′″-(1,4,7,10-tetraazacyclododecane-1,4,7,10-tetrayl)tetraacetic acid or 1,4,7,10-tetraazacyclododecane-1,4,7,10-tetraacetic acid;
EDC: (1-ethyl-3-(3-dimethylaminopropyl)carbodiimide hydrochloride);
EtOAc or EA: ethyl acetate;
Fu catalyst complex: tris(dibenzylideneacetone)dipalladium(0) tri-tert-butylphosphonium tetrafluoroborate
HATU: 1-[Bis(dimethylamino)methylene]-1H-1,2,3-triazolo[4,5-b]pyridinium 3-oxide hexafluorophosphate;
HCl: hydrochloric acid or hydrochloride;
Hex: hexanes;
H₂O: water;
HOBt: hydroxybenzotriazole;
InCl₃: indium trichloride;
K₂CO₃: potassium carbonate;
KOAc: potassium acetate;
KOt-Bu: potassium t-butoxide;
K₃PO₄: potassium phosphate;
LCMS: Liquid chromatography-mass spectrometry;
LuCl₃: lutetium (III) chloride;
MPLC: Medium pressure liquid chromatography;
MS: mass spectrometry;
NaH: sodium hydride;
NaHCO₃: sodium bicarbonate;
NaIO₄: sodium periodate;
NaN[(CH₃)₃Si]₂: sodium bis(trimethylsilyl)amide
Na₂SO₄: sodium sulfate;
Pd(dppf)Cl₂: [1,1′-Bis(diphenylphosphino)ferrocene]dichloropalladium(II);
Pd(DTBPF)Cl₂: [1,1′-Bis(di-tert-butylphosphino)ferrocene]dichloropalladium(II);
PE: petroleum ether;
PPh₃: triphenyl phosphine;
RuCl₃: ruthenium(III) chloride;
Prep-HPLC: preparative high-performance liquid chromatography;
TFA: trifluoroacetic acid;
THF: tetrahydrofuran;
rt: room temperature;
hrs: hours; h or hr: hour;
min: minute;
mg: milligrams;
kg: kilograms;
mL or ml: milliliter;
Eq: equivalents;
mmol: millimole;
mol: moles;
UV: ultraviolet

### Synthesis of Compounds

### EXAMPLES

### Example 1: (Compound 1) 2,2′,2″-(10-(21-(2-(4-chloro-3-(4-cyano-6-(trifluoromethyl)pyridin-3-yl)-N-methylbenzamido)phenoxy)-2,19-dioxo-6,9,12,15-tetraoxa-3,18-diazahenicosyl)-1,4,7,10-tetraazacyclododecane-1,4,7-triyl)triacetic acid-2,2,2-trifluoroacetaldehyde (1/1)

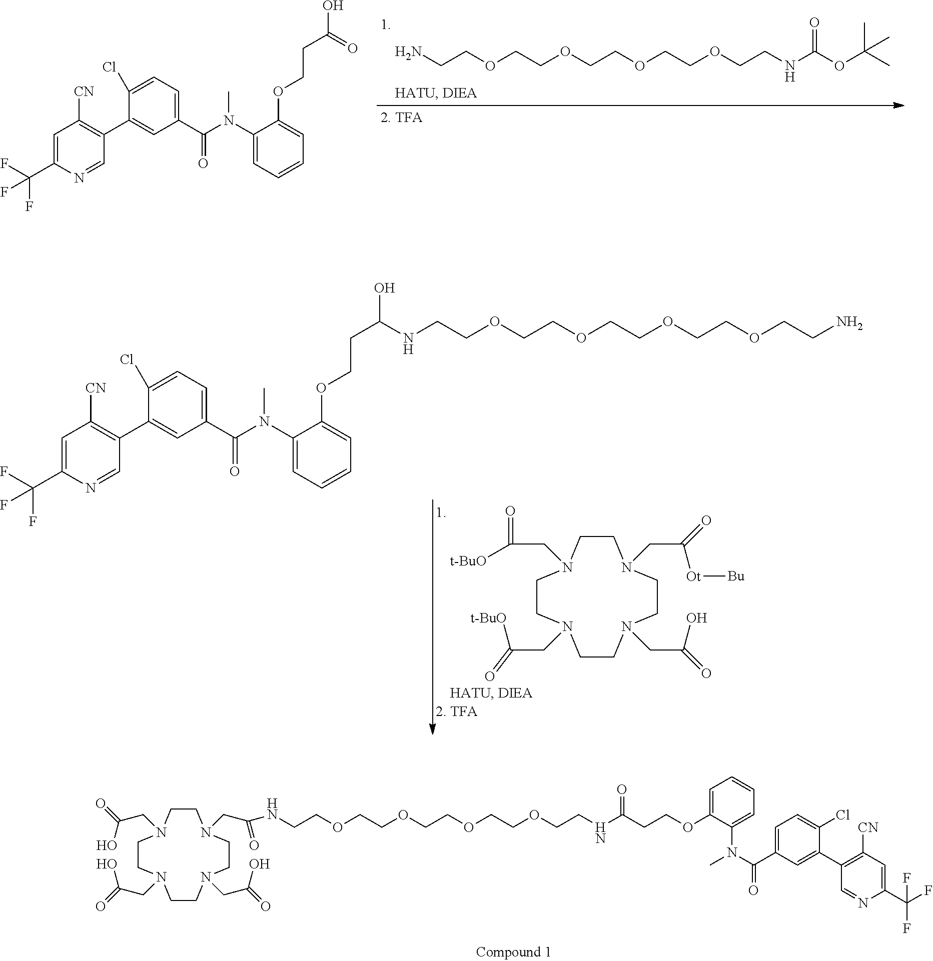

Step 1.: To a DMF (2 mL) solution of 3-(2-(4-chloro-3-(4-cyano-6-(trifluoromethyl)pyridin-3-yl)-N-methylbenzamido)phenoxy)propanoic acid (0.25 g, 1 Eq, 0.50 mmol) was added HATU (0.29 g, 1.5 Eq, 0.75 mmol), DIPEA (0.19 g, 0.26 mL, 3 Eq, 1.5 mmol) and tert-butyl (14-amino-3,6,9,12-tetraoxatetradecyl)carbamate (0.25 g, 1.5 Eq, 0.74 mmol). The resulting mixture was stirred at ambient temperature for 0.5 h. The crude reaction mixture was purified by C18 reverse phase chromatography eluting with MeCN (0.1% TFA)/water (0.1% TFA) (5-55%). Pure fractions were combined and dried under reduced pressure to afford the TFA salt of tert-butyl (18-(2-(4-chloro-3-(4-cyano-6-(trifluoromethyl)pyridin-3-yl)-N-methylbenzamido)phenoxy)-16-oxo-3,6,9,12-tetraoxa-15-azaoctadecyl)carbamate (40 mg, 8.7%). MS: Calc'd for C₄₁H₄₈ClF₆N₅O₁: 935.29, found [M+H-TFA]: 822.5.

Step 2.: To a DCM (0.6 mL) solution of tert-butyl (18-(2-(4-chloro-3-(4-cyano-6-(trifluoromethyl)pyridin-3-yl)-N-methylbenzamido)phenoxy)-16-oxo-3,6,9,12-tetraoxa-15-azaoctadecyl)carbamate-2,2,2-trifluoroacetaldehyde (1/1) (40 mg, 1 Eq, 43 μmol) was added TFA (0.9 g, 0.6 mL, 2e+2 Eq, 8 mmol). The resulting mixture was stirred at ambient temperature for 0.5 h. The crude reaction mixture was concentrated to afford the TFA salt of N-(2-((1-amino-16-oxo-3,6,9,12-tetraoxa-15-azaoctadecan-18-yl)oxy)phenyl)-4-chloro-3-(4-cyano-6-(trifluoromethyl)pyridin-3-yl)-N-methylbenzamide. This material was used in the next step without further purification. MS: Calc'd for C₃₄H₃₉ClF₃N₅O₇: 721.25, found [M+H]: 721.8.

Step 3.: To a DMF (0.6 mL) solution of 2-(4,7,10-tris(2-(tert-butoxy)-2-oxoethyl)-1,4,7,10-tetraazacyclododecan-1-yl)acetic acid (25 mg, 1.0 Eq, 43 μmol) was added DIPEA (0.2 g, 0.3 mL, 4e+1 Eq, 2 mmol), HATU (25 mg, 1.5 Eq, 65 μmol) and N-(2-((1-amino-16-oxo-3,6,9,12-tetraoxa-15-azaoctadecan-18-yl)oxy)phenyl)-4-chloro-3-(4-cyano-6-(trifluoromethyl)pyridin-3-yl)-N-methylbenzamide-2,2,2-trifluoroacetaldehyde (1/1) (35 mg, 1 Eq, 0.043 mmol) in 0.3 mL DMF. The resulting mixture was stirred at ambient temperature for 1 h. The crude reaction mixture was purified by C18 reverse phase chromatography eluting with MeCN (0.1% TFA)/water (0.1% TFA) (5-55%). Pure fractions were combined and dried under reduced pressure to afford the TFA salt of tri-tert-butyl 2,2′,2″-(10-(21-(2-(4-chloro-3-(4-cyano-6-(trifluoromethyl)pyridin-3-yl)-N-methylbenzamido)phenoxy)-2,19-dioxo-6,9,12,15-tetraoxa-3,18-diazahenicosyl)-1,4,7,10-tetraazacyclododecane-1,4,7-triyl)triacetate (16.6 mg, 28%). MS: Calc'd for C₆₄H₉₀ClF₆N₉O₁₆: 1389.61, found [(M-TFA/2)+H]: 639.0.

Step 4.: To a DCM (0.6 mL) of tri-tert-butyl 2,2′,2″-(10-(21-(2-(4-chloro-3-(4-cyano-6-(trifluoromethyl)pyridin-3-yl)-N-methylbenzamido)phenoxy)-2,19-dioxo-6,9,12,15-tetraoxa-3,18-diazahenicosyl)-1,4,7,10-tetraazacyclododecane-1,4,7-triyl)triacetate-2,2,2-trifluoroacetaldehyde (1/1) (16.6 mg, 12.1 μmol, 28%) was added TFA (0.6 mL). The resulting mixture was stirred at ambient temperature for 0.5 h. LCMS showed mostly starting material and mono-deprotection product. The crude reaction mixture was concentrated and re-dissolved in 0.7 mL TFA. The resulting mixture was heated at 60° C. for 1 h. The crude reaction mixture was purified by C18 reverse phase chromatography eluting with MeCN (0.1% TFA)/water (0.1% TFA) (5-55%). Pure fractions were combined and dried under reduced pressure to provide 2,2′,2″-(10-(21-(2-(4-chloro-3-(4-cyano-6-(trifluoromethyl)pyridin-3-yl)-N-methylbenzamido)phenoxy)-2,19-dioxo-6,9,12,15-tetraoxa-3,18-diazahenicosyl)-1,4,7,10-tetraazacyclododecane-1,4,7-triyl)triacetic acid-2,2,2-trifluoroacetaldehyde (1/1). Calc'd for C₅₂H₆₆ClF₆N₉O₁₆: 1221.42, found [M+H]: 1108.7.

### Synthesis of Compound 1M-Lu

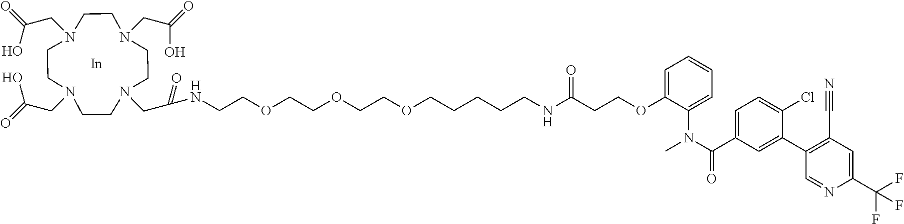

To a mixture of 2,2′,2″-(10-(21-(2-(4-chloro-3-(4-cyano-6-(trifluoromethyl)pyridin-3-yl)-N-methylbenzamido)phenoxy)-2,19-dioxo-6,9,12,15-tetraoxa-3,18-diazahenicosyl)-1,4,7,10-tetraazacyclododecane-1,4,7-triyl)triacetic acid-2,2,2-trifluoroacetaldehyde (1/1) (4.5 mg, 1 Eq, 3.7 μmol) and lutetium(III) chloride (3.1 mg, 3 Eq, 11 μmol) was added acetonitrile (0.2 mL), water (0.2 mL) and a saturated NaHCO₃ solution (0.1 mL). The resulting mixture was stirred at 80° C. for 2 hours. The crude reaction mixture was purified by C18 reverse phase chromatography eluting with MeCN (0.1% TFA)/water (0.1% TFA) (5-55%). Pure fractions were combined and dried under reduced pressure to give the TFA salt of the desired product. Calc'd for C₅₀H₆₂ClF₃LuN₉O₁₄: 1279.35, found [M+H]: 1280.5.

### Example 2: (Compound 2) 2,2′,2″-(10-(33-(2-(4-chloro-3-(4-cyano-6-(trifluoromethyl)-90-yridine-3-yl)-N-methylbenzamido)phenoxy)-2,31-dioxo-6,9,12,15,18,21,24,27-octaoxa-3,30-diazatritriacontyl)-1,4,7,10-tetraazacyclododecane-1,4,7-triyl)triacetic acid

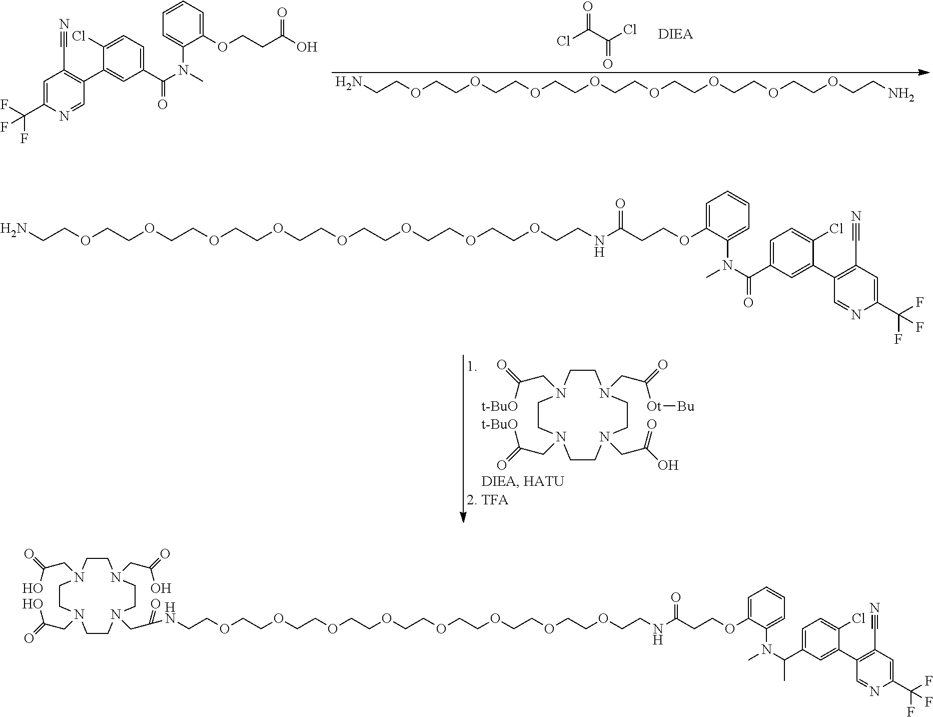

Step 1.: To a DCM (2 mL) solution of 3-(2-(4-chloro-3-(4-cyano-6-(trifluoromethyl)pyridin-3-yl)-N-methylbenzamido)phenoxy)propanoic acid (150 mg, 1.0 Eq, 298 μmol) was added oxalyl dichloride (75.6 mg, 51.1 μL, 2 Eq, 595 μmol), and then 2 drops of DMF was added at 0° C. The resulting mixture was stirred at the same temperature for 0.5 hours. The reaction mixture was concentrated to remove excess oxalyl chloride and then re-dissolved in DCM (2 mL), followed by addition of 3,6,9,12,15,18,21,24-octaoxahexacosane-1,26-diamine (147 mg, 1.2 Eq, 357 μmol) and N-ethyl-N-isopropylpropan-2-amine (231 mg, 311 μL, 6 Eq, 1.79 mmol) at 0° C. The resulting mixture was stirred at room temperature for 0.5 hours. The crude reaction mixture was concentrated and purified by C18 reverse column and concentrated to give N-(2-((1-amino-28-oxo-3,6,9,12,15,18,21,24-octaoxa-27-azatriacontan-30-yl)oxy)phenyl)-4-chloro-3-(4-cyano-6-(trifluoromethyl)pyridin-3-yl)-N-methylbenzamide (109.7 mg, 122.1 mol, 41.0%) as a TFA salt. Calc'd for C₄₂H₅₅ClF₃N₅O₁₁: 897.35, found [M+H]: 899.0.

Step 2. To a DMF (1 mL) solution of 2-(4,7,10-tris(2-(tert-butoxy)-2-oxoethyl)-1,4,7,10-tetraazacyclododecan-1-yl)acetic acid (63.06 mg, 1 Eq, 110.1 μmol) was added HATU (62.79 mg, 1.5 Eq, 165.1 μmol), DIPEA (56.92 mg, 77 μL, 4 Eq, 440.4 μmol) and N-(2-((1-amino-28-oxo-3,6,9,12,15,18,21,24-octaoxa-27-azatriacontan-30-yl)oxy)phenyl)-4-chloro-3-(4-cyano-6-(trifluoromethyl)pyridin-3-yl)-N-methylbenzamide-2,2,2-trifluoroacetaldehyde (1/1) (109.7 mg, 1 Eq, 110.1 μmol). The resulting mixture was stirred at ambient temperature for 0.5 hours. The reaction mixture was diluted with ethyl acetate, washed with water and brine, then concentrated to provide crude tritert-butyl 2,2′,2″-(10-(33-(2-(4-chloro-3-(4-cyano-6-(trifluoromethyl)pyridin-3-yl)-N-methylbenzamido)phenoxy)-2,31-dioxo-6,9,12,15,18,21,24,27-octaoxa-3,30-diazatritriacontyl)-1,4,7,10-tetraazacyclododecane-1,4,7-triyl)triacetate as a light brown solid. Calc'd for C₇₀H₁₀₅ClF₃N₉O₁₈: 1451.72, found [M+H]: 1453.2.

Step 3. A solution of tri-tert-butyl 2,2′,2″-(10-(33-(2-(4-chloro-3-(4-cyano-6-(trifluoromethyl)pyridin-3-yl)-N-methylbenzamido)phenoxy)-2,31-dioxo-6,9,12,15,18,21,24,27-octaoxa-3,30-diazatritriacontyl)-1,4,7,10-tetraazacyclododecane-1,4,7-triyl)triacetate-2,2,2-trifluoroacetaldehyde (1/1) (160 mg, 1 Eq, 103 μmol) in TFA (1.5 g, 1.0 mL, 1.3e+2 Eq, 13 mmol) was stirred at 20° C. for 1 hour. The crude reaction mixture was concentrated, and the remaining residue was purified by C18 reverse phase chromatography eluting with MeCN (0.1% TFA)/water (0.1% TFA). Pure fractions were combined and concentrated to give 2,2′,2″-(10-(33-(2-(4-chloro-3-(4-cyano-6-(trifluoromethyl)pyridin-3-yl)-N-methylbenzamido)phenoxy)-2,31-dioxo-6,9,12,15,18,21,24,27-octaoxa-3,30-diazatritriacontyl)-1,4,7,10-tetraazacyclododecane-1,4,7-triyl)triacetic acid as a TFA salt. Calc'd for C₅₈H₈₁ClF₃N₉O₁₈: 1283.53, found [M+H]: 1285.0.

### Synthesis of Compound 2M-Lu

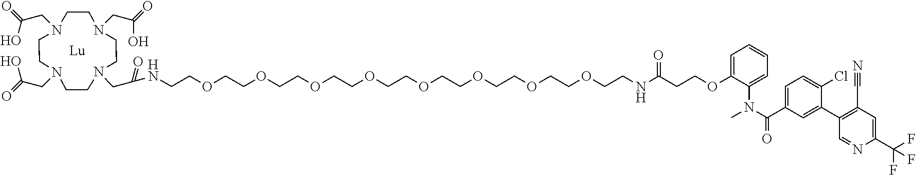

To a mixture of 2,2′,2″-(10-(33-(2-(4-chloro-3-(4-cyano-6-(trifluoromethyl)pyridin-3-yl)-N-methylbenzamido)phenoxy)-2,31-dioxo-6,9,12,15,18,21,24,27-octaoxa-3,30-diazatritriacontyl)-1,4,7,10-tetraazacyclododecane-1,4,7-triyl)triacetic acid-2,2,2-trifluoroacetaldehyde (1/1) (50 mg, 1 Eq, 36 μmol) and acetonitrile (0.5 mL) was added water (0.5 mL) and a saturated solution of NaHCO₃ (0.2 mL, 1 Eq, 36 μmol). The resulting mixture was heated at 80° C. for 2 hours. The crude reaction mixture was purified by C18 reverse phase chromatography eluting with MeCN (0.1% TFA)/water (0.1% TFA) (5-55%). Pure fractions were combined and concentrated to give the desired product as a TFA salt. Calc'd for C₆₀H₈₂ClF₆LuN₉O₂₀: 1572.47, found [M-TFA+H]: 1457.9.

### Synthesis of Compound 2M-In

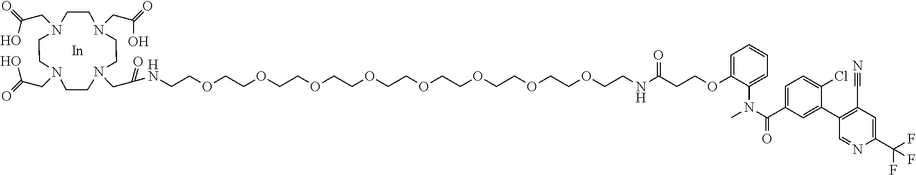

A mixture of 2,2′,2″-(10-(33-(2-(4-chloro-3-(4-cyano-6-(trifluoromethyl)pyridin-3-yl)-N-methylbenzamido)phenoxy)-2,31-dioxo-6,9,12,15,18,21,24,27-octaoxa-3,30-diazatritriacontyl)-1,4,7,10-tetraazacyclododecane-1,4,7-triyl)triacetic acid-2,2,2-trifluoroacetaldehyde (1/1) (45 mg, 1 Eq, 33 μmol) indium(III) chloride (25 mg, 3.5 Eq, 0.11 mmol) and sodium hydrogen carbonate (14 mg, 5 Eq, 0.16 mmol) in acetonitrile (0.5 mL) and water (0.5 mL) was stirred at 20° C. for 0.5 hour. The crude reaction mixture was purified by C18 reverse phase chromatography eluting with MeCN (0.1% TFA)/water (0.1% TFA). Pure fractions were combined and concentrated to provide indium(III) 2,2′,2″-(10-(33-(2-(4-chloro-3-(4-cyano-6-(trifluoromethyl)pyridin-3-yl)-N-methylbenzamido)phenoxy)-2,31-dioxo-6,9,12,15,18,21,24,27-octaoxa-3,30-diazatritriacontyl)-1,4,7,10-tetraazacyclododecane-1,4,7-triyl)triacetate as a TFA salt. Calc'd for C₅₈H₇₈ClF₃N₉O₁₈: 1395.41, found [M+H]: 1397.9.

### Example 3: (Compound 3) 2,2′,2″-(10-(29-(2-(4-chloro-3-(4-cyano-6-(trifluoromethyl)-93-yridine-3-yl)-N-methylbenzamido)phenoxy)-2-oxo-6,9,12,15,18,21,24,27-octaoxa-3-azanonacosyl)-1,4,7,10-tetraazacyclododecane-1,4,7-triyl)triacetic acid

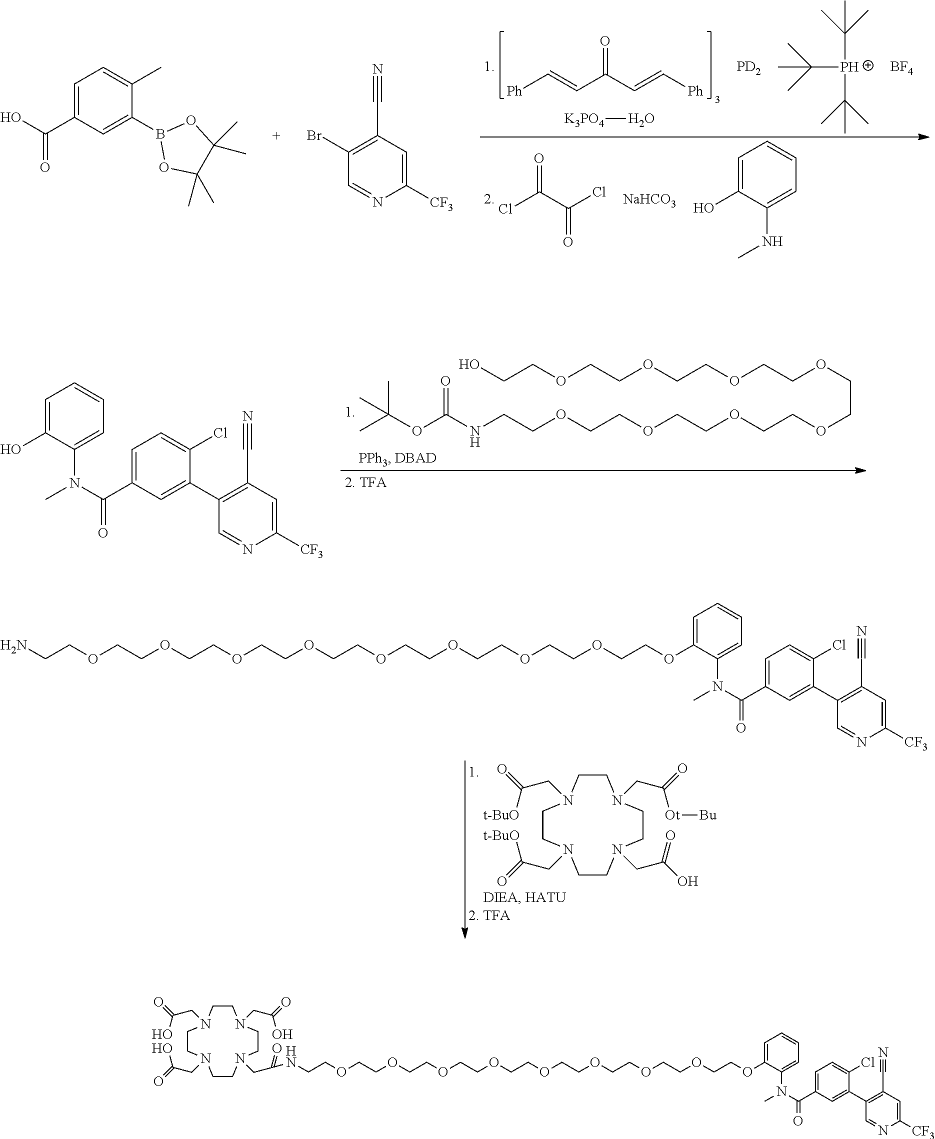

Step 1.: To a mixture of 5-bromo-2-(trifluoromethyl)isonicotinonitrile (30.0 mg, 1 Eq, 120 μmol), 4-chloro-3-(4,4,5,5-tetramethyl-1,3,2-dioxaborolan-2-yl)benzoic acid (67.5 mg, 2 Eq, 239 μmol), potassium phosphate hydrate (82.6 mg, 3 Eq, 359 μmol) and Fu catalyst complex (7.78 mg, 0.05 Eq, 5.98 μmol) was added THF (2 mL) and water (0.2 mL) under atmospheric nitrogen. The resulting mixture was bubbled with N₂ for 1 minute, then the reaction vessel was sealed and stirred at 25° C. for 1 hour. The crude reaction mixture was diluted with ethyl acetate (5.0 mL), washed with water and brine and concentrated. The residue was purified by silica gel chromatography to provide 4-chloro-3-(4-cyano-6-(trifluoromethyl)pyridin-3-yl)benzoic acid as a light brown solid. Calc'd for C₁₄H₆ClF₃N₂O₂: 326.00, found [M+H]: 327.3.

Step 2.: To a DCM (1 mL) solution of 4-chloro-3-(4-cyano-6-(trifluoromethyl)pyridin-3-yl)benzoic acid (54.0 mg, 1 Eq, 165 μmol) was added oxalyl dichloride (42.0 mg, 28.4 μL, 2 Eq, 331 μmol), and then 2 drops of DMF was added at 0° C. The resulting mixture was stirred at the same temperature for 0.5 hours. Upon completion of the reaction, the reaction mixture was concentrated to remove excess oxalyl chloride and then redissolved in acetonitrile (0.5 mL). The acetonitrile solution was added dropwise to a solution of 2-(methylamino)phenol (24.4 mg, 1.2 Eq, 198 μmol) and sodium bicarbonate (76.4 mg, 5.5 Eq, 909 μmol) in Acetonitrile (0.5 mL) and water (0.5 mL) at 0° C. The resulting mixture was stirred at room temperature for 0.5 hours. The crude reaction mixture was concentrated and purified by C18 reverse column and concentrated to provide 4-chloro-3-(4-cyano-6-(trifluoromethyl)pyridin-3-yl)-N-(2-hydroxyphenyl)-N-methylbenzamide as a TFA salt. Calc'd for C₂₁H₁₃ClF₃N₃O₂: 431.06, found [M+H]: 432.4.

Step 3.: To a mixture of 4-chloro-3-(4-cyano-6-(trifluoromethyl)pyridin-3-yl)-N-(2-hydroxyphenyl)-N-methylbenzamide (20.0 mg, 1.0 Eq, 46.3 μmol) and tert-butyl (26-hydroxy-3,6,9,12,15,18,21,24-octaoxahexacosyl)carbamate (26.2 mg, 1.1 Eq, 50.9 μmol) in dry THF (0.6 mL) was added triphenylphosphine (24.3 mg, 2 Eq, 92.6 μmol) and di-tert-butyl azodicarboxylate (21.3 mg, 2 Eq, 92.6 μmol). The reaction mixture was stirred at 20° C. for 0.5 hours. Calc'd for C₄₄H₅₈ClF₃N₄O₁₂: 926.36, found [M+H]: 927.8.

Step 4.: To a DCM (0.5 mL) solution of tert-butyl (26-(2-(4-chloro-3-(4-cyano-6-(trifluoromethyl)pyridin-3-yl)-N-methylbenzamido)phenoxy)-3,6,9,12,15,18,21,24-octaoxahexacosyl)carbamate (43 mg, 1 Eq, 46 μmol) was added TFA (0.7 g, 0.5 mL, 1e+2 Eq, 6 mmol). The resulting mixture was stirred at ambient temperature for 0.5 h. Upon completion of the reaction, the reaction mixture was concentrated and dried to give crude product N-(2-((26-amino-3,6,9,12,15,18,21,24-octaoxahexacosyl)oxy)phenyl)-4-chloro-3-(4-cyano-6-(trifluoromethyl)pyridin-3-yl)-N-methylbenzamide (41 mg, 50 μmol, 110%), which was used directly in the next step without further purification. Calc'd for C₃₉H₅₀ClF₃N₄O₁₀: 826.32, found [M+H]: 827.7.

Step 5.: To a solution of 2-(4,7,10-tris(2-(tert-butoxy)-2-oxoethyl)-1,4,7,10-tetraazacyclododecan-1-yl)acetic acid (23.3 mg, 1 Eq, 40.7 μmol) and 2-(3H-[1,2,3]triazolo[4,5-b]pyridin-3-yl)-1,1,3,3-tetramethylisouronium hexafluorophosphate(V) (31.0 mg, 2 Eq, 81.5 mol) in DMF (0.5 mL) was added N-(2-((26-amino-3,6,9,12,15,18,21,24-octaoxahexacosyl)oxy)phenyl)-4-chloro-3-(4-cyano-6-(trifluoromethyl)pyridin-3-yl)-N-methylbenzamide-2,2,2-trifluoroacetaldehyde (1/2) (41.7 mg, 1 Eq, 40.7 μmol) followed by N-ethyl-N-isopropylpropan-2-amine (26.3 mg, 35.5 μL, 5 Eq, 204 μmol). The reaction mixture was stirred at room temperature for 2 hours. The reaction mixture was diluted with ethyl acetate, washed with water and brine, concentrated and purified by gradient column chromatography (EtOAc-Hex) to provide the target compound tri-tert-butyl 2,2′,2″-(10-(29-(2-(4-chloro-3-(4-cyano-6-(trifluoromethyl)pyridin-3-yl)-N-methylbenzamido)phenoxy)-2-oxo-6,9,12,15,18,21,24,27-octaoxa-3-azanonacosyl)-1,4,7,10-tetraazacyclododecane-1,4,7-triyl)triacetate as a brown solid. Calc'd for C₆₇H₁₀₀ClF₃N₈O₁₇: 1380.68, found [M/2+H]: 691.5.

Step 6.: 6,9,12,15,18,21,24,27-octaoxa-3-azanonacosyl)-1,4,7,10-tetraazacyclododecane-1,4,7-triyl)triacetate (56.3 mg, 1 Eq, 40.7 μmol) was treated with 2,2,2-trifluoroacetic acid (4.64 mg, 1 Eq, 40.7 μmol) at 20° C. for 2 hours. The crude reaction mixture was concentrated and then was purified by C18 reverse phase chromatography eluting with MeCN (0.1% TFA)/water (0.1% TFA). Pure fractions were dried and combined to provide 2,2′,2″-(10-(29-(2-(4-chloro-3-(4-cyano-6-(trifluoromethyl)pyridin-3-yl)-N-methylbenzamido)phenoxy)-2-oxo-6,9,12,15,18,21,24,27-octaoxa-3-azanonacosyl)-1,4,7,10-tetraazacyclododecane-1,4,7-triyl)triacetic acid as a TFA salt. Calc'd for C₅₅H₇₆ClF₃N₈O₁₇: 1212.50, found [M+H]: 1213.8.

### Synthesis of Compound 3M-In

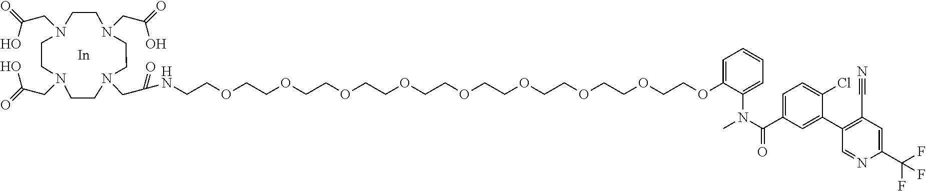

A mixture of 2,2′,2″-(10-(29-(2-(4-chloro-3-(4-cyano-6-(trifluoromethyl)pyridin-3-yl)-N-methylbenzamido)phenoxy)-2-oxo-6,9,12,15,18,21,24,27-octaoxa-3-azanonacosyl)-1,4,7,10-tetraazacyclododecane-1,4,7-triyl)triacetic acid (20 mg, 1 Eq, 16 μmol), indium(III) chloride (13 mg, 3.5 Eq, 58 μmol) and sodium hydrogen carbonate (6.9 mg, 5 Eq, 82 μmol) in acetonitrile (0.4 mL) and water (0.4 mL) was stirred at 20° C. for 2 hours. The crude reaction mixture was purified by C18 reverse phase chromatography eluting with MeCN (0.1% TFA)/water (0.1% TFA). Pure fractions were combined and concentrated to provide indium(III) 2,2′,2″-(10-(29-(2-(4-chloro-3-(4-cyano-6-(trifluoromethyl)pyridin-3-yl)-N-methylbenzamido)phenoxy)-2-oxo-6,9,12,15,18,21,24,27-octaoxa-3-azanonacosyl)-1,4,7,10-tetraazacyclododecane-1,4,7-triyl)triacetate as a TFA salt. Calc'd for C₅₅H₇₃ClF₃N₈O₁₇: 1324.38, found [M+H]: 1326.1.

### Example 4: (Compound 4) 2,2′,2″-(10-(29-((4,6-dimethoxy-5-(5-((3,3,6-trimethyl-2,3-dihydro-1H-inden-5-yl)oxy)furan-2-carboxamido)pyrimidin-2-yl)oxy)-2-oxo-6,9,12,15,18,21,24,27-octaoxa-3-azanonacosyl)-1,4,7,10-tetraazacyclododecane-1,4,7-triyl)triacetic acid

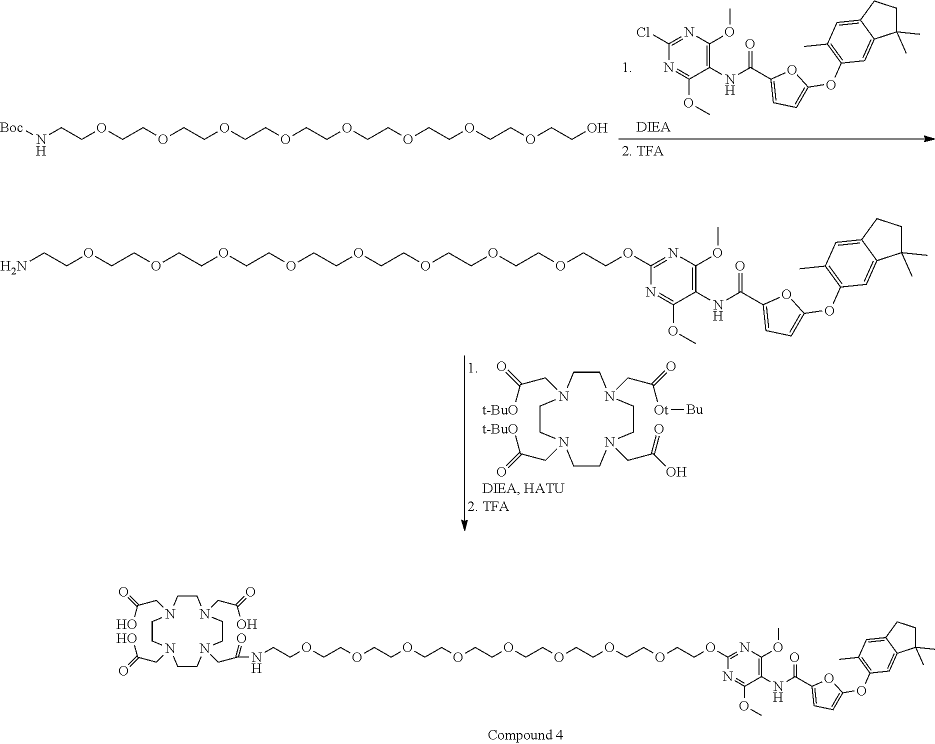

Step 1.: A mixture of N-(2-chloro-4,6-dimethoxypyrimidin-5-yl)-5-((3,3,6-trimethyl-2,3-dihydro-1H-inden-5-yl)oxy)furan-2-carboxamide (60.0 mg, 1 Eq, 131 μmol), tert-butyl (26-hydroxy-3,6,9,12,15,18,21,24-octaoxahexacosyl)carbamate (80.8 mg, 1.2 Eq, 157 μmol) and DIPEA (169 mg, 228 μL, 10 Eq, 1.31 mmol) in DMSO (1 mL) was heated at 120° C. for 3 hours. H₂O was added to the reaction mixture, then the aqueous layer was extracted with EtOAc, washed with brine, concentrated and purified by Isco silica gel chromatography (0-100% EtOAc/hexanes) to provide tert-butyl (26-((4,6-dimethoxy-5-(5-((3,3,6-trimethyl-2,3-dihydro-1H-inden-5-yl)oxy)furan-2-carboxamido)pyrimidin-2-yl)oxy)-3,6,9,12,15,18,21,24-octaoxahexacosyl)carbamate as a yellow solid. Calc'd for C₄₆H₇₀N₄O₁₆: 934.48, found [M+H]: 934.9.

Step 2.: To a solution of tert-butyl (26-((4,6-dimethoxy-5-(5-((3,3,6-trimethyl-2,3-dihydro-1H-inden-5-yl)oxy)furan-2-carboxamido)pyrimidin-2-yl)oxy)-3,6,9,12,15,18,21,24-octaoxahexacosyl)carbamate (123.0 mg, 1 Eq, 131.5 μmol) in DCM (0.6 mL) was added TFA (0.7 g, 0.5 mL, 5e+1 Eq, 6 mmol). The reaction was stirred at 20° C. for 30 min. The crude reaction mixture was concentrated and then was purified by C18 reverse phase chromatography eluting with MeCN (0.1% TFA)/water (0.1% TFA). Pure fractions were dried and combined, to provide N-(2-((26-amino-3,6,9,12,15,18,21,24-octaoxahexacosyl)oxy)-4,6-dimethoxypyrimidin-5-yl)-5-((3,3,6-trimethyl-2,3-dihydro-1H-inden-5-yl)oxy)furan-2-carboxamide (100.9 mg, 120.8 mol, 91.87%) as a TFA salt. Calc'd for C₄₃H₆₃F₃N₄O₁₆: 948.42, found [(M-TFA)/2+H]: 418.4.

Step 3.: To a solution of 2-(4,7,10-tris(2-(tert-butoxy)-2-oxoethyl)-1,4,7,10-tetraazacyclododecan-1-yl)acetic acid (62.36 mg, 1.0 Eq, 108.9 μmol) and 2-(3H-[1,2,3]triazolo[4,5-b]pyridin-3-yl)-1,1,3,3-tetramethylisouronium hexafluorophosphate(V) (82.80 mg, 2.0 Eq, 217.8 μmol) in DMF (0.8 mL) was added N-ethyl-N-isopropylpropan-2-amine (56.29 mg, 75.9 μL, 4.0 Eq, 435.5 μmol) followed by N-(2-((26-amino-3,6,9,12,15,18,21,24-octaoxahexacosyl)oxy)-4,6-dimethoxypyrimidin-5-yl)-5-((3,3,6-trimethyl-2,3-dihydro-1H-inden-5-yl)oxy)furan-2-carboxamide (100.0 mg, 1.1 Eq, 119.8 μmol). The reaction mixture was stirred at 25° C. for 0.5 hours. The reaction mixture was diluted with ethyl acetate, washed with water and brine, concentrated to provide crude tri-tert-butyl 2,2′,2″-(10-(29-((4,6-dimethoxy-5-(5-((3,3,6-trimethyl-2,3-dihydro-1H-inden-5-yl)oxy)furan-2-carboxamido)pyrimidin-2-yl)oxy)-2-oxo-6,9,12,15,18,21,24,27-octaoxa-3-azanonacosyl)-1,4,7,10-tetraazacyclododecane-1,4,7-triyl)triacetate as a light brown solid. Calc'd for C₆₉H₁₁₂N₈O₂₁: 1388.79, found [M+H]: 1390.1.

Step 4.: A solution of tri-tert-butyl 2,2′,2″-(10-(29-((4,6-dimethoxy-5-(5-((3,3,6-trimethyl-2,3-dihydro-1H-inden-5-yl)oxy)furan-2-carboxamido)pyrimidin-2-yl)oxy)-2-oxo-6,9,12,15,18,21,24,27-octaoxa-3-azanonacosyl)-1,4,7,10-tetraazacyclododecane-1,4,7-triyl)triacetate (151 mg, 1 Eq, 109 μmol) in TFA (0.3 g, 0.2 mL, 2e+1 Eq, 3 mmol) was stirred at 25° C. for 0.5 hours. The crude reaction mixture was concentrated and then was purified by C18 reverse phase chromatography eluting with MeCN (0.1% TFA)/water (0.1% TFA). Pure fractions were dried and combined to provide 2,2′,2″-(10-(29-((4,6-dimethoxy-5-(5-((3,3,6-trimethyl-2,3-dihydro-1H-inden-5-yl)oxy)furan-2-carboxamido)pyrimidin-2-yl)oxy)-2-oxo-6,9,12,15,18,21,24,27-octaoxa-3-azanonacosyl)-1,4,7,10-tetraazacyclododecane-1,4,7-triyl)triacetic acid as a TFA salt. Calc'd for C₅₇H₈₈N₈O₂₁: 1220.60, found [M+H]: 1221.9.

### Synthesis of Compound 4M-In

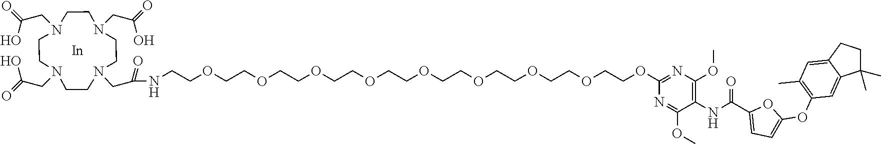

A solution of 2,2′,2″-(10-(29-((4,6-dimethoxy-5-(5-((3,3,6-trimethyl-2,3-dihydro-1H-inden-5-yl)oxy)furan-2-carboxamido)pyrimidin-2-yl)oxy)-2-oxo-6,9,12,15,18,21,24,27-octaoxa-3-azanonacosyl)-1,4,7,10-tetraazacyclododecane-1,4,7-triyl)triacetic acid (40.0 mg, 1 Eq, 32.8 mol), sodium bicarbonate (27.5 mg, 10 Eq, 328 μmol) and indium(III) chloride (21.7 mg, 3 Eq, 98.3 μmol) in acetonitrile (0.5 mL) and water (0.5 mL) was stirred at 20° C. for 2 hours. The crude reaction mixture was purified directly by C18 reverse phase chromatography eluting with MeCN (0.1% TFA)/water (0.1% TFA). Pure fractions were dried and combined to provide indium(III) 2,2′,2″-(10-(29-((4,6-dimethoxy-5-(5-((3,3,6-trimethyl-2,3-dihydro-1H-inden-5-yl)oxy)furan-2-carboxamido)pyrimidin-2-yl)oxy)-2-oxo-6,9,12,15,18,21,24,27-octaoxa-3-azanonacosyl)-1,4,7,10-tetraazacyclododecane-1,4,7-triyl)triacetate (30.9 mg, 23.2 μmol, 70.8%) as a TFA salt. Calc'd for C₅₇H₈₅InN₈O₂₁: 1332.49, found [M+H]: 1333.1.

### Example 5: (Compound 5) 2,2′,2″-(10-(29-(5-chloro-4-(4-cyano-6-(trifluoromethyl)pyridin-3-yl)-2-((2-methoxyphenyl) (methyl)carbamoyl)phenoxy)-2-oxo-6,9,12,15,18,21,24,27-octaoxa-3-azanonacosyl)-1,4,7,10-tetraazacyclododecane-1,4,7-triyl)triacetic acid

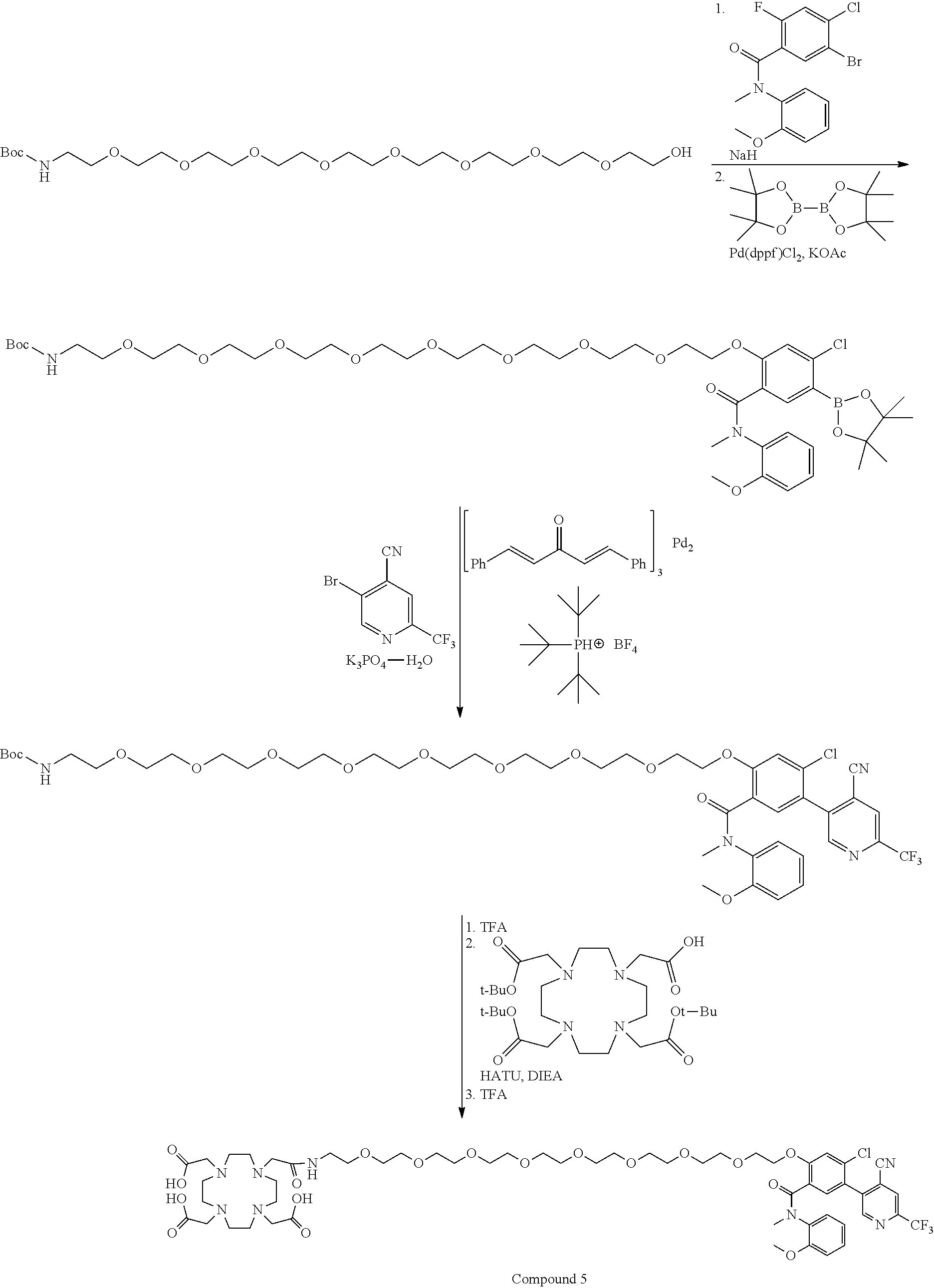

Step 1.: A solution of 5-bromo-4-chloro-2-fluoro-N-(2-methoxyphenyl)-N-methylbenzamide (160.9 mg, 1 Eq, 431.8 μmol) in DMF (3 mL) was cooled to 0° C., then sodium hydride (60.45 mg, 60% wt, 3.5 Eq, 1.511 mmol) was added under N₂, The reaction mixture was stirred at 0° C. for 5 minutes, then tert-butyl (26-hydroxy-3,6,9,12,15,18,21,24-octaoxahexacosyl)carbamate (266.1 mg, 1.2 Eq, 518.2 μmol) was then added. The reaction mixture was allowed to warm to room temperature, stirred for 30 minutes, then the reaction was completed. The reaction was carefully quenched by addition of water, then the aqueous layer was extracted with EtOAc, washed with water and brine, concentrated and purified by Isco silica gel chromatography to provide tert-butyl (26-(4-bromo-5-chloro-2-((2-methoxyphenyl)(methyl)carbamoyl)phenoxy)-3,6,9,12,15,18,21,24-octaoxahexacosyl)carbamate as a colorless oil. Calc'd for C₃₈H₅₈BrClN₂O₁₃: 864.28, found [M+H]: 865.5.

Step 2.: A mixture of tert-butyl (26-(4-bromo-5-chloro-2-((2-methoxyphenyl)(methyl)carbamoyl)phenoxy)-3,6,9,12,15,18,21,24-octaoxahexacosyl)carbamate (100 mg, 1 Eq, 115 μmol), 4,4,4′,4′,5,5,5′,5′-octamethyl-2,2′-bi(1,3,2-dioxaborolane) (35.2 mg, 1.2 Eq, 139 μmol), potassium acetate (34.0 mg, 3 Eq, 346 μmol) and Pd(dppf)Cl₂ (6.76 mg, 0.08 Eq, 9.24 μmol) in 1,4-dioxane (500 mL) was bubbled with N₂ for 1 min and the resulting mixture was sealed and heated at 80° C. for 12 h. The crude reaction mixture was diluted with water and extracted with EtOAc, washed with brine, then concentrated. The crude product was purified by silica gel chromatography to provide tert-butyl (26-(5-chloro-2-((2-methoxyphenyl)(methyl)carbamoyl)-4-(4,4,5,5-tetramethyl-1,3,2-dioxaborolan-2-yl)phenoxy)-3,6,9,12,15,18,21,24-octaoxahexacosyl)carbamate as a light brown solid. Calc'd for C₄₄H₇₀BClN₂O₁₅: 912.46, found [M+H]: 914.1.

Step 3.: To a mixture of tert-butyl (26-(5-chloro-2-((2-methoxyphenyl)(methyl)carbamoyl)-4-(4,4,5,5-tetramethyl-1,3,2-dioxaborolan-2-yl)phenoxy)-3,6,9,12,15,18,21,24-octaoxahexacosyl)carbamate (100 mg, 1 Eq, 109 μmol), 5-bromo-2-(trifluoromethyl)isonicotinonitrile (33.0 mg, 1.2 Eq, 131 μmol), potassium phosphate hydrate (75.6 mg, 3 Eq, 328 μmol) and Fu catalyst complex (14.3 mg, 0.1 Eq, 10.9 μmol) was added THF (0.5 mL) and water (0.1 mL) under atmospheric nitrogen. The resulting mixture was bubbled with N₂ for 1 minute, then the reaction vessel was sealed and heated at 60° C. for 3 hours. The crude reaction mixture was cooled to room temperature and diluted with ethyl acetate (5.0 mL), washed with water and brine, dried over anhydrous Na₂SO₄ and concentrated. The residue was purified by silica gel chromatography to provide tert-butyl (26-(5-chloro-4-(4-cyano-6-(trifluoromethyl)pyridin-3-yl)-2-((2-methoxyphenyl)(methyl)carbamoyl)phenoxy)-3,6,9,12,15,18,21,24-octaoxahexacosyl)carbamate as a light brown solid. Calc'd for C₄₅H₆₀ClF₃N₄O₁₃: 956.38, found [M+H]: 957.5.

Step 4.: To a DCM (0.6 mL) solution of tert-butyl (26-(5-chloro-4-(4-cyano-6-(trifluoromethyl)pyridin-3-yl)-2-((2-methoxyphenyl) (methyl)carbamoyl)phenoxy)-3,6,9,12,15,18,21,24-octaoxahexacosyl)carbamate (105 mg, 1 Eq, 110 μmol) was added TFA (0.7 g, 0.5 mL, 6e+1 Eq, 6 mmol). The resulting mixture was stirred at ambient temperature for 0.5 h. The crude reaction mixture was concentrated and then was purified by C18 reverse phase chromatography eluting with MeCN (0.1% TFA)/water (0.1% TFA). Pure fractions were dried and combined, to provide 2-((26-amino-3,6,9,12,15,18,21,24-octaoxahexacosyl)oxy)-4-chloro-5-(4-cyano-6-(trifluoromethyl)pyridin-3-yl)-N-(2-methoxyphenyl)-N-methylbenzamide as a TFA salt. Calc'd for C₄₀H₅₂ClF₃N₄O₁₁: 856.33, found [M+H]: 857.8.

Step 5.: To a solution of 2-(4,7,10-tris(2-(tert-butoxy)-2-oxoethyl)-1,4,7,10-tetraazacyclododecan-1-yl)acetic acid (17.6 mg, 1 Eq, 30.7 μmol) and 2-(3H-[1,2,3]triazolo[4,5-b]pyridin-3-yl)-1,1,3,3-tetramethylisouronium hexafluorophosphate(V) (23.3 mg, 2 Eq, 61.4 mol) in DMF (0.5 mL) was added N-ethyl-N-isopropylpropan-2-amine (19.8 mg, 26.7 μL, 5 Eq, 153 μmol) followed by 2-((26-amino-3,6,9,12,15,18,21,24-octaoxahexacosyl)oxy)-4-chloro-5-(4-cyano-6-(trifluoromethyl)pyridin-3-yl)-N-(2-methoxyphenyl)-N-methylbenzamide (26.3 mg, 1 Eq, 30.7 μmol). The reaction mixture was stirred at room temperature for 2 hours. The reaction mixture was diluted with ethyl acetate, washed with water and brine, concentrated and purified by gradient column chromatography (EtOAc-Hex) to provide the target compound tri-tert-butyl 2,2′,2″-(10-(29-(5-chloro-4-(4-cyano-6-(trifluoromethyl)pyridin-3-yl)-2-((2-methoxyphenyl) (methyl)carbamoyl)phenoxy)-2-oxo-6,9,12,15,18,21,24,27-octaoxa-3-azanonacosyl)-1,4,7,10-tetraazacyclododecane-1,4,7-triyl)triacetate as a white solid. Calc'd for C₆₈H₁₀₂ClF₃N₈O₁₈: 1410.70, found [M+H]: 1412.8.

Step 6.: tri-tert-butyl 2,2′,2″-(10-(29-(5-chloro-4-(4-cyano-6-(trifluoromethyl)pyridin-3-yl)-2-((2-methoxyphenyl)(methyl)carbamoyl)phenoxy)-2-oxo-6,9,12,15,18,21,24,27-octaoxa-3-azanonacosyl)-1,4,7,10-tetraazacyclododecane-1,4,7-triyl)triacetate (43.3 mg, 1 Eq, 30.7 μmol) was dissolved in 2,2,2-trifluoroacetic acid (3.50 mg, 1 Eq, 30.7 μmol). The resulting mixture was stirred at ambient temperature for 1 h. The crude reaction mixture was concentrated and then was purified by C18 reverse phase chromatography eluting with MeCN (0.1% TFA)/water (0.1% TFA) to provide 2,2′,2″-(10-(29-(5-chloro-4-(4-cyano-6-(trifluoromethyl)pyridin-3-yl)-2-((2-methoxyphenyl) (methyl)carbamoyl)phenoxy)-2-oxo-6,9,12,15,18,21,24,27-octaoxa-3-azanonacosyl)-1,4,7,10-tetraazacyclododecane-1,4,7-triyl)triacetic acid as a TFA salt. Calc'd for C₅₆H₇₈ClF₃N₈O₁₈: 1242.51, found [M+H]: 1244.0.

### Synthesis of Compound 5M-In

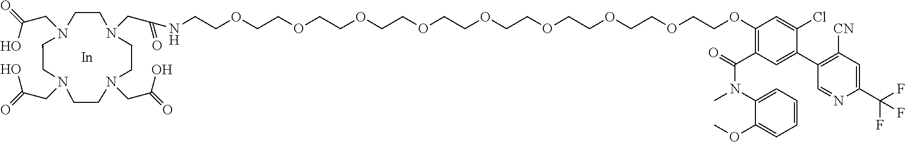

To an acetonitrile (0.3 mL) and water (0.3 mL) solution of 2,2′,2″-(10-(29-(5-chloro-4-(4-cyano-6-(trifluoromethyl)pyridin-3-yl)-2-((2-methoxyphenyl)(methyl)carbamoyl)phenoxy)-2-oxo-6,9,12,15,18,21,24,27-octaoxa-3-azanonacosyl)-1,4,7,10-tetraazacyclododecane-1,4,7-triyl)triacetic acid (12 mg, 1 Eq, 9.6 μmol) and sodium hydrogen carbonate (8.1 mg, 10 Eq, 96 mol) was added indium(III) chloride (6.4 mg, 3 Eq, 29 μmol). The resulting mixture was stirred at ambient temperature for 2 h. The crude reaction mixture was concentrated and then was purified by C18 reverse phase chromatography eluting with MeCN (0.1% TFA)/water (0.1% TFA). Pure fractions were dried and combined to provide indium(III) 2,2′,2″-(10-(29-(5-chloro-4-(4-cyano-6-(trifluoromethyl)pyridin-3-yl)-2-((2-methoxyphenyl) (methyl)carbamoyl)phenoxy)-2-oxo-6,9,12,15,18,21,24,27-octaoxa-3-azanonacosyl)-1,4,7,10-tetraazacyclododecane-1,4,7-triyl)triacetate (8.7 mg, 6.4 μmol, 67%) as a TFA salt. Calc'd for C₅₆H₇₅ClF₃InN₈O₁₈: 1354.39, found [M+H]: 1356.8.

### Example 6: (Compound 6) 2,2′,2″-(10-(34-(5-chloro-4-(4-cyano-6-(trifluoromethyl)pyridin-3-yl)-2-((2-methoxyphenyl)(methyl)carbamoyl)phenoxy)-2,31-dioxo-6,9,12,15,18,21,24,27-octaoxa-3,30-diazatetratriacontyl)-1,4,7,10-tetraazacyclododecane-1,4,7-triyl)triacetic acid

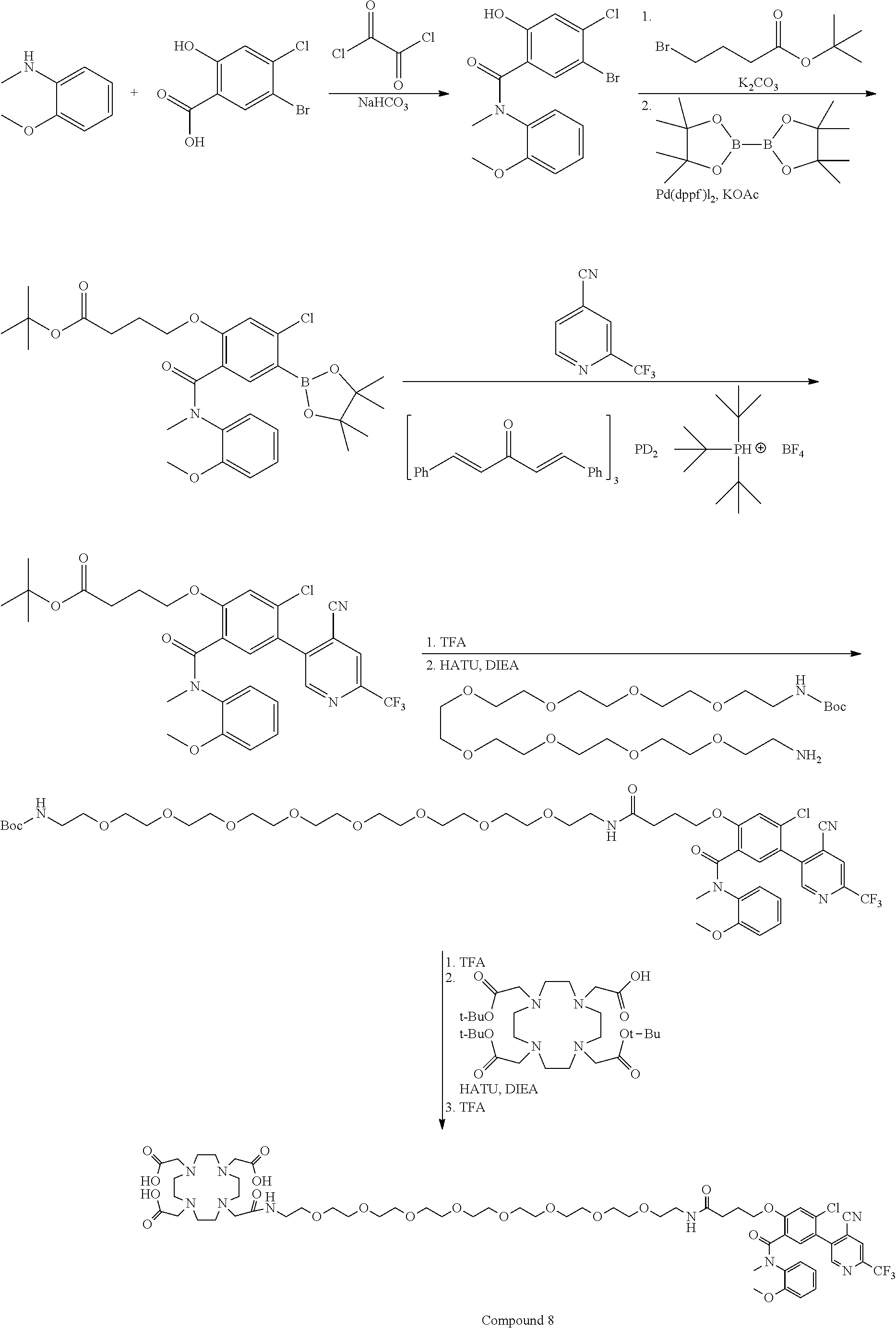

Step 1.: To a suspension of 5-bromo-4-chloro-2-hydroxybenzoic acid (817 mg, 1 Eq, 3.25 mmol) in DCM (15 mL) was added oxalyl chloride (825 mg, 569 μL, 2.0 Eq, 6.50 mmol), and then a few drops of DMF was added at 20° C. The resulting mixture was stirred at 20° C. for 1 hour, until the reaction mixture became clear to indicate that the reaction was complete. The reaction mixture was concentrated to remove excess oxalyl chloride and then re-dissolved in acetonitrile (6 mL). The acetonitrile solution was then slowly added to a solution of 2-methoxy-N-methylaniline (490 mg, 1.1 Eq, 3.57 mmol) and sodium bicarbonate (1.36 g, 5 Eq, 16.2 mmol) in acetonitrile (6 mL) and water (6 mL). The resulting mixture was stirred at room temperature for 2 hours. The mixture was extracted with EtOAc, then washed with 4N HCl to removed unreacted 2-methoxy-N-methylaniline (490 mg, 1.1 Eq, 3.57 mmol). The reaction mixture was concentrated and purified by Isco silica gel chromatography to give 5-bromo-4-chloro-2-hydroxy-N-(2-methoxyphenyl)-N-methylbenzamide (618 mg, 1.67 mmol, 51.3%). Calc'd for C₁₅H₁₃BrClNO₃: 368.98, found [M+H]: 370.1.

Step 2.: tert-Butyl 4-bromobutanoate (1.09 g, 869 μL, 3 Eq, 4.90 mmol) was added to the solution of 5-bromo-4-chloro-2-hydroxy-N-(2-methoxyphenyl)-N-methylbenzamide (605 mg, 1 Eq, 1.63 mmol) and potassium carbonate (677 mg, 3 Eq, 4.90 mmol) in DMF (6 mL), then stirred at 80° C. for 2 hours until the reaction was complete. The reaction mixture was cooled to room temperature, water was added then the aqueous layer was extracted with EtOAc, washed with brine, concentrated and purified by Isco silica gel chromatography to provide tert-butyl 4-(4-bromo-5-chloro-2-((2-methoxyphenyl)(methyl)carbamoyl)phenoxy)butanoate (797 mg, 1.55 mmol, 95.2%) as a colorless oil. Calc'd for C₂₃H₂₇BrClNO₅: 511.08, found [M+H]: 514.3.

Step 3.: A mixture of tert-butyl 4-(4-bromo-5-chloro-2-((2-methoxyphenyl)(methyl)carbamoyl)phenoxy)butanoate (797 mg, 1 Eq, 1.55 mmol), 4,4,4′,4′,5,5,5′,5′-octamethyl-2,2′-bi(1,3,2-dioxaborolane) (474 mg, 1.2 Eq, 1.86 mmol), potassium acetate (458 mg, 3 Eq, 4.66 mmol) and Pd(dppf)Cl₂ (91.0 mg, 0.08 Eq, 124 μmol) in 1,4-dioxane (8 mL) was bubbled with N₂ for 1 min and the resulting mixture was sealed and heated at 100° C. for 1 h. The crude reaction mixture was diluted with water and extracted with EtOAc, washed with brine, concentrated and purified by silica gel chromatography to provide tert-butyl 4-(5-chloro-2-((2-methoxyphenyl)(methyl)carbamoyl)-4-(4,4,5,5-tetramethyl-1,3,2-dioxaborolan-2-yl)phenoxy)butanoate (642 mg, 1.15 mmol, 73.8%) as a light brown oil. Calc'd for C₂₉H₃₉BClNO₇: 559.25, found [M+H]: 560.7.

Step 4.: To a mixture of 5-bromo-2-(trifluoromethyl)isonicotinonitrile (150 mg, 1.1 Eq, 598 μmol), tert-butyl 4-(5-chloro-2-((2-methoxyphenyl) (methyl)carbamoyl)-4-(4,4,5,5-tetramethyl-1,3,2-dioxaborolan-2-yl)phenoxy)butanoate (304 mg, 1 Eq, 543 μmol), potassium phosphate hydrate (375 mg, 3 Eq, 1.63 mmol) and Fu catalyst complex (70.7 mg, 0.1 Eq, 54.3 mol) was added THF (2 mL) and water (0.2 mL) under atmospheric nitrogen. The resulting mixture was bubbled with N₂ for 1 minute, then the reaction vessel was sealed and heated at 80° C. for 1 hour. The crude reaction mixture was cooled to room temperature and diluted with ethyl acetate (5.0 mL), washed with water and brine, dried over anhydrous Na₂SO₄ and concentrated. The residue was purified by silica gel chromatography to provide tert-butyl 4-(5-chloro-4-(4-cyano-6-(trifluoromethyl)pyridin-3-yl)-2-((2-methoxyphenyl)(methyl)carbamoyl)phenoxy)butanoate as a light brown solid. Calc'd for C₅H₂₉ClF₃N₃O₁₈: 603.17, found [M+H]: 604.7.

Step 5.: To a DCM (0.5 mL) solution of tert-butyl 4-(5-chloro-4-(4-cyano-6-(trifluoromethyl)pyridin-3-yl)-2-((2-methoxyphenyl) (methyl)carbamoyl)phenoxy)butanoate (1 Eq) was added TFA (0.7 g, 0.5 mL, 1 Eq, 6 mmol). The resulting mixture was stirred at ambient temperature for 1 hour. The crude reaction mixture was concentrated and then was purified by Isco silica gel chromatography to provide 4-(5-chloro-4-(4-cyano-6-(trifluoromethyl)pyridin-3-yl)-2-((2-methoxyphenyl)(methyl)carbamoyl)phenoxy)butanoic acid as a light yellow solid. Calc'd for C₂₆H₂₁ClF₃N₃O₅: 547.11, found [M+H]: 548.6.

Step 6.: To a solution of 4-(5-chloro-4-(4-cyano-6-(trifluoromethyl)pyridin-3-yl)-2-((2-methoxyphenyl) (methyl)carbamoyl)phenoxy)butanoic acid (67.1 mg, 1 Eq, 122 μmol) and 2-(3H-[1,2,3]triazolo[4,5-b]pyridin-3-yl)-1,1,3,3-tetramethylisouronium hexafluorophosphate(V) (69.8 mg, 1.5 Eq, 184 μmol) in DMF (0.6 mL) was added tertbutyl (26-amino-3,6,9,12,15,18,21,24-octaoxahexacosyl)carbamate (75.3 mg, 1.2 Eq, 147 μmol) followed by N-ethyl-N-isopropylpropan-2-amine (47.5 mg, 64.0 μL, 3 Eq, 367 μmol). The reaction mixture was stirred at room temperature for 2 hours. The reaction mixture was diluted with ethyl acetate, washed with water and brine, concentrated and purified by gradient column chromatography (EtOAc-Hex) to provide tert-butyl (31-(5-chloro-4-(4-cyano-6-(trifluoromethyl)pyridin-3-yl)-2-((2-methoxyphenyl)(methyl)carbamoyl)phenoxy)-28-oxo-3,6,9,12,15,18,21,24-octaoxa-27-azahentriacontyl)carbamate as a white solid. Calc'd for C₄₉H₆₇ClF₃N₅O₁₄: 1041.43, found [M+H]: 1042.8.

Step 7.: To a solution of tert-butyl (31-(5-chloro-4-(4-cyano-6-(trifluoromethyl)pyridin-3-yl)-2-((2-methoxyphenyl) (methyl)carbamoyl)phenoxy)-28-oxo-3,6,9,12,15,18,21,24-octaoxa-27-azahentriacontyl)carbamate (128 mg, 1 Eq, 123 μmol) in DCM (0.5 mL) was added TFA (0.7 g, 0.5 mL, 5e+1 Eq, 6 mmol). The resulting mixture was stirred at ambient temperature for 0.5 h until the reaction was complete. The reaction mixture was concentrated and dried to give crude 2-((1-amino-28-oxo-3,6,9,12,15,18,21,24-octaoxa-27-azahentriacontan-31-yl)oxy)-4-chloro-5-(4-cyano-6-(trifluoromethyl)pyridin-3-yl)-N-(2-methoxyphenyl)-N-methylbenzamide (83.5 mg, 88.6 μmol, 72.2%), which was used directly in the next step without any further purification. Calc'd for C₄₄H₅₉ClF₃N₅O₁₂: 941.38, found [M+H]: 943.0.

Step 8.: To a solution of 2-(4,7,10-tris(2-(tert-butoxy)-2-oxoethyl)-1,4,7,10-tetraazacyclododecan-1-yl)acetic acid (60.9 mg, 1.2 Eq, 106 μmol) and 2-(3H-[1,2,3]triazolo[4,5-b]pyridin-3-yl)-1,1,3,3-tetramethylisouronium hexafluorophosphate(V) (67.4 mg, 2 Eq, 177 mol) in DMF (0.5 mL) was added 2-((1-amino-28-oxo-3,6,9,12,15,18,21,24-octaoxa-27-azahentriacontan-31-yl)oxy)-4-chloro-5-(4-cyano-6-(trifluoromethyl)pyridin-3-yl)-N-(2-methoxyphenyl)-N-methylbenzamide (83.5 mg, 1 Eq, 88.6 μmol) followed by N-ethyl-N-isopropylpropan-2-amine (57.3 mg, 77.2 μL, 5 Eq, 443 μmol). The reaction mixture was stirred at room temperature for 2 hours. The reaction mixture was diluted with ethyl acetate, washed with water and brine, then concentrated to provide crude tri-tert-butyl 2,2′,2″-(10-(34-(5-chloro-4-(4-cyano-6-(trifluoromethyl)pyridin-3-yl)-2-((2-methoxyphenyl) (methyl)carbamoyl)phenoxy)-2,31-dioxo-6,9,12,15,18,21,24,27-octaoxa-3,30-diazatetratriacontyl)-1,4,7,10-tetraazacyclododecane-1,4,7-triyl)triacetate. Calc'd for C₇₂H₁₀₉ClF₃N₉O₁₉: 1495.75, found [M+H]: 1498.2.

Step 9.: To a DCM (0.6 mL) solution of tri-tert-butyl 2,2′,2″-(10-(34-(5-chloro-4-(4-cyano-6-(trifluoromethyl)pyridin-3-yl)-2-((2-methoxyphenyl) (methyl)carbamoyl)phenoxy)-2,31-dioxo-6,9,12,15,18,21,24,27-octaoxa-3,30-diazatetratriacontyl)-1,4,7,10-tetraazacyclododecane-1,4,7-triyl)triacetate (133 mg, 1 Eq, 88.8 μmol) was added 2,2,2-trifluoroacetic acid (10.1 mg, 1 mL, 1 Eq, 88.8 μmol). The resulting mixture was stirred at ambient temperature for 0.5 h. The crude reaction mixture was concentrated and then was purified by C18 reverse phase chromatography eluting with MeCN (0.1% TFA)/water (0.1% TFA). Pure fractions were dried and combined to provide 2,2′,2″-(10-(34-(5-chloro-4-(4-cyano-6-(trifluoromethyl)pyridin-3-yl)-2-((2-methoxyphenyl)(methyl)carbamoyl)phenoxy)-2,31-dioxo-6,9,12,15,18,21,24,27-octaoxa-3,30-diazatetratriacontyl)-1,4,7,10-tetraazacyclododecane-1,4,7-triyl)triacetic acid as a TFA salt. Calc'd for C₆₀H₈₅ClF₃N₉O₁₉: 1327.56, found [M+H]: 1329.1.

### Synthesis of Compound 6M-In

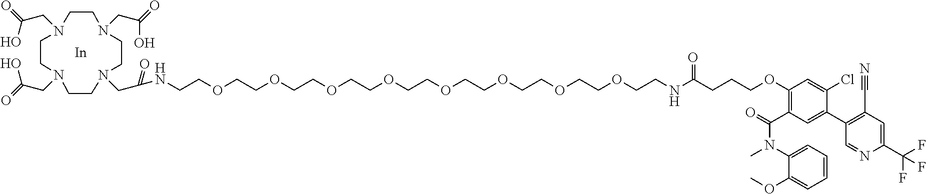

A solution of 2,2′,2″-(10-(34-(5-chloro-4-(4-cyano-6-(trifluoromethyl)pyridin-3-yl)-2-((2-methoxyphenyl)(methyl)carbamoyl)phenoxy)-2,31-dioxo-6,9,12,15,18,21,24,27-octaoxa-3,30-diazatetratriacontyl)-1,4,7,10-tetraazacyclododecane-1,4,7-triyl)triacetic acid (45 mg, 1 Eq, 34 μmol), sodium bicarbonate (28 mg, 10 Eq, 0.34 mmol) and indium(III) chloride (22 mg, 3 Eq, 0.10 mmol) in acetonitrile (0.3 mL) and water (0.3 mL) was stirred at 20° C. for 2 hours. The crude reaction mixture was purified directly by C18 reverse phase chromatography eluting with MeCN (0.1% TFA)/water (0.1% TFA). Pure fractions were dried and combined to provide indium(III) 2,2′,2″-(10-(34-(5-chloro-4-(4-cyano-6-(trifluoromethyl)pyridin-3-yl)-2-((2-methoxyphenyl)(methyl)carbamoyl)phenoxy)-2,31-dioxo-6,9,12,15,18,21,24,27-octaoxa-3,30-diazatetratriacontyl)-1,4,7,10-tetraazacyclododecane-1,4,7-triyl)triacetate as a TFA salt. Calc'd for C₆₀H₈₂ClF₃InN₉O₁₉: 1439.44, found [M+H]: 1440.9.

### Example 7: (Compound 7) 2,2′,2″-(10-(29-(5-chloro-4-(4-cyano-6-(trifluoromethyl)pyridin-3-yl)-2-((4,4-dimethyl-3,4-dihydroquinolin-1(2H)-yl)sulfonyl)phenoxy)-2-oxo-6,9,12,15,18,21,24,27-octaoxa-3-azanonacosyl)-1,4,7,10-tetraazacyclododecane-1,4,7-triyl)triacetic acid

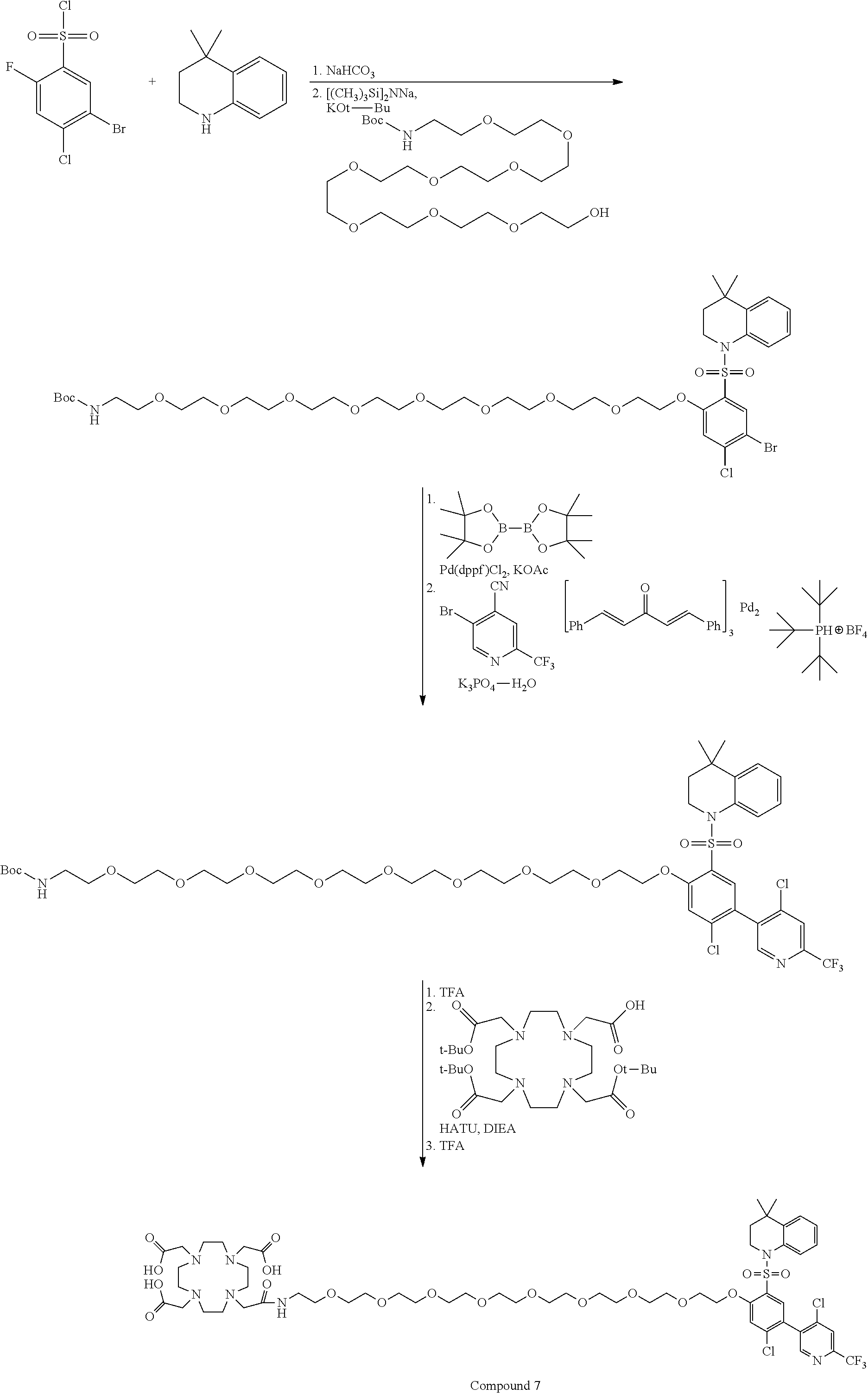

Step 1.: A solution of 5-bromo-4-chloro-2-fluorobenzenesulfonyl chloride (450.0 mg, 1 Eq, 1.461 mmol) in acetonitrile (5 mL) was added dropwise to the suspension of 4,4-dimethyl-1,2,3,4-tetrahydroquinoline (282.8 mg, 1.2 Eq, 1.754 mmol) and sodium hydrogen carbonate (613.8 mg, 5 Eq, 7.307 mmol) in water (5 mL) and acetonitrile (5 mL). The reaction mixture was stirred at 20° C. for 16 hours. The mixture was extracted by EtOAc, then the organic layer was washed with 1N HCl and brine. The organic solution was concentrated and purified by Isco silica gel chromatography to provide 1-((5-bromo-4-chloro-2-fluorophenyl)sulfonyl)-4,4-dimethyl-1,2,3,4-tetrahydroquinoline (532 mg, 1.23 mmol, 84.1%) as a light yellow solid. Calc'd for C₁₇H₁₆BrClFNO₂S: 430.98, found [M+H]: 432.5.

Step 2.: Sodium bis(trimethylsilyl)amide (44.5 mg, 243 μL, 1 molar, 1.5 Eq, 243 μmol) was added to a solution of 1-((5-bromo-4-chloro-2-fluorophenyl)sulfonyl)-4,4-dimethyl-1,2,3,4-tetrahydroquinoline (70.0 mg, 1 Eq, 162 μmol) and tert-butyl (26-hydroxy-3,6,9,12,15,18,21,24-octaoxahexacosyl)carbamate (99.7 mg, 1.2 Eq, 194 μmol) in DMF (0.6 mL). The reaction mixture was stirred at 20° C. for 2 hours, water was added then the aqueous solution was extracted with EtOAc, washed with brine, concentrated and purified by Isco silica gel chromatography (MeOH/DCM) to provide tert-butyl (26-(4-bromo-5-chloro-2-((4,4-dimethyl-3,4-dihydroquinolin-1(2H)-yl)sulfonyl)phenoxy)-3,6,9,12,15,18,21,24-octaoxahexacosyl)carbamate as a light brown oil. Calc'd for C₄₀H₆₂BrClN₂O₁₃S: 926.35, found [M+H]: 928.1.

Step 3.: A mixture of tert-butyl (26-(4-bromo-5-chloro-2-((4,4-dimethyl-3,4-dihydroquinolin-1(2H)-yl)sulfonyl)phenoxy)-3,6,9,12,15,18,21,24-octaoxahexacosyl)carbamate (250 mg, 1 Eq, 270 μmol), 4,4,4′,4′,5,5,5′,5′-octamethyl-2,2′-bi(1,3,2-dioxaborolane) (103 mg, 1.5 Eq, 405 μmol), potassium acetate (79.5 mg, 3 Eq, 810 μmol) and Pd(dppf)Cl₂ (15.8 mg, 0.08 Eq, 21.6 μmol) in 1,4-dioxane (2 mL) was bubbled with N₂ and the resulting mixture was sealed and heated at 100° C. for 1 h. The crude reaction mixture was diluted with water and extracted with EtOAc, washed with brine, then concentrated. The crude product was purified by silica gel chromatography to provide tert-butyl (26-(5-chloro-2-((4,4-dimethyl-3,4-dihydroquinolin-1(2H)-yl)sulfonyl)-4-(4,4,5,5-tetramethyl-1,3,2-dioxaborolan-2-yl)phenoxy)-3,6,9,12,15,18,21,24-octaoxahexacosyl)carbamate (75 mg, 77 μmol, 29%) as a light brown oil. Calc'd for C₄₆H₇₄BClN₂O₁₅S: 972.46.50, found [M+H]: 973.9.

Step 4.: To a mixture of 5-bromo-2-(trifluoromethyl)isonicotinonitrile (70.0 mg, 1.2 Eq, 279 μmol), tert-butyl (26-(5-chloro-2-((4,4-dimethyl-3,4-dihydroquinolin-1(2H)-yl)sulfonyl)-4-(4,4,5,5-tetramethyl-1,3,2-dioxaborolan-2-yl)phenoxy)-3,6,9,12,15,18,21,24-octaoxahexacosyl)carbamate (226 mg, 1 Eq, 232 μmol), potassium phosphate hydrate (161 mg, 3 Eq, 697 μmol) and Fu catalyst complex (30.3 mg, 0.1 Eq, 23.2 μmol) was added THF (1 mL) and water (0.3 mL) under atmospheric nitrogen. The resulting mixture was bubbled with N₂ for 1 minute, then the reaction vessel was sealed and heated at 60° C. for 1 hour. The crude reaction mixture was cooled to room temperature and diluted with ethyl acetate, washed with water and brine and concentrated. The residue was purified by silica gel chromatography to provide tert-butyl (26-(5-chloro-4-(4-cyano-6-(trifluoromethyl)pyridin-3-yl)-2-((4,4-dimethyl-3,4-dihydroquinolin-1(2H)-yl)sulfonyl)phenoxy)-3,6,9,12,15,18,21,24-octaoxahexacosyl)carbamate as a light brown solid. Calc'd for C₄₇H₆₄ClF₃N₄O₁₃S: 1016.38, found [M+H]: 1017.7.

Step 5.: To a DCM (0.6 mL) solution of tert-butyl (26-(5-chloro-4-(4-cyano-6-(trifluoromethyl)pyridin-3-yl)-2-((4,4-dimethyl-3,4-dihydroquinolin-1(2H)-yl)sulfonyl)phenoxy)-3,6,9,12,15,18,21,24-octaoxahexacosyl)carbamate (236 mg, 1 Eq, 232 mol) was added TFA (0.7 g, 0.5 mL, 3e+1 Eq, 6 mmol). The resulting mixture was stirred at ambient temperature for 0.5 h. The crude reaction mixture was concentrated and then was purified by Isco silica gel chromatography to provide 5-(4-((26-amino-3,6,9,12,15,18,21,24-octaoxahexacosyl)oxy)-2-chloro-5-((4,4-dimethyl-3,4-dihydroquinolin-1(2H)-yl)sulfonyl)phenyl)-2-(trifluoromethyl)isonicotinonitrile (75 mg, 82 μmol, 35%) as a light yellow solid. Calc'd for C₄₂H₅₆ClF₃N₄O₁₁S: 916.33, found [M+H]: 917.7.

Step 6.: To a solution of 2-(4,7,10-tris(2-(tert-butoxy)-2-oxoethyl)-1,4,7,10-tetraazacyclododecan-1-yl)acetic acid (54.7 mg, 1.2 Eq, 95.5 μmol) and 2-(3H-[1,2,3]triazolo[4,5-b]pyridin-3-yl)-1,1,3,3-tetramethylisouronium hexafluorophosphate(V) (60.5 mg, 2 Eq, 159 μmol) in DMF (0.5 mL) was added 5-(4-((26-amino-3,6,9,12,15,18,21,24-octaoxahexacosyl)oxy)-2-chloro-5-((4,4-dimethyl-3,4-dihydroquinolin-1(2H)-yl)sulfonyl)phenyl)-2-(trifluoromethyl)isonicotinonitrile (73.0 mg, 1 Eq, 79.6 μmol) followed by N-ethyl-N-isopropylpropan-2-amine (51.4 mg, 69.3 μL, 5 Eq, 398 μmol). The reaction mixture was stirred at room temperature for 2 hours. The reaction mixture was diluted with ethyl acetate, washed with water and brine, concentrated and purified by gradient column chromatography (EtOAc-Hex) to give the target compound tri-tert-butyl 2,2′,2″-(10-(29-(5-chloro-4-(4-cyano-6-(trifluoromethyl)pyridin-3-yl)-2-((4,4-dimethyl-3,4-dihydroquinolin-1(2H)-yl)sulfonyl)phenoxy)-2-oxo-6,9,12,15,18,21,24,27-octaoxa-3-azanonacosyl)-1,4,7,10-tetraazacyclododecane-1,4,7-triyl)triacetate as a white solid. Calc'd for C₇₀H₁₀₆ClF₃N₈O₁₈S: 1470.70, found [M/2+H]: 735.34.

Step 7.: To a DCM (0.6 mL) solution of tri-tert-butyl 2,2′,2″-(10-(29-(5-chloro-4-(4-cyano-6-(trifluoromethyl)pyridin-3-yl)-2-((4,4-dimethyl-3,4-dihydroquinolin-1(2H)-yl)sulfonyl)phenoxy)-2-oxo-6,9,12,15,18,21,24,27-octaoxa-3-azanonacosyl)-1,4,7,10-tetraazacyclododecane-1,4,7-triyl)triacetate (117 mg, 1 Eq, 79.5 μmol) was added 2,2,2-trifluoroacetic acid (9.06 mg, 1 mL, 1 Eq, 79.5 μmol). The resulting mixture was stirred at ambient temperature for 0.5 h. The crude reaction mixture was concentrated and then was purified by C18 reverse phase chromatography eluting with MeCN (0.1% TFA)/water (0.1% TFA). Pure fractions were dried and combined to provide 2,2′,2″-(10-(29-(5-chloro-4-(4-cyano-6-(trifluoromethyl)pyridin-3-yl)-2-((4,4-dimethyl-3,4-dihydroquinolin-1(2H)-yl)sulfonyl)phenoxy)-2-oxo-6,9,12,15,18,21,24,27-octaoxa-3-azanonacosyl)-1,4,7,10-tetraazacyclododecane-1,4,7-triyl)triacetic acid as a TFA salt. Calc'd for C₅₈H₈₂ClF₃N₈O₁₈S: 1302.84, found [M+H]: 1304.0.

### Synthesis of Compound 7M-In

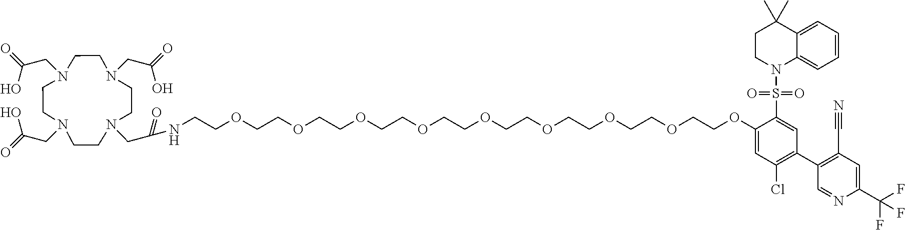 [SMILES: CC1(C)CCN(S(=O)(=O)c2cc(-c3cnc(C(F)(F)F)cc3C#N)c(Cl)cc2OCCOCCOCCOCCOCCOCCOCCOCCOCCNC(=O)CN2CCN(CC(=O)O)CCN(CC(=O)O)CCN(CC(=O)O)CC2)c2ccccc21]

A solution of 2,2′,2″-(10-(29-(5-chloro-4-(4-cyano-6-(trifluoromethyl)pyridin-3-yl)-2-((4,4-dimethyl-3,4-dihydroquinolin-1(2H)-yl)sulfonyl)phenoxy)-2-oxo-6,9,12,15,18,21,24,27-octaoxa-3-azanonacosyl)-1,4,7,10-tetraazacyclododecane-1,4,7-triyl)triacetic acid (50.0 mg, 1 Eq, 38.3 μmol), sodium bicarbonate (32.2 mg, 10 Eq, 383 μmol) and indium(III) chloride (25.4 mg, 3 Eq, 115 μmol) in acetonitrile (0.3 mL) and water (0.3 mL) was stirred at 20° C. for 2 hours. The crude reaction mixture was purified directly by C18 reverse phase chromatography eluting with MeCN (0.1% TFA)/water (0.1% TFA). Pure fractions were dried and combined to provide indium(III) 2,2′,2″-(10-(29-(5-chloro-4-(4-cyano-6-(trifluoromethyl)pyridin-3-yl)-2-((4,4-dimethyl-3,4-dihydroquinolin-1(2H)-yl)sulfonyl)phenoxy)-2-oxo-6,9,12,15,18,21,24,27-octaoxa-3-azanonacosyl)-1,4,7,10-tetraazacyclododecane-1,4,7-triyl)triacetate as a TFA salt. Calc'd for C₅₈H₇₉F₃InN₈O₁₈S: 1414.39, found [M+H]: 1416.9.

### Example 8: (Compound 8) 2,2′,2″-(10-(33-(2-(4-chloro-3-(4-cyano-6-(trifluoromethyl)pyridin-3-yl)-N-methylbenzamido)-3-methylphenoxy)-2,31-dioxo-6,9,12,15,18,21,24,27-octaoxa-3,30-diazatritriacontyl)-1,4,7,10-tetraazacyclododecane-1,4,7-triyl)triacetic acid

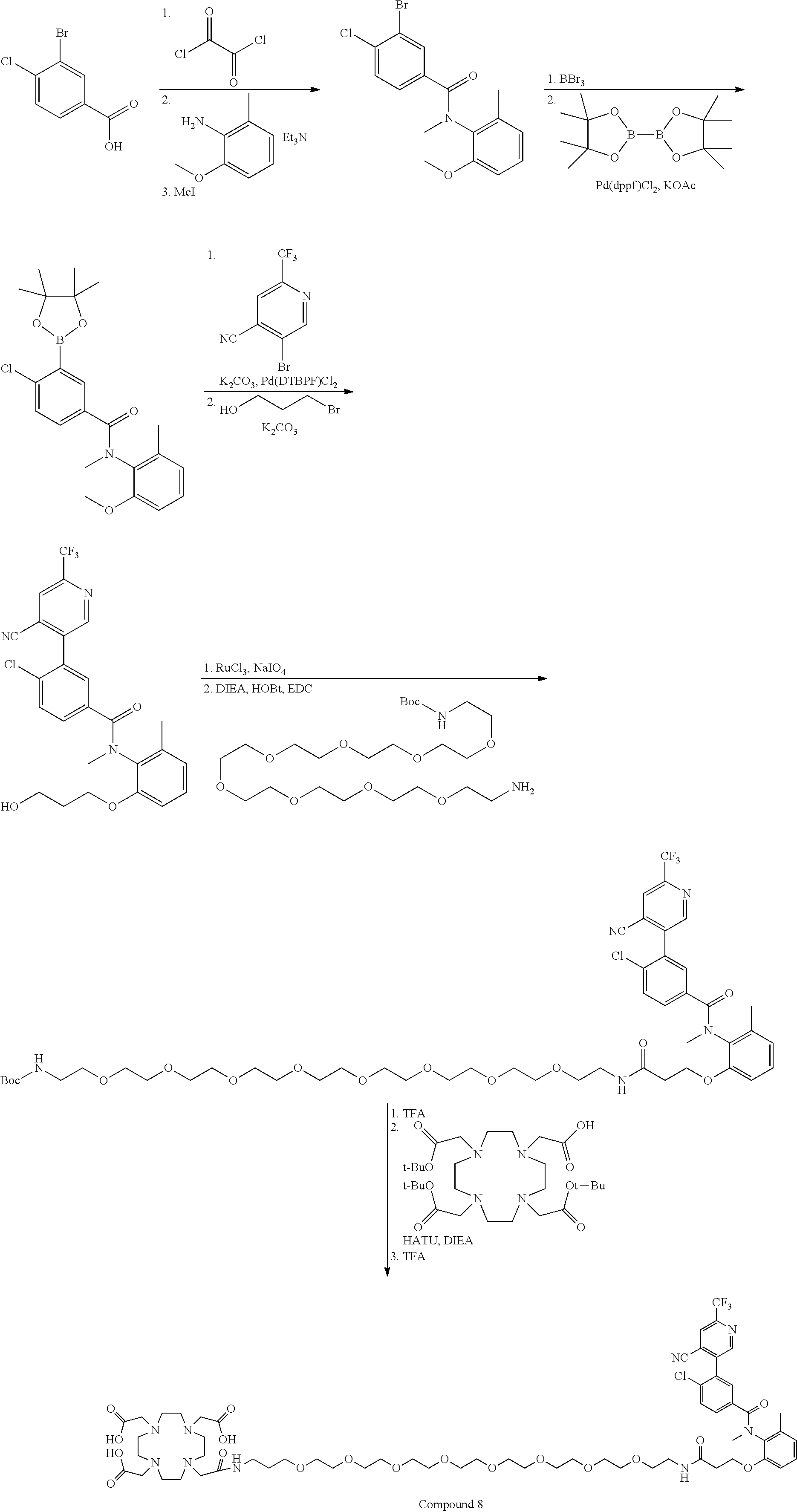

Step 1.: Into a 40-mL vial, was placed a mixture of 3-bromo-4-chlorobenzoic acid (1.50 g, 1 Eq, 6.37 mmol), DMF (20 mg, 21 μL, 0.043 Eq, 0.27 mmol) and DCM (20 mL), to which was added dropwise a mixture of oxalyl chloride (2.43 g, 1.69 mL, 3.01 Eq, 19.1 mmol) and DCM (20 mL). The reaction mixture was stirred at 25° C. for 16 hours. The mixture was concentrated under reduced pressure to afford 3-bromo-4-chlorobenzoyl chloride (1.65 g, 6.2 mmol, 97%, 95% Purity) as a yellow crude solid, which was used directly for the next step without any purification.

Step 2.: Into a 40-mL vial, was placed a mixture of 3-bromo-4-chlorobenzoyl chloride (1.65 g, 95% wt, 1 Eq, 6.17 mmol) and DCM (20 mL), to which was carefully added 2-methoxy-6-methylaniline (891 mg, 1.05 Eq, 6.50 mmol) and TEA (1.32 g, 1.82 mL, 2.11 Eq, 13.0 mmol) at 0° C. The reaction mixture was stirred at 25° C. for 16 hours. The mixture was quenched with water (50 mL), extracted with DCM (50 mL×3), then the combined organic layers were washed with brine (100 mL), dried over anhydrous Na₂SO₄ and concentrated under reduced pressure. The mixture was purified by silica gel column and eluted with PE/EtOAc (the ratio of EtOAc from 0% to 65% in 10 min) to afford 3-bromo-4-chloro-N-(2-methoxy-6-methylphenyl)benzamide (2.03 g, 5.72 mmol, 92.7%) as a yellow solid. Calc'd for C₁₅H₁₃BrClNO₂: 352.98, found [M+H]: 354.0, 356.0.

Step 3.: Into a 40-mL vial, was placed a mixture of 3-bromo-4-chloro-N-(2-methoxy-6-methylphenyl)benzamide (2.03 g, 1 Eq, 5.72 mmol) and DMF (30 mL), to which was added NaH (340 mg, 60% wt, 1.5 Eq, 8.5 mmol) at 0° C. The reaction mixture was stirred at 0° C. for 10 minutes, then iodomethane (2.00 g, 2.46 Eq, 14.1 mmol) was added dropwise and the reaction mixture was stirred at 25° C. for 1 hour. The mixture was quenched carefully with water (150 mL), extracted with EtOAc (50 mL×3), then the combined organic layers were washed with water (50 mL×3) and brine (100 mL), dried over anhydrous Na₂SO₄ and concentrated under reduced pressure to provide 3-bromo-4-chloro-N-(2-methoxy-6-methylphenyl)-N-methylbenzamide (2.10 g, 5.1 mmol, 90%, 90% Purity) as a yellow solid. Calc'd for C₁₆H₁₅BrClNO₂: 367.00, found [M+H]: 368.0, 370.0.

Step 4.: Into a 50-mL three-necked round bottom flask, purged and maintained under an inert atmosphere of nitrogen, was placed a mixture of 3-bromo-4-chloro-N-(2-methoxy-6-methylphenyl)-N-methylbenzamide (2.10 g, 1 Eq, 5.70 mmol) in DCM (30 mL). The reaction mixture was cooled to −78° C., then a solution of tribromoborane (4.28 g, 17.1 mL, 1 molar, 3 Eq, 17.1 mmol) (1.0 M in DCM) was added dropwise. The reaction mixture was stirred at −78° C. for 0.5 hour, then the reaction mixture was allowed warm to 25° C. and stirred for an additional 16 hours. The mixture was quenched with water (100 mL), then the pH value was adjusted to 5 by addition of a 1N NaOH solution. The mixture was extracted with DCM (100 mL×2), then the combined organic layers were washed with brine (100 mL), dried over anhydrous Na₂SO₄ and concentrated under reduced pressure to provide 3-bromo-4-chloro-N-(2-hydroxy-6-methylphenyl)-N-methylbenzamide (1.85 g, 5.22 mmol, 91.6%) as an off-white solid. Calc'd for C₁₅H₁₃BrClNO₂: 352.98, found [M+H]: 354.0, 356.0.

Step 5.: Into a 40-mL vial, purged and maintained under an inert atmosphere of nitrogen, was placed a mixture of 3-bromo-4-chloro-N-(2-hydroxy-6-methylphenyl)-N-methylbenzamide (480 mg, 1 Eq, 1.35 mmol), bis(pinacolato)diborane (516 mg, 1.50 Eq, 2.03 mmol), Pd(dppf)Cl₂ (100 mg, 0.101 Eq, 137 μmol), potassium acetate (400 mg, 255 μL, 3.01 Eq, 4.08 mmol) and dioxane (10 mL). The reaction mixture was stirred at 90° C. for 16 hours. The mixture was quenched with water (50 mL), extracted with EtOAc (50 mL×3), then the combined organic layers were washed with brine (50 mL), dried over anhydrous Na₂SO₄ and concentrated under reduced pressure. The mixture was directly purified by silica gel column eluted with PE/EtOAc (the ratio of EtOAc from 0% to 65% in 10 min) to provide 4-chloro-N-(2-hydroxy-6-methylphenyl)-N-methyl-3-(4,4,5,5-tetramethyl-1,3,2-dioxaborolan-2-yl)benzamide (510 mg, 0.89 mmol, 66%, 70% Purity) as a yellow solid. Calc'd for C₂₁H₂₅BClNO₄: 401.16, found [M+H]: 402.2, 404.2.

Step 6.: Into a 40-mL vial, purged and maintained with an inert atmosphere of nitrogen, was placed a mixture of 4-chloro-N-(2-hydroxy-6-methylphenyl)-N-methyl-3-(4,4,5,5-tetramethyl-1,3,2-dioxaborolan-2-yl)benzamide (500 mg, 70% wt, 1 Eq, 871 μmol), 5-bromo-2-(trifluoromethyl)isonicotinonitrile (300 mg, 1.37 Eq, 1.20 mmol), Pd(DTBPF)Cl₂ (30 mg, 0.053 Eq, 46 μmol), K₂CO₃ (360 mg, 2.99 Eq, 2.60 mmol) and toluene (10 mL). The reaction mixture was stirred at 60° C. for 1 hour. The mixture was directly purified by silica gel column eluted with (the ratio of EtOAc from 0% to 65% in 10 min). This resulted in 4-chloro-3-(4-cyano-6-(trifluoromethyl)pyridin-3-yl)-N-(2-hydroxy-6-methylphenyl)-N-methylbenzamide (380 mg, 852 mol, 97.8%) as yellow solid. Calc'd for C₂₂H₁₅ClF₃N₃O₂: 445.08, found [M+H]: 446.1, 448.1.

Step 7.: Into a 40-mL vial, was placed a mixture of 4-chloro-3-(4-cyano-6-(trifluoromethyl)pyridin-3-yl)-N-(2-hydroxy-6-methylphenyl)-N-methylbenzamide (360 mg, 1 Eq, 807 μmol), K₂CO₃ (446 mg, 4.00 Eq, 3.23 mmol) and DMF (7 mL), to which was added 3-bromopropan-1-ol (337 mg, 3.00 Eq, 2.42 mmol). The reaction mixture was stirred at 25° C. for 16 hours. The mixture was directly purified by MPLC using the following conditions: Column, C18 120 g Column; mobile phase, water (0.1% FA) and ACN (30% ACN up to 98% in 10 min); Total flow rate, 70 mL/min; Detector, UV 220 nm. The collected fractions were concentrated to afford 4-chloro-3-(4-cyano-6-(trifluoromethyl)pyridin-3-yl)-N-(2-(3-hydroxypropoxy)-6-methylphenyl)-N-methylbenzamide (210 mg, 417 μmol, 51.6%) as a yellow oil. Calc'd for C₂₅H₂₁ClF₃N₃O₃: 503.12, found [M+H]: 504.2, 506.2.

Step 8.: Into a 40-mL vial, was placed a mixture of 4-chloro-3-(4-cyano-6-(trifluoromethyl)pyridin-3-yl)-N-(2-(3-hydroxypropoxy)-6-methylphenyl)-N-methylbenzamide (210 mg, 1 Eq, 417 μmol), sodium periodate (267 mg, 3.00 Eq, 1.25 mmol), DCM (2 mL) and MeCN (2 mL), to which was added ruthenium trichloride (4.0 mg, 1.3 μL, 0.046 Eq, 19 μmol). The reaction mixture was stirred at 25° C. for 15 min. The mixture was directly purified by MPLC with the following conditions: Column, C18 120 g Column; mobile phase, water (0.1% FA) and ACN (30% ACN up to 98% in 10 min); Total flow rate, 70 mL/min; Detector, UV 220 nm. The collected fractions were concentrated to afford 3-(2-(4-chloro-3-(4-cyano-6-(trifluoromethyl)pyridin-3-yl)-N-methylbenzamido)-3-methylphenoxy)propanoic acid (150 mg, 0.23 mmol, 56%, 80% Purity) as a yellow oil. Calc'd for C₂₅H₁₉ClF₃N₃O₄: 517.10, found [M+H]: 518.2, 520.2.

Step 9.: Into an 8-mL vial, was placed a mixture of 3-(2-(4-chloro-3-(4-cyano-6-(trifluoromethyl)pyridin-3-yl)-N-methylbenzamido)-3-methylphenoxy)propanoic acid (50 mg, 80% wt, 1 Eq, 77 μmol), HOBt (59 mg, 5.0 Eq, 0.39 mmol), EDC (44 mg, 3.0 Eq, 0.23 mmol), DIEA (30 mg, 3.0 Eq, 0.23 mmol) and DMF (1.0 mL). The mixture was stirred at 25° C. for 30 min, then tert-butyl (26-amino-3,6,9,12,15,18,21,24-octaoxahexacosyl)carbamate (60 mg, 1.5 Eq, 0.12 mmol) was added. The reaction mixture was stirred at 25° C. for 1 hour. The mixture was directly purified by MPLC with the following conditions: Column, C18 120 g Column; mobile phase, Water (0.1% FA) and ACN (30% ACN up to 98% in 10 min); Total flow rate, 70 mL/min; Detector, UV 220 nm. The collected fractions were concentrated to provide tert-butyl (30-(2-(4-chloro-3-(4-cyano-6-(trifluoromethyl)pyridin-3-yl)-N-methylbenzamido)-3-methylphenoxy)-28-oxo-3,6,9,12,15,18,21,24-octaoxa-27-azatriacontyl)carbamate (36 mg, 36 μmol, 46%) as a light yellow solid. Calc'd for C₄₈H₆₅ClF₃N₅O₁₃: 1011.42, found [M+H]: 1012.7, 1014.7.

Step 10.: Into an 8-mL vial, was placed a mixture of tert-butyl (30-(2-(4-chloro-3-(4-cyano-6-(trifluoromethyl)pyridin-3-yl)-N-methylbenzamido)-3-methylphenoxy)-28-oxo-3,6,9,12,15,18,21,24-octaoxa-27-azatriacontyl)carbamate (35 mg, 1 Eq, 35 μmol) and DCM (2.0 mL), to which was added TFA (0.5 mL). The reaction mixture was stirred at 25° C. for 1 hour. The mixture was concentrated, and the crude product was directly purified by Prep-HPLC with the following conditions: Column, C18 120 g Column; mobile phase, Water (0.05% FA) and ACN (30% ACN up to 98% in 6 min, 98% ACN to 98% in 3 min); Total flow rate, 70 mL/min; Detector, UV 220 nm. The collected fractions were concentrated to provide N-(2-((1-amino-28-oxo-3,6,9,12,15,18,21,24-octaoxa-27-azatriacontan-30-yl)oxy)-6-methylphenyl)-4-chloro-3-(4-cyano-6-(trifluoromethyl)pyridin-3-yl)-N-methylbenzamide (24 mg, 26 μmol, 76%) as a light yellow oil. Calc'd for C₄₃H₅₇ClF₃N₅O₁₁: 911.37, found [M+H]: 912.8, 914.8.

Step 11. Into an 8-mL vial, was placed a mixture of 2-(4,7,10-tris(2-(tert-butoxy)-2-oxoethyl)-1,4,7,10-tetraazacyclododecan-1-yl)acetic acid (29 mg, 2.0 Eq, 51 μmol), HATU (20 mg, 2.1 Eq, 53 μmol), DIEA (20 mg, 6.1 Eq, 0.15 mmol) and DMF (1.0 mL). The mixture was stirred at 25° C. for 30 min, then N-(2-((1-amino-28-oxo-3,6,9,12,15,18,21,24-octaoxa-27-azatriacontan-30-yl)oxy)-6-methylphenyl)-4-chloro-3-(4-cyano-6-(trifluoromethyl)pyridin-3-yl)-N-methylbenzamide (23 mg, 1 Eq, 25 μmol) was added. The reaction mixture was stirred at 25° C. for 1 hour. The mixture was directly purified by MPLC using the following conditions: Column, C18 120 g, Column; mobile phase, Water (0.1% FA) and ACN (30% ACN up to 98% in 10 min); Total flow rate, 70 mL/min; Detector, UV 220 nm. The collected fractions were concentrated to provide tri-tert-butyl 2,2′,2″-(10-(33-(2-(4-chloro-3-(4-cyano-6-(trifluoromethyl)pyridin-3-yl)-N-methylbenzamido)-3-methylphenoxy)-2,31-dioxo-6,9,12,15,18,21,24,27-octaoxa-3,30-diazatritriacontyl)-1,4,7,10-tetraazacyclododecane-1,4,7-triyl)triacetate (16 mg, 11 μmol, 43%) as a light yellow solid. Calc'd for C₇₁H₁₀₇ClF₃N₉O₁₈: 1465.74, found [M/2+H]: 734.7.

Step 12. Into an 8-mL vial, was placed a mixture of tri-tert-butyl 2,2′,2″-(10-(33-(2-(4-chloro-3-(4-cyano-6-(trifluoromethyl)pyridin-3-yl)-N-methylbenzamido)-3-methylphenoxy)-2,31-dioxo-6,9,12,15,18,21,24,27-octaoxa-3,30-diazatritriacontyl)-1,4,7,10-tetraazacyclododecane-1,4,7-triyl)triacetate (15 mg, 1 Eq, 10 μmol) and TFA (0.5 mL). The reaction mixture was stirred at 25° C. for 1 hour. The mixture was concentrated under reduced pressure to provide 2,2′,2″-(10-(33-(2-(4-chloro-3-(4-cyano-6-(trifluoromethyl)pyridin-3-yl)-N-methylbenzamido)-3-methylphenoxy)-2,31-dioxo-6,9,12,15,18,21,24,27-octaoxa-3,30-diazatritriacontyl)-1,4,7,10-tetraazacyclododecane-1,4,7-triyl)triacetic acid (7.0 mg, 5.4 μmol, 53%) as a yellow crude oil, which was used directly for next step without any purification. Calc'd for C₅₉H₈₃ClF₃N₉O₁₈: 1297.55, found [M/2+H]: 650.4.

### Synthesis of Compound 8M-In

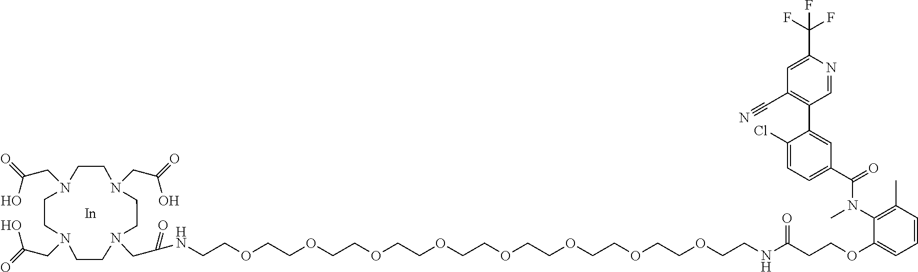

Step 13.: Into a 2-mL vial, was placed a mixture of 2,2′,2″-(10-(33-(2-(4-chloro-3-(4-cyano-6-(trifluoromethyl)pyridin-3-yl)-N-methylbenzamido)-3-methylphenoxy)-2,31-dioxo-6,9,12,15,18,21,24,27-octaoxa-3,30-diazatritriacontyl)-1,4,7,10-tetraazacyclododecane-1,4,7-triyl)triacetic acid (7.0 mg, 1 Eq, 5.4 μmol), NaHCO₃ (5.0 mg, 11 Eq, 60 μmol), indium(III) chloride (5.0 mg, 4.2 Eq, 23 μmol), MeCN (0.30 mL) and water (0.10 mL). The reaction mixture was stirred at 80° C. for 1 hour. Acetonitrile (4 mL) was added to the mixture, then the organic and aqueous layers were separated. The organic phase was purified by prep-HPLC: Column, Sunfire Prep C18 OBD Column, 50*250 mm, 5 m 10 nm; Mobile Phase A: Water (0.05% TFA), Mobile Phase B: ACN; Flow rate: 90 mL/min; Gradient: 20% B to 65% B in 12 min. The collected fractions were directly lyophilized to provide indium(III) 2,2′,2″-(10-(33-(2-(4-chloro-3-(4-cyano-6-(trifluoromethyl)pyridin-3-yl)-N-methylbenzamido)-3-methylphenoxy)-2,31-dioxo-6,9,12,15,18,21,24,27-octaoxa-3,30-diazatritriacontyl)-1,4,7,10-tetraazacyclododecane-1,4,7-triyl)triacetate TFA salt (4.4 mg, 2.9 μmol, 54%) as an off-white solid. Calc'd for C₆₁H₈₁ClF₆InN₉O₂₀: 1523.43, found [M+H-TFA]: 1410.8.

### Example 9: (Compound 9) 2,2′,2″-(10-(29-(5-chloro-4-(4-cyano-6-(trifluoromethyl)pyridin-3-yl)-2-(N-(2-methoxyphenyl)-N-methylsulfamoyl)phenoxy)-2-oxo-6,9,12,15,18,21,24,27-octaoxa-3-azanonacosyl)-1,4,7,10-tetraazacyclododecane-1,4,7-triyl)triacetic acid

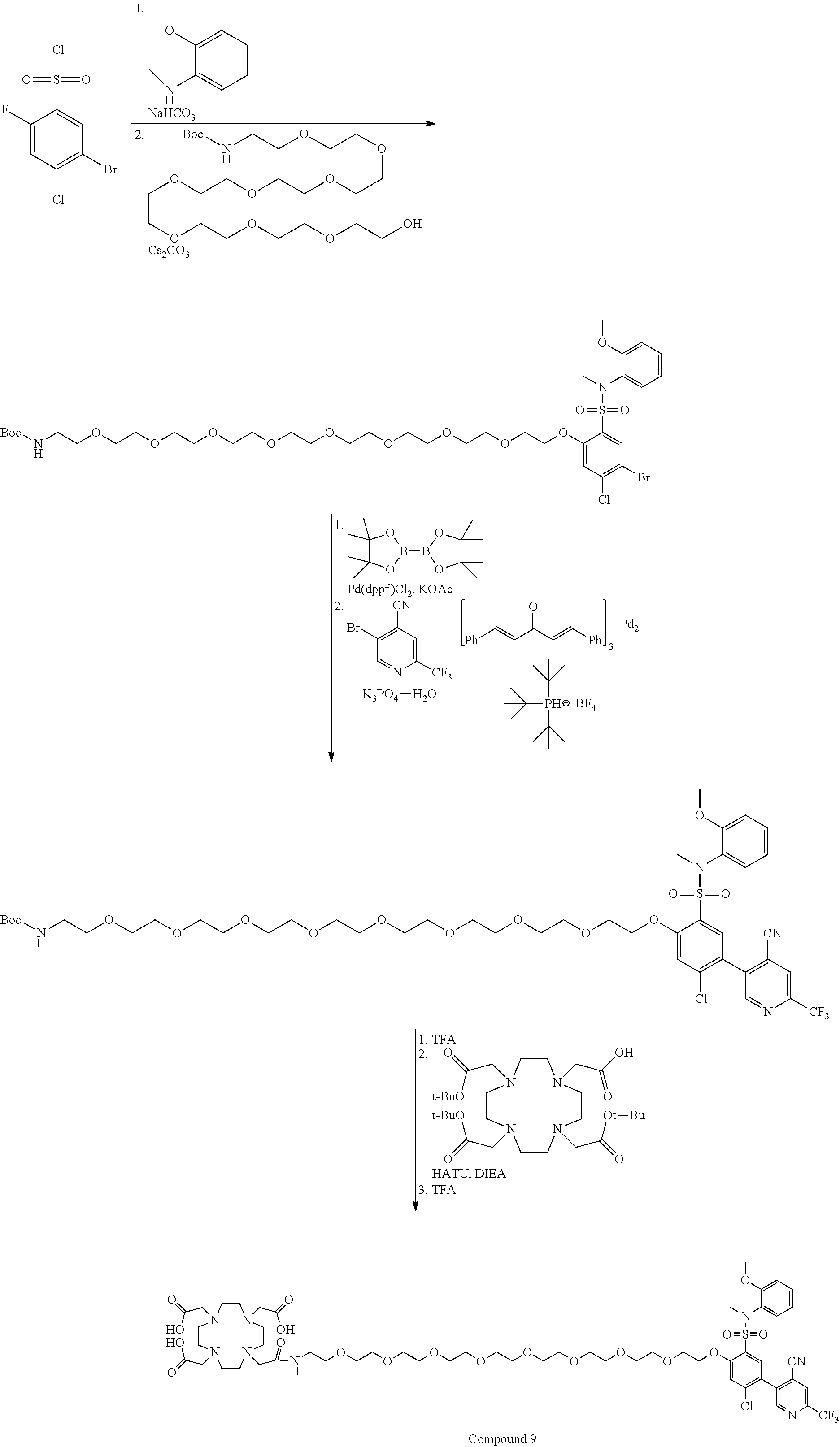

Step 1.: A solution of 5-bromo-4-chloro-2-fluorobenzenesulfonyl chloride (550 mg, 1.0 Eq, 1.79 mmol) in acetonitrile (5 mL) was added dropwise to a suspension of 2-methoxy-N-methylaniline hydrochloride (310 mg, 1.0 Eq, 1.79 mmol) and sodium bicarbonate (68.2 mg, 5 Eq, 812 μmol) in water (0.4 mL) and acetonitrile (0.4 mL). The reaction mixture was stirred at 20° C. for 1 hour. Then the mixture was extracted with EtOAc and the organic layer was washed with 1N HCl and brine. The organic layer was concentrated and purified by Isco silica gel chromatography to provide 5-bromo-4-chloro-2-fluoro-N-(2-methoxyphenyl)benzenesulfonamide as a light yellow solid (518 mg, 71%). Calc'd for C₁₄H₁₂BrClFNO₃S: 406.94, found [M+H]: 410.0.

Step 2.: tert-Butyl (26-hydroxy-3,6,9,12,15,18,21,24-octaoxahexacosyl)carbamate (302 mg, 1.2 Eq, 587 μmol) was added to a solution of 5-bromo-4-chloro-2-fluoro-N-(2-methoxyphenyl)-N-methylbenzenesulfonamide (20 mg, 1 Eq, 51 μmol) and cesium carbonate (478 mg, 3.0 Eq, 1.47 mmol) in DMF (6 mL), and the reaction mixture was stirred at 80° C. for 2 hours. Upon completion, the reaction was cooled to room temperature, water was added and the aqueous solution was extracted with EtOAc, washed with brine, concentrated and purified by flash chromatography to give provide tert-butyl(26-(4-bromo-5-chloro-2-(N-(2-methoxyphenyl)-N-methylsulfamoyl)phenoxy)-3,6,9,12,15,18,21,24-octaoxahexacosyl)carbamate (386 mg, 428 mol, 87.4%) as a colorless oil (386 mg, 87.4%). Calc'd for C₃₇H₅₈BrClN₂O₁₄S: 900.25, found [M/2+H]: 901.4.

Step 3.: A mixture of tert-butyl (26-(4-bromo-5-chloro-2-(N-(2-methoxyphenyl)-N-methylsulfamoyl)phenoxy)-3,6,9,12,15,18,21,24-octaoxahexacosyl)carbamate (150 mg, 1 Eq, 166 μmol), 4,4,4′,4′,5,5,5′,5′-octamethyl-2,2′-bi(1,3,2-dioxaborolane) (63.3 mg, 1.5 Eq, 249 mol), potassium acetate (48.9 mg, 3 Eq, 499 μmol) and Pd(dppf)Cl₂ (9.73 mg, 0.08 Eq, 13.3 mol) in DMF (1 mL) was bubbled with N₂ for 1 min and the reaction vessel containing the resulting mixture was sealed and heated at 100° C. for 1 h. The crude reaction mixture was diluted with water and extracted with EtOAc, washed with brine, then concentrated. The crude product was purified by silica gel chromatography to provide tert-butyl (26-(5-chloro-2-(N-(2-methoxyphenyl)-N-methylsulfamoyl)-4-(4,4,5,5-tetramethyl-1,3,2-dioxaborolan-2-yl)phenoxy)-3,6,9,12,15,18,21,24-octaoxahexacosyl)carbamate (108 mg, 114 μmol, 68.4%) as a light brown oil. Calc'd for C₄₃H₇₀BClN₂O₁₆S: 948.42, found [M+H]: 950.2.

Step 4.: To a mixture of tert-butyl (26-(5-chloro-2-(N-(2-methoxyphenyl)-N-methylsulfamoyl)-4-(4,4,5,5-tetramethyl-1,3,2-dioxaborolan-2-yl)phenoxy)-3,6,9,12,15,18,21,24-octaoxahexacosyl)carbamate (108 mg, 1 Eq, 114 μmol), 5-bromo-2-(trifluoromethyl)isonicotinonitrile (28.6 mg, 1 Eq, 114 μmol), potassium phosphate hydrate (78.6 mg, 3 Eq, 341 μmol) and Fu catalyst complex (14.8 mg, 0.1 Eq, 11.4 μmol) was added THF (1 mL) and water (0.1 mL) under nitrogen. The resulting mixture was bubbled with N₂ for 1 minute, then the reaction vessel was sealed and heated at 80° C. for 1 hour. The crude reaction mixture was cooled to room temperature and diluted with ethyl acetate (5.0 mL), washed with water and brine, then concentrated to give crude product tert-butyl (26-(5-chloro-4-(4-cyano-6-(trifluoromethyl)pyridin-3-yl)-2-(N-(2-methoxyphenyl)-N-methylsulfamoyl)phenoxy)-3,6,9,12,15,18,21,24-octaoxahexacosyl)carbamate as a dark brown oil. Calc'd for C₄₄H₆₀ClF₃N₄O₁₄S: 992.34, found [M+H]: 994.0.

Step 5.: To a solution of tert-butyl (26-(5-chloro-4-(4-cyano-6-(trifluoromethyl)pyridin-3-yl)-2-(N-(2-methoxyphenyl)-N-methylsulfamoyl)phenoxy)-3,6,9,12,15,18,21,24-octaoxahexacosyl)carbamate (110 mg, 1 Eq, 111 μmol) in DCM (0.6 mL) was added TFA (0.7 g, 0.5 mL, 6e+1 Eq, 6 mmol). The resulting mixture was stirred at ambient temperature for 0.5 h. The crude reaction mixture was concentrated and purified by C18 reverse phase chromatography eluting with MeCN (0.1% TFA)/water (0.1% TFA). Pure fractions were dried and combined to provide 2-((26-amino-3,6,9,12,15,18,21,24-octaoxahexacosyl)oxy)-4-chloro-5-(4-cyano-6-(trifluoromethyl)pyridin-3-yl)-N-(2-methoxyphenyl)-N-methylbenzenesulfonamide (46.3 mg, 51.8 μmol, 46.8%) as a TFA salt. Calc'd for C₃₉H₅₂ClF₃N₄O₁₂S: 892.29, found [M+H]: 893.6.

Step 6.: To a solution of 2-(4,7,10-tris(2-(tert-butoxy)-2-oxoethyl)-1,4,7,10-tetraazacyclododecan-1-yl)acetic acid (33.8 mg, 1.2 Eq, 59.0 μmol) and 2-(3H-[1,2,3]triazolo[4,5-b]pyridin-3-yl)-1,1,3,3-tetramethylisouronium hexafluorophosphate(V) (37.4 mg, 2 Eq, 98.3 μmol) in DMF (0.5 mL) was added 2-((26-amino-3,6,9,12,15,18,21,24-octaoxahexacosyl)oxy)-4-chloro-5-(4-cyano-6-(trifluoromethyl)pyridin-3-yl)-N-(2-methoxyphenyl)-N-methylbenzenesulfonamide (43.9 mg, 1 Eq, 49.1 μmol) followed by N-ethyl-N-isopropylpropan-2-amine (31.8 mg, 42.8 μL, 5 Eq, 246 μmol). The reaction mixture was stirred at room temperature for 2 hours. The reaction mixture was diluted with ethyl acetate, washed with water and brine, then concentrated to provide crude tri-tertbutyl 2,2′,2″-(10-(29-(5-chloro-4-(4-cyano-6-(trifluoromethyl)pyridin-3-yl)-2-(N-(2-methoxyphenyl)-N-methylsulfamoyl)phenoxy)-2-oxo-6,9,12,15,18,21,24,27-octaoxa-3-azanonacosyl)-1,4,7,10-tetraazacyclododecane-1,4,7-triyl)triacetate. Calc'd for C₆₇H₁₀₂ClF₃N₈O₁₉S: 1446.66, found [M+H]: 1448.6.

Step 7.: To a DCM (0.6 mL) solution of tri-tert-butyl 2,2′,2″-(10-(29-(5-chloro-4-(4-cyano-6-(trifluoromethyl)pyridin-3-yl)-2-(N-(2-methoxyphenyl)-N-methylsulfamoyl)phenoxy)-2-oxo-6,9,12,15,18,21,24,27-octaoxa-3-azanonacosyl)-1,4,7,10-tetraazacyclododecane-1,4,7-triyl)triacetate (71.2 mg, 1 Eq, 49.2 μmol) was added 2,2,2-trifluoroacetic acid (5.61 mg, 1 mL, 1 Eq, 49.2 μmol). The resulting mixture was stirred at ambient temperature for 0.5 h. The crude reaction mixture was concentrated and then was purified by C18 reverse phase chromatography eluting with MeCN (0.1% TFA)/water (0.1% TFA). Pure fractions were dried and combined to provide 2,2′,2″-(10-(29-(5-chloro-4-(4-cyano-6-(trifluoromethyl)pyridin-3-yl)-2-(N-(2-methoxyphenyl)-N-methylsulfamoyl)phenoxy)-2-oxo-6,9,12,15,18,21,24,27-octaoxa-3-azanonacosyl)-1,4,7,10-tetraazacyclododecane-1,4,7-triyl)triacetic acid as a TFA salt. Calc'd for C₅₅H₇₈ClF₃N₈O₁₉S: 1278.47, found [M+H]: 1279.9.

### Synthesis of Compound 9M-In

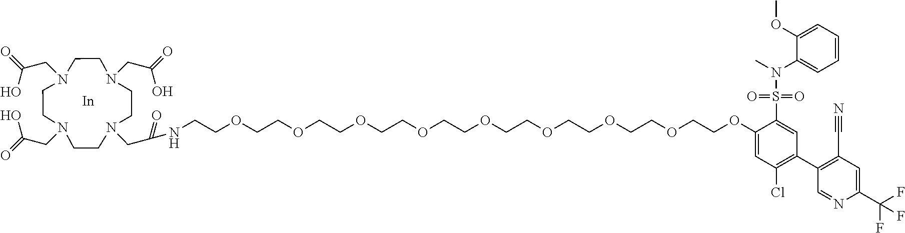

A solution of 2,2′,2″-(10-(29-(5-chloro-4-(4-cyano-6-(trifluoromethyl)pyridin-3-yl)-2-(N-(2-methoxyphenyl)-N-methylsulfamoyl)phenoxy)-2-oxo-6,9,12,15,18,21,24,27-octaoxa-3-azanonacosyl)-1,4,7,10-tetraazacyclododecane-1,4,7-triyl)triacetic acid (25 mg, 1 Eq, 20 μmol), sodium bicarbonate (16 mg, 10 Eq, 0.20 mmol) and indium(III) chloride (13 mg, 3 Eq, 59 μmol) in acetonitrile (0.3 mL) and water (0.3 mL) was stirred at 20° C. for 2 hours. The crude reaction mixture was purified directly by C18 reverse phase chromatography eluting with MeCN (0.1% TFA)/water (0.1% TFA). Pure fractions were dried and combined to provide indium(III) 2,2′,2″-(10-(29-(5-chloro-4-(4-cyano-6-(trifluoromethyl)pyridin-3-yl)-2-(N-(2-methoxyphenyl)-N-methylsulfamoyl)phenoxy)-2-oxo-6,9,12,15,18,21,24,27-octaoxa-3-azanonacosyl)-1,4,7,10-tetraazacyclododecane-1,4,7-triyl)triacetate as a TFA salt. Calc'd for C₅₅H₇₅ClF₃InN₈O₁₉S: 1390.35, found [M+H]: 1391.7.

### Example 10: (Compound 10) 2,2′,2″-(10-((R)-8-((R)-4-carboxy-2-(4-(4-iodophenyl)butanamido)butanamido)-40-(2-(4-chloro-3-(4-cyano-6-(trifluoromethyl)pyridin-3-yl)-N-methylbenzamido)phenoxy)-2,9,38-trioxo-13,16,19,22,25,28,31,34-octaoxa-3,10,37-triazatetracontyl)-1,4,7,10-tetraazacyclododecane-1,4,7-triyl)triacetic acid

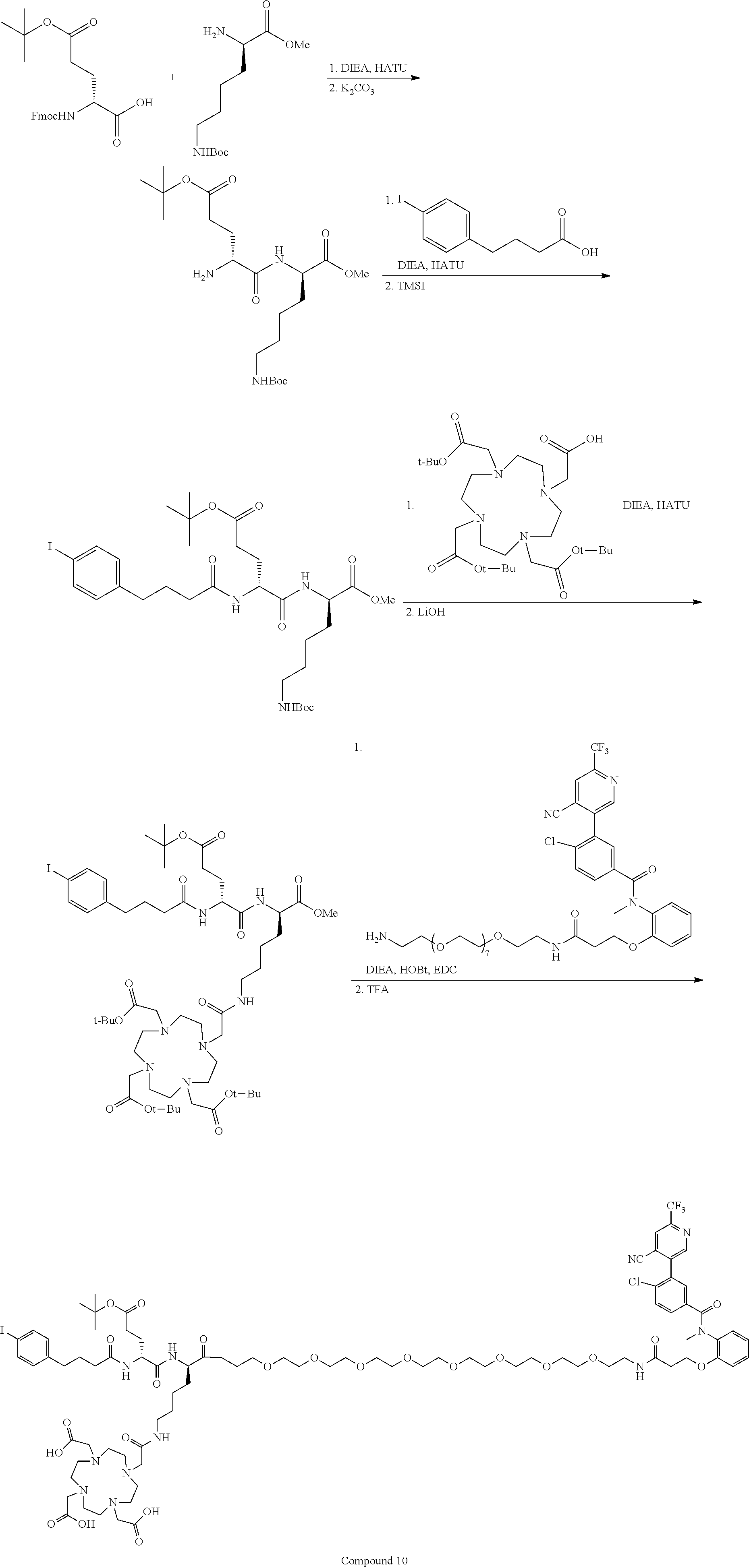

Step 1.: Into a 100-mL round bottom flask, was placed a mixture of (R)-2-((((9H-fluoren-9-yl)methoxy)carbonyl)amino)-5-(tert-butoxy)-5-oxopentanoic acid (2.50 g, 1.0 Eq, 5.88 mmol), HATU (2.68 g, 1.20 Eq, 7.05 mmol), DIEA (2.28 g, 3.00 Eq, 17.6 mmol) and DMF (30 mL). The reaction mixture was stirred at 25° C. for 30 min, then methyl N6-(tert-butoxycarbonyl)-D-lysinate hydrochloride (2.00 g, 1.15 Eq, 6.74 mmol) was added. The reaction mixture was stirred at 25° C. for 1 hour. The mixture was quenched with water (150 mL), extracted with EtOAc (80 mL×3), then the combined organic layers were washed with water (50 mL×3), brine (100 mL), dried over anhydrous Na₂SO₄ and concentrated under reduced pressure. The crude product was purified by silica gel column eluted with PE/EtOAc (the ratio of EtOAc from 0% to 65% in 10 min). The collected fractions were concentrated to provide methyl N2-((R)-2-((((9H-fluoren-9-yl)methoxy)carbonyl)amino)-5-(tert-butoxy)-5-oxopentanoyl)-N6-(tert-butoxycarbonyl)-D-lysinate (4.30 g, 5.8 mmol, 99%, 90% Purity) as a light yellow solid. Calc'd for C₃₆H₄₉N₃O₉: 667.35, found [M+Na]: 690.3.

Step 2.: Into a 40 mL vial was added a mixture of methyl N2-((R)-2-((((9H-fluoren-9-yl)methoxy)carbonyl)amino)-5-(tert-butoxy)-5-oxopentanoyl)-N6-(tert-butoxycarbonyl)-D-lysinate (1.5 g, 1 Eq, 2.2 mmol), K₂CO₃ (6.2 g, 20 Eq, 45 mmol) and MeCN (20 mL). The mixture was stirred at 50° C. for 3 hours. The mixture was quenched with water (50 mL), then extracted with DCM (50 mL×3). The combined organic layers were washed with water (50 mL×3), brine (50 mL), dried over anhydrous Na₂SO₄ and concentrated under reduced pressure. The mixture was concentrated to provide methyl N2-((R)-2-amino-5-(tertbutoxy)-5-oxopentanoyl)-N6-(tert-butoxycarbonyl)-D-lysinate (1.2 g, 2.7 mmol, 120%) as a light yellow oil, which was used in the next step without further purification. Calc'd for C₂₁H₃₉N₃O₇ 445.28, found [M+Na]: 446.3.

Step 3.: To a mixture of 4-(4-iodophenyl)butanoic acid (0.94 g, 1.2 Eq, 3.2 mmol), HATU (1.3 g, 1.3 Eq, 3.4 mmol), DIEA (1.0 g, 2.9 Eq, 7.7 mmol) and DMF (10 mL), was added methyl N2-((R)-2-amino-5-(tert-butoxy)-5-oxopentanoyl)-N6-(tertbutoxycarbonyl)-D-lysinate (1.2 g, 1 Eq, 2.7 mmol). The mixture was stirred at 25° C. for 1 hour. The mixture was purified by Prep-HPLC with the following conditions: Column, C18 19*150 mm Sum; mobile phase, water (0.05% FA) and ACN (30.0% ACN up to 98.0% in 7 min); Total flow rate, 70 mL/min; Detector, UV 220 nm. The collected fractions were combined and concentrated under vacuum to provide methyl N2-((R)-5-(tert-butoxy)-2-(4-(4-iodophenyl)butanamido)-5-oxopentanoyl)-N6-(tert-butoxycarbonyl)-D-lysinate (1.1 g, 1.5 mmol, 57%) as a light yellow solid. Calc'd for C₃₁H₄₈IN₃O₈: 717.25, found [M+H]: 718.4.

Step 4.: Into an 8-mL vial, was placed a mixture of methyl N2-((R)-5-(tert-butoxy)-2-(4-(4-iodophenyl)butanamido)-5-oxopentanoyl)-N6-(tertbutoxycarbonyl)-D-lysinate (450 mg, 1 Eq, 627 μmol) and MeCN (3 mL), to which was added TMS-I (140 mg, 95.2 μL, 1.12 Eq, 700 mol). The reaction was stirred at 25° C. for 20 min. The mixture was directly purified by Prep-HPLC with the following conditions: Column, C18 120 g Column; mobile phase, Water (0.1% FA) and ACN (30% ACN up to 98% in 7 min); Total flow rate, 70 mL/min; Detector, UV 220 nm. The collected fractions were concentrated to provide methyl ((R)-5-(tert-butoxy)-2-(4-(4-iodophenyl)butanamido)-5-oxopentanoyl)-D-lysinate (410 mg, 664 μmol, 106%) as a colorless solid. Calc'd for C₂₆H₄₀IN₃O₆: 617.20, found [M+H]: 618.3.

Step 5.: Into a 40-mL vial, was placed a mixture of 2-(4,7,10-tris(2-(tert-butoxy)-2-oxoethyl)-1,4,7,10-tetraazacyclododecan-1-yl)acetic acid (400 mg, 1.20 Eq, 698 μmol), DIEA (230 mg, 3.05 Eq, 1.78 mmol), HATU (290 mg, 1.31 Eq, 763 μmol) and DMF (5 mL). The mixture was stirred at 20° C. for 5 min then tri-tert-butyl 2,2′,2″-(10-(2-(((R)-5-((R)-5-(tert-butoxy)-2-(4-(4-iodophenyl)butanamido)-5-oxopentanamido)-6-methoxy-6-oxohexyl)amino)-2-oxoethyl)-1,4,7,10-tetraazacyclododecane-1,4,7-triyl)triacetate (250 mg, 213 μmol, 36.6%) was added. The reaction mixture was stirred at 20° C. for 1 hour. The mixture was directly purified by Prep-HPLC with the following conditions: Column, C18 120 g Column; mobile phase, Water (0.05% FA) and ACN (30% ACN up to 98% in 15 min); Total flow rate, 90 mL/min; Detector, UV 220 nm. The collected fractions were concentrated to provide tri-tert-butyl 2,2′,2″-(10-(2-(((R)-5-((R)-5-(tert-butoxy)-2-(4-(4-iodophenyl)butanamido)-5-oxopentanamido)-6-methoxy-6-oxohexyl)amino)-2-oxoethyl)-1,4,7,10-tetraazacyclododecane-1,4,7-triyl)triacetate (250 mg, 213 mol, 36.6%) as a colorless oil. Calc'd for C₅₄H₉₀IN₇O₁₃: 1171.56, found [M+H]: 1173.3.

Step 6.: Into an 8-mL vial, was placed a mixture of tri-tert-butyl 2,2′,2″-(10-(2-(((R)-5-((R)-5-(tert-butoxy)-2-(4-(4-iodophenyl)butanamido)-5-oxopentanamido)-6-methoxy-6-oxohexyl)amino)-2-oxoethyl)-1,4,7,10-tetraazacyclododecane-1,4,7-triyl)triacetate (240 mg, 1 Eq, 205 μmol), LiOH (15 mg, 3.1 Eq, 0.63 mmol), MeOH (3 mL) and H₂O (1 mL). The reaction mixture was stirred at 20° C. for 2 hours. The mixture was purified by Prep-HPLC with the following conditions: Column, C18 120 g, 19*150 mm Sum; mobile phase, Water (0.05% FA) and ACN (30.0% ACN up to 98.0% in 3 min); Total flow rate, 70 mL/min; Detector, UV 220 nm. The collected fractions were combined and concentrated to provide N2-((R)-5-(tert-butoxy)-2-(4-(4-iodophenyl)butanamido)-5-oxopentanoyl)-N6-(2-(4,7,10-tris(2-(tert-butoxy)-2-oxoethyl)-1,4,7,10-tetraazacyclododecan-1-yl)acetyl)-D-lysine (213 mg, 184 μmol, 89.8%) as a colorless oil. Calc'd for C₅₃H₈₈IN₇O₁₃: 1157.55, found [M+H]: 1158.6.

Step 7.: Into an 8-mL vial, was placed a mixture of N2-((R)-5-(tert-butoxy)-2-(4-(4-iodophenyl)butanamido)-5-oxopentanoyl)-N6-(2-(4,7,10-tris(2-(tert-butoxy)-2-oxoethyl)-1,4,7,10-tetraazacyclododecan-1-yl)acetyl)-D-lysine (30 mg, 1 Eq, 26 μmol), 3-(((ethylimino)methylene)amino)-N,N-dimethylpropan-1-amine hydrochloride (25 mg, 5.0 Eq, 0.13 mmol), 1H-benzo[d][1,2,3]triazol-1-ol hydrate (20 mg, 5.0 Eq, 0.13 mmol), N-ethyl-N-isopropylpropan-2-amine (33 mg, 9.9 Eq, 0.26 mmol), and DMF (1 mL). The mixture was stirred at 25° C. for 5 min then N-(2-((1-amino-28-oxo-3,6,9,12,15,18,21,24-octaoxa-27-azatriacontan-30-yl)oxy)phenyl)-4-chloro-3-(4-cyano-6-(trifluoromethyl)pyridin-3-yl)-N-methylbenzamide (from Example 2, Step 1, 28 mg, 1.2 Eq, 31 μmol) was added. The reaction mixture was stirred at 25° C. for 2 hours. The mixture was directly purified by Prep-HPLC with the following conditions: Column, C18 120 g Column; mobile phase, water (0.05% FA) and ACN (30% ACN up to 98% in 15 min); Total flow rate, 90 mL/min; Detector, UV 220 nm. The collected fractions were concentrated to provide tri-tert-butyl 2,2′,2″-(10-((R)-8-((R)-5-(tert-butoxy)-2-(4-(4-iodophenyl)butanamido)-5-oxopentanamido)-40-(2-(4-chloro-3-(4-cyano-6-(trifluoromethyl)pyridin-3-yl)-N-methylbenzamido)phenoxy)-2,9,38-trioxo-13,16,19,22,25,28,31,34-octaoxa-3,10,37-triazatetracontyl)-1,4,7,10-tetraazacyclododecanetetraazacyclododecane-1,4,7-triyl)triacetate (20 mg, 9.8 μmol, 38%) as a white solid. Calc'd for C₉₅H₁₄₁ClF₃IN₁₂O₂₃: 2036.89, found [M/2+H]: 1020.3.

Step 8.: Into an 8-mL vial, was placed a mixture of tri-tert-butyl 2,2′,2″-(10-((R)-8-((R)-5-(tert-butoxy)-2-(4-(4-iodophenyl)butanamido)-5-oxopentanamido)-40-(2-(4-chloro-3-(4-cyano-6-(trifluoromethyl)pyridin-3-yl)-N-methylbenzamido)phenoxy)-2,9,38-trioxo-13,16,19,22,25,28,31,34-octaoxa-3,10,37-triazatetracontyl)-1,4,7,10-tetraazacyclododecane-1,4,7-triyl)triacetate (20 mg, 1 Eq, 9.8 μmol) and TFA (0.2 mL). The mixture was stirred at 20° C. for 1 hour. The mixture was concentrated and purified by Column: Sunfire Prep C18 OBD Column, 50*250 mm, 5 m 10 nm; Mobile Phase A: Water (0.05% TFA), Mobile Phase B: ACN; Flow rate: 90 mL/min; Gradient: 25% B to 55% B in 8 min. The collected fractions were concentrated to provide 2,2′,2″-(10-((R)-8-((R)-4-carboxy-2-(4-(4-iodophenyl)butanamido)butanamido)-40-(2-(4-chloro-3-(4-cyano-6-(trifluoromethyl)pyridin-3-yl)-N-methylbenzamido)phenoxy)-2,9,38-trioxo-13,16,19,22,25,28,31,34-octaoxa-3,10,37-triazatetracontyl)-1,4,7,10-tetraazacyclododecane-1,4,7-triyl)triacetic acid-2,2,2-trifluoroacetic acid (1/1) (2.7 mg, 1.4 μmol, 14%) as an off-white solid. Calc'd for C₈₁H₁₁₀ClF₆IN₁₂O₂₅: 1926.63, found [M-TFA+H]: 1814.0.

### Example 11: (Compound 11) 2-[4,7-bis(carboxymethyl)-10-[({26-[3-(2-{N-methyl-4-chloro-3-[4-cyano-6-(trifluoromethyl)pyridin-3-yl]benzamido}phenoxy)propanamido]-3,6,9,12,15,18,21,24-octaoxahexacosan-1-yl}carbamoyl)methyl]-1,4,7,10-tetraazacyclododecan-1-yl]acetic acid

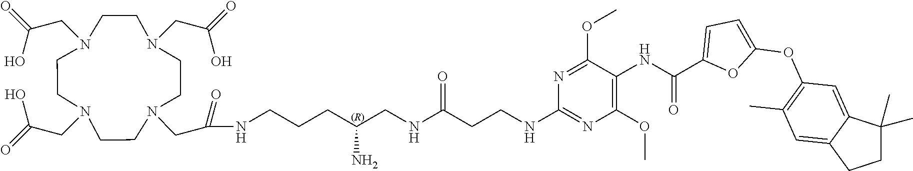

### Example 12: (Compound 12) (R)-2,2′,2″-(10-(2-((1-carboxy-4-(3-((4,6-dimethoxy-5-(5-((3,3,6-trimethyl-2,3-dihydro-1H-inden-5-yl)oxy)furan-2-carboxamido)pyrimidin-2-yl)amino)propanamido)butyl)amino)-2-oxoethyl)-1,4,7,10-tetraazacyclododecane-1,4,7-triyl)triacetic acid

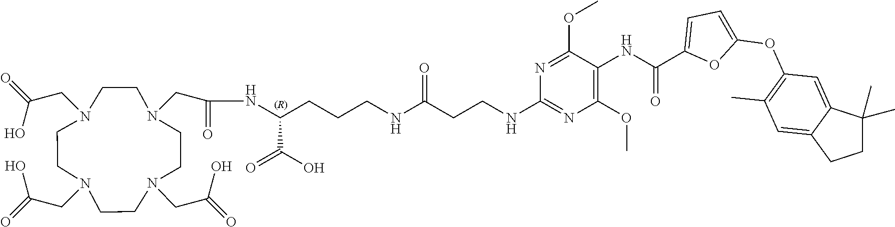

### Example 13: (Compound 13) 2,2′,2″-(10-(20-((4,6-dimethoxy-5-(5-((3,3,6-trimethyl-2,3-dihydro-1H-inden-5-yl)oxy)furan-2-carboxamido)pyrimidin-2-yl)amino)-2-oxo-6,9,12,15,18-pentaoxa-3-azaicosyl)-1,4,7,10-tetraazacyclododecane-1,4,7-triyl)triacetic acid

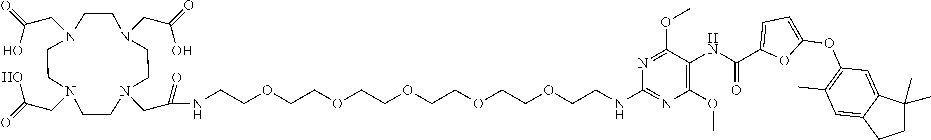 [SMILES: COc1nc(NCCOCCOCCOCCOCCOCCNC(=O)CN2CCN(CC(=O)O)CCN(CC(=O)O)CCN(CC(=O)O)CC2)nc(OC)c1NC(=O)c1ccc(Oc2cc3c(cc2C)CCC3(C)C)o1]

### Example 14: (Compound 14) 2,2′,2″-(10-(14-((4,6-dimethoxy-5-(5-((3,3,6-trimethyl-2,3-dihydro-1H-inden-5-yl)oxy)furan-2-carboxamido)pyrimidin-2-yl)amino)-2-oxo-6,9,12-trioxa-3-azatetradecyl)-1,4,7,10-tetraazacyclododecane-1,4,7-triyl)triacetic acid

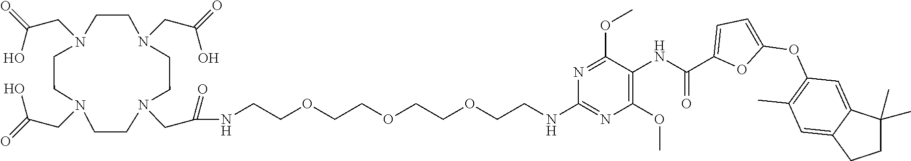 [SMILES: COc1nc(NCCOCCOCCOCCNC(=O)CN2CCN(CC(=O)O)CCN(CC(=O)O)CCN(CC(=O)O)CC2)nc(OC)c1NC(=O)c1ccc(Oc2cc3c(cc2C)CCC3(C)C)o1]

### Example 15: (Compound 15) 2,2′,2″-(10-(2-((2-((2-(3-((4,6-dimethoxy-5-(5-((3,3,6-trimethyl-2,3-dihydro-1H-inden-5-yl)oxy)furan-2-carboxamido)pyrimidin-2-yl)amino)propanamido)ethyl)(methyl)amino)ethyl)amino)-2-oxoethyl)-1,4,7,10-tetraazacyclododecane-1,4,7-triyl)triacetic acid

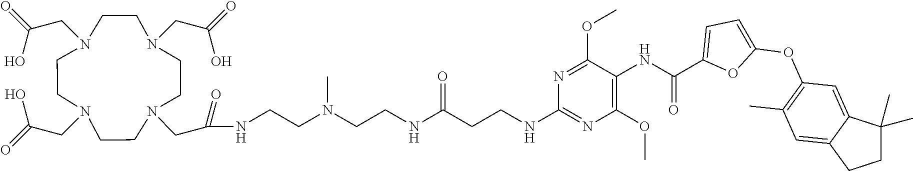 [SMILES: COc1nc(NCCC(=O)NCCN(C)CCNC(=O)CN2CCN(CC(=O)O)CCN(CC(=O)O)CCN(CC(=O)O)CC2)nc(OC)c1NC(=O)c1ccc(Oc2cc3c(cc2C)CCC3(C)C)o1]

### Example 16: (Compound 16) (R)-2,2′,2″-(10-(2-((1-carboxy-4-(3-((4,6-dimethoxy-5-(5-((3,3,6-trimethyl-2,3-dihydro-1H-inden-5-yl)oxy)furan-2-carboxamido)pyrimidin-2-yl)thio)propanamido)butyl)amino)-2-oxoethyl)-1,4,7,10-tetraazacyclododecane-1,4,7-triyl)triacetic acid

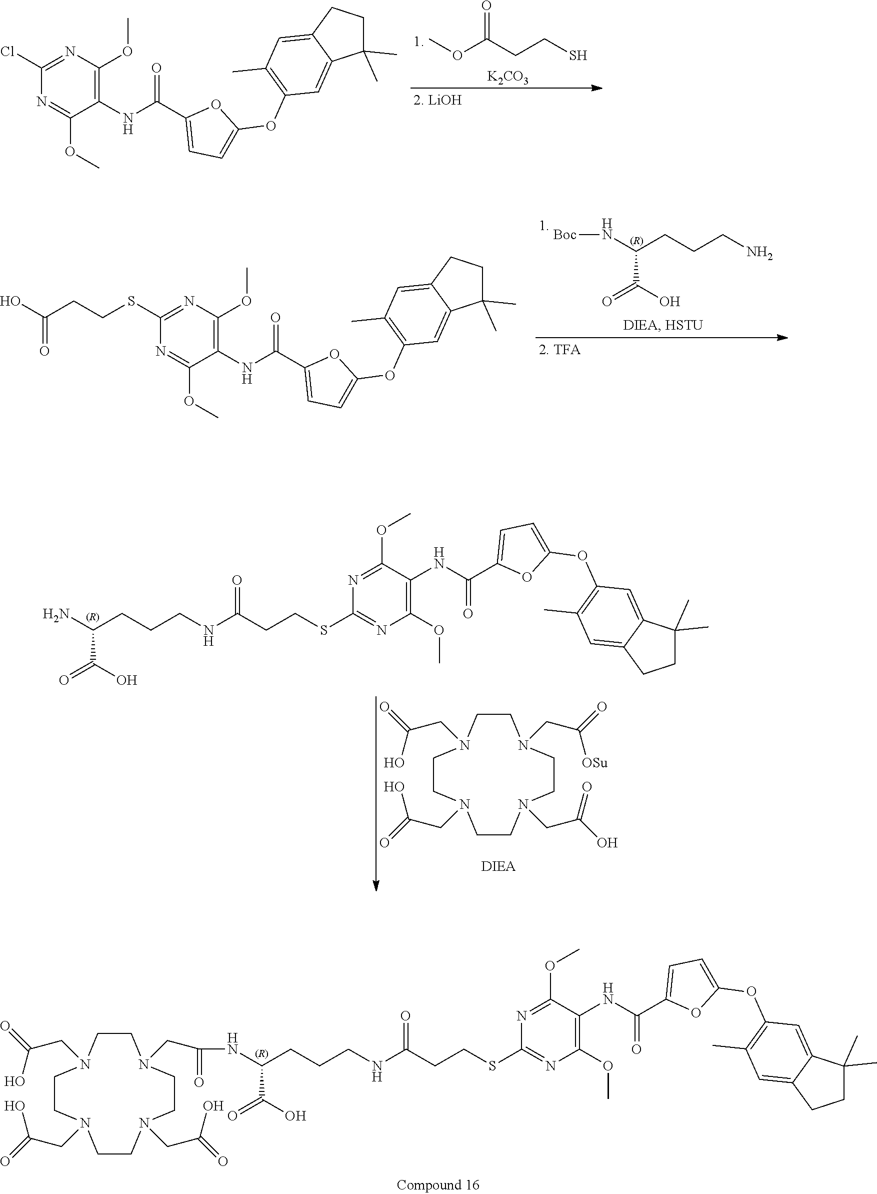

Step 1.: Into an 8-mL vial, was placed a mixture of N-(2-chloro-4,6-dimethoxypyrimidin-5-yl)-5-((3,3,6-trimethyl-2,3-dihydro-1H-inden-5-yl)oxy)furan-2-carboxamide (600 mg, 1 Eq, 1.31 mmol), methyl 3-mercaptopropanoate (315 mg, 2.00 Eq, 2.62 mmol), potassium carbonate (543 mg, 3.00 Eq, 3.93 mmol) and MeCN (8 mL). The reaction mixture was stirred at 80° C. for 2 hours. The mixture was directly purified by MPLC, then the collected fractions were concentrated under reduced pressure to provide methyl 3-((4,6-dimethoxy-5-(5-((3,3,6-trimethyl-2,3-dihydro-1H-inden-5-yl)oxy)furan-2-carboxamido)pyrimidin-2-yl)thio)propanoate (500 mg, 923 μmol, 70.5%) as a yellow solid. [M+H]=542.1.

Step 2.: Into a 40-mL vial, was placed a mixture of methyl 3-((4,6-dimethoxy-5-(5-((3,3,6-trimethyl-2,3-dihydro-1H-inden-5-yl)oxy)furan-2-carboxamido)pyrimidin-2-yl)thio)propanoate (500 mg, 1 Eq, 923 μmol), LiOH (221 mg, 10.0 Eq, 9.23 mmol), MeOH (8 mL) and H₂O (3 mL). The reaction mixture was stirred at 25° C. for 2 hours. The reaction mixture was concentrated under reduced pressure to remove most of the MeOH, then the residue was diluted with water (50 mL) and the pH value was adjusted to 6.0 by addition of a saturated NaHSO₄ solution. The aqueous solution was extracted with DCM (50 mL×3), dried over anhydrous Na₂SO₄, then concentrated under reduced pressure to afford 3-((4,6-dimethoxy-5-(5-((3,3,6-trimethyl-2,3-dihydro-1H-inden-5-yl)oxy)furan-2-carboxamido)pyrimidin-2-yl)thio)propanoic acid (450 mg, 853 μmol, 92.4%) as a light yellow solid, which was used directly for the next step without any purification. [M+H]=528.1.

Step 3.: Into a 40-mL vial, was placed a mixture of 3-((4,6-dimethoxy-5-(5-((3,3,6-trimethyl-2,3-dihydro-1H-inden-5-yl)oxy)furan-2-carboxamido)pyrimidin-2-yl)thio)propanoic acid (100 mg, 1 Eq, 190 μmol), HSTU (88.5 mg, 1.30 Eq, 246 μmol), DIEA (122 mg, 4.99 Eq, 946 μmol) and DMF (2 mL). The reaction mixture was stirred at 20° C. for 10 minutes, then (R)-5-amino-2-((tert-butoxycarbonyl)amino)pentanoic acid (52.8 mg, 1.20 Eq, 227 μmol) was added and the reaction mixture was stirred at 25° C. for an additional 2 hours. The mixture was directly purified by MPLC to provide (R)-2-((tert-butoxycarbonyl)amino)-5-(3-((4,6-dimethoxy-5-(5-((3,3,6-trimethyl-2,3-dihydro-1H-inden-5-yl)oxy)furan-2-carboxamido)pyrimidin-2-yl)thio)propanamido)pentanoic acid (110 mg, 148 μmol, 78.2%) as a yellow solid. [M+Na]=766.3.

Step 4.: Into an 8-mL vial, was placed a mixture of (R)-2-((tert-butoxycarbonyl)amino)-5-(3-((4,6-dimethoxy-5-(5-((3,3,6-trimethyl-2,3-dihydro-1H-inden-5-yl)oxy)furan-2-carboxamido)pyrimidin-2-yl)thio)propanamido)pentanoic acid (70 mg, 1 Eq, 94 mol) and DCM (1 mL), to which was added TFA (0.3 mL). The reaction mixture was stirred at 25° C. for 1 hour. The mixture was concentrated under reduced pressure, to provide (R)-2-amino-5-(3-((4,6-dimethoxy-5-(5-((3,3,6-trimethyl-2,3-dihydro-1H-inden-5-yl)oxy)furan-2-carboxamido)pyrimidin-2-yl)thio)propanamido)pentanoic acid (70 mg, 93 μmol, 98%) as a yellow oil, which was used directly in the next step without any further purification. [M+H]=642.3.

Step 5.: Into an 8-mL vial, was placed a mixture of (R)-2-amino-5-(3-((4,6-dimethoxy-5-(5-((3,3,6-trimethyl-2,3-dihydro-1H-inden-5-yl)oxy)furan-2-carboxamido)pyrimidin-2-yl)thio)propanamido)pentanoic acid (60 mg, 1 Eq, 93 μmol), (R)-5-amino-2-((tert-butoxycarbonyl)amino)pentanoic acid (52.8 mg, 1.20 Eq, 227 μmol), DIEA (36 mg, 3.0 Eq, 0.28 mmol) and DMF (1 mL). The reaction mixture was stirred at 25° C. for 2 hours. The crude product was purified by Prep-HPLC to provide (R)-2,2′,2″-(10-(2-((1-carboxy-4-(3-((4,6-dimethoxy-5-(5-((3,3,6-trimethyl-2,3-dihydro-1H-inden-5-yl)oxy)furan-2-carboxamido)pyrimidin-2-yl)thio)propanamido)butyl)amino)-2-oxoethyl)-1,4,7,10-tetraazacyclododecane-1,4,7-triyl)triacetic acid-2,2,2-trifluoroacetic acid (1/1) (39.5 mg, 34.6 mol, 37%) as a white solid. Calc'd for C₄₉H₆₆F₃N₉O₁₇S: 1141.42, found [M+H-TFA]: 1028.4.

### Example 17: (Compound 17) 2,2′,2″-(10-(2-((2-((2-(3-((4,6-dimethoxy-5-(5-((3,3,6-trimethyl-2,3-dihydro-1H-inden-5-yl)oxy)furan-2-carboxamido)pyrimidin-2-yl)thio)propanamido)ethyl)(methyl)amino)ethyl)amino)-2-oxoethyl)-1,4,7,10-tetraazacyclododecane-1,4,7-triyl)triacetic acid

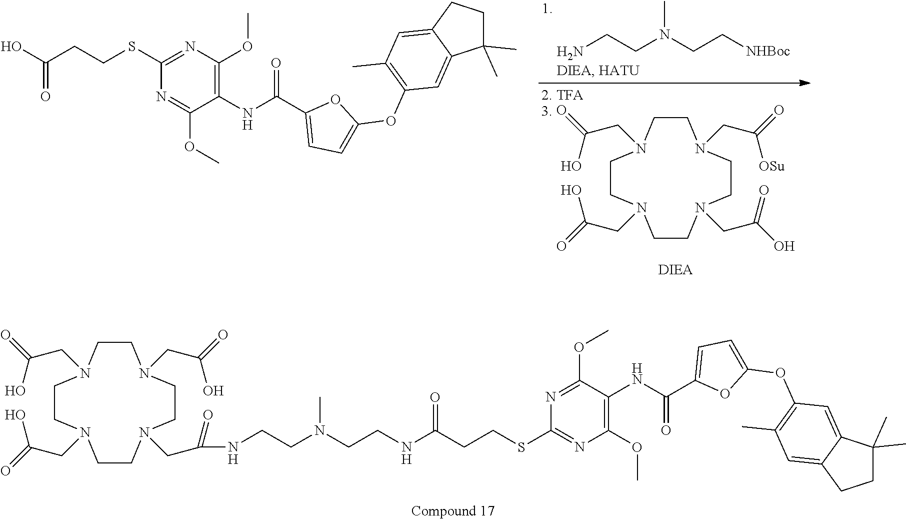

Step 1.: Into a 40-mL vial, was placed a mixture of 3-((4,6-dimethoxy-5-(5-((3,3,6-trimethyl-2,3-dihydro-1H-inden-5-yl)oxy)furan-2-carboxamido)pyrimidin-2-yl)thio)propanoic acid (200 mg, 1 Eq, 379 μmol), HATU (187 mg, 1.30 Eq, 492 μmol), DIEA (245 mg, 5.01 Eq, 1.90 mmol) and DMF (2 mL). The reaction mixture was stirred at 20° C. for 10 minutes, then tert-butyl (2-((2-aminoethyl)(methyl)amino)ethyl)carbamate (107 mg, 1.30 Eq, 492 μmol) was added and the reaction mixture was stirred at 25° C. for an additional 1 hour. The mixture was directly purified by MPLC to provide tert-butyl (2-((2-(3-((4,6-dimethoxy-5-(5-((3,3,6-trimethyl-2,3-dihydro-1H-inden-5-yl)oxy)furan-2-carboxamido)pyrimidin-2-yl)thio)propanamido)ethyl)(methyl)amino)ethyl)carbamate (210 mg, 289 μmol, 76.2%) as a white solid. [M+H]=727.4.

Step 2.: Into an 8-mL vial, was placed a mixture of tert-butyl (2-((2-(3-((4,6-dimethoxy-5-(5-((3,3,6-trimethyl-2,3-dihydro-1H-inden-5-yl)oxy)furan-2-carboxamido)pyrimidin-2-yl)thio)propanamido)ethyl)(methyl)amino)ethyl)carbamate (80 mg, 1 Eq, 0.11 mmol) and DCM (1 mL), to which was added TFA (0.3 mL). The reaction mixture was stirred at 25° C. for 1 hour. The mixture was concentrated under reduced pressure to provide N-(2-((3-((2-((2-aminoethyl)(methyl)amino)ethyl)amino)-3-oxopropyl)thio)-4,6-dimethoxypyrimidin-5-yl)-5-((3,3,6-trimethyl-2,3-dihydro-1H-inden-5-yl)oxy)furan-2-carboxamide (70 mg, 95 μmol, 86%) as a yellow oil, which was used directly in the next step without further purification. [M+H]=627.3.

Step 3.: N-(2-((3-((2-((2-aminoethyl)(methyl)amino)ethyl)amino)-3-oxopropyl)thio)-4,6-dimethoxypyrimidin-5-yl)-5-((3,3,6-trimethyl-2,3-dihydro-1H-inden-5-yl)oxy)furan-2-carboxamide (70 mg, 1 Eq, 0.11 mmol) was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA (72 mg, 5.0 Eq, 0.56 mmol) in a manner similar to Example 16, Step 5 to provide 2,2′,2″-(10-(2-((2-((2-(3-((4,6-dimethoxy-5-(5-((3,3,6-trimethyl-2,3-dihydro-1H-inden-5-yl)oxy)furan-2-carboxamido)pyrimidin-2-yl)thio)propanamido)ethyl)(methyl)amino)ethyl)amino)-2-oxoethyl)-1,4,7,10-tetraazacyclododecane-1,4,7-triyl)triacetic acid-2,2,2-trifluoroacetic acid (1/1) (21.9 mg, 19.4 μmol, 17%) as a white solid. Calc'd for C₄₉H₆₉F₃N₁₀O₁₅S: 1126.46, found [M+H-TFA]: 1013.4.

### Example 18: (Compound 18) (R)-2,2′,2″-(10-(2-((4-amino-5-((3-((4,6-dimethoxy-5-(5-((3,3,6-trimethyl-2,3-dihydro-1H-inden-5-yl)oxy)furan-2-carboxamido)pyrimidin-2-yl)thio)propyl)amino)-5-oxopentyl)amino)-2-oxoethyl)-1,4,7,10-tetraazacyclododecane-1,4,7-triyl)triacetic acid

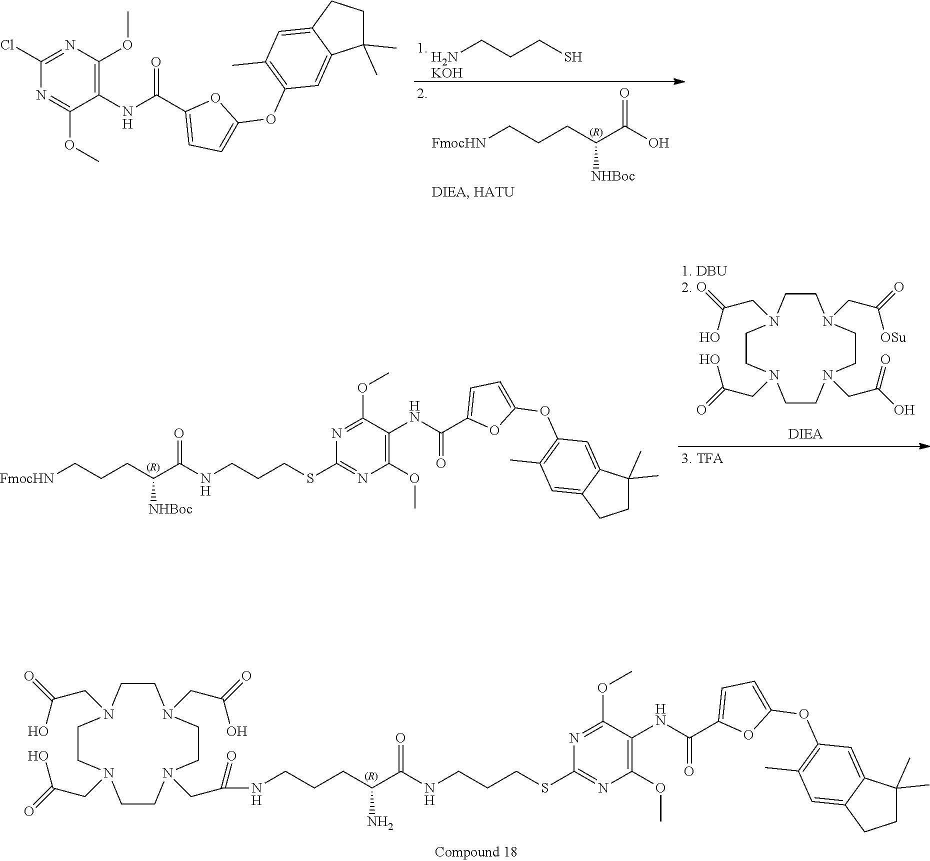

Step 1.: Into a 8-mL vial, was placed a mixture of N-(2-chloro-4,6-dimethoxypyrimidin-5-yl)-5-((3,3,6-trimethyl-2,3-dihydro-1H-inden-5-yl)oxy)furan-2-carboxamide (200 mg, 1 Eq, 437 μmol), 3-aminopropane-1-thiol (44 mg, 1.1 Eq, 0.48 mmol), KOH (73 mg, 3.0 Eq, 1.3 mmol) EtOH (2 mL), and H₂O (0.7 mL). The reaction mixture was stirred at 80° C. for 2 hours. The mixture was directly purified by MPLC to provide N-(2-((3-aminopropyl)thio)-4,6-dimethoxypyrimidin-5-yl)-5-((3,3,6-trimethyl-2,3-dihydro-1H-inden-5-yl)oxy)furan-2-carboxamide (80 mg, 0.16 mmol, 36%) as a yellow solid. [M+H]=513.5.

Step 2.: N-(2-((3-aminopropyl)thio)-4,6-dimethoxypyrimidin-5-yl)-5-((3,3,6-trimethyl-2,3-dihydro-1H-inden-5-yl)oxy)furan-2-carboxamide was treated with (R)-5-((((9H-fluoren-9-yl)methoxy)carbonyl)amino)-2-((tert-butoxycarbonyl)amino)pentanoic acid (85 mg, 1.2 Eq, 0.19 mmol), HATU and DIEA in a manner similar to Example 17, Step 1 to provide (9H-fluoren-9-yl)methyl tert-butyl (5-((3-((4,6-dimethoxy-5-(5-((3,3,6-trimethyl-2,3-dihydro-1H-inden-5-yl)oxy)furan-2-carboxamido)pyrimidin-2-yl)thio)propyl)amino)-5-oxopentane-1,4-diyl)(R)-dicarbamate (55 mg, 58 μmol, 37%) as a white solid. [M+Na]=971.6.

Step 3.: Into an 8-mL vial, was placed a mixture of (9H-fluoren-9-yl)methyl tert-butyl (5-((3-((4,6-dimethoxy-5-(5-((3,3,6-trimethyl-2,3-dihydro-1H-inden-5-yl)oxy)furan-2-carboxamido)pyrimidin-2-yl)thio)propyl)amino)-5-oxopentane-1,4-diyl)(R)-dicarbamate (55 mg, 1 Eq, 58 μmol) and DMF (1 mL), to which was added DBU (0.12 g, 0.12 mL, 14 Eq, 0.79 mmol). The reaction mixture was stirred at 25° C. for 2 h, to provide tert-butyl (R)-(5-amino-1-((3-((4,6-dimethoxy-5-(5-((3,3,6-trimethyl-2,3-dihydro-1H-inden-5-yl)oxy)furan-2-carboxamido)pyrimidin-2-yl)thio)propyl)amino)-1-oxopentan-2-yl)carbamate as a crude product, which was used in the next step without further purification. [M+H]=727.6.

Step 4.: tert-butyl (R)-(5-amino-1-((3-((4,6-dimethoxy-5-(5-((3,3,6-trimethyl-2,3-dihydro-1H-inden-5-yl)oxy)furan-2-carboxamido)pyrimidin-2-yl)thio)propyl)amino)-1-oxopentan-2-yl)carbamate was treated with 2,2′,2″-(10-(2-((2,5-dioxopyrrolidin-1-yl)oxy)-2-oxoethyl)-1,4,7,10-tetraazacyclododecane-1,4,7-triyl)triacetic acid and DIEA (72 mg, 5.0 Eq, 0.56 mmol) in a manner similar to Example 16, Step 5 to provide (R)-2,2′,2″-(10-(2-((4-((tert-butoxycarbonyl)amino)-5-((3-((4,6-dimethoxy-5-(5-((3,3,6-trimethyl-2,3-dihydro-1H-inden-5-yl)oxy)furan-2-carboxamido)pyrimidin-2-yl)thio)propyl)amino)-5-oxopentyl)amino)-2-oxoethyl)-1,4,7,10-tetraazacyclododecane-1,4,7-triyl)triacetic acid (50 mg, 45 μmol, 73%) as a white solid. [M+H-TFA]=1113.8.

Step 5.: Into an 8-mL vial, was placed a mixture of (R)-2,2′,2″-(10-(2-((4-((tert-butoxycarbonyl)amino)-5-((3-((4,6-dimethoxy-5-(5-((3,3,6-trimethyl-2,3-dihydro-1H-inden-5-yl)oxy)furan-2-carboxamido)pyrimidin-2-yl)thio)propyl)amino)-5-oxopentyl)amino)-2-oxoethyl)-1,4,7,10-tetraazacyclododecane-1,4,7-triyl)triacetic acid (45 mg, 1 Eq, 40 mol) and DCM (1 mL), to which was added TFA (0.3 mL). The reaction mixture was stirred at 25° C. for 1 hour. The mixture was concentrated under reduced pressure and the crude product was purified by Prep-HPLC to provide (R)-2,2′,2″-(10-(2-((4-amino-5-((3-((4,6-dimethoxy-5-(5-((3,3,6-trimethyl-2,3-dihydro-1H-inden-5-yl)oxy)furan-2-carboxamido)pyrimidin-2-yl)thio)propyl)amino)-5-oxopentyl)amino)-2-oxoethyl)-1,4,7,10-tetraazacyclododecane-1,4,7-triyl)triacetic acid-2,2,2-trifluoroacetic acid (1/1) (22.3 mg, 19.8 μmol, 49%) as a white solid. Calc'd for C₄₉H₆₉F₃N₁₀O₁₅S: 1126.46, found [M+H-TFA]: 1013.5.

### Example 19: (Compound 19) (R)-2,2′,2″-(10-(7-amino-18-((4,6-dimethoxy-5-(5-((3,3,6-trimethyl-2,3-dihydro-1H-inden-5-yl)oxy)furan-2-carboxamido)pyrimidin-2-yl)thio)-12-methyl-2,8,16-trioxo-3,9,12,15-tetraazaoctadecyl)-1,4,7,10-tetraazacyclododecane-1,4,7-triyl)triacetic acid

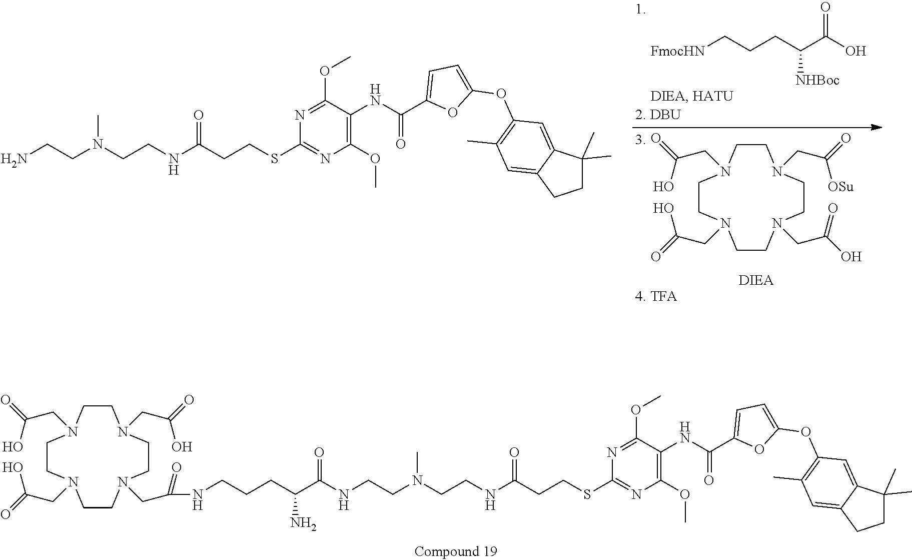

Step 1.: N-(2-((3-((2-((2-aminoethyl)(methyl)amino)ethyl)amino)-3-oxopropyl)thio)-4,6-dimethoxypyrimidin-5-yl)-5-((3,3,6-trimethyl-2,3-dihydro-1H-inden-5-yl)oxy)furan-2-carboxamide (130 mg, 1.0 Eq, 207 μmol) was treated with (R)-5-((((9H-fluoren-9-yl)methoxy)carbonyl)amino)-2-((tert-butoxycarbonyl)amino)pentanoic acid, HATU and DIEA in a manner similar to Example 17, Step 1 to provide (9H-fluoren-9-yl)methyl tert-butyl (5-((2-((2-(3-((4,6-dimethoxy-5-(5-((3,3,6-trimethyl-2,3-dihydro-1H-inden-5-yl)oxy)furan-2-carboxamido)pyrimidin-2-yl)thio)propanamido)ethyl)(methyl)amino)ethyl)amino)-5-oxopentane-1,4-diyl)(R)-dicarbamate (110 mg, 103 μmol, 49.9%) as a white solid. [M+H]=1063.5.

Step 2.: (9H-fluoren-9-yl)methyl tert-butyl (5-((2-((2-(3-((4,6-dimethoxy-5-(5-((3,3,6-trimethyl-2,3-dihydro-1H-inden-5-yl)oxy)furan-2-carboxamido)pyrimidin-2-yl)thio)propanamido)ethyl)(methyl)amino)ethyl)amino)-5-oxopentane-1,4-diyl)(R)-dicarbamate was treated with DBU in a manner similar to Example 18, Step 3 to provide crude tert-butyl (R)-(5-amino-1-((2-((2-(3-((4,6-dimethoxy-5-(5-((3,3,6-trimethyl-2,3-dihydro-1H-inden-5-yl)oxy)furan-2-carboxamido)pyrimidin-2-yl)thio)propanamido)ethyl)(methyl)amino)ethyl)amino)-1-oxopentan-2-yl)carbamate (85 mg, 86 mol, 91%), which was used in the next step without further purification. [M+H]=841.1.

Steps 3 and 4.: (R)-2,2′,2″-(10-(7-amino-18-((4,6-dimethoxy-5-(5-((3,3,6-trimethyl-2,3-dihydro-1H-inden-5-yl)oxy)furan-2-carboxamido)pyrimidin-2-yl)thio)-12-methyl-2,8,16-trioxo-3,9,12,15-tetraazaoctadecyl)-1,4,7,10-tetraazacyclododecane-1,4,7-triyl)triacetic acid-2,2,2-trifluoroacetic acid (1/1) was prepared from tert-butyl (R)-(5-amino-1-((2-((2-(3-((4,6-dimethoxy-5-(5-((3,3,6-trimethyl-2,3-dihydro-1H-inden-5-yl)oxy)furan-2-carboxamido)pyrimidin-2-yl)thio)propanamido)ethyl)(methyl)amino)ethyl)amino)-1-oxopentan-2-yl)carbamate in a manner similar to Example 18, Steps 4 and 5. The product was obtained as a white solid (66.0 mg, 53.2 μmol, 82%). Calc'd for C₅₄H₇₉F₃N₁₂O₁₆S: 1240.54, found [M+H-TFA]: 1127.6.

### Example 20: Parenteral Pharmaceutical Composition

To prepare a parenteral pharmaceutical composition suitable for administration by injection (subcutaneous, intravenous), 0.001-500 mg of a compound Formula (I), (II), (IIIa), (IIIb), (IIIc), (IIId), or (IIIe), or a pharmaceutically acceptable salt or solvate thereof, is dissolved in sterile water and then mixed with 10 mL of 0.9% sterile saline. A suitable buffer is optionally added as well as optional acid or base to adjust the pH. The mixture is incorporated into a dosage unit form suitable for administration by injection.

### Biology Examples

### GnRHR Assays

### Functional Assay for GnRHR

General overview: GnRHR is a G_{q/11}-coupled receptor that mediates the action of the GnRH hormone by activating the phosphatidylinositol-calcium second messenger system. Activation of the GnRHR induces the accumulation of inositol monophosphate, a stable metabolite of IP-3, that can be characterized as a measure of agonistic activity (increase in IP-One) or antagonistic activity (blockade of IP-One accumulation) by compounds of the invention. One example of an intracellular IP-One assay used to characterize GnRHR antagonists is described below.

### IP-One Assay Protocol

24 hours prior to the assay, 30,000 FlpIn T-Rex 293 Cells (ThermoFisher #R78007) stably expressing the functional human GnRH receptor upon induction with tetracycline were plated in a 96-well tissue culture-treated plate in FlpIn T-Rex 293 Growth Medium [DMEM (Corning #10-013-CM) supplemented with 10% fetal bovine serum (Gemini Bio-Products #900-208), 100 U/mL penicillin; 100 μg/mL streptomycin; 2 mM L-glutamine (Gemini Bio-Products #400-110)] and 50 ng/mL tetracycline hydrocholoride (Sigma, T7660). Cells were cultured at 37° C., 5% CO₂ and 95% humidity. On the day of the assay, the growth media was discarded and the cells were treated with 50 μL of a dose response curve of GnRH (Bachem #4033013) in the presence of various concentrations of fixed compound in assay buffer [10 mM HEPES (Biopioneer Cat #C0113) pH 7.4, 1 mM CaCl₂) (Fisher Scientific BP510-100), 0.5 mM MgCl₂ (Sigma M8266-100G), 4.2 mM KCl (Fisher Scientific P330-500), 146 mM NaCl (Spectrum Chemical #SO155), 5.5 mM Glucose (Sigma G7528), 50 mM LiCl (Fisher Scientific L121-100), 0.1% bovine serum albumin (Fisher Scientific Cat #BP1600)] and incubated for 1 hour at 37° C. (the final concentrations of GnRH ranged from 0-250 nM and the final concentrations of the compound ranged from 0-10,000 nM). 50 μL of lysis buffer (HRTF IP-one kit, Cisbio) was added on top of the treatment to lyse the cells. The lysate was transferred to 384-well plates and IP-one detection and visualization antibodies were added and incubated for 1-24 hours at room temperature. The time-resolved fluorescent signal was read with a Tecan M1000Pro (Tecan) multiplate reader. The intracellular IP-one concentrations were calculated by regression to a standard curve and were plotted vs. the concentration of the GnRH agonists in the presence of various concentrations of antagonist and the Ks of the compounds were calculated using standard curve-fitting methods. All data manipulations were performed using GraphPad Prism v8 (GraphPad, San Diego, CA).

For some analogs, the incubation time was 2 h or 4 h, as noted in Table A.

Illustrative biological activity of compounds is demonstrated in the following Tables.

TABLE A
Representative Activity

| Cmpd No. | Compound Structure |
|---|---|
| 2 | 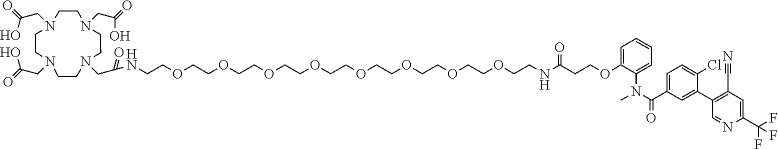 [SMILES: CN(C(=O)c1ccc(Cl)c(-c2cnc(C(F)(F)F)cc2C#N)c1)c1ccccc1OCCC(=O)NCCOCCOCCOCCOCCOCCOCCOCCOCCNC(=O)CN1CCN(CC(=O)O)CCN(CC(=O)O)CCN(CC(=O)O)CC1] |
| 2M- | In115 Complex of 2 |
| In115 | |
| 3 | 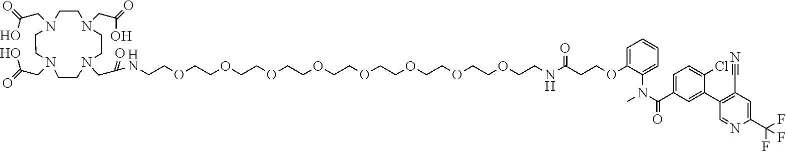 [SMILES: CN(C(=O)c1ccc(Cl)c(-c2cnc(C(F)(F)F)cc2C#N)c1)c1ccccc1OCCC(=O)NCCOCCOCCOCCOCCOCCOCCOCCOCCNC(=O)CN1CCN(CC(=O)O)CCN(CC(=O)O)CCN(CC(=O)O)CC1] |
| 3M-In | In Complex of 3 |
| 4 | 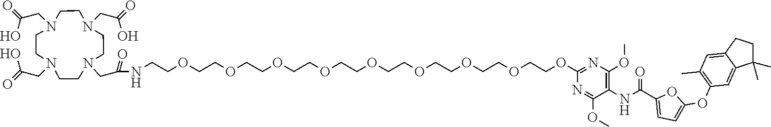 [SMILES: COc1nc(OCCOCCOCCOCCOCCOCCOCCOCCOCCNC(=O)CN2CCN(CC(=O)O)CCN(CC(=O)O)CCN(CC(=O)O)CC2)nc(OC)c1NC(=O)c1ccc(Oc2cc3c(cc2C)CCC3(C)C)o1] |
| 4M-In | In Complex of 4 |
| 5 | 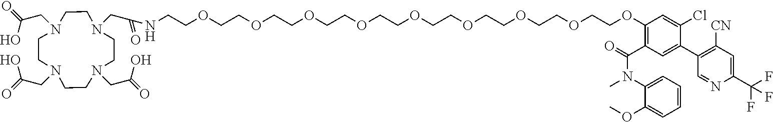 [SMILES: COc1ccccc1N(C)C(=O)c1cc(-c2cnc(C(F)(F)F)cc2C#N)c(Cl)cc1OCCOCCOCCOCCOCCOCCOCCOCCOCCNC(=O)CN1CCN(CC(=O)O)CCN(CC(=O)O)CCN(CC(=O)O)CC1] |
| 5M-In | In Complex of 5 |
| 6 | 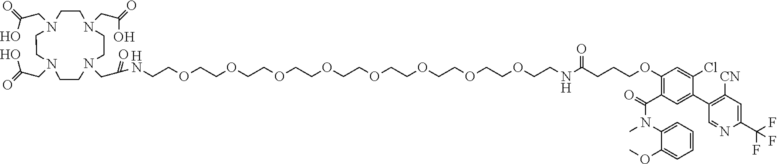 [SMILES: COc1ccccc1N(C)C(=O)c1cc(-c2cnc(C(F)(F)F)cc2C#N)c(Cl)cc1OCCCC(=O)NCCOCCOCCOCCOCCOCCOCCOCCOCCNC(=O)CN1CCN(CC(=O)O)CCN(CC(=O)O)CCN(CC(=O)O)CC1] |
| 6M-In | In Complex of 6 |
| 7 | 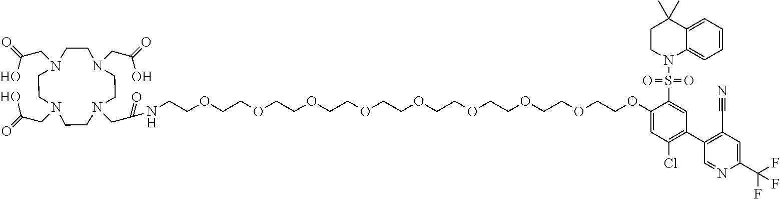 [SMILES: CC1(C)CCN(S(=O)(=O)c2cc(-c3cnc(C(F)(F)F)cc3C#N)c(Cl)cc2OCCOCCOCCOCCOCCOCCOCCOCCOCCNC(=O)CN2CCN(CC(=O)O)CCN(CC(=O)O)CCN(CC(=O)O)CC2)c2ccccc21] |
| 7M-In | Complex of 7 |
| 8 | 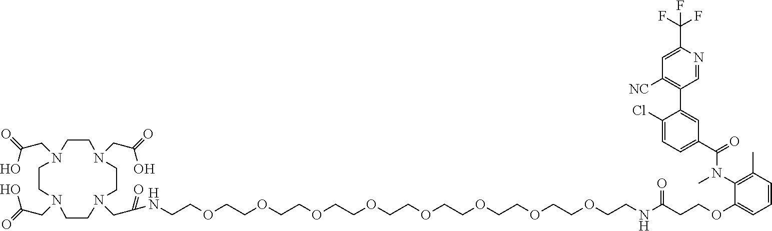 [SMILES: Cc1cccc(OCCC(=O)NCCOCCOCCOCCOCCOCCOCCOCCOCCNC(=O)CN2CCN(CC(=O)O)CCN(CC(=O)O)CCN(CC(=O)O)CC2)c1N(C)C(=O)c1ccc(Cl)c(-c2cnc(C(F)(F)F)cc2C#N)c1] |
| 16 | 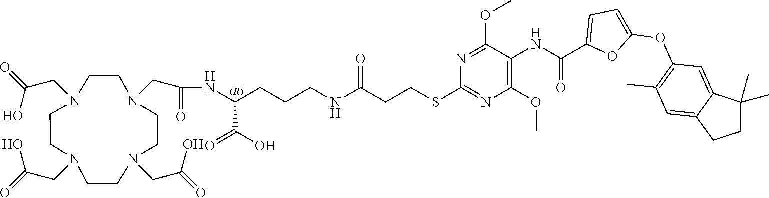 |
| 17 | 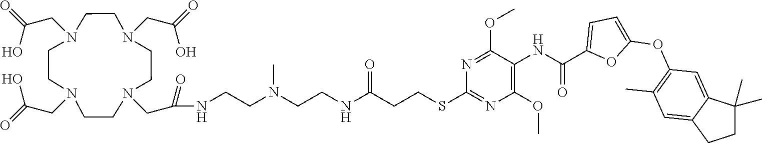 [SMILES: COc1nc(SCCC(=O)NCCN(C)CCNC(=O)CN2CCN(CC(=O)O)CCN(CC(=O)O)CCN(CC(=O)O)CC2)nc(OC)c1NC(=O)c1ccc(Oc2cc3c(cc2C)CCC3(C)C)o1] |
| 18 | 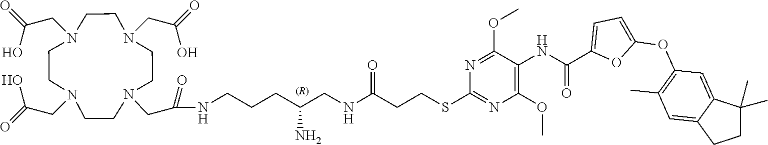 |
| 19 | 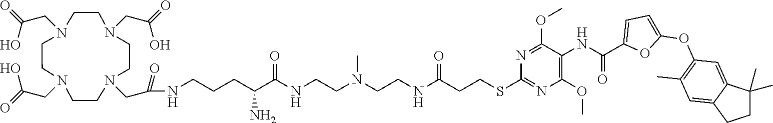 [SMILES: COc1nc(SCCC(=O)NCCN(C)CCNC(=O)[C@H](N)CCCNC(=O)CN2CCN(CC(=O)O)CCN(CC(=O)O)CCN(CC(=O)O)CC2)nc(OC)c1NC(=O)c1ccc(Oc2cc3c(cc2C)CCC3(C)C)o1] |

| | hGnRHR Binding Ki* | | |
|---|---|---|---|
| Cmpd No. | 60 min (nM) | 4 h (nM) | 2 h (nM) |
| 2 | A | | |
| 2M-In115 | A | | |
| 3M-In | A | A | |
| 4 | | | A |
| 4M-In | A | A | |
| 5M-In | A | A | |
| 6M-In | A | A | |
| 7 | | | A |
| 7M-In | A | A | |
| 8 | A | A | |
| 16 | | | A |
| 17 | | | A |
| 18 | | | A |
| 19 | | | A |

*A is <10 nM; B is 10-100 nM; C is >100 nM

Other embodiments are set forth in the following claims, along with the full scope of equivalents to which such claims are entitled.

What is claimed is:

1. A compound of Formula (I), or a pharmaceutically acceptable salt thereof, 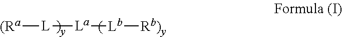
wherein:
L^{a} is absent or a branched linker;
each L is independently selected from absent or a linker;
each L^{b} is independently selected from absent or a linker;
each R^{a} is independently selected from a chelating moiety or a radionuclide complex thereof;
each R^{b} is independently a small molecule modulator of the gonadotropin-releasing hormone receptor (GnRHR); and
each y is independently 1, 2 or 3.

2. The compound of claim 1, or a pharmaceutically acceptable salt thereof, wherein each y is 1.

3. The compound of claim 1 or 2, or a pharmaceutically acceptable salt thereof, wherein R^{b} is a small molecule antagonist of GnRHR.

4. The compound of any one of claims 1-3, or a pharmaceutically acceptable salt thereof, wherein R^{b} comprises a furan pyrimidine, a pyridine-chloro-benzamide, a pyridine-chloro-benzenesulfamide, a uracil, a thieno[2,3-d]pyrimidine-2,4(1H,3H)-dione, or a thieno[3,4-d]pyrimidine-2,4(1H,3H)-dione.

5. The compound of any one of claims 1-3, or a pharmaceutically acceptable salt thereof, wherein R^{b} comprises a furan pyrimidine.

6. The compound of any one of claims 1-3, or a pharmaceutically acceptable salt thereof, wherein R^{b} comprises a pyridine-chloro-benzamide or a pyridine-chloro-benzenesulfamide.

7. The compound of any one of claims 1-6, or a pharmaceutically acceptable salt thereof, wherein the compound has the structure of Formula (II), or a pharmaceutically acceptable salt thereof: 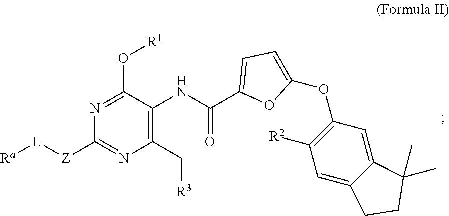
wherein:
R¹ is H, substituted or unsubstituted —C₁-C₄ alkyl, or substituted or unsubstituted —C₁-C₄ haloalkyl;
R² is H, halo, substituted or unsubstituted —C₁-C₆ alkyl, or substituted or unsubstituted —C₁-C₄ haloalkyl;
R³ is H, substituted or unsubstituted —C₁-C₄ alkyl, or substituted or unsubstituted —C₁-C₄ haloalkyl;
Z is absent, —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—;
R⁴ is H or unsubstituted —C₁-C₄ alkyl;
L is a linker; and
R^{a} is a chelating moiety or a radionuclide complex thereof.

8. The compound of claim 7, or a pharmaceutically acceptable salt thereof, wherein R¹ is H; Z is absent, —O—, —S—, or —N(R⁴)—; and R⁴ is H or —C₁-C₄ alkyl.

9. The compound of claim 7, or a pharmaceutically acceptable salt thereof, wherein R¹ is —CH₃; Z is absent, —O—, —S—, or —N(R⁴)—; and R⁴ is H or —C₁-C₄ alkyl.

10. The compound of any one of claims 7-9, or a pharmaceutically acceptable salt thereof, wherein R² is —CH₃.

11. The compound of any one of claims 7-10, or a pharmaceutically acceptable salt thereof, wherein R³ is H.

12. The compound of any one of claims 7-10, or a pharmaceutically acceptable salt thereof, wherein R³ is —CH₃.

13. The compound of any one of claims 7-12, or a pharmaceutically acceptable salt thereof, wherein R⁴ is H.

14. The compound of any one of claims 1-6, or a pharmaceutically acceptable salt thereof, wherein the compound has the structure of Formula (IIIa) or Formula (IIIb), or a pharmaceutically acceptable salt thereof: 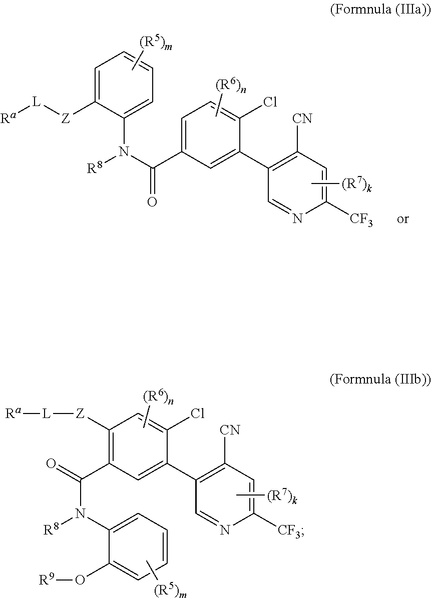
wherein:
each R⁵ is independently selected from F, Cl, Br, I, —CN, substituted or unsubstituted —C₁-C₆ alkyl, or substituted or unsubstituted —C₁-C₆ alkoxy;
each R⁶ is independently selected from F, Cl, Br, I, —CN, substituted or unsubstituted —C₁-C₆ alkyl, or substituted or unsubstituted —C₁-C₆ alkoxy;
each R⁷ is independently selected from F, Cl, Br, I, —CN, substituted or unsubstituted —C₁-C₆ alkyl, or substituted or unsubstituted —C₁-C₆ alkoxy;
R⁸ is H or substituted or unsubstituted —C₁-C₄ alkyl;
R⁹ is H or substituted or unsubstituted —C₁-C₄ alkyl;
Z is absent, —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—;
R⁴ is H or unsubstituted —C₁-C₄ alkyl;
L is a linker;
R^{a} is a chelating moiety or a radionuclide complex thereof;
m is 0, 1, 2, 3, or 4;
n is 0, 1, 2, or 3; and
k is 0, 1, or 2.

15. The compound of claim 14, or a pharmaceutically acceptable salt thereof, wherein R⁸ is —CH₃.

16. The compound of claim 14 or 15, or a pharmaceutically acceptable salt thereof, wherein R⁹ is —CH₃.

17. The compound of any one of claims 1-6, or a pharmaceutically acceptable salt thereof, wherein the compound has the structure of Formula IIIc, or a pharmaceutically acceptable salt thereof: 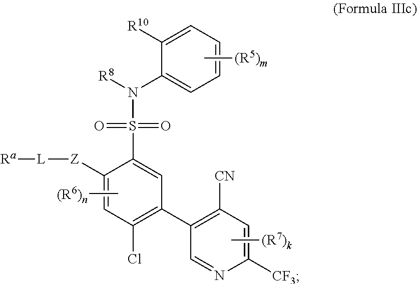
wherein:
each R⁵ is independently selected from F, Cl, Br, I, —CN, substituted or unsubstituted —C₁-C₆ alkyl, or substituted or unsubstituted —C₁-C₆ alkoxy;
each R⁶ is independently selected from F, Cl, Br, I, —CN, substituted or unsubstituted —C₁-C₆ alkyl, or substituted or unsubstituted —C₁-C₆ alkoxy;
each R⁷ is independently selected from F, Cl, Br, I, —CN, substituted or unsubstituted —C₁-C₆ alkyl, or substituted or unsubstituted —C₁-C₆ alkoxy;
R⁸ is H or —C₁-C₄ alkyl;
R¹⁰ is H, —OH, —C₁-C₄ alkyl, or —O—C₁-C₄ alkyl; or
R⁸ and R¹⁰ are taken together with the intervening atoms connecting R⁸ to R¹⁰ to form a substituted or unsubstituted 4- to 7-membered heterocyclic ring;
Z is absent, —C₁-C₆ alkylene, —C₁-C₆ alkylene-O—, —O—C₁-C₆ alkylene-, —C(═O)NR⁴—, —NR⁴C(═O)—, —O—, —NR⁴—, —S—, —S(═O)—, —SO₂—, or —NHC(═O)NH—;
R⁴ is H or unsubstituted —C₁-C₄ alkyl;
L is a linker;
R^{a} is a chelating moiety or a radionuclide complex thereof;
m is 0, 1, 2, 3, or 4;
n is 0, 1, 2, or 3; and
k is 0, 1, or 2.

18. The compound of claim 17, or a pharmaceutically acceptable salt thereof, wherein R⁸ is CH₃.

19. The compound of claim 17 or 18, or a pharmaceutically acceptable salt thereof, wherein R¹⁰ is —OCH₃.

20. The compound of claim 17, or a pharmaceutically acceptable salt thereof, wherein R⁸ and R¹⁰ are taken together with the intervening atoms connecting R⁸ to R¹⁰ to form a substituted or unsubstituted 5-membered heterocyclic ring, substituted or unsubstituted 6-membered ring, or substituted or unsubstituted 7-membered heterocyclic ring; wherein the heterocyclic ring is optionally substituted with 1 to 3 substituents independently selected from F, Cl, —C₁-C₄ alkyl, or —O—C₁-C₄ alkyl.

21. The compound of claim 17, or a pharmaceutically acceptable salt thereof, wherein the compound has the structure of Formula (IIId), or a pharmaceutically acceptable salt thereof:

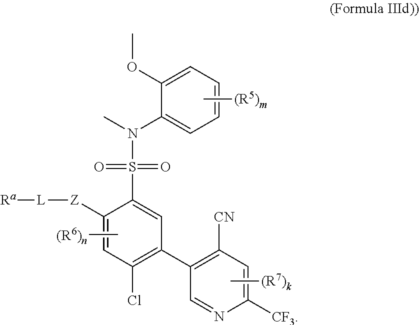

22. The compound of claim 17, or a pharmaceutically acceptable salt thereof, wherein the compound has the structure of Formula (IIIe), or a pharmaceutically acceptable salt thereof: 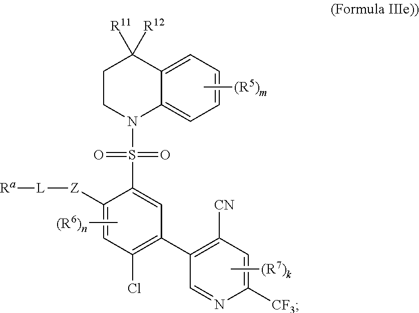
wherein R¹¹ and R¹² are each independently selected from H, F, or —C₁-C₄ alkyl; or
R¹¹ and R¹² can come together to form a substituted or unsubstituted —C₃-C₆ cycloalkyl ring.

23. The compound of claim 22, or a pharmaceutically acceptable salt thereof, wherein R¹¹ and R¹² are —CH₃.

24. The compound of any one of claims 14-23, or a pharmaceutically acceptable salt thereof, wherein m is 0.

25. The compound of any one of claims 14-24, or a pharmaceutically acceptable salt thereof, wherein n is 0.

26. The compound of any one of claims 14-25, or a pharmaceutically acceptable salt thereof, wherein k is 0.

27. The compound of any one of claims 7-26, or a pharmaceutically acceptable salt thereof, wherein Z is —O—.

28. The compound of any one of claims 7-26, or a pharmaceutically acceptable salt thereof, wherein Z is —NH—.

29. The compound of any one of claims 7-26, or a pharmaceutically acceptable salt thereof, wherein Z is —S—.

30. The compound of any one of claims 1-29, or a pharmaceutically acceptable salt thereof, wherein R^{a} is a chelating moiety independently selected from the group consisting of:
1,4,7,10-tetraazacyclododecane-1,4,7,10-tetraacetic acid (DOTA);
2,2′,2″-(10-(2-amino-2-oxoethyl)-1,4,7,10-tetraazacyclododecane-1,4,7-triyl)triacetic acid (PSC);
1,4,7,10-tetraazacyclododecane-1,4,7-triacetic acid (DO3A);
1,4,7,10-tetraazacyclododecane-1,7-diacetic acid (DO2A);
α,α′,α″,α′″-tetramethyl-1,4,7,10-tetraazacyclododecane-1,4,7,10-tetraacetic acid (DOTMA);
1,4,7,10-tetrakis(carbamoylmethyl)-1,4,7,10-tetraazacyclododecane (DOTAM);
1,4,7,10-tetraazacyclododecane-1,4,7,10-tetrapropionic acid (DOTPA);
2,2′,2″-(10-(2-amino-2-oxoethyl)-1,4,7,10-tetraazacyclododecane-1,4,7-triyl)triacetic acid;
benzyl-1,4,7,10-tetraazacyclododecane-1,4,7,10-tetraacetic acid (Bn-DOTA);
p-hydroxy-benzyl-1,4,7,10-tetraazacyclododecane-1,4,7,10-tetraacetic acid (p-OH-Bn-DOTA);
6,6′-(((pyridine-2,6-diylbis(methylene))bis((carboxymethyl)azanediyl))bis(methylene))dipicolinic acid (H₄pypa);
H₄pypa-benzyl;
6,6′,6″,6′″-(((pyridine-2,6-diylbis(methylene))bis(azanetriyl))tetrakis(methylene))-tetrapicolinic acid (H₄py4pa);
H₄py4pa-benzyl;
2,2′,2″-(1,4,7-triazacyclononane-1,4,7-triyl)triacetic acid (NOTA);
6,6′-((1,4,10,13-tetraoxa-7,16-diazacyclooctadecane-7,16-diyl)bis(methylene))dipicolinic acid (macropa);
2,2′,2″,2′″-(1,10-dioxa-4,7,13,16-tetraazacyclooctadecane-4,7,13,16-tetrayl)tetraacetic acid (crown);
6,6′-((ethane-1,2-diylbis((carboxymethyl)azanediyl))bis(methylene))dipicolinic acid (H₄octapa);
H₄octapa-benzyl; and
3,6,9,12-tetrakis(carboxymethyl)-3,6,9,12-tetraazatetradecanedioic acid (TTHA);
or a radionuclide complex thereof.

31. The compound of any one of claims 1-29, or a pharmaceutically acceptable salt thereof, wherein R^{a} is a chelating moiety selected from the group consisting of:
1,4,7,10-tetraazacyclododecane-1,4,7,10-tetraacetic acid (DOTA) and
1,4,7,10-tetraazacyclododecane-1,4,7-triacetic acid (DO3A); or a radionuclide complex thereof.

32. The compound of any one of claims 1-29, or a pharmaceutically acceptable salt thereof, wherein R^{a} is a chelating moiety selected from the group consisting of: 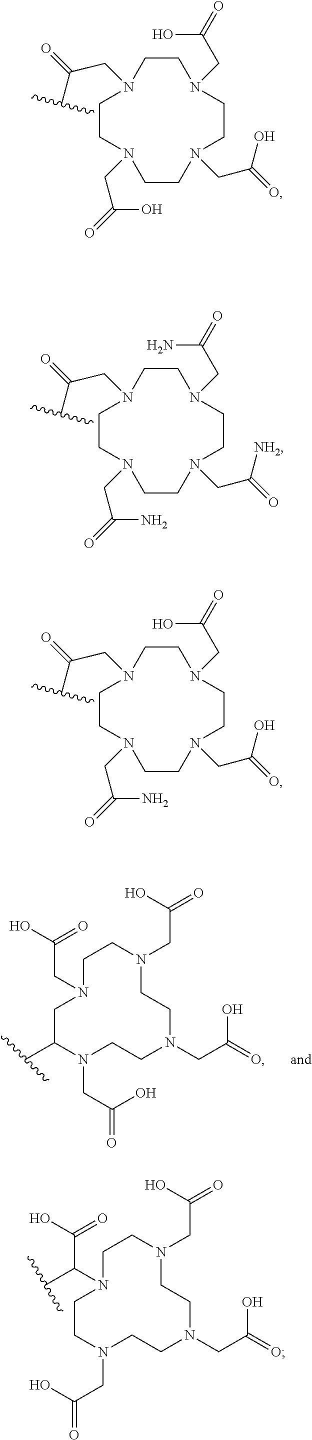 [SMILES: *C(=O)CN1CCN(CC(=O)O)CCN(CC(=O)O)CCN(CC(=O)O)CC1 ; *C(=O)CN1CCN(CC(N)=O)CCN(CC(N)=O)CCN(CC(N)=O)CC1 ; *C(=O)CN1CCN(CC(N)=O)CCN(CC(=O)O)CCN(CC(=O)O)CC1 ; *C1CN(CC(=O)O)CCN(CC(=O)O)CCN(CC(=O)O)CCN1CC(=O)O ; "and" ; *C(C(=O)O)N1CCN(CC(=O)O)CCN(CC(=O)O)CCN(CC(=O)O)CC1]
or a radionuclide complex thereof.

33. The compound of any one of claims 1-29, or a pharmaceutically acceptable salt thereof, wherein R^{a} is 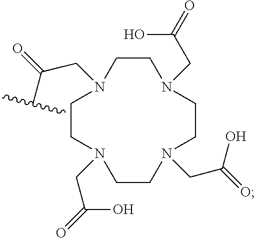 [SMILES: *C(=O)CN1CCN(CC(=O)O)CCN(CC(=O)O)CCN(CC(=O)O)CC1]
or a radionuclide complex thereof.

34. The compound of any one of claims 1-29, or a pharmaceutically acceptable salt thereof, wherein R^{a} is: 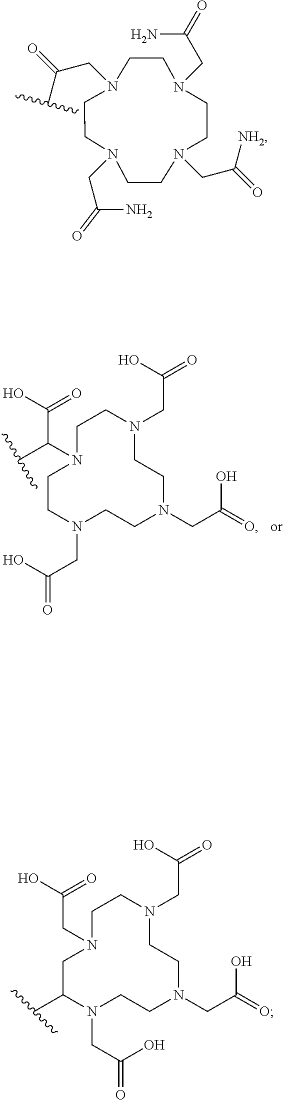 [SMILES: *C(=O)CN1CCN(CC(N)=O)CCN(CC(N)=O)CCN(CC(N)=O)CC1 ; *C(C(=O)O)N1CCN(CC(=O)O)CCN(CC(=O)O)CCN(CC(=O)O)CC1 ; *C1CN(CC(=O)O)CCN(CC(=O)O)CCN(CC(=O)O)CCN1CC(=O)O]
or a radionuclide complex thereof.

35. The compound of any one of claims 7-34, or a pharmaceutically acceptable salt thereof, wherein:
each L is independently selected from: -L²-, -L³-, -L⁴-, -L⁵-, -L⁶-, -L⁷-, -L²-L³-, -L²-L³-R¹⁹, -L²-L⁴-, -L²-L⁷-, -L⁴-L⁶-, -L⁴-L⁷-, -L⁶-L⁷-, -L²-L⁴-L⁷-, -L²-L⁵-L⁷-, -L²-L⁶-L⁷-, - L³-L⁴-L⁷-, -L⁴-L⁵-L⁷-, or -L2-L3-L4-L5-L6-L7-;
L² is absent, substituted or unsubstituted —C₁-C₂₀ alkylene, substituted or unsubstituted —C₁-C₂₀ alkylene-NR¹³—, substituted or unsubstituted —C₁-C₂₀ alkylene-C(═O)—, substituted or unsubstituted —C₁-C₂₀ alkylene-C(═O)NR¹³—, substituted or unsubstituted —C₁-C₂₀ alkylene-NR¹³C(═O)—, substituted or unsubstituted —C₁-C₂₀ alkylene-C(═O)NR¹³CH₂NR¹³—, substituted or unsubstituted —C₁-C₂₀ alkylene-NR¹³C(═O)CH₂NR¹³—, substituted or unsubstituted 2 to 20 membered heteroalkylene, —(CH₂CH₂O)_{z}—, —(OCH₂CH₂)_{z}—, —(CH₂CH₂O)_{w}—CH₂CH₂—, —CH₂CH₂NR¹³—(CH₂CH₂O)_{w}—, —(CH₂CH₂O)_{w}—CH₂CH₂NR¹³—, —CH₂CH₂NHC(═O)—(CH₂CH₂O)_{w}, —(CH₂CH₂O)_{w}—CH₂CH₂NR¹³C(═O)—, —CH₂CH₂C(═O)NR¹³—(CH₂CH₂O)_{w}—, —CH₂CH₂NR¹³C(═O)CH₂—(OCH₂CH₂)_{w} or —(CH₂CH₂O)_{w}—CH₂CH₂C(═O)NR¹³—;
R¹³ is H or unsubstituted —C₁-C₄ alkyl;
w is 1, 2, 3, 4, 5, 6, 7 or 8;
z is 1, 2, 3, 4, 5, 6, 7, 8, 9, or 10;
L³ is absent, a natural or unnatural amino acid or peptide that is formed from two or more independently selected natural and unnatural amino acids, wherein when two or more amino acids are present then the N atom of the amide linking the amino acids is optionally substituted with —C₁-C₆ alkyl;
L⁴ is absent, substituted or unsubstituted 2 to 10 membered heteroalkylene, —CH₂—(OCH₂CH₂)ᵥ—, —(CH₂CH₂O)ᵥ—CH₂CH₂—, —(CH₂CH₂O)ᵥCH₂CH₂NR¹⁴C(═O)(CH₂CH₂O)ᵥCH₂CH₂—, —(CH₂CH₂O)ᵥCH₂CH₂C(═O)NR¹⁴(CH₂CH₂O)ᵥCH₂CH₂—, —C(═O)CH₂CH₂, —CH₂CH₂C(═O)—, —CH₂CH₂NR¹⁴CH₂CH₂, —CH₂CH₂NHC(═O)—CH—CH₂CH₂C(═O)NHR¹⁴, or —C₁-C₆ alkylene that is optionally substituted with 1 or 2 groups independently selected from —OR¹⁵, —NR¹⁵₂, —CO₂R¹⁵, —O(CH₂CH₂O)ₛ—CH₃, —NR^{1′}(CH₂CH₂O)ₛ—CH₃, —NR¹⁵C(═O)(CH₂CH₂O)ₛ—CH₃, or —CH₂OCH₂CH₂CO₂R¹⁵;
R¹⁴ is H, —C₁-C₆ alkyl, or a sugar alcohol or derivative thereof;
each R¹⁵ is independently selected from H or unsubstituted —C₁-C₄ alkyl;
v is 1, 2, 3, 4, 5, 6, 7 or 8;
s is 1, 2, 3, 4, 5, or 6;
L⁵ is absent, —O—, —S—, —S(═O)—, —S(═O)₂, —NR¹⁶—, —CH(═NH)—, —CH(═N—NH)—, —CCH₃(═NH)—, —CCH₃(═N—NH)—, —C(═O)NR¹⁶—, —NR¹⁶C(═O), —NR¹⁶C(═O)O—, —NR¹⁶C(═O)NR¹⁶—, —OC(═O)NR¹⁶, or —NHC(═O)—C₁-C₆ alkylene that is optionally substituted with 1 or 2 groups independently selected from —OR¹⁵, —NR¹⁵₂ or —CO₂R¹⁵;
each R¹⁶ is independently selected from H or unsubstituted —C₁-C₄ alkyl;
L⁶ is absent or -L⁸-L⁹-L¹⁰-;
L⁸ is absent, —(CH₂)ᵣ—, —NR¹⁷—, —NR¹⁷—(CH₂)ᵣ—, —(CH₂)ᵣ—C(═O)—, —C(═O)—(CH₂)ᵣ—, —(CH₂)ᵣ—NR¹⁷—, —(CH₂)ᵣ—NR¹⁷C(═O)—, —(CH₂)ᵣ—C(═O)NR¹⁷—, —CH(NHR¹⁷)—(CH₂)ᵣ—C(═O)—, —NR¹⁷C(═O)—(CH₂)ᵣ—, and —C(═O)NR¹⁷—(CH₂)ᵣ—;
r is 0, 1, 2, or 3;
L⁹ is substituted or unsubstituted cycloalkylene, substituted or unsubstituted heterocycloalkylene, substituted or unsubstituted arylene, or substituted or unsubstituted heteroarylene;
L¹⁰ is absent, —(CH₂)_{q}—, —NR¹⁸—, —NR¹⁸—(CH₂)_{q}—, —(CH₂)_{q}—C(═O)—, —C(═O)—(CH₂)_{q}—, —(CH₂)_{q}—NR¹⁸—, —(CH₂)_{q}—NR¹⁸C(═O)—, —(CH₂)_{q}—C(═O)NR¹⁸—, —CH(NHR¹⁸)—(CH₂)_{q}—C(═O)—, —NR¹⁸C(═O)—(CH₂)_{q}—, —C(═O)NR¹⁸—(CH₂)_{q}—, and —NR¹⁸—(CH₂)_{q}—NR¹⁸;
q is 0, 1, 2, or 3;
R¹⁷ and R¹⁸ are each independently selected from H, —C₁-C₆ alkyl, —C₁-C₆ alkyl-CO₂H, —(CH₂CH₂O)ₚ—CH₃, —C(═O)—(CH₂CH₂O)ₚ—CH₃, or —(CH₂CH₂O)ₚ—CH₂CH₂CO₂H;
p is 1, 2, 3, 4, 5, or 6;
L⁷ is absent, —NH—, —N(CH₃)—, —O—NH—, or substituted or unsubstituted N-heterocycloalkylene, or —O—NH=(substituted or unsubstituted N-heterocycloalkylene);
R¹⁹ is —C(═O)(CH₂)ᵢ-substituted or unsubstituted phenyl; and
i is 1, 2, 3, 4, 5, or 6.

36. The compound of any one of claims 7-34, or a pharmaceutically acceptable salt thereof, wherein:
each L is independently selected from: -L²-, -L³-, -L⁴-, -L⁵-, -L⁶-, -L⁷-, -L²-L³-, -L²-L³-R¹⁹, -L²-L⁴-, -L²-L⁷-, -L⁴-L⁶-, -L⁴-L⁷-, -L⁶-L⁷-, -L²-L⁴-L⁷-, -L²-L⁵-L⁷-, -L²-L⁶-L⁷-, - L³-L⁴-L⁷-, -L⁴-L⁵-L⁷-, or -L²-L³-L⁴-L⁵-L⁶-L⁷-;
L² is substituted or unsubstituted —C₁-C₂₀ alkylene-C(═O)NR¹³—, substituted or unsubstituted —C₁-C₂₀ alkylene-NR¹³C(═O)—, or —(CH₂CH₂O)_{w}—CH₂CH₂—;
R¹³ is H or unsubstituted —C₁-C₄ alkyl;
w is 1, 2, 3, 4, 5, 6, 7 or 8;
L³ is absent or a natural or unnatural amino acid or peptide that is formed from two or more independently selected natural and unnatural amino acids;
L⁴ is absent, —(CH₂CH₂O)ᵥ—CH₂CH₂—, —CH₂CH₂NR¹⁴CH₂CH₂, or —C₁-C₆ alkylene that is optionally substituted with 1 or 2 groups independently selected from —OR¹⁵, —NR¹², or —CO₂R¹⁵;
R¹⁴ is H or —C₁-C₆ alkyl;
each R¹⁵ is independently selected from H or unsubstituted —C₁-C₄ alkyl;
v is 1, 2, 3,4, 5, 6, 7 or 8;
L⁵ is absent;
L⁶ is absent;
L⁷ is —NH—;
R¹⁹ is —C(═O)(CH₂)ᵢ-substituted or unsubstituted phenyl; and
i is 1, 2, 3, 4, 5, or 6.

37. The compound of claim 35 or 36, or a pharmaceutically acceptable salt thereof, wherein L is -L²-L⁷-.

38. The compound of claim 35 or 36, or a pharmaceutically acceptable salt thereof, wherein L is -L²-L⁴-L⁷-.

39. The compound of any one of claims 35-38, or a pharmaceutically acceptable salt thereof, wherein L² is substituted or unsubstituted —C₁-C₆alkylene-NHC(═O)— or substituted or unsubstituted —C₁-C₆alkylene-C(═O)NH—.

40. The compound of any one of claims 35-38, or a pharmaceutically acceptable salt thereof, wherein L² is —(CH₂CH₂O)_{w}—CH₂CH₂—.

41. The compound of any one of claims 35-38 or 40, or a pharmaceutically acceptable salt thereof, wherein w is 1, 2, 3, 4 or 5.

42. The compound of any one of claims 35-38 or 40, or a pharmaceutically acceptable salt thereof, wherein w is 8.

43. The compound of claim 35 or 36, or a pharmaceutically acceptable salt thereof, wherein L³ is absent.

44. The compound of claim 35 or 36, or a pharmaceutically acceptable salt thereof, wherein L⁴ is absent.

45. The compound of claim 35, 36 or 38-42, or a pharmaceutically acceptable salt thereof, wherein L⁴ is —(CH₂CH₂O)ᵥ—CH₂CH₂—.

46. The compound of any one of claims 35, 36, 38-43, or 45, or a pharmaceutically acceptable salt thereof, wherein v is 4.

47. The compound of any one of claims 35, 36, 38-43, or 45, or a pharmaceutically acceptable salt thereof, wherein v is 8.

48. The compound of any one of claims 35, 36, or 38-43, or a pharmaceutically acceptable salt thereof, wherein L⁴ is —CH₂CH₂NR¹⁴CH₂CH₂.

49. The compound of any one of claims 35, 36, or 38-43, or a pharmaceutically acceptable salt thereof, wherein L⁴ is —C₁-C₆ alkylene.

50. The compound of any one of claims 35, 36, or 38-43, or a pharmaceutically acceptable salt thereof, wherein L⁴ is —C₁-C₆ alkylene substituted with 1 —OR¹⁵, —NR¹⁵₂ or —CO₂R¹⁵.

51. The compound of claim 50, wherein R¹⁵ is H.

52. The compound of claim 50, wherein each R¹⁵ is independently selected from H or —CH₃.

53. The compound of claim 35, 36, 40-52, a pharmaceutically acceptable salt thereof, wherein L⁵ is absent.

54. The compound of claim 35, 36, 40-53, or a pharmaceutically acceptable salt thereof, wherein L⁶ is absent.

55. The compound of claim 35, 36, 40-54, or a pharmaceutically acceptable salt thereof, wherein L⁷ is —NH—.

56. The compound of claim 35 or 36, or a pharmaceutically acceptable salt thereof, wherein
L¹ is -L²-L⁷-;
L² is —(CH₂CH₂O)_{w}—CH₂CH₂—; and
L⁷ is —NH—.

57. The compound of claim 35 or 36, or a pharmaceutically acceptable salt thereof, wherein
L¹ is L²-L⁴-L⁷;
L² is substituted or unsubstituted —C₁-C₂₀ alkylene-C(═O)NR¹³— or substituted or unsubstituted —C₁-C₂₀ alkylene-NR¹³C(═O)—;
L⁴ is —(CH₂CH₂O)ᵥ—CH₂CH₂—, —CH₂CH₂NR¹⁴CH₂CH₂—, or optionally substituted —C₁-C₆ alkylene; and
L⁷ is —NH—.

58. The compound of any one of claims 1-34, or a pharmaceutically acceptable salt thereof, wherein -L-R^{a} is:

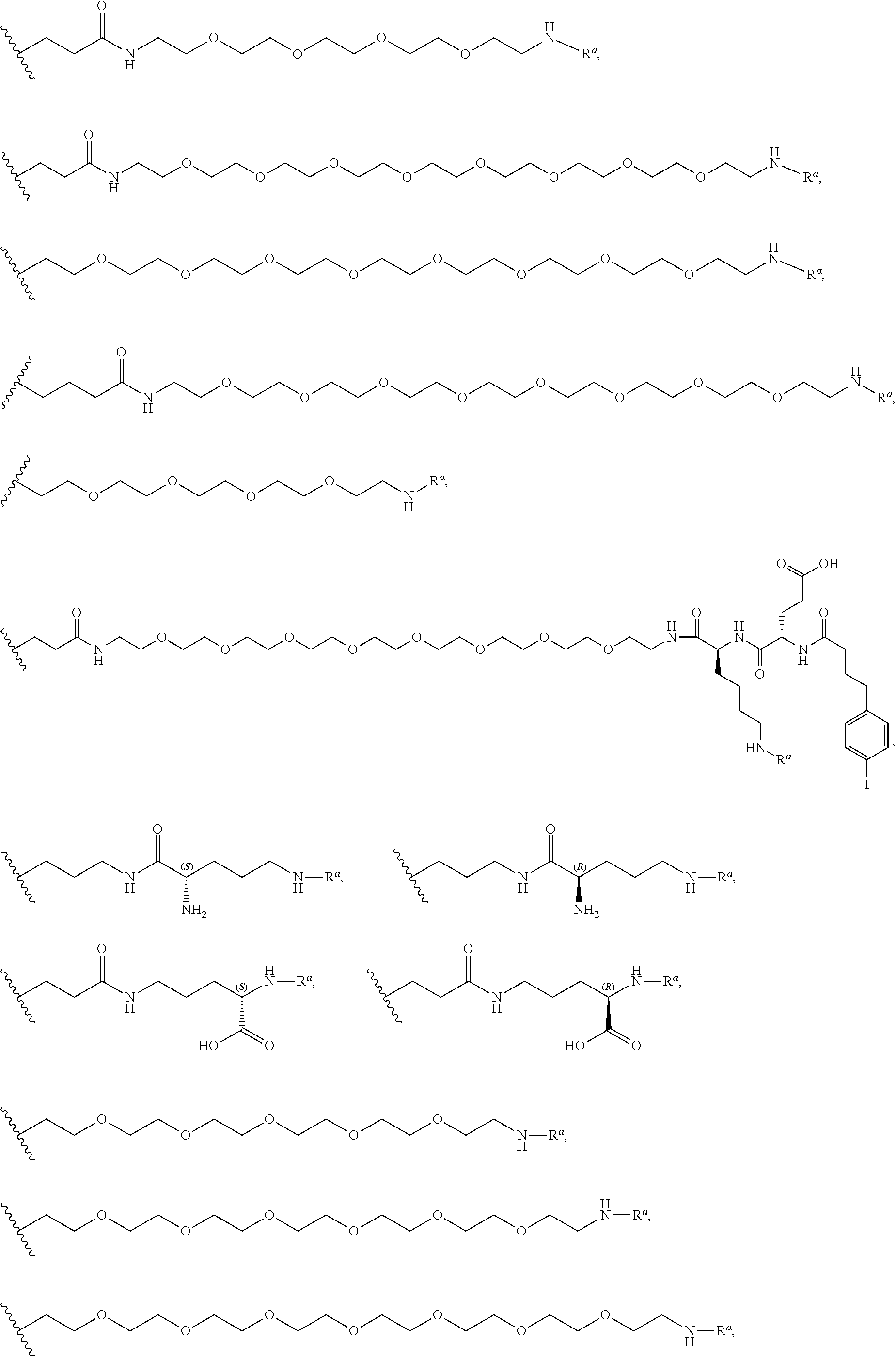

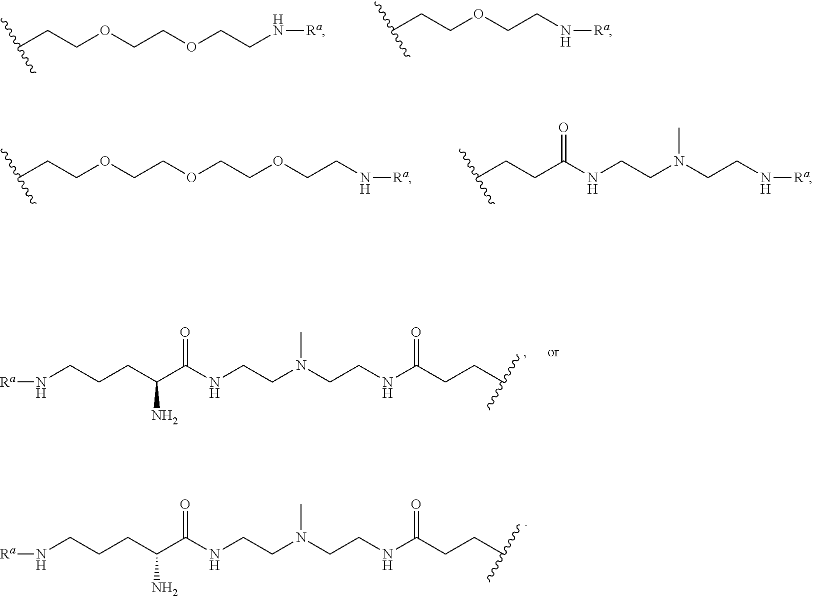

59. The compound of any one of claims 1-34, or a pharmaceutically acceptable salt thereof, wherein -L-R^{a} is:

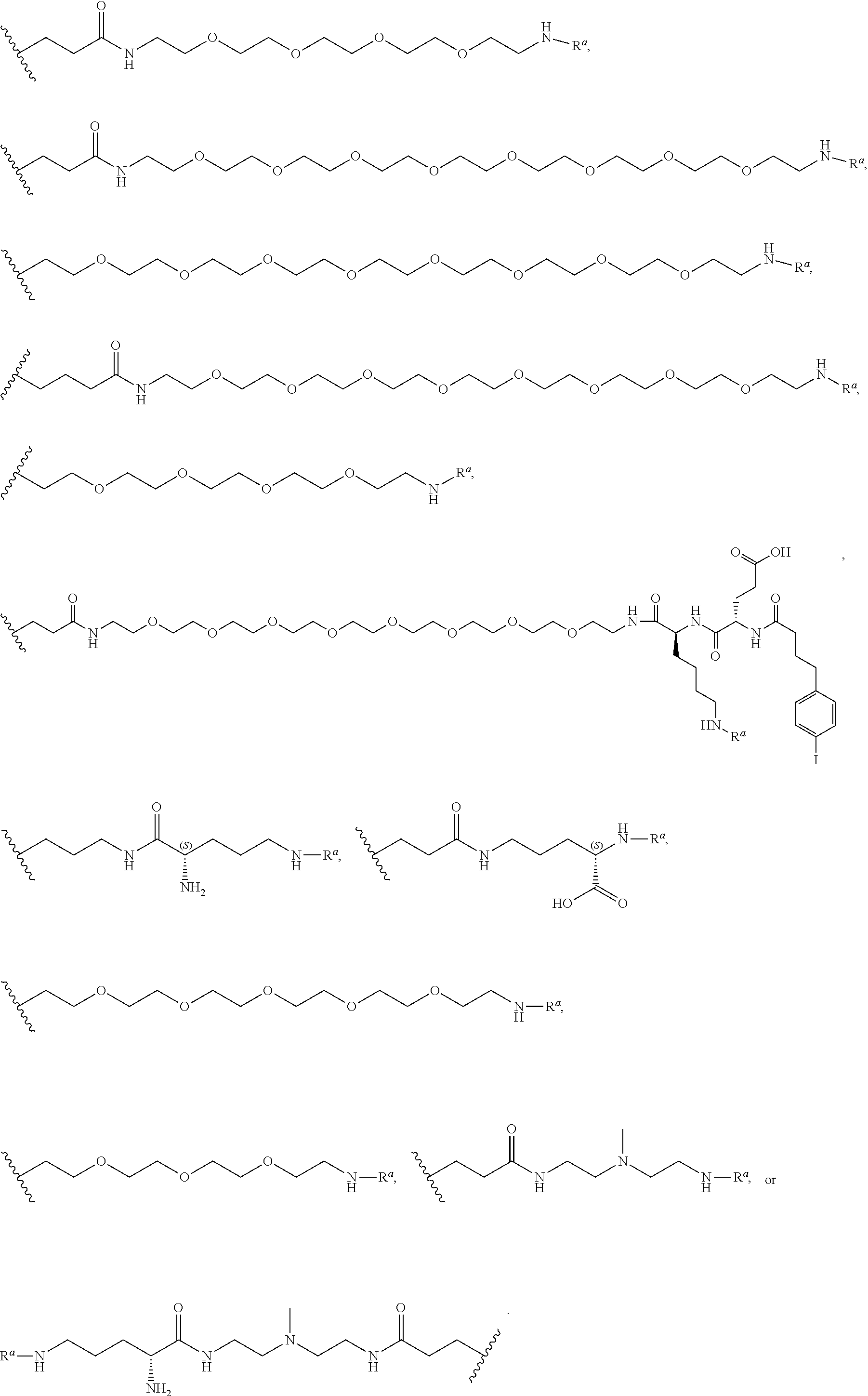

60. The compound of any one of claims 1-34, or a pharmaceutically acceptable salt thereof, wherein -L-R^{a} is: 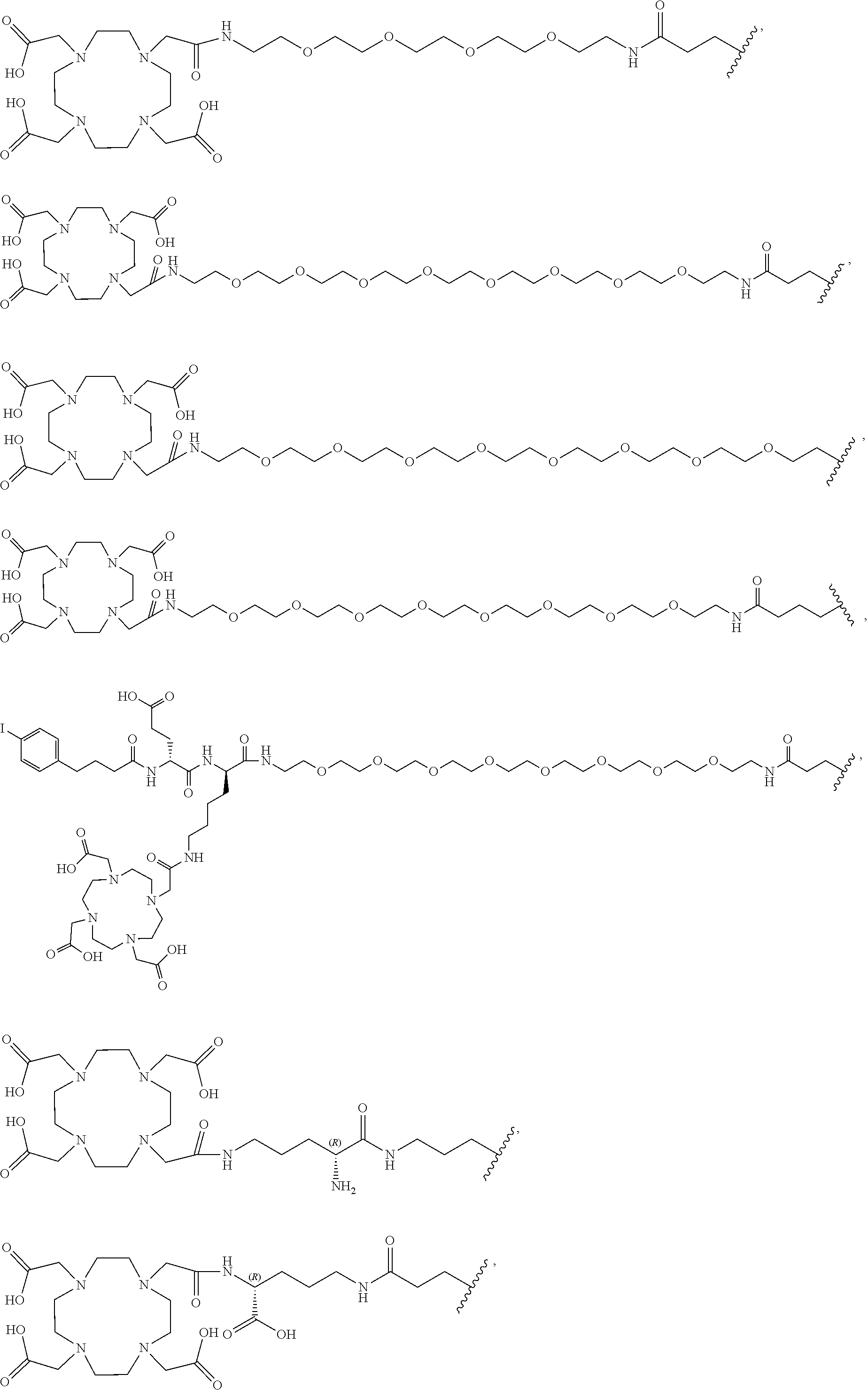 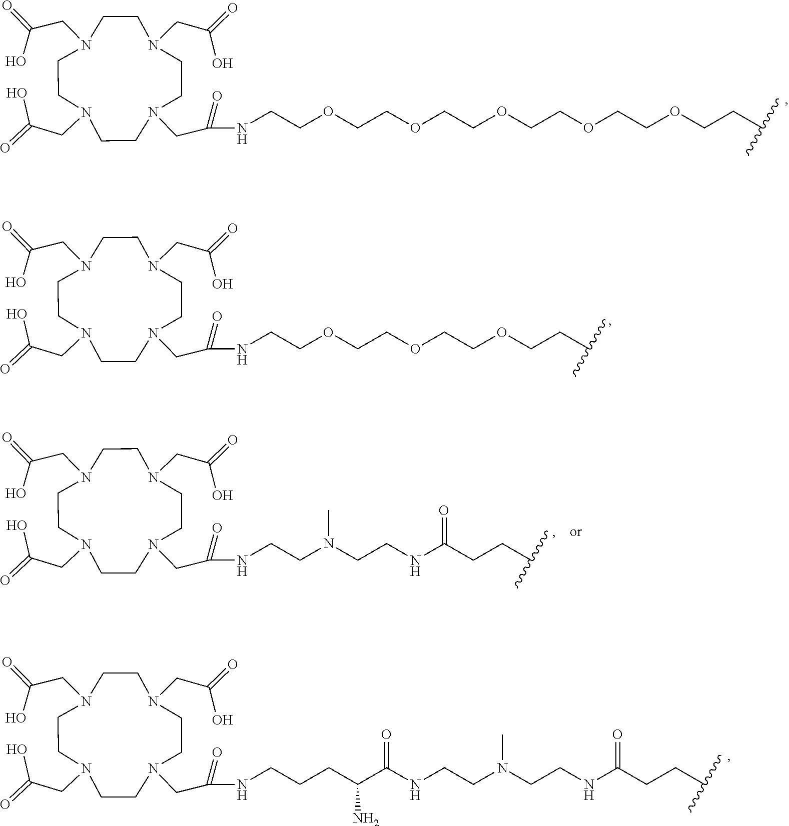 [SMILES: *CCOCCOCCOCCOCCOCCNC(=O)CN1CCN(CC(=O)O)CCN(CC(=O)O)CCN(CC(=O)O)CC1 ; *CCOCCOCCOCCNC(=O)CN1CCN(CC(=O)O)CCN(CC(=O)O)CCN(CC(=O)O)CC1 ; *CCC(=O)NCCN(C)CCNC(=O)CN1CCN(CC(=O)O)CCN(CC(=O)O)CCN(CC(=O)O)CC1 ; *CCC(=O)NCCN(C)CCNC(=O)[C@H](N)CCCNC(=O)CN1CCN(CC(=O)O)CCN(CC(=O)O)CCN(CC(=O)O)CC1]
or a radionuclide complex thereof.

61. The compound of claim 7, or a pharmaceutically acceptable salt thereof, wherein the compound of Formula (II) has one of the following structures, or a pharmaceutically acceptable salt thereof: 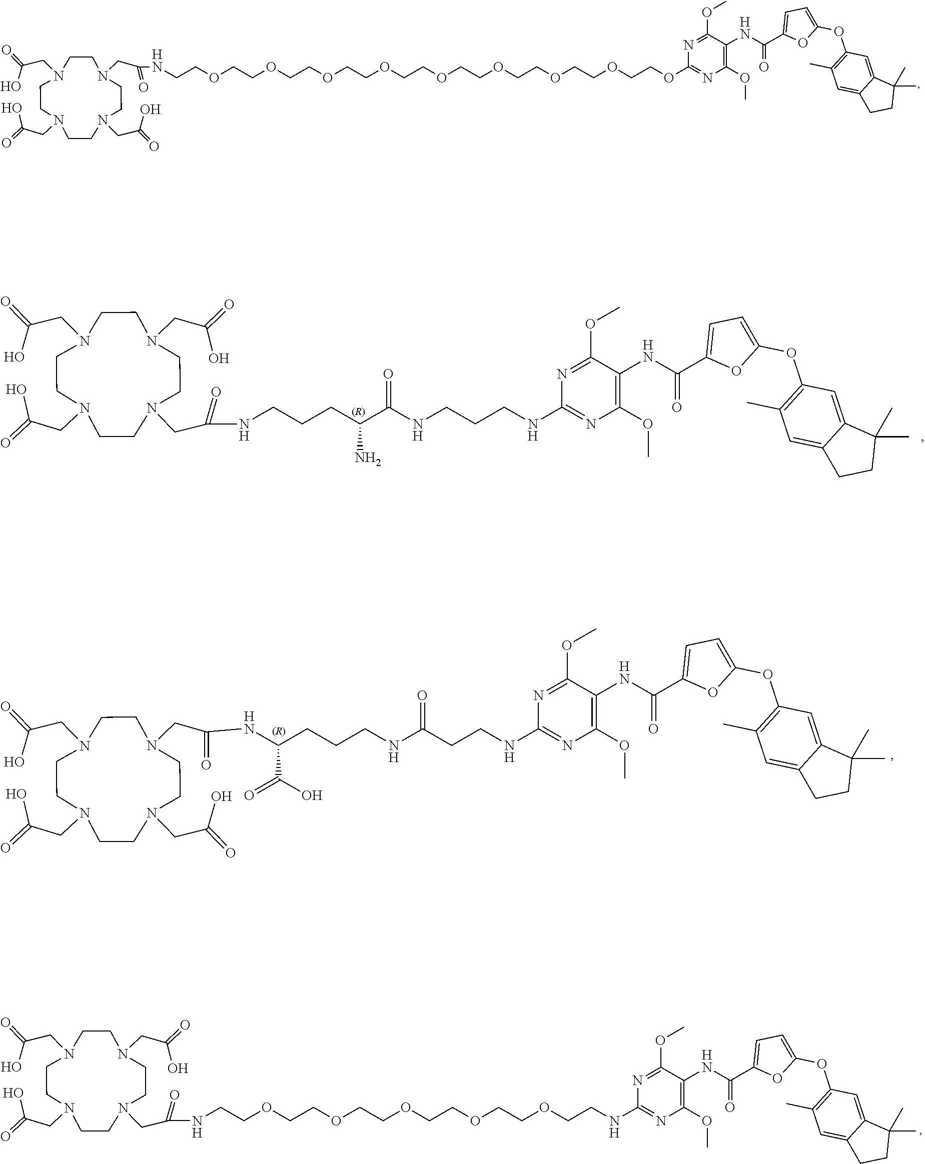 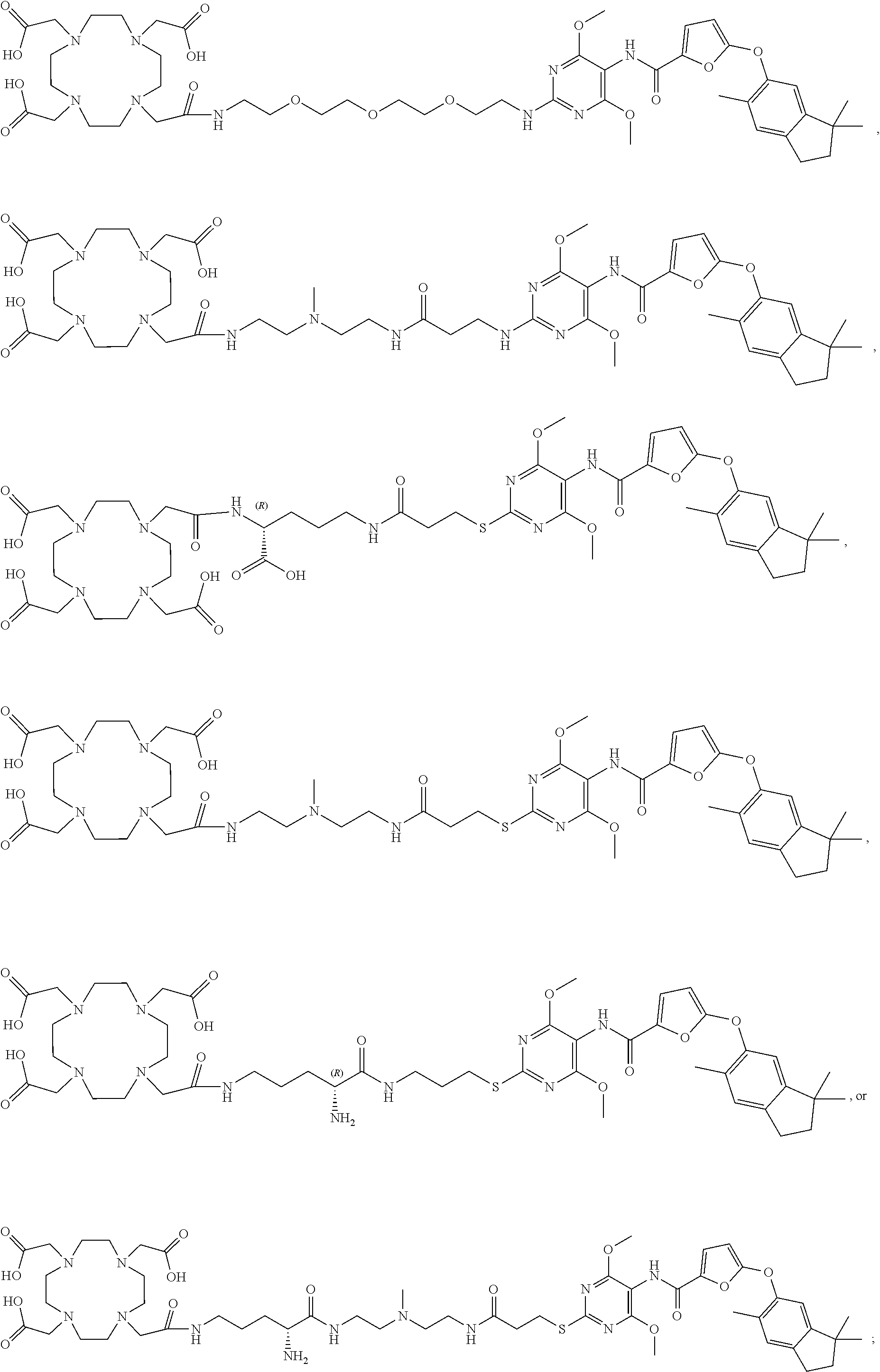
or a radionuclide complex thereof.

62. The compound of claim 14, or a pharmaceutically acceptable salt thereof, wherein the compound of Formula (IIIa) or (IIIb) has one of the following structures, or a pharmaceutically acceptable salt thereof: 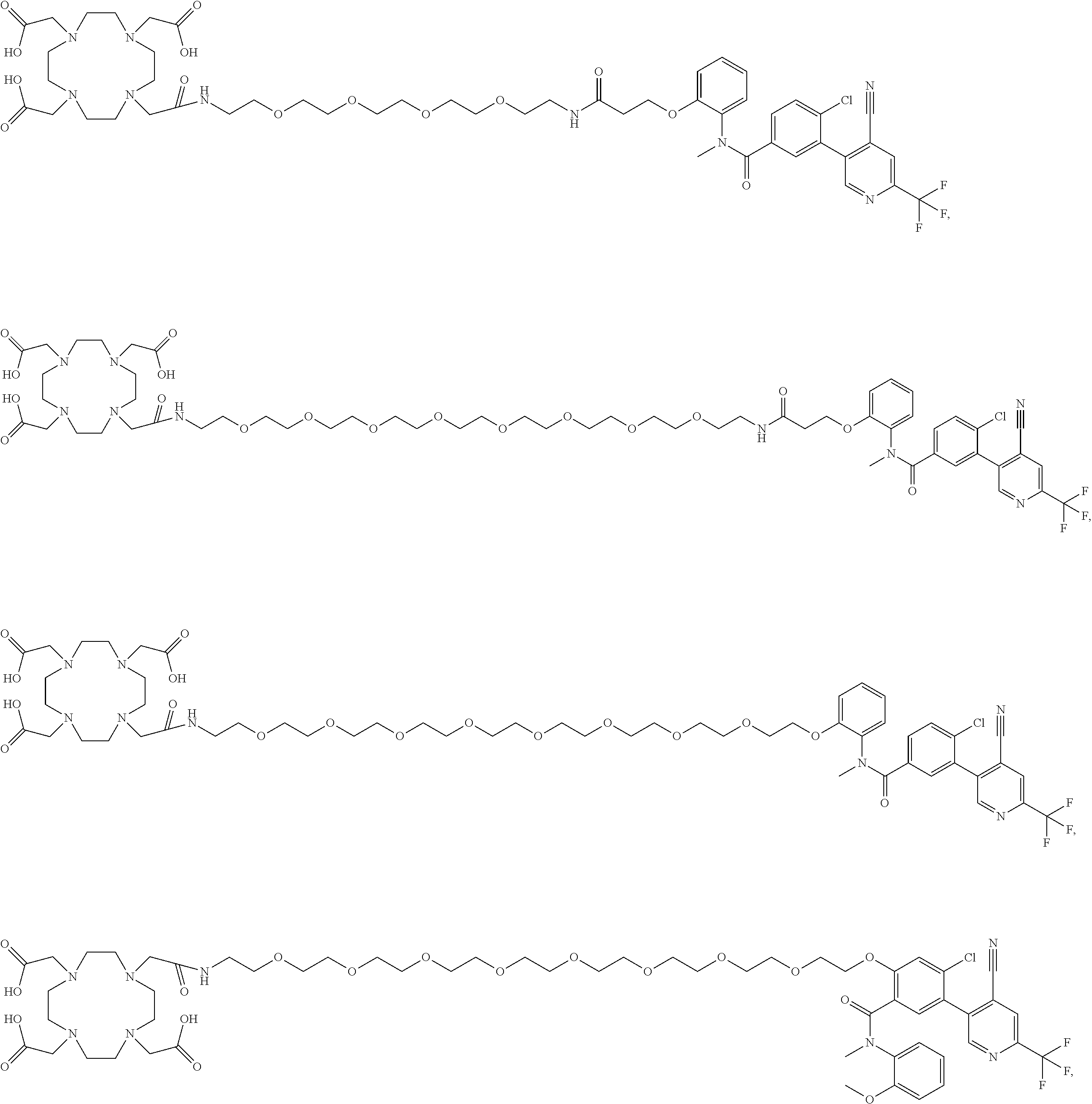 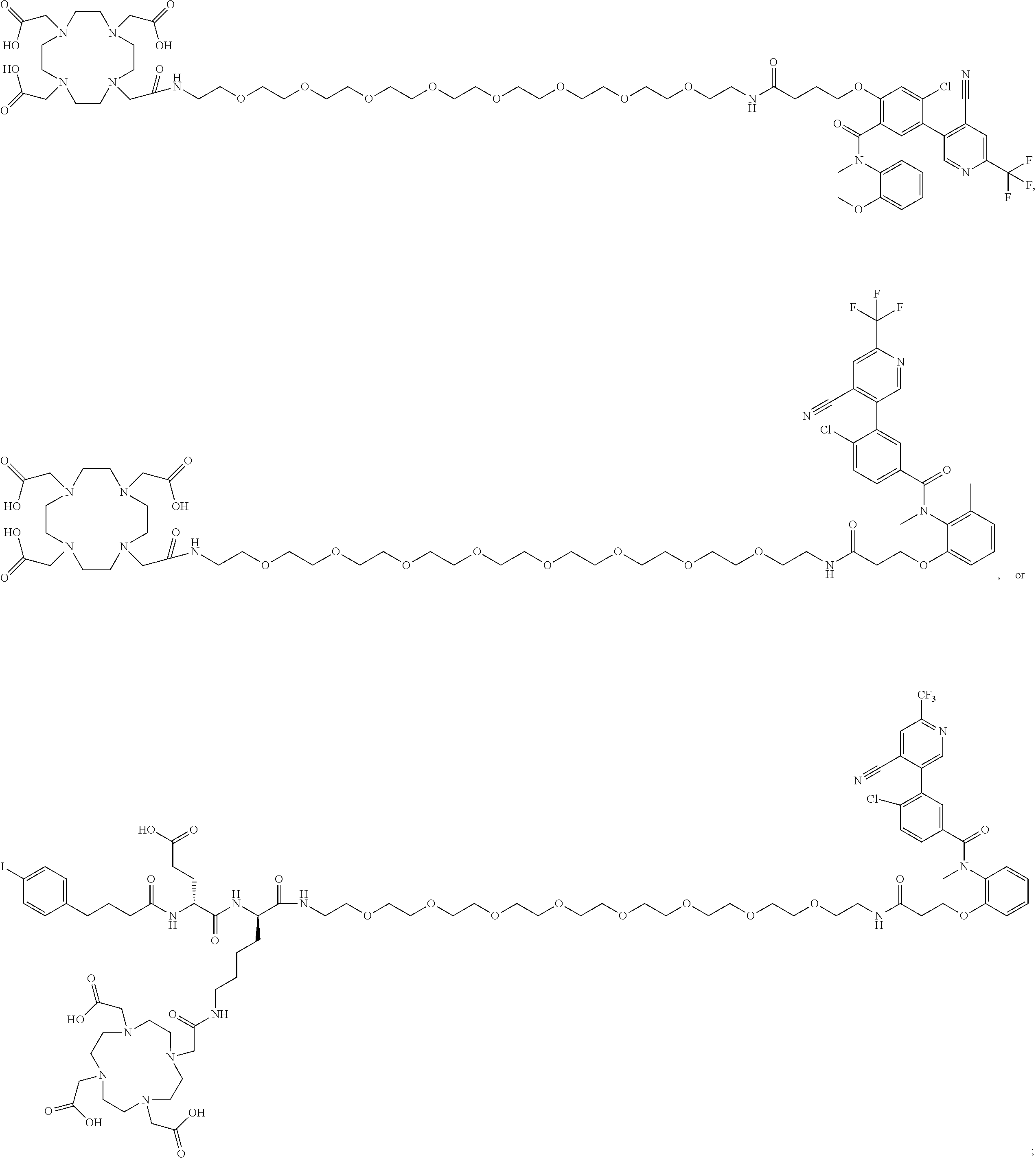 [SMILES: COc1ccccc1N(C)C(=O)c1cc(-c2cnc(C(F)(F)F)cc2C#N)c(Cl)cc1OCCCC(=O)NCCOCCOCCOCCOCCOCCOCCOCCOCCNC(=O)CN1CCN(CC(=O)O)CCN(CC(=O)O)CCN(CC(=O)O)CC1 ; Cc1cccc(OCCC(=O)NCCOCCOCCOCCOCCOCCOCCOCCOCCNC(=O)CN2CCN(CC(=O)O)CCN(CC(=O)O)CCN(CC(=O)O)CC2)c1N(C)C(=O)c1ccc(Cl)c(-c2cnc(C(F)(F)F)cc2C#N)c1 ; CN(C(=O)c1ccc(Cl)c(-c2cnc(C(F)(F)F)cc2C#N)c1)c1ccccc1OCCC(=O)NCCOCCOCCOCCOCCOCCOCCOCCOCCNC(=O)[C@@H](CCCCNC(=O)CN1CCN(CC(=O)O)CCN(CC(=O)O)CCN(CC(=O)O)CC1)NC(=O)[C@@H](CCC(=O)O)NC(=O)CCCc1ccc(I)cc1]
or a radionuclide complex thereof.

63. The compound of claim 17, or a pharmaceutically acceptable salt thereof, wherein the compound of Formula (IIIc) has one of the following structures, or a pharmaceutically acceptable salt thereof: 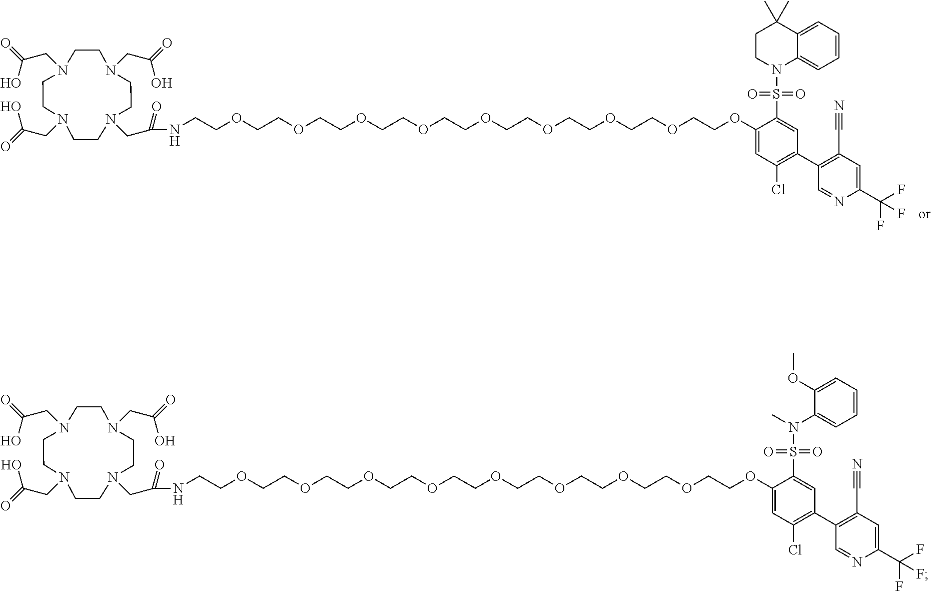 [SMILES: CC1(C)CCN(S(=O)(=O)c2cc(-c3cnc(C(F)(F)F)cc3C#N)c(Cl)cc2OCCOCCOCCOCCOCCOCCOCCOCCOCCNC(=O)CN2CCN(CC(=O)O)CCN(CC(=O)O)CCN(CC(=O)O)CC2)c2ccccc21 ; COc1ccccc1N(C)S(=O)(=O)c1cc(-c2cnc(C(F)(F)F)cc2C#N)c(Cl)cc1OCCOCCOCCOCCOCCOCCOCCOCCOCCNC(=O)CN1CCN(CC(=O)O)CCN(CC(=O)O)CCN(CC(=O)O)CC1]
or a radionuclide complex thereof.

64. The compound of any one of claims 1-63, or a pharmaceutically acceptable salt thereof, wherein: the radionuclide of the radionuclide complex is a lanthanide or an actinide.

65. The compound of any one of claims 1-63, or a pharmaceutically acceptable salt thereof, wherein: the radionuclide of the radionuclide complex is actinium, bismuth, cesium, cobalt, copper, dysprosium, erbium, gold, indium, iridium, gallium, lead, lutetium, manganese, palladium, platinum, radium, rhenium, samarium, strontium, technetium, ytterbium, yttrium, or zirconium.

66. The compound of any one of claims 1-63, or a pharmaceutically acceptable salt thereof, wherein: the radionuclide of the radionuclide complex is a diagnostic or therapeutic radionuclide.

67. The compound of any one of claims 1-63, or a pharmaceutically acceptable salt thereof, wherein: the radionuclide of the radionuclide complex is an Auger electron-emitting radionuclide, α-emitting radionuclide, β-emitting radionuclide, or γ-emitting radionuclide.

68. The compound of any one of claims 1-63, or a pharmaceutically acceptable salt thereof, wherein the radionuclide of the radionuclide complex is:
an Auger electron-emitting radionuclide that is 111-indium (¹¹¹In), 67-gallium (⁶⁷Ga), 68-gallium (⁶⁸Ga), 99m-technetium (^{99m}Tc), or 195m-platinum (^{195m}Pt); or
an α-emitting radionuclide that is 225-actinium (²²⁵Ac), 213-bismuth (²¹³Bi), 223-Radium (²²³Ra), or 212-lead (²¹²Pb); or
a β-emitting radionuclide that is 90-yttrium (⁹⁰Y), 177-lutetium (¹⁷⁷Lu), 186-rhenium (¹⁸⁶Re), 188-rhenium (¹⁸⁸Re), 64-copper (⁶⁴Cu), 67-copper (⁶⁷Cu), 153-samarium (¹⁵³Sm), 89-strontium (⁸⁹Sr), 198-gold (¹⁹⁸Au), 169-Erbium (¹⁶⁹Er), 165-dysprosium (¹⁶⁵Dy), 99m-technetium (^{99m}Tc), 89-zirconium (⁸⁹Zr), or 52-manganese (⁵²Mn); or
a γ-emitting radionuclide that is 60-cobalt (⁶⁰Co), 103-palladium (¹⁰³Pd), 137-cesium (¹³⁷Cs), 169-ytterbium (¹⁶⁹Yb) 192-iridium (¹⁹²Ir), or 226-radium (²²⁶Ra).

69. The compound of any one of claims 1-63, or a pharmaceutically acceptable salt thereof, wherein: the radionuclide of the radionuclide complex is 111-indium (¹¹¹In), 115-indium (¹¹⁵In), 67-gallium (⁶⁷Ga), 68-gallium (⁶⁸Ga), 69-gallium (⁶⁹Ga), 71-gallium (⁷¹Ga), 225-actinium (²²⁵Ac), 175-lutetium (¹⁷⁵Lu), 177-lutetium (¹⁷⁷Lu), 204-lead (²⁰⁴Pb), 206-lead (²⁰⁶Pb), 207-lead (²⁰⁷Pb), 208-lead (²⁰⁸Pb), 212-lead (²¹²Pb), 63-copper (⁶³Cu), 64-copper (⁶⁴Cu), 65-copper (⁶⁵Cu), or 67-copper (⁶⁷Cu).

70. The compound of any one of claims 1-63, or a pharmaceutically acceptable salt thereof, wherein: the radionuclide of the radionuclide complex is 111-indium (¹¹¹In), 115-indium (¹¹⁵In), 67-gallium (⁶⁷Ga), 68-gallium (⁶⁸Ga), 71-gallium (⁷¹Ga), 225-actinium (²²⁵Ac), 175-lutetium (¹⁷⁵Lu), or 177-lutetium (¹⁷⁷Lu).

71. A pharmaceutical composition comprising a compound of any one of claims 1-70, or a pharmaceutically acceptable salt thereof, and at least one pharmaceutically acceptable excipient.

72. The pharmaceutical composition of claim 71, wherein the pharmaceutical composition is formulated for administration to a mammal by intravenous administration.

73. A method for the treatment of cancer comprising administering to a mammal with cancer an effective amount of a compound of any one of claims 1-70, or a pharmaceutically acceptable salt thereof.

74. The method of claim 73, wherein the cancer comprises tumors and the tumor overexpress the gonadotropin-releasing hormone receptor (GnRHR).

75. The method of claim 73 or claim 74, wherein the cancer is ovarian cancer, breast cancer, endometrial cancer, or prostate cancer.

76. The method of claim 73 or claim 74, wherein the cancer is breast cancer.

77. The method of claim 73 or claim 74, wherein the cancer is endometrial cancer.

78. A method of killing tumors in a mammal that overexpress the gonadotropin-releasing hormone receptor (GnRHR) comprising administering to the mammal a compound of any one of claims 1-69, or a pharmaceutically acceptable salt thereof, wherein the compound of any one of claims 1-70, or a pharmaceutically acceptable salt thereof, comprises a therapeutic radionuclide.

79. The method of claim 78, wherein the mammal has been diagnosed with ovarian cancer, breast cancer, endometrial cancer, or prostate cancer.

80. The method of claim 78, wherein the mammal has been diagnosed with breast cancer.

81. The method of claim 78, wherein the mammal has been diagnosed with endometrial cancer.

82. A method for identifying tumors expressing the gonadotropin-releasing hormone receptor (GnRHR) in a mammal comprising administering to the mammal a compound of any one of claims 1-70, or a pharmaceutically acceptable salt thereof; and performing positron emission tomography (PET) analysis, single-photon emission computerized tomography (SPECT), or magnetic resonance imaging (MRI); wherein the compound of any one of claims 1-70, or a pharmaceutically acceptable salt thereof, comprises a diagnostic radionuclide.

83. A method for the in vivo imaging of tissues or organs in a mammal with tumors expressing the gonadotropin-releasing hormone receptor (GnRHR) comprising administering to the mammal a compound of any one of claims 1-70, or a pharmaceutically acceptable salt thereof; and performing positron emission tomography (PET) analysis, single-photon emission computerized tomography (SPECT), or magnetic resonance imaging (MRI); wherein the compound of any one of claims 1-70, or a pharmaceutically acceptable salt thereof, comprises a diagnostic radionuclide.
